(19) United States
(12) Patent Application Publication
(10) Pub. No.: US 2022/0105162 A1
(43) Pub. Date: Apr. 7, 2022

(54) T-CELL MODULATORY MULTIMERIC POLYPEPTIDES AND METHODS OF USE THEREOF

(71) Applicant: Cue Biopharma, Inc., Cambridge, MA (US)

(72) Inventors: Ronald D. Seidel, III, Cambridge, MA (US); Rodolfo J. Chaparro, Cambidge, MA (US)

(21) Appl. No.: 17/394,972

(22) Filed: Aug. 5, 2021

Related U.S. Application Data
(63) Continuation of application No. PCT/US2020/021138, filed on Mar. 5, 2020.
(60) Provisional application No. 62/814,721, filed on Mar. 6, 2019.

(51) Int. Cl. A61K 39/00; A61K 38/17

(52) U.S. Cl. CPC A61K 39/0008; A61K 2039/6056; A61K 2039/605; A61K 38/1774

(57) ABSTRACT

The present disclosure provides T-cell modulatory antigen-presenting polypeptides, including single-chain antigen-presenting polypeptides and multimeric antigen-presenting polypeptides. The present disclosure provides nucleic acids comprising nucleotide sequences encoding T-cell modulatory antigen-presenting polypeptides of the present disclosure, as well as cells genetically modified with the nucleic acids. A T-cell modulatory antigen-presenting polypeptide of the present disclosure is useful for modulating activity of a T cell. Thus, the present disclosure provides methods of modulating activity of a T cell.

### CROSS-REFERENCE

This application claims the benefit of U.S. Provisional Patent Application No. 62/814,721, filed Mar. 6, 2019, which application is incorporated herein by reference in its entirety.

### INCORPORATION BY REFERENCE OF SEQUENCE LISTING PROVIDED AS A TEXT FILE

A Sequence Listing is provided herewith as a text file, “CUEB-120WO_SeqList_RevDec2021_ST25” created on Dec. 14, 2021 and having a size of 717 KB. The contents of the text file are incorporated by reference herein in their entirety.

### INTRODUCTION

Central to the proper functioning of the mammalian immune system are the coordinated activities and communications between two specialized cell types, antigen-presenting cells (“APCs”) and T cells. APCs serve to capture and break the proteins from foreign organisms, or abnormal proteins (e.g., from genetic mutation in cancer cells), into smaller fragments suitable as signals for scrutiny by the larger immune system, including T cells. In particular, APCs break down proteins into small peptide fragments, which are then paired with proteins of the major histocompatibility complex (“MHC”) and displayed on the cell surface. Cell surface display of an MHC together with a peptide fragment, also known as a T cell epitope, provides the underlying scaffold surveilled by T cells, allowing for specific recognition. The peptide fragments can be pathogen-derived, tumor-derived, or derived from natural host proteins (self-proteins). Moreover, APCs can recognize other foreign components, such as bacterial toxins, viral proteins, viral DNA, viral RNA, etc., whose presence denotes an escalated threat level. The APCs relay this information to T cells through additional costimulatory signals in order to generate a more effective response.

T cells recognize peptide-major histocompatibility complex (“pMHC”) complexes through a specialized cell surface receptor, the T cell receptor (“TCR”). The TCR is unique to each T cell; as a consequence, each T cell is highly specific for a particular pMHC target. In order to adequately address the universe of potential threats, a very large number (˜40,000,000) of distinct T cells with distinct TCRs exist in the human body. Further, any given T cell, specific for a particular T cell peptide, is initially a very small fraction of the total T cell population. Although normally dormant and in limited numbers, T cells bearing specific TCRs can be readily activated and amplified by APCs to generate highly potent T cell responses that involve many millions of T cells. Such activated T cell responses are capable of attacking and clearing viral infections, bacterial infections, and other cellular threats including tumors, as illustrated below. Conversely, the broad, non-specific activation of overly active T cell responses against self or shared antigens can give rise to T cells inappropriately attacking and destroying healthy tissues or cells.

MHC proteins are referred to as human leukocyte antigens (HLA) in humans. HLA class II gene loci include HLA-DM (HLA-DMA and HLA-DMB that encode HLA-DM α chain and HLA-DM β chain, respectively), HLA-DO (HLA-DOA and HLA-DOB that encode HLA-DO α chain and HLA-DO β chain, respectively), HLA-DP (HLA-DPA and HLA-DPB that encode HLA-DP α chain and HLA-DP β chain, respectively), HLA-DQ (HLA-DQA and HLA-DQB that encode HLA-DQ α chain and HLA-DQ β chain, respectively), and HLA-DR (HLA-DRA and HLA-DRB that encode HLA-DR α chain and HLA-DR β chain, respectively).

### SUMMARY

The present disclosure provides T-cell modulatory antigen-presenting polypeptides, including single-chain antigen-presenting polypeptides and multimeric antigen-presenting polypeptides. The present disclosure provides nucleic acids comprising nucleotide sequences encoding T-cell modulatory antigen-presenting polypeptides of the present disclosure, as well as cells genetically modified with the nucleic acids. A T-cell modulatory antigen-presenting polypeptide of the present disclosure is useful for modulating activity of a T cell. Thus, the present disclosure provides methods of modulating activity of a T cell.

### BRIEF DESCRIPTION OF THE DRAWINGS

FIG. 1A-4C provide schematic depictions of examples of T-cell modulatory antigen-presenting polypeptides of the present disclosure.

FIG. 5A-5B provide an amino acid sequences of an immunoglobulin heavy chain CH1 domain (FIG. 5A; SEQ ID NO: 322) and a human kappa light chain constant region (FIG. 5B; SEQ ID NO: 323).

FIG. 6 provides an amino acid sequence of an HLA Class II DRA α chain (SEQ ID NO: 324).

FIG. 7A-7J provide amino acid sequences of HLA Class II DRB1 β chains (from top to bottom: SEQ ID NOs: 325-334).

FIG. 8A-8C provide amino acid sequences of HLA Class II DRB3 β chains (from top to bottom: SEQ ID NOs: 335-337).

FIG. 9 provides an amino acid sequence of an HLA Class II DRB4 β chain (SEQ ID NO: 338).

FIG. 10 provides an amino acid sequence of an HLA Class II DRB5 β chain (SEQ ID NO: 339).

FIG. 11 provides an amino acid sequence of an HLA Class II DMA α chain (SEQ ID NO: 340).

FIG. 12 provides an amino acid sequence of an HLA Class II DMB β chain (SEQ ID NO: 341).

FIG. 13 provides an amino acid sequence of an HLA Class II DOA α chain (SEQ ID NO: 342).

FIG. 14 provides an amino acid sequence of an HLA Class II DOB β chain (SEQ ID NO: 343).

FIG. 15 provides an amino acid sequence of an HLA Class II DPA1 α chain (SEQ ID NO: 344).

FIG. 16 provides an amino acid sequence of an HLA Class II DPB1 β chain (SEQ ID NO: 345).

FIG. 17 provides an amino acid sequence of an HLA Class II DQA1 α chain (SEQ ID NO: 346).

FIG. 18 provides an amino acid sequence of an HLA Class II DQA2 α chain (SEQ ID NO: 347).

FIG. 19A-19B provide amino acid sequences of HLA Class II DQB1 β chains (from top to bottom: SEQ ID NOs: 348-349).

FIG. 20A-20B provide amino acid sequence of HLA Class II DQB2 β chains (from top to bottom: SEQ ID NOs: 350-351).

FIG. 21A-21G provide amino acid sequences of immunoglobulin Fc polypeptides (from top to bottom: SEQ ID NOs: 352-363).

FIG. 22A-22L provide schematic depictions of exemplary multimeric T-cell modulatory antigen-presenting polypeptides of the present disclosure.

FIG. 23A-23I provide schematic depictions of exemplary single-chain T-cell modulatory antigen-presenting polypeptides of the present disclosure.

FIG. 24 depicts production of exemplary antigen-presenting polypeptides of the present disclosure.

FIG. 25A-25B provide the amino acid sequence (FIG. 25A; SEQ ID NO: 364) of an exemplary polypeptide chain of a multimeric T-cell modulator antigen-presenting polypeptide, and a nucleotide sequence (FIG. 25B; SEQ ID NO 365) encoding same.

FIG. 26A-26B provide the amino acid sequence (FIG. 26A; SEQ ID NO: 366) of an exemplary polypeptide chain of a multimeric T-cell modulator antigen-presenting polypeptide, and a nucleotide sequence (FIG. 26B; SEQ ID NO: 367) encoding same.

FIG. 27A-27B provide the amino acid sequence (FIG. 27A; SEQ ID NO: 368) of an exemplary single-chain T-cell modulator antigen-presenting polypeptide, and a nucleotide sequence (FIG. 27B; SEQ ID NO: 369) encoding same.

FIG. 28A-28B provide the amino acid sequence (FIG. 28A; SEQ ID NO: 370) of an exemplary single-chain T-cell modulator antigen-presenting polypeptide, and a nucleotide sequence (FIG. 28B; SEQ ID NO: 371) encoding same.

FIG. 29A-29B provide the amino acid sequence (FIG. 29A; SEQ ID NO: 372) of an exemplary single-chain T-cell modulator antigen-presenting polypeptide, and a nucleotide sequence (FIG. 29B; SEQ ID NO: 373) encoding same.

FIG. 30A-30B provide the amino acid sequence (FIG. 30A; SEQ ID NO: 374) of an exemplary polypeptide chain of a multimeric T-cell modulator antigen-presenting polypeptide, and a nucleotide sequence (FIG. 30B; SEQ ID NO: 375) encoding same.

FIG. 31A-31B provide the amino acid sequence (FIG. 31A; SEQ ID NO: 376) of an exemplary polypeptide chain of a multimeric T-cell modulator antigen-presenting polypeptide, and a nucleotide sequence (FIG. 31B; SEQ ID NO: 377) encoding same.

FIG. 32A-32B provide the amino acid sequence (FIG. 32A; SEQ ID NO: 378) of an exemplary polypeptide chain of a multimeric T-cell modulator antigen-presenting polypeptide, and a nucleotide sequence (FIG. 32B; SEQ ID NO: 379) encoding same.

FIG. 33A-33B provide the amino acid sequence (FIG. 33A; SEQ ID NO: 380) of an exemplary polypeptide chain of a multimeric T-cell modulator antigen-presenting polypeptide, and a nucleotide sequence (FIG. 33B; SEQ ID NO: 381) encoding same.

FIG. 34A-34B provide the amino acid sequence (FIG. 34A; SEQ ID NO: 382) of an exemplary polypeptide chain of a multimeric T-cell modulator antigen-presenting polypeptide, and a nucleotide sequence (FIG. 34B; SEQ ID NO: 383) encoding same.

FIG. 35A-35B provide the amino acid sequence (FIG. 35A; SEQ ID NO: 384) of an exemplary polypeptide chain of a multimeric T-cell modulator antigen-presenting polypeptide, and a nucleotide sequence (FIG. 35B; SEQ ID NO: 385) encoding same.

FIG. 36 provides a schematic depiction of MHC Class II alpha- and beta-chains with a peptide.

FIG. 37A-37C provide schematic depictions of examples of antigen-presenting polypeptides (APPs).

FIG. 38A-38B provide schematic depictions of APPs without immunomodulatory (MOD) polypeptides (FIG. 38A) and with a MOD polypeptide (FIG. 38B). The unmarked rectangle in FIG. 38 represents a dimerization domain (e.g., a bZIP polypeptide). In FIG. 8, the arrows pointing to the dashed lines indicate possible positions of a MOD polypeptide(s).

FIG. 39 provides a table showing associations of HLA class II alleles and haplotypes with risk of autoimmune disease. The table also provides autoantigens associated with the diseases listed.

### DEFINITIONS

The terms “polynucleotide” and “nucleic acid,” used interchangeably herein, refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones.

A polynucleotide or polypeptide has a certain percent “sequence identity” to another polynucleotide or polypeptide, meaning that, when aligned, that percentage of bases or amino acids are the same, and in the same relative position, when comparing the two sequences. Sequence identity can be determined in a number of different ways. To determine sequence identity, sequences can be aligned using various convenient methods and computer programs (e.g., BLAST, T-COFFEE, MUSCLE, MAFFT, etc.), available over the world wide web at sites including ncbi.nlm.nili.gov/BLAST, ebi.ac.uk/Tools/msa/tcoffee/, ebi.ac.uk/Tools/msa/muscle/, mafft.cbrc.jp/alignment/software/. See, e.g., Altschul et al. (1990), J. Mol. Bioi. 215:403-10.

The term “conservative amino acid substitution” refers to the interchangeability in proteins of amino acid residues having similar side chains. For example, a group of amino acids having aliphatic side chains consists of glycine, alanine, valine, leucine, and isoleucine; a group of amino acids having aliphatic-hydroxyl side chains consists of serine and threonine; a group of amino acids having amide containing side chains consisting of asparagine and glutamine; a group of amino acids having aromatic side chains consists of phenylalanine, tyrosine, and tryptophan; a group of amino acids having basic side chains consists of lysine, arginine, and histidine; a group of amino acids having acidic side chains consists of glutamate and aspartate; and a group of amino acids having sulfur containing side chains consists of cysteine and methionine. Exemplary conservative amino acid substitution groups are: valine-leucine-isoleucine, phenylalanine-tyrosine, lysine-arginine, alanine-valine-glycine, and asparagine-glutamine.

The term “binding,” as used herein (e.g. with reference to binding of a T-cell modulatory antigen-presenting polypeptide to a polypeptide (e.g., a T-cell receptor) on a T cell), refers to a non-covalent interaction between two molecules. Non-covalent binding refers to a direct association between two molecules, due to, for example, electrostatic, hydrophobic, ionic, and/or hydrogen-bond interactions, including interactions such as salt bridges and water bridges. Non-covalent binding interactions are generally characterized by a dissociation constant (K_{D}) of less than 10⁻⁶ M, less than 10⁻⁷ M, less than 10⁻⁸ M, less than 10⁻⁹ M, less than 10⁻¹⁰ M, less than 10⁻¹¹ M, less than 10⁻¹² M, less than 10⁻¹³ M, less than 10⁻¹⁴ M, or less than 10⁻¹⁵ M. “Affinity” refers to the strength of non-covalent binding, increased binding affinity being correlated with a lower K_{D}. “Specific binding” generally refers to binding with an affinity of at least about 10⁻⁷ M or greater, e.g., 5×10⁻⁷ M, 10⁻⁸M, 5×10⁻⁸M, 10⁻⁹ M, and greater. “Non-specific binding” generally refers to binding (e.g., the binding of a ligand to a moiety other than its designated binding site or receptor) with an affinity of less than about 10⁻⁷ M (e.g., binding with an affinity of 10⁻⁶ M, 10⁻⁵ M, 10⁻⁴ M). However, in some contexts, e.g., binding between a TCR and a peptide/MHC complex, “specific binding” can be in the range of from 1 μM to 100 μM, or from 100 μM to 1 mM. “Covalent binding” or “covalent bond,” as used herein, refers to the formation of one or more covalent chemical binds between two different molecules.

The term “immunological synapse” or “immune synapse” as used herein generally refers to the natural interface between two interacting immune cells of an adaptive immune response including, e.g., the interface between an antigen-presenting cell (APC) or target cell and an effector cell, e.g., a lymphocyte, an effector T cell, a natural killer cell, and the like. An immunological synapse between an APC and a T cell is generally initiated by the interaction of a T cell antigen receptor and major histocompatibility complex molecules, e.g., as described in Bromley et al., Annu Rev Immunol. 2001; 19:375-96; the disclosure of which is incorporated herein by reference in its entirety.

“T cell” includes all types of immune cells expressing CD3, including T-helper cells (CD4⁺ cells), cytotoxic T-cells (CD8⁺ cells), T-regulatory cells (Treg), and NK-T cells.

The term “immunomodulatory polypeptide” (also referred to as a “co-stimulatory polypeptide”), as used herein, includes a polypeptide on an antigen presenting cell (APC) (e.g., a dendritic cell, a B cell, and the like), or a portion of the polypeptide on an APC, that specifically binds a cognate co-immunomodulatory polypeptide on a T cell, thereby providing a signal which, in addition to the primary signal provided by, for instance, binding of a TCR/CD3 complex with a major histocompatibility complex (MHC) polypeptide loaded with peptide, mediates a T cell response, including, but not limited to, proliferation, activation, differentiation, and the like. An immunomodulatory polypeptide can include, but is not limited to, CD7, B7-1 (CD80), B7-2 (CD86), PD-L1, PD-L2, 4-1BBL, OX40L, Fas ligand (FasL), inducible costimulatory ligand (ICOS-L), intercellular adhesion molecule (ICAM), CD30L, CD40, CD70, CD83, HLA-G, lymphotoxin beta receptor, 3/TR6, ILT3, ILT4, HVEM, an agonist or antibody that binds Toll ligand receptor and a ligand that specifically binds with B7-H3. A co-stimulatory polypeptide also encompasses, inter alia, an antibody that specifically binds with a cognate co-stimulatory molecule present on a T cell, such as, but not limited to, IL-2, CD27, CD28, 4-1BB, OX40, CD30, CD40, PD-1, ICOS, lymphocyte function-associated antigen-1 (LFA-1), CD2, LIGHT, NKG2C, B7-H3, and a ligand that specifically binds to CD83.

As noted above, an “immunomodulatory polypeptide” (also referred to herein as a “MOD”) specifically binds a cognate co-immunomodulatory polypeptide on a T cell.

An “immunomodulatory domain” (“MOD”) of a TMAPP of the present disclosure binds a cognate co-immunomodulatory polypeptide, which may be present on a target T cell.

“Heterologous,” as used herein, means a nucleotide or polypeptide that is not found in the native nucleic acid or protein, respectively.

“Recombinant,” as used herein, means that a particular nucleic acid (DNA or RNA) is the product of various combinations of cloning, restriction, polymerase chain reaction (PCR) and/or ligation steps resulting in a construct having a structural coding or non-coding sequence distinguishable from endogenous nucleic acids found in natural systems. DNA sequences encoding polypeptides can be assembled from cDNA fragments or from a series of synthetic oligonucleotides, to provide a synthetic nucleic acid which is capable of being expressed from a recombinant transcriptional unit contained in a cell or in a cell-free transcription and translation system.

The terms “recombinant expression vector,” or “DNA construct” are used interchangeably herein to refer to a DNA molecule comprising a vector and at least one insert. Recombinant expression vectors are usually generated for the purpose of expressing and/or propagating the insert(s), or for the construction of other recombinant nucleotide sequences. The insert(s) may or may not be operably linked to a promoter sequence and may or may not be operably linked to DNA regulatory sequences.

As used herein, the term “affinity” refers to the equilibrium constant for the reversible binding of two agents (e.g., an antibody and an antigen) and is expressed as a dissociation constant (K_{D}). Affinity can be at least 1-fold greater, at least 2-fold greater, at least 3-fold greater, at least 4-fold greater, at least 5-fold greater, at least 6-fold greater, at least 7-fold greater, at least 8-fold greater, at least 9-fold greater, at least 10-fold greater, at least 20-fold greater, at least 30-fold greater, at least 40-fold greater, at least 50-fold greater, at least 60-fold greater, at least 70-fold greater, at least 80-fold greater, at least 90-fold greater, at least 100-fold greater, or at least 1,000-fold greater, or more, than the affinity of an antibody for unrelated amino acid sequences. Affinity of an antibody to a target protein can be, for example, from about 100 nanomolar (nM) to about 0.1 nM, from about 100 nM to about 1 picomolar (pM), or from about 100 nM to about 1 femtomolar (fM) or more. As used herein, the term “avidity” refers to the resistance of a complex of two or more agents to dissociation after dilution.

The term “binding” refers to a direct association between two molecules, due to, for example, covalent, electrostatic, hydrophobic, and ionic and/or hydrogen-bond interactions, including interactions such as salt bridges and water bridges. “Specific binding” refers to binding with an affinity of at least about 10⁻⁷ M or greater, e.g., 5×10⁻⁷ M, 10⁻⁸M, 5×10⁻⁸M, and greater. “Non-specific binding” refers to binding with an affinity of less than about 10⁻⁷ M, e.g., binding with an affinity of 10⁻⁶ M, 10⁻⁵ M, 10⁻⁴ M, etc.

The terms “treatment”, “treating” and the like are used herein to generally mean obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” as used herein covers any treatment of a disease or symptom in a mammal, and includes: (a) preventing the disease or symptom from occurring in a subject which may be predisposed to acquiring the disease or symptom but has not yet been diagnosed as having it; (b) inhibiting the disease or symptom, i.e., arresting its development; and/or (c) relieving the disease, i.e., causing regression of the disease. The therapeutic agent may be administered before, during or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. The subject therapy will desirably be administered during the symptomatic stage of the disease, and in some cases after the symptomatic stage of the disease.

The terms “individual,” “subject,” “host,” and “patient,” are used interchangeably herein and refer to any mammalian subject for whom diagnosis, treatment, or therapy is desired. Mammals include, e.g., humans, non-human primates, rodents (e.g., rats; mice), lagomorphs (e.g., rabbits), ungulates (e.g., cows, sheep, pigs, horses, goats, and the like), etc.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a Treg” includes a plurality of such Tregs and reference to “the MHC Class II alpha chain” includes reference to one or more MHC Class II alpha chains and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

The present disclosure provides T-cell modulatory antigen-presenting polypeptides (TMAPPs) that comprise: a) a first polypeptide comprising: i) a peptide epitope; and ii) a first MHC Class II polypeptide; and b) a second polypeptide comprising a second MHC Class II polypeptide, where the first and/or the second polypeptides comprises one or more immunomodulatory polypeptides. The present disclosure provides nucleic acids comprising nucleotide sequences encoding TMAPPs of the present disclosure, as well as cells genetically modified with the nucleic acids. A TMAPP of the present disclosure is useful for modulating activity of a T cell. Thus, the present disclosure provides methods of modulating activity of a T cell.

The present disclosure provides an antigen-presenting polypeptide (APP), where an APP of the present disclosure does not include an immunomodulatory polypeptide. An APP of the present disclosure can be a single chain polypeptide or a multi-chain (multimeric) polypeptide. An APP of the present disclosure is useful for diagnostic applications and therapeutic applications.

### T-Cell Modulatory Antigen-Presenting Polypeptides

The present disclosure provides T-cell modulatory antigen-presenting polypeptides (TMAPPs), including single-chain TMAPPs and multimeric TMAPPs. In some cases, a TMAPP of the present disclosure comprises two polypeptide chains and is sometimes referred to herein as a “multimeric T-cell modulatory antigen-presenting polypeptide.” In some cases, a TMAPP of the present disclosure comprises a single polypeptide chain. A TMAPP of the present disclosure is also referred to as a “synTac polypeptide.”

A TMAPP of the present disclosure comprises one or more immunomodulatory polypeptides. In some cases, a TMAPP of the present disclosure comprises a single immunomodulatory polypeptide. In some cases, a TMAPP of the present disclosure comprises two or more immunomodulatory polypeptides (e.g., 2, 3, 4, or 5 immunomodulatory polypeptides).

In some cases, a TMAPP of the present disclosure comprises two or more immunomodulatory polypeptides. In some cases, where a TMAPP of the present disclosure comprises a first polypeptide and a second polypeptide, the two or more immunomodulatory polypeptides are present in the first polypeptide chain only. In some cases, where a TMAPP of the present disclosure comprises a first polypeptide and a second polypeptide, the two or more immunomodulatory polypeptides are present in the second polypeptide chain only. In some cases, where a TMAPP of the present disclosure comprises a first polypeptide and a second polypeptide, at least one of the two or more immunomodulatory polypeptides are present in the first polypeptide chain; and at least one of the two or more immunomodulatory polypeptides are present in the second polypeptide chain.

In some cases, where a TMAPP of the present disclosure comprises two immunomodulatory polypeptides, the two immunomodulatory polypeptides have the same amino acid sequence, i.e., the TMAPP comprises two copies of an immunomodulatory polypeptide. In some cases, where a TMAPP of the present disclosure comprises two immunomodulatory polypeptides, the two immunomodulatory polypeptides do not have the same amino acid sequence; e.g., one of the two immunomodulatory polypeptides comprises a first amino acid sequence and the second of the two immunomodulatory polypeptides comprises a second amino acid sequence, where the first and the second amino acid sequences are not identical. In some cases, the first and the second amino acid sequences differ from one another in amino acid sequence by from 1 amino acid to 10 amino acids, from 10 amino acids to 25 amino acids, or more than 25 amino acids. In some cases, the first and the second amino acid sequences share less than 98%, less than 95%, less than 90%, less than 85%, less than 80%, less than 75%, or less than 70%, amino acid sequence identity with one another.

A TMAPP of the present disclosure modulates activity of a T cell. In some cases, a TMAPP of the present disclosure reduces activity of an autoreactive T cell and/or an autoreactive B cell. In some cases, a TMAPP of the present disclosure increases the number and/or activity of a regulator T cell (Treg), resulting in reduced activity of an autoreactive T cell and/or an autoreactive B cell.

Immunomodulatory polypeptides that are suitable for inclusion in a TMAPP of the present disclosure include, but are not limited to, IL-2, transforming growth factor-beta (TGFβ), JAG1, CD7, B7-1 (CD80), B7-2 (CD86), PD-L1, PD-L2, 4-1BBL, OX40L, Fas ligand (FasL), inducible costimulatory ligand (ICOS-L), intercellular adhesion molecule (ICAM), CD30L, CD40, CD70, CD83, HLA-G, MICA, MICB, HVEM, lymphotoxin beta receptor, 3/TR6, ILT3, and ILT4. In some cases, an immunomodulatory polypeptide suitable for inclusion in a TMAPP of the present disclosure is a variant that comprises from 1 to 10 amino acid substitutions relative to a wild-type or naturally-occurring immunomodulatory polypeptide, and that exhibits reduced affinity to its cognate co-immunomodulatory polypeptide (e.g., a co-immunomodulatory polypeptide present on the surface of a T cell), compared to the affinity of the wild-type or naturally-occurring immunomodulatory polypeptide for the cognate co-immunomodulatory polypeptide.

### Multimeric T-Cell Modulatory Antigen-Presenting Polypeptides

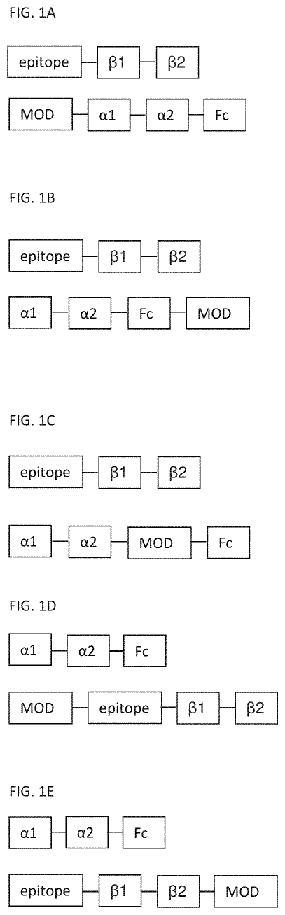

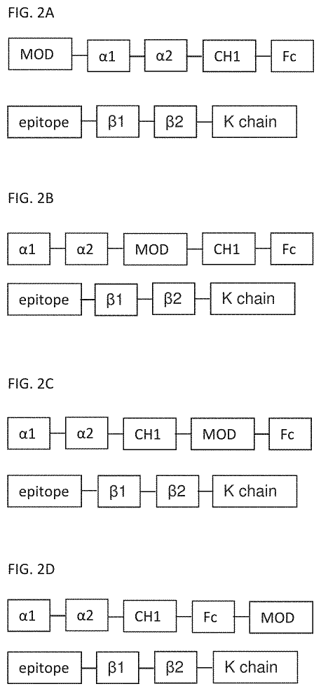

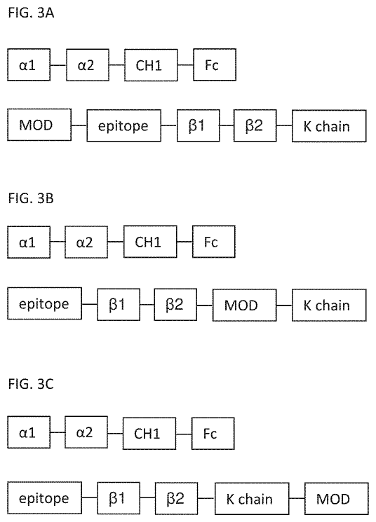

A TMAPP of the present disclosure comprises: i) a peptide epitope (a peptide recognized and bound by a TCR); ii) an MHC Class II α chain polypeptide; iii) an MHC Class II R chain polypeptide; and iv) an immunomodulatory polypeptide (also referred to herein as a “MOD polypeptide” or a “MOD domain”) In some cases, the TMAPP comprises two polypeptide chains; such a TMAPP is referred to herein as a multimeric TMAPP. A TMAPP of the present disclosure can further include one or both of: a dimerizer polypeptide; and an immunoglobulin scaffold (e.g., an Ig Fc polypeptide) or a non-immunoglobulin scaffold. Non-limiting example of multimeric TMAPPs of the present disclosure is schematically depicted in FIG. 1A-1E, FIG. 2A-2D, and FIG. 3A-3C.

In some cases, a TMAPP of the present disclosure comprises a single immunomodulatory polypeptide. In some cases, a TMAPP of the present disclosure comprises 2 copies of an immunomodulatory polypeptide. In some cases, a TMAPP of the present disclosure comprises 3 copies of an immunomodulatory polypeptide. Where a TMAPP of the present disclosure comprises 2 or 3 copies of an immunomodulatory polypeptide, in some cases, the 2 or 3 copies are in tandem. Where a TMAPP of the present disclosure comprises 2 or 3 copies of an immunomodulatory polypeptide, in some cases, the 2 or 3 copies are separated from one another by a linker.

A TMAPP of the present disclosure can include one or more linkers, where the one or more linkers are between one or more of: i) an MHC Class II polypeptide and an Ig Fc polypeptide, where such a linker is referred to herein as “L1”; ii) an immunomodulatory polypeptide and an MHC Class II polypeptide, where such a linker is referred to herein as “L2”; iii) a first immunomodulatory polypeptide and a second immunomodulatory polypeptide, where such a linker is referred to herein as “L3”; iv) a peptide antigen (“epitope”) and an MHC Class II polypeptide; v) an MHC Class II polypeptide and a dimerization polypeptide (e.g., a first or a second member of a dimerizing pair); and vi) a dimerization polypeptide (e.g., a first or a second member of a dimerizing pair) and an IgFc polypeptide. In some cases, an L1 linker comprises (GGGGS)n (SEQ ID NO: 1), where n is 1, 2, 3, 4, 5, 6, 7, or 8. In some cases, an L2 linker comprises (GGGGS)n (SEQ ID NO: 1), where n is 1, 2, 3, 4, 5, 6, 7, or 8. In some cases, an L3 linker comprises (GGGGS)n (SEQ ID NO: 1), where n is 1, 2, 3, 4, 5, 6, 7, or 8. In some cases, a linker comprises the amino acid sequence GGSAAAGG (SEQ ID NO: 2).

In some cases, a TMAPP of the present disclosure comprises: a) a first polypeptide comprising, in order from N-terminus to C-terminus: i) a peptide antigen (an “epitope”) that is recognized (e.g., is capable of being recognized and bound) by a TCR; ii) an MHC Class II β1 polypeptide; and iii) an MHC Class II β2 polypeptide; and b) a second polypeptide comprising: i) an immunomodulatory polypeptide; ii) an MHC Class II α1 polypeptide; and iii) an MHC Class II α2 polypeptide. In some cases, a TMAPP of the present disclosure comprises: a) a first polypeptide comprising, in order from N-terminus to C-terminus: i) a peptide antigen (an “epitope”) that is recognized (e.g., is capable of being recognized and bound) by a TCR; ii) an MHC Class II β1 polypeptide; and iii) an MHC Class II β2 polypeptide; and b) a second polypeptide comprising: i) an immunomodulatory polypeptide; ii) an MHC Class II α1 polypeptide; iii) an MHC Class II α2 polypeptide; and iv) an Ig Fc polypeptide. The arrangement (N-terminus to C-terminus order) of the immunomodulatory polypeptide, the MHC Class II α1 polypeptide, the MHC Class II α2 polypeptide, and the Ig Fc polypeptide, can vary. As one example of a possible arrangement, in some cases, a TMAPP of the present disclosure comprises: a) a first polypeptide comprising, in order from N-terminus to C-terminus: i) a peptide antigen (an “epitope”) that is recognized (e.g., is capable of being recognized and bound) by a TCR; ii) an MHC Class II β1 polypeptide; and iii) an MHC Class II β2 polypeptide; and b) a second polypeptide comprising, in order from N-terminus to C-terminus: i) an immunomodulatory polypeptide; ii) an MHC Class II α1 polypeptide; iii) an MHC Class II α2 polypeptide; and iv) an Ig Fc polypeptide. This arrangement is depicted schematically in FIG. 1A. As another example of a possible arrangement, in some cases, a TMAPP of the present disclosure comprises: a) a first polypeptide comprising, in order from N-terminus to C-terminus: i) a peptide antigen (an “epitope”) that is recognized (e.g., is capable of being recognized and bound) by a TCR; ii) an MHC Class II β1 polypeptide; and iii) an MHC Class II β2 polypeptide; and b) a second polypeptide comprising, in order from N-terminus to C-terminus: i) an MHC Class II α1 polypeptide; ii) an MHC Class II α2 polypeptide; iii) an Ig Fc polypeptide; and iv) an immunomodulatory polypeptide. This arrangement is depicted schematically in FIG. 1B. As another example of a possible arrangement, in some cases, a TMAPP of the present disclosure comprises: a) a first polypeptide comprising, in order from N-terminus to C-terminus: i) a peptide antigen (an “epitope”) that is recognized (e.g., is capable of being recognized and bound) by a TCR; ii) an MHC Class II β1 polypeptide; and iii) an MHC Class II β2 polypeptide; and b) a second polypeptide comprising, in order from N-terminus to C-terminus: i) an MHC Class II α1 polypeptide; ii) an MHC Class II α2 polypeptide; iii) an immunomodulatory polypeptide; and iv) an Ig Fc polypeptide. This arrangement is depicted schematically in FIG. 1C. The immunomodulatory polypeptide(s) can be on the same polypeptide chain as the MHC Class II α1 and α2 polypeptides, as illustrated schematically in FIG. 1A-1C. Alternatively, the peptide antigen (“epitope”) can be on the same polypeptide chain as the MHC Class II β1 and β2 polypeptides, as illustrated schematically in FIG. 1D and FIG. 1E. For example, in some cases, a TMAPP of the present disclosure comprises: a) a first polypeptide comprising, in order from N-terminus to C-terminus: i) an immunomodulatory polypeptide; ii) a peptide antigen (an “epitope”) that is recognized (e.g., is capable of being recognized and bound) by a TCR; iii) an MHC Class II β1 polypeptide; and iv) an MHC Class II β2 polypeptide; and b) a second polypeptide comprising, in order from N-terminus to C-terminus: i) an MHC Class II α1 polypeptide; ii) an MHC Class II α2 polypeptide; and iii) an Ig Fc polypeptide. This arrangement is depicted schematically in FIG. 1D. As another example, in some cases, a TMAPP of the present disclosure comprises: a) a first polypeptide comprising, in order from N-terminus to C-terminus: i) a peptide antigen (an “epitope”) that is recognized (e.g., is capable of being recognized and bound) by a TCR; ii) an MHC Class II β1 polypeptide; iii) an MHC Class II β2 polypeptide; and iv) an immunomodulatory polypeptide; and b) a second polypeptide comprising, in order from N-terminus to C-terminus: i) an MHC Class II α1 polypeptide; ii) an MHC Class II α2 polypeptide; and iii) an Ig Fc polypeptide. This arrangement is depicted schematically in FIG. 1E. In any one of the above embodiments, the TMAPP can include a single immunomodulatory polypeptide. In any one of the above embodiments, the TMAPP can include 2 copies of the immunomodulatory polypeptide; the 2 copies can be in tandem, or can be separated by a linker. In any one of the above embodiments, the TMAPP can include 3 copies of the immunomodulatory polypeptide; the 3 copies can be in tandem, or can be separated by a linker. Where a TMAPP of the present disclosure comprises two immunomodulatory polypeptides, in some cases, the first immunomodulatory polypeptide is linked to the second immunomodulatory polypeptide by a linker (an “L3” linker); e.g., a linker of from about 2 amino acids to 50 amino acids in length. Suitable L3 linkers include (GGGGS)n (SEQ ID NO: 1), where n is 1, 2, 3, 4, 5, 6, 7, or 8. In some cases, the TMAPP comprises a linker (an “L1”) between the MHC polypeptide and the Ig Fc polypeptide; where exemplary suitable linkers include (GGGGS)n (SEQ ID NO: 1), where n is 1, 2, 3, 4, 5, 6, 7, or 8. In some cases, the TMAPP comprises a linker (an “L2”) between the immunomodulatory polypeptide and the MHC polypeptide, where exemplary suitable linkers include (GGGGS)n (SEQ ID NO: 1), where n is 1, 2, 3, 4, 5, 6, 7, or 8. In some cases, where the TMAPP comprises two immunomodulatory polypeptides, in some cases, the two immunomodulatory polypeptides are separated by a linker (an “L3); where exemplary suitable linkers include (GGGGS)n (SEQ ID NO: 1), where n is 1, 2, 3, 4, 5, 6, 7, or 8. In some cases, the linker between any two components of the TMAPP comprises the amino acid sequence GGSAAAGG (SEQ ID NO: 2). In any of the above embodiments, in some cases, the Ig Fc is an IgG1 Fc polypeptide. In any of the above embodiments, in some cases, the Ig Fc is an IgG4 Fc polypeptide. In any of the above embodiments, in some cases, the immunomodulatory polypeptide is a PD-L1 polypeptide. In any of the above embodiments, in some cases, the immunomodulatory polypeptide is a TGF-β polypeptide. In any of the above embodiments, in some cases, the immunomodulatory polypeptide is a FasL polypeptide. In some cases, the epitope is an auto-epitope (an epitope of a self antigen).

The two polypeptide chains of a TMAPP of the present disclosure can be covalently linked, e.g., via a disulfide bond. The two polypeptide chains of a TMAPP of the present disclosure can also associate with one another non-covalently. The two polypeptide chains of a TMAPP of the present disclosure can be linked via interaction between a first dimerization domain present in the first polypeptide, and a second dimerization domain present in the second polypeptide. For example, the first polypeptide chain of a TMAPP of the present disclosure can include an Ig CH1 polypeptide as a first dimerization domain; and the second polypeptide chain of a TMAPP of the present disclosure can include the constant region of an immunoglobulin κ chain, as the second dimerization domain.

A suitable Ig CH1 polypeptide has a length of from about 90 amino acids to about 120 amino acids (e.g., from about 90 amino acids to about 95 amino acids, from about 95 amino acids to about 100 amino acids, from about 100 amino acids to about 105 amino acids, from about 105 amino acids to about 110 amino acids, from about 110 amino acids to about 115 amino acids, or from about 110 amino acids to about 120 amino acids); and can comprise an amino acid sequence having at least 60%, at least 70%, at least 80%, at least 90%, at least 95%, at least 98%, at least 99%, or 100%, amino acid sequence identity to the following CH1 amino acid sequence:

(SEQ ID NO: 3)
ASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKV
EPKSCDKT.

A suitable Ig κ chain constant region polypeptide has a length of from about 90 amino acids to about 120 amino acids (e.g., from about 90 amino acids to about 95 amino acids, from about 95 amino acids to about 100 amino acids, from about 100 amino acids to about 105 amino acids, from about 105 amino acids to about 110 amino acids, from about 110 amino acids to about 115 amino acids, or from about 110 amino acids to about 120 amino acids); and can comprise an amino acid sequence having at least 60%, at least 70%, at least 80%, at least 90%, at least 95%, at least 98%, at least 99%, or 100%, amino acid sequence identity to the following κ chain constant region amino acid sequence:

(SEQ ID NO: 4)
TVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVT
KSFNRGEC.

In some cases, a TMAPP of the present disclosure comprises: a) a first polypeptide comprising, in order from N-terminus to C-terminus: i) a peptide antigen (an “epitope”) that is recognized (e.g., is capable of being recognized and bound) by a TCR; ii) an MHC Class II β1 polypeptide; iii) an MHC Class II β2 polypeptide; and iv) an Ig κ chain constant region polypeptide; and b) a second polypeptide comprising: i) an immunomodulatory polypeptide; ii) an MHC Class II α1 polypeptide; iii) an MHC Class II α2 polypeptide; and iv) a CH1 polypeptide. In some cases, a TMAPP of the present disclosure comprises: a) a first polypeptide comprising, in order from N-terminus to C-terminus: i) a peptide antigen (an “epitope”) that is recognized (e.g., is capable of being recognized and bound) by a TCR; ii) an MHC Class II β1 polypeptide; iii) an MHC Class II β2 polypeptide; and iv) an Ig κ chain constant region polypeptide; and b) a second polypeptide comprising: i) an immunomodulatory polypeptide; ii) an MHC Class II α1 polypeptide; iii) an MHC Class II α2 polypeptide; iv) a CH1 polypeptide; and v) an Ig Fc polypeptide. In some cases, a TMAPP of the present disclosure comprises: a) a first polypeptide comprising, in order from N-terminus to C-terminus: i) a peptide antigen (an “epitope”) that is recognized (e.g., is capable of being recognized and bound) by a TCR; ii) an MHC Class II β1 polypeptide; iii) an MHC Class II β2 polypeptide; and iv) an Ig κ chain constant region polypeptide; and b) a second polypeptide comprising, in order from N-terminus to C-terminus: i) an immunomodulatory polypeptide; ii) an MHC Class II α1 polypeptide; iii) an MHC Class II α2 polypeptide; iv) a CH1 polypeptide; and v) an Ig Fc polypeptide. An example of such a TMAPP is depicted schematically in FIG. 2A. In some cases, a TMAPP of the present disclosure comprises: a) a first polypeptide comprising, in order from N-terminus to C-terminus: i) a peptide antigen (an “epitope”) that is recognized (e.g., is capable of being recognized and bound) by a TCR; ii) an MHC Class II β1 polypeptide; iii) an MHC Class II β2 polypeptide; and iv) an Ig κ chain constant region polypeptide; and b) a second polypeptide comprising, in order from N-terminus to C-terminus: i) an MHC Class II α1 polypeptide; ii) an MHC Class II α2 polypeptide; iii) an immunomodulatory polypeptide; iv) a CH1 polypeptide; and v) an Ig Fc polypeptide. An example of such a TMAPP is depicted schematically in FIG. 2B. As another example of a possible arrangement, in some cases, a TMAPP of the present disclosure comprises: a) a first polypeptide comprising, in order from N-terminus to C-terminus: i) a peptide antigen (an “epitope”) that is recognized (e.g., is capable of being recognized and bound) by a TCR; ii) an MHC Class II β1 polypeptide; iii) an MHC Class II β2 polypeptide; and iv) an Ig κ constant region polypeptide; and b) a second polypeptide comprising, in order from N-terminus to C-terminus: i) an MHC Class II α1 polypeptide; ii) an MHC Class II α2 polypeptide; iii) a CH1 polypeptide; iv) an immunomodulatory polypeptide; and v) an Ig Fc polypeptide. This arrangement is depicted schematically in FIG. 2C. In some cases, a TMAPP of the present disclosure comprises: a) a first polypeptide comprising, in order from N-terminus to C-terminus: i) a peptide antigen (an “epitope”) that is recognized (e.g., is capable of being recognized and bound) by a TCR; ii) an MHC Class II β1 polypeptide; iii) an MHC Class II β2 polypeptide; and iv) an Ig κ chain constant region polypeptide; and b) a second polypeptide comprising, in order from N-terminus to C-terminus: i) an MHC Class II α1 polypeptide; ii) an MHC Class II α2 polypeptide; iii) a CH1 polypeptide; iv an Ig Fc polypeptide; and v) an immunomodulatory polypeptide. An example of such a TMAPP is depicted schematically in FIG. 2D. In any one of the above embodiments, the TMAPP can include a single immunomodulatory polypeptide. In any one of the above embodiments, the TMAPP can include 2 copies of the immunomodulatory polypeptide; the 2 copies can be in tandem, or can be separated by a linker. In any one of the above embodiments, the TMAPP can include 3 copies of the immunomodulatory polypeptide; the 3 copies can be in tandem, or can be separated by a linker. Where a TMAPP of the present disclosure comprises two immunomodulatory polypeptides, in some cases, the first immunomodulatory polypeptide is linked to the second immunomodulatory polypeptide by a linker (an “L3” linker); e.g., a linker of from about 2 amino acids to 50 amino acids in length. Suitable L3 linkers include (GGGGS)n (SEQ ID NO: 1), where n is 1, 2, 3, 4, 5, 6, 7, or 8. In some cases, the TMAPP comprises a linker (an “L1”) between the MHC polypeptide and the Ig Fc polypeptide; where exemplary suitable linkers include (GGGGS)n (SEQ ID NO: 1), where n is 1, 2, 3, 4, 5, 6, 7, or 8. In some cases, the TMAPP comprises a linker (an “L2”) between the immunomodulatory polypeptide and the MHC polypeptide, where exemplary suitable linkers include (GGGGS)n (SEQ ID NO: 1), where n is 1, 2, 3, 4, 5, 6, 7, or 8. In some cases, where the TMAPP comprises two immunomodulatory polypeptides, in some cases, the two immunomodulatory polypeptides are separated by a linker (an “L3); where exemplary suitable linkers include (GGGGS)n (SEQ ID NO: 1), where n is 1, 2, 3, 4, 5, 6, 7, or 8. In some cases, the linker between any two components of the TMAPP comprises the amino acid sequence GGSAAAGG (SEQ ID NO: 2). In any of the above embodiments, in some cases, the Ig Fc is an IgG1 Fc polypeptide. In any of the above embodiments, in some cases, the Ig Fc is an IgG4 Fc polypeptide. In any of the above embodiments, in some cases, the immunomodulatory polypeptide is a PD-L1 polypeptide. In any of the above embodiments, in some cases, the immunomodulatory polypeptide is a TGF-β polypeptide. In any of the above embodiments, in some cases, the immunomodulatory polypeptide is a FasL polypeptide. In some cases, the epitope is an auto-epitope (an epitope of a self antigen).

In some cases, a TMAPP of the present disclosure comprises: a) a first polypeptide comprising: i) a peptide antigen (an “epitope”) that is recognized (e.g., is capable of being recognized and bound) by a TCR; ii) an MHC Class II β1 polypeptide; iii) an MHC Class II β2 polypeptide; iv) an immunomodulatory polypeptide; and v) an Ig κ chain constant region polypeptide; and b) a second polypeptide comprising: i) an MHC Class II α1 polypeptide; ii) an MHC Class II α2 polypeptide; and iii) a CH1 polypeptide. In some cases, a TMAPP of the present disclosure comprises: a) a first polypeptide comprising: i) a peptide antigen (an “epitope”) that is recognized (e.g., is capable of being recognized and bound) by a TCR; ii) an MHC Class II β1 polypeptide; iii) an MHC Class II β2 polypeptide; iv) an immunomodulatory polypeptide; and v) an Ig κ chain constant region polypeptide; and b) a second polypeptide comprising: i) an MHC Class II α1 polypeptide; ii) an MHC Class II α2 polypeptide; iii) a CH1 polypeptide; and v) an Ig Fc polypeptide. As an example, in some cases, a TMAPP of the present disclosure comprises, in order from N-terminus to C-terminus: i) an immunomodulatory polypeptide; ii) a peptide antigen (an “epitope”) that is recognized (e.g., is capable of being recognized and bound) by a TCR; iii) an MHC Class II β1 polypeptide; iv) an MHC Class II β2 polypeptide; and v) an Ig κ chain constant region polypeptide; and b) a second polypeptide comprising, in order from N-terminus to C-terminus: i) an MHC Class II α1 polypeptide; ii) an MHC Class II α2 polypeptide; iii) a CH1 polypeptide; and v) an Ig Fc polypeptide. Such a TMAPP is depicted schematically in FIG. 3A. As another example, in some cases, a TMAPP of the present disclosure comprises, in order from N-terminus to C-terminus: i) a peptide antigen (an “epitope”) that is recognized (e.g., is capable of being recognized and bound) by a TCR; ii) an MHC Class II β1 polypeptide; iii) an MHC Class II β2 polypeptide; iv) an immunomodulatory polypeptide; and v) an Ig κ chain constant region polypeptide; and b) a second polypeptide comprising, in order from N-terminus to C-terminus: i) an MHC Class II α1 polypeptide; ii) an MHC Class II α2 polypeptide; iii) a CH1 polypeptide; and v) an Ig Fc polypeptide. Such a TMAPP is depicted schematically in FIG. 3B. As another example, in some cases, a TMAPP of the present disclosure comprises, in order from N-terminus to C-terminus: i) a peptide antigen (an “epitope”) that is recognized (e.g., is capable of being recognized and bound) by a TCR; ii) an MHC Class II β1 polypeptide; iii) an MHC Class II β2 polypeptide; iv) an Ig κ chain constant region polypeptide; and v) an immunomodulatory polypeptide; and b) a second polypeptide comprising, in order from N-terminus to C-terminus: i) an MHC Class II α1 polypeptide; ii) an MHC Class II α2 polypeptide; iii) a CH1 polypeptide; and v) an Ig Fc polypeptide. Such a TMAPP is depicted schematically in FIG. 3C. In any one of the above embodiments, the TMAPP can include a single immunomodulatory polypeptide. In any one of the above embodiments, the TMAPP can include 2 copies of the immunomodulatory polypeptide; the 2 copies can be in tandem, or can be separated by a linker. In any one of the above embodiments, the TMAPP can include 3 copies of the immunomodulatory polypeptide; the 3 copies can be in tandem, or can be separated by a linker. Where a TMAPP of the present disclosure comprises two immunomodulatory polypeptides, in some cases, the first immunomodulatory polypeptide is linked to the second immunomodulatory polypeptide by a linker (an “L3” linker); e.g., a linker of from about 2 amino acids to 50 amino acids in length. Suitable L3 linkers include (GGGGS)n (SEQ ID NO: 1), where n is 1, 2, 3, 4, 5, 6, 7, or 8. In some cases, the TMAPP comprises a linker (an “L1”) between the MHC polypeptide and the Ig Fc polypeptide; where exemplary suitable linkers include (GGGGS)n (SEQ ID NO: 1), where n is 1, 2, 3, 4, 5, 6, 7, or 8. In some cases, the TMAPP comprises a linker (an “L2”) between the immunomodulatory polypeptide and the MHC polypeptide, where exemplary suitable linkers include (GGGGS)n (SEQ ID NO: 1), where n is 1, 2, 3, 4, 5, 6, 7, or 8. In some cases, where the TMAPP comprises two immunomodulatory polypeptides, in some cases, the two immunomodulatory polypeptides are separated by a linker (an “L3); where exemplary suitable linkers include (GGGGS)n (SEQ ID NO: 1), where n is 1, 2, 3, 4, 5, 6, 7, or 8. In some cases, the linker between any two components of the TMAPP comprises the amino acid sequence GGSAAAGG (SEQ ID NO: 2). In any of the above embodiments, in some cases, the Ig Fc is an IgG1 Fc polypeptide. In any of the above embodiments, in some cases, the Ig Fc is an IgG4 Fc polypeptide. In any of the above embodiments, in some cases, the immunomodulatory polypeptide is a PD-L1 polypeptide. In any of the above embodiments, in some cases, the immunomodulatory polypeptide is a TGF-β polypeptide. In any of the above embodiments, in some cases, the immunomodulatory polypeptide is a FasL polypeptide. In some cases, the epitope is an auto-epitope (an epitope of a self antigen).

In some cases, a TMAPP of the present disclosure comprises: a) a first polypeptide comprising, in order from N-terminus to C-terminus: i) a peptide antigen (an “epitope”) that is recognized (e.g., is capable of being recognized and bound) by a TCR; ii) an MHC Class II β1 polypeptide; iii) an MHC Class II α1 polypeptide; and iv) an MHC Class II α2 polypeptide; and b) a second polypeptide comprising: i) an immunomodulatory polypeptide; and ii) an MHC Class II β2 polypeptide. In some cases, the second polypeptide comprises, in order from N-terminus to C-terminus: i) an immunomodulatory polypeptide; and ii) an MHC Class II β2 polypeptide. In some cases, a TMAPP of the present disclosure comprises: a) a first polypeptide comprising, in order from N-terminus to C-terminus: i) a peptide antigen (an “epitope”) that is recognized (e.g., is capable of being recognized and bound) by a TCR; ii) an MHC Class II β1 polypeptide; iii) an MHC Class II α1 polypeptide; iv) an MHC Class II α2 polypeptide; and v) an immunoglobulin or non-immunoglobulin scaffold polypeptide; and b) a second polypeptide comprising: i) an immunomodulatory polypeptide; and ii) an MHC Class II β2 polypeptide. In some cases, the second polypeptide comprises, in order from N-terminus to C-terminus: i) an immunomodulatory polypeptide; and ii) an MHC Class II β2 polypeptide. In some cases, a TMAPP of the present disclosure comprises: a) a first polypeptide comprising, in order from N-terminus to C-terminus: i) a peptide antigen (an “epitope”) that is recognized (e.g., is capable of being recognized and bound) by a TCR; ii) an MHC Class II β1 polypeptide; iii) an MHC Class II α1 polypeptide; iv) an MHC Class II α2 polypeptide; and v) an Ig Fc polypeptide; and b) a second polypeptide comprising: i) an immunomodulatory polypeptide; and ii) an MHC Class II β2 polypeptide. In some cases, the second polypeptide comprises, in order from N-terminus to C-terminus: i) an immunomodulatory polypeptide; and ii) an MHC Class II β2 polypeptide. In some cases, a TMAPP of the present disclosure comprises: a) a first polypeptide comprising, in order from N-terminus to C-terminus: i) a peptide antigen (an “epitope”) that is recognized (e.g., is capable of being recognized and bound) by a TCR; ii) an MHC Class II β1 polypeptide; iii) an MHC Class II α1 polypeptide; iv) an MHC Class II α2 polypeptide; and v) a first member of a dimerizer pair; and b) a second polypeptide comprising: i) an immunomodulatory polypeptide; ii) an MHC Class II β2 polypeptide; iii) a second member of the dimerizer pair. In some cases, the second polypeptide comprises, in order from N-terminus to C-terminus: i) an immunomodulatory polypeptide; ii) an MHC Class II β2 polypeptide; iii) a second member of the dimerizer pair. In some cases, a TMAPP of the present disclosure comprises: a) a first polypeptide comprising, in order from N-terminus to C-terminus: i) a peptide antigen (an “epitope”) that is recognized (e.g., is capable of being recognized and bound) by a TCR; ii) an MHC Class II β1 polypeptide; iii) an MHC Class II α1 polypeptide; iv) an MHC Class II α2 polypeptide; and v) a first leucine zipper polypeptide; and b) a second polypeptide comprising: i) an immunomodulatory polypeptide; ii) an MHC Class II β2 polypeptide; and iii) a second leucine zipper polypeptide. In some cases, the second polypeptide comprises, in order from N-terminus to C-terminus: i) an immunomodulatory polypeptide; ii) an MHC Class II β2 polypeptide; and iii) a second leucine zipper polypeptide. In some cases, a TMAPP of the present disclosure comprises: a) a first polypeptide comprising, in order from N-terminus to C-terminus: i) a peptide antigen (an “epitope”) that is recognized (e.g., is capable of being recognized and bound) by a TCR; ii) an MHC Class II β1 polypeptide; iii) an MHC Class II α1 polypeptide; iv) an MHC Class II α2 polypeptide; v) a first leucine zipper polypeptide; and vi) an Ig Fc polypeptide; and b) a second polypeptide comprising: i) an immunomodulatory polypeptide; ii) an MHC Class II β2 polypeptide; and iii) a second leucine zipper polypeptide. In some cases, the second polypeptide comprises, in order from N-terminus to C-terminus: i) an immunomodulatory polypeptide; ii) an MHC Class II β2 polypeptide; and iii) a second leucine zipper polypeptide. In any one of the above embodiments, the TMAPP can include a single immunomodulatory polypeptide. In any one of the above embodiments, the TMAPP can include 2 copies of the immunomodulatory polypeptide; the 2 copies can be in tandem, or can be separated by a linker. In any one of the above embodiments, the TMAPP can include 3 copies of the immunomodulatory polypeptide; the 3 copies can be in tandem, or can be separated by a linker. For example, in some cases, a TMAPP of the present disclosure comprises: a) a first polypeptide comprising, in order from N-terminus to C-terminus: i) a peptide antigen (an “epitope”) that is recognized (e.g., is capable of being recognized and bound) by a TCR; ii) an MHC Class II β1 polypeptide; iii) an MHC Class II α1 polypeptide; iv) an MHC Class II α2 polypeptide; v) a first leucine zipper polypeptide; and vi) an Ig Fc polypeptide; and b) a second polypeptide comprising: i) a first immunomodulatory polypeptide; ii) a second immunomodulatory polypeptide; iii) an MHC Class II β2 polypeptide; and iv) a second leucine zipper polypeptide. In some cases, the second polypeptide comprises, in order from N-terminus to C-terminus: i) a first immunomodulatory polypeptide; ii) a second immunomodulatory polypeptide; iii) an MHC Class II β2 polypeptide; and iv) a second leucine zipper polypeptide. In some cases, the first and the second immunomodulatory polypeptides comprise the same amino acid sequences. As another example, in some cases, a TMAPP of the present disclosure comprises: a) a first polypeptide comprising, in order from N-terminus to C-terminus: i) a peptide antigen (an “epitope”) that is recognized (e.g., is capable of being recognized and bound) by a TCR; ii) an MHC Class II β1 polypeptide; iii) an MHC Class II α1 polypeptide; iv) an MHC Class II α2 polypeptide; and v) an Ig Fc polypeptide; and b) a second polypeptide comprising: i) a first immunomodulatory polypeptide; ii) a second immunomodulatory polypeptide; and iii) an MHC Class II β2 polypeptide. In some cases, the second polypeptide comprises, in order from N-terminus to C-terminus: i) a first immunomodulatory polypeptide; ii) a second immunomodulatory polypeptide; and iii) an MHC Class II β2 polypeptide. In some cases, the first and the second immunomodulatory polypeptides comprise the same amino acid sequences. In some cases, a TMAPP of the present disclosure comprises: a) a first polypeptide comprising, in order from N-terminus to C-terminus: i) a peptide antigen (an “epitope”) that is recognized (e.g., is capable of being recognized and bound) by a TCR; ii) an MHC Class II β1 polypeptide; iii) an MHC Class II α1 polypeptide; and iv) an MHC Class II α2 polypeptide; and b) a second polypeptide comprising: i) an immunomodulatory polypeptide; ii) an MHC Class II β2 polypeptide; and iii) an Ig Fc polypeptide. In some cases, the second polypeptide comprises, in order from N-terminus to C-terminus: i) an immunomodulatory polypeptide; ii) an MHC Class II β2 polypeptide; and iii) an Ig Fc polypeptide. In some cases, a TMAPP of the present disclosure comprises: a) a first polypeptide comprising, in order from N-terminus to C-terminus: i) a peptide antigen (an “epitope”) that is recognized (e.g., is capable of being recognized and bound) by a TCR; ii) an MHC Class II β1 polypeptide; iii) an MHC Class II α1 polypeptide; and iv) an MHC Class II α2 polypeptide; and b) a second polypeptide comprising: i) a first immunomodulatory polypeptide; ii) a second immunomodulatory polypeptide; iii) an MHC Class II β2 polypeptide; iv) an Ig Fc polypeptide. In some cases, the second polypeptide comprises, in order from N-terminus to C-terminus: i) a first immunomodulatory polypeptide; ii) a second immunomodulatory polypeptide; iii) an MHC Class II β2 polypeptide; iv) an Ig Fc polypeptide. In some cases, the first and the second immunomodulatory polypeptides comprise the same amino acid sequence. In some cases, a TMAPP of the present disclosure comprises: a) a first polypeptide comprising, in order from N-terminus to C-terminus: i) an immunomodulatory polypeptide; ii) an MHC Class II β1 polypeptide; iii) an MHC Class II α1 polypeptide; and iv) an MHC Class II α2 polypeptide; and b) a second polypeptide comprising: i) a peptide antigen (an “epitope”) that is recognized (e.g., is capable of being recognized and bound) by a TCR; ii) an MHC Class II β2 polypeptide; and iii) an Ig Fc polypeptide. In some cases, the second polypeptide comprises, in order from N-terminus to C-terminus: i) a peptide antigen (an “epitope”) that is recognized (e.g., is capable of being recognized and bound) by a TCR; ii) an MHC Class II β2 polypeptide; and iii) an Ig Fc polypeptide. In some cases, a TMAPP of the present disclosure comprises: a) a first polypeptide comprising, in order from N-terminus to C-terminus: i) a first immunomodulatory polypeptide; ii) a second immunomodulatory polypeptide; iii) an MHC Class II β1 polypeptide; iv) an MHC Class II α1 polypeptide; and v) an MHC Class II α2 polypeptide; and b) a second polypeptide comprising, in order from N-terminus to C-terminus: i) a peptide antigen (an “epitope”) that is recognized (e.g., is capable of being recognized and bound) by a TCR; ii) an MHC Class II β2 polypeptide; and iii) an Ig Fc polypeptide. In some cases, the second polypeptide comprises, in order from N-terminus to C-terminus: i) a peptide antigen (an “epitope”) that is recognized (e.g., is capable of being recognized and bound) by a TCR; ii) an MHC Class II β2 polypeptide; and iii) an Ig Fc polypeptide. In some cases, the first and the second immunomodulatory polypeptides comprise the same amino acid sequence. Where a TMAPP of the present disclosure comprises two immunomodulatory polypeptides, in some cases, the first immunomodulatory polypeptide is linked to the second immunomodulatory polypeptide by a linker (an “L3” linker); e.g., a linker of from about 2 amino acids to 50 amino acids in length. Suitable L3 linkers include (GGGGS)n (SEQ ID NO: 1), where n is 1, 2, 3, 4, 5, 6, 7, or 8. In some cases, the TMAPP comprises a linker (an “L1”) between the MHC polypeptide and the Ig Fc polypeptide; where exemplary suitable linkers include (GGGGS)n (SEQ ID NO: 1), where n is 1, 2, 3, 4, 5, 6, 7, or 8. In some cases, the TMAPP comprises a linker (an “L2”) between the immunomodulatory polypeptide and the MHC polypeptide, where exemplary suitable linkers include (GGGGS)n (SEQ ID NO: 1), where n is 1, 2, 3, 4, 5, 6, 7, or 8. In some cases, where the TMAPP comprises two immunomodulatory polypeptides, in some cases, the two immunomodulatory polypeptides are separated by a linker (an “L3); where exemplary suitable linkers include (GGGGS)n (SEQ ID NO: 1), where n is 1, 2, 3, 4, 5, 6, 7, or 8. In some cases, the linker between any two components of the TMAPP comprises the amino acid sequence GGSAAAGG (SEQ ID NO: 2). In any of the above embodiments, in some cases, the Ig Fc is an IgG1 Fc polypeptide. In any of the above embodiments, in some cases, the Ig Fc is an IgG4 Fc polypeptide. In any of the above embodiments, in some cases, the immunomodulatory polypeptide is a PD-L1 polypeptide. In any of the above embodiments, in some cases, the immunomodulatory polypeptide is a TGF-β polypeptide. In any of the above embodiments, in some cases, the immunomodulatory polypeptide is a FasL polypeptide. In some cases, the epitope is an auto-epitope (an epitope of a self antigen).

In some cases, a TMAPP of the present disclosure comprises: a) a first polypeptide comprising, in order from N-terminus to C-terminus: i) a peptide antigen (an “epitope”) that is recognized (e.g., is capable of being recognized and bound) by a TCR; ii) an MHC Class II α1 polypeptide; iii) an MHC Class II α2 polypeptide; and iv) an immunoglobulin or non-immunoglobulin scaffold polypeptide; and b) a second polypeptide comprising, in order from N-terminus to C-terminus: i) an immunomodulatory polypeptide; ii) an MHC Class II β1 polypeptide; and iii) an MHC Class II β2 polypeptide. In some cases, a TMAPP of the present disclosure comprises: a) a first polypeptide comprising, in order from N-terminus to C-terminus: i) an immunomodulatory polypeptide; ii) an MHC Class II α1 polypeptide; and iii) an MHC Class II α2 polypeptide; and iv) an immunoglobulin or non-immunoglobulin scaffold polypeptide; and b) a second polypeptide comprising, in order from N-terminus to C-terminus: i) a peptide antigen (an “epitope”) that is recognized (e.g., is capable of being recognized and bound) by a TCR; ii) an MHC Class II β1 polypeptide; and iii) an MHC Class II β2 polypeptide. In some cases, a TMAPP of the present disclosure comprises: a) a first polypeptide comprising, in order from N-terminus to C-terminus: i) a peptide antigen (an “epitope”) that is recognized (e.g., is capable of being recognized and bound) by a TCR; ii) an MHC Class II α1 polypeptide; and iii) an MHC Class II α2 polypeptide; and b) a second polypeptide comprising, in order from N-terminus to C-terminus: i) an immunomodulatory polypeptide; ii) an MHC Class II β1 polypeptide; iii) an MHC Class II β2 polypeptide; and iv) an immunoglobulin or non-immunoglobulin scaffold polypeptide. In some cases, a TMAPP of the present disclosure comprises: a) a first polypeptide comprising, in order from N-terminus to C-terminus: i) an immunomodulatory polypeptide; ii) an MHC Class II α1 polypeptide; and iii) an MHC Class II α2 polypeptide; and b) a second polypeptide comprising, in order from N-terminus to C-terminus: i) a peptide antigen (an “epitope”) that is recognized (e.g., is capable of being recognized and bound) by a TCR; ii) an MHC Class II β1 polypeptide; iii) an MHC Class II β2 polypeptide; and iv) an immunoglobulin or non-immunoglobulin scaffold polypeptide. In some cases, a TMAPP of the present disclosure comprises: a) a first polypeptide comprising, in order from N-terminus to C-terminus: i) a peptide antigen (an “epitope”) that is recognized (e.g., is capable of being recognized and bound) by a TCR; ii) an MHC Class II α1 polypeptide; iii) an MHC Class II α2 polypeptide; iv) an immunoglobulin or non-immunoglobulin scaffold polypeptide; and v) a first member of a dimerizer pair (e.g., a first leucine zipper polypeptide); and b) a second polypeptide comprising, in order from N-terminus to C-terminus: i) an immunomodulatory polypeptide; ii) an MHC Class II β1 polypeptide; iii) an MHC Class II β2 polypeptide; and iv) a second member of a dimerizer pair (e.g., a second leucine zipper polypeptide). In some cases, a TMAPP of the present disclosure comprises: a) a first polypeptide comprising, in order from N-terminus to C-terminus: i) an immunomodulatory polypeptide; ii) an MHC Class II α1 polypeptide; iii) an MHC Class II α2 polypeptide; iv) an immunoglobulin or non-immunoglobulin scaffold polypeptide; and v) and v) a first member of a dimerizer pair (e.g., a first leucine zipper polypeptide); and b) a second polypeptide comprising, in order from N-terminus to C-terminus: i) a peptide antigen (an “epitope”) that is recognized (e.g., is capable of being recognized and bound) by a TCR; ii) an MHC Class II β1 polypeptide; iii) an MHC Class II β2 polypeptide; and iv) a second member of a dimerizer pair (e.g., a second leucine zipper polypeptide). In some cases, a TMAPP of the present disclosure comprises: a) a first polypeptide comprising, in order from N-terminus to C-terminus: i) a peptide antigen (an “epitope”) that is recognized (e.g., is capable of being recognized and bound) by a TCR; ii) an MHC Class II α1 polypeptide; iii) an MHC Class II α2 polypeptide; and iv) a first member of a dimerizer pair (e.g., a first leucine zipper polypeptide); and b) a second polypeptide comprising, in order from N-terminus to C-terminus: i) an immunomodulatory polypeptide; ii) an MHC Class II β1 polypeptide; iii) an MHC Class II β2 polypeptide; iv) an immunoglobulin or non-immunoglobulin scaffold polypeptide; and v) a second member of a dimerizer pair (e.g., a second leucine zipper polypeptide). In some cases, a TMAPP of the present disclosure comprises: a) a first polypeptide comprising, in order from N-terminus to C-terminus: i) an immunomodulatory polypeptide; ii) an MHC Class II α1 polypeptide; iii) an MHC Class II α2 polypeptide; and iv) a first member of a dimerizer pair (e.g., a first leucine zipper polypeptide); and b) a second polypeptide comprising, in order from N-terminus to C-terminus: i) a peptide antigen (an “epitope”) that is recognized (e.g., is capable of being recognized and bound) by a TCR; ii) an MHC Class II β1 polypeptide; iii) an MHC Class II β2 polypeptide; iv) an immunoglobulin or non-immunoglobulin scaffold polypeptide; and v) a second member of a dimerizer pair (e.g., a second leucine zipper polypeptide). In any one of the above embodiments, the TMAPP can include 2 copies of the immunomodulatory polypeptide; the 2 copies can be in tandem, or can be separated by a linker. In any one of the above embodiments, the TMAPP can include 3 copies of the immunomodulatory polypeptide; the 3 copies can be in tandem, or can be separated by a linker. In some cases, the TMAPP comprises a linker (an “L1”) between the MHC polypeptide and the Ig Fc polypeptide; where exemplary suitable linkers include (GGGGS)n (SEQ ID NO: 1), where n is 1, 2, 3, 4, 5, 6, 7, or 8. In some cases, the TMAPP comprises a linker (an “L2”) between the immunomodulatory polypeptide and the MHC polypeptide, where exemplary suitable linkers include (GGGGS)n (SEQ ID NO: 1), where n is 1, 2, 3, 4, 5, 6, 7, or 8. In some cases, where the TMAPP comprises two immunomodulatory polypeptides, in some cases, the two immunomodulatory polypeptides are separated by a linker (an “L3); where exemplary suitable linkers include (GGGGS)n (SEQ ID NO: 1), where n is 1, 2, 3, 4, 5, 6, 7, or 8. In some cases, the linker between any two components of the TMAPP comprises the amino acid sequence GGSAAAGG (SEQ ID NO: 2). In any of the above embodiments, in some cases, the Ig Fc is an IgG1 Fc polypeptide. In any of the above embodiments, in some cases, the Ig Fc is an IgG4 Fc polypeptide. In any of the above embodiments, in some cases, the immunomodulatory polypeptide is a PD-L1 polypeptide. In any of the above embodiments, in some cases, the immunomodulatory polypeptide is a TGF-β polypeptide. In any of the above embodiments, in some cases, the immunomodulatory polypeptide is a FasL polypeptide. In some cases, the epitope is an auto-epitope (an epitope of a self antigen).

In some cases, a TMAPP of the present disclosure comprises: a) a first polypeptide comprising, in order from N-terminus to C-terminus: i) a peptide antigen (an “epitope”) that is recognized (e.g., is capable of being recognized and bound) by a TCR; ii) an MHC Class II β1 polypeptide; iii) an MHC Class II β2 polypeptide; and iv) an immunomodulatory polypeptide; and b) a second polypeptide comprising, in order from N-terminus to C-terminus: i) an MHC Class II α1 polypeptide; and ii) an MHC Class II α2 polypeptide. In some cases, a TMAPP of the present disclosure comprises: a) a first polypeptide comprising, in order from N-terminus to C-terminus: i) a peptide antigen (an “epitope”) that is recognized (e.g., is capable of being recognized and bound) by a TCR; ii) an MHC Class II β1 polypeptide; iii) an MHC Class II β2 polypeptide; and iv) an immunomodulatory polypeptide; and b) a second polypeptide comprising, in order from N-terminus to C-terminus: i) an MHC Class II α1 polypeptide; ii) an MHC Class II α2 polypeptide; and iii) an immunoglobulin or non-immunoglobulin scaffold polypeptide. In some cases, a TMAPP of the present disclosure comprises: a) a first polypeptide comprising, in order from N-terminus to C-terminus: i) a peptide antigen (an “epitope”) that is recognized (e.g., is capable of being recognized and bound) by a TCR; ii) an MHC Class II β1 polypeptide; iii) an MHC Class II β2 polypeptide; and iv) an immunomodulatory polypeptide; and b) a second polypeptide comprising, in order from N-terminus to C-terminus: i) an MHC Class II α1 polypeptide; ii) an MHC Class II α2 polypeptide; and iii) an Ig Fc polypeptide. In some cases, a TMAPP of the present disclosure comprises: a) a first polypeptide comprising, in order from N-terminus to C-terminus: i) a peptide antigen (an “epitope”) that is recognized (e.g., is capable of being recognized and bound) by a TCR; ii) an MHC Class II β1 polypeptide; iii) an MHC Class II β2 polypeptide; iv) an immunomodulatory polypeptide; and v) a first member of a dimerizer pair; and b) a second polypeptide comprising, in order from N-terminus to C-terminus: i) an MHC Class II α1 polypeptide; ii) an MHC Class II α2 polypeptide; and iii) a second member of the dimerizer pair. In some cases, a TMAPP of the present disclosure comprises: a) a first polypeptide comprising, in order from N-terminus to C-terminus: i) a peptide antigen (an “epitope”) that is recognized (e.g., is capable of being recognized and bound) by a TCR; ii) an MHC Class II β1 polypeptide; iii) an MHC Class II β2 polypeptide; iv) an immunomodulatory polypeptide; and v) a first leucine zipper polypeptide; and b) a second polypeptide comprising, in order from N-terminus to C-terminus: i) an MHC Class II α1 polypeptide; ii) an MHC Class II α2 polypeptide; and iii) a second leucine zipper polypeptide. In any one of the above embodiments, the TMAPP can include a single immunomodulatory polypeptide. In any one of the above embodiments, the TMAPP can include 2 copies of the immunomodulatory polypeptide; the 2 copies can be in tandem, or can be separated by a linker. In any one of the above embodiments, the TMAPP can include 3 copies of the immunomodulatory polypeptide; the 3 copies can be in tandem, or can be separated by a linker. In some cases, the TMAPP comprises a linker (an “L1”) between the MHC polypeptide and the Ig Fc polypeptide; where exemplary suitable linkers include (GGGGS)n (SEQ ID NO: 1), where n is 1, 2, 3, 4, 5, 6, 7, or 8. In some cases, the TMAPP comprises a linker (an “L2”) between the immunomodulatory polypeptide and the MHC polypeptide, where exemplary suitable linkers include (GGGGS)n (SEQ ID NO: 1), where n is 1, 2, 3, 4, 5, 6, 7, or 8. In some cases, where the TMAPP comprises two immunomodulatory polypeptides, in some cases, the two immunomodulatory polypeptides are separated by a linker (an “L3); where exemplary suitable linkers include (GGGGS)n (SEQ ID NO: 1), where n is 1, 2, 3, 4, 5, 6, 7, or 8. In some cases, the linker between any two components of the TMAPP comprises the amino acid sequence GGSAAAGG (SEQ ID NO: 2). In any of the above embodiments, in some cases, the Ig Fc is an IgG1 Fc polypeptide. In any of the above embodiments, in some cases, the Ig Fc is an IgG4 Fc polypeptide. In any of the above embodiments, in some cases, the immunomodulatory polypeptide is a PD-L1 polypeptide. In any of the above embodiments, in some cases, the immunomodulatory polypeptide is a TGF-β polypeptide. In any of the above embodiments, in some cases, the immunomodulatory polypeptide is a FasL polypeptide. In some cases, the epitope is an auto-epitope (an epitope of a self antigen).

In some cases, a TMAPP of the present disclosure comprises: a) a first polypeptide comprising, in order from N-terminus to C-terminus: i) a peptide antigen (an “epitope”) that is recognized (e.g., is capable of being recognized and bound) by a TCR; ii) an MHC Class II β1 polypeptide; and iii) an MHC Class II β2 polypeptide; and b) a second polypeptide comprising, in order from N-terminus to C-terminus: i) an immunomodulatory polypeptide; ii) an MHC Class II α1 polypeptide; and iii) an MHC Class II α2 polypeptide. In some cases, a TMAPP of the present disclosure comprises: a) a first polypeptide comprising, in order from N-terminus to C-terminus: i) a peptide antigen (an “epitope”) that is recognized (e.g., is capable of being recognized and bound) by a TCR; ii) an MHC Class II β1 polypeptide; and iii) an MHC Class II β2 polypeptide; and b) a second polypeptide comprising, in order from N-terminus to C-terminus: i) an immunomodulatory polypeptide; ii) an MHC Class II α1 polypeptide; iii) an MHC Class II α2 polypeptide; and iv) an immunoglobulin or non-immunoglobulin scaffold polypeptide. In some cases, a TMAPP of the present disclosure comprises: a) a first polypeptide comprising, in order from N-terminus to C-terminus: i) a peptide antigen (an “epitope”) that is recognized (e.g., is capable of being recognized and bound) by a TCR; ii) an MHC Class II β1 polypeptide; and iii) an MHC Class II β2 polypeptide; and b) a second polypeptide comprising, in order from N-terminus to C-terminus: i) an immunomodulatory polypeptide; ii) an MHC Class II α1 polypeptide; iii) an MHC Class II α2 polypeptide; and iv) an Ig Fc polypeptide. In some cases, a TMAPP of the present disclosure comprises: a) a first polypeptide comprising, in order from N-terminus to C-terminus: i) a peptide antigen (an “epitope”) that is recognized (e.g., is capable of being recognized and bound) by a TCR; ii) an MHC Class II β1 polypeptide; iii) an MHC Class II β2 polypeptide; and iv) a first member of a dimerizer pair; and b) a second polypeptide comprising, in order from N-terminus to C-terminus: i) an immunomodulatory polypeptide; ii) an MHC Class II α1 polypeptide; iii) an MHC Class II α2 polypeptide; and iv) a second member of the dimerizer pair. In some cases, a TMAPP of the present disclosure comprises: a) a first polypeptide comprising, in order from N-terminus to C-terminus: i) a peptide antigen (an “epitope”) that is recognized (e.g., is capable of being recognized and bound) by a TCR; ii) an MHC Class II β1 polypeptide; iii) an MHC Class II β2 polypeptide; and iv) a first leucine zipper polypeptide; and b) a second polypeptide comprising, in order from N-terminus to C-terminus: i) an immunomodulatory polypeptide; ii) an MHC Class II α1 polypeptide; iii) an MHC Class II α2 polypeptide; and iv) a second leucine zipper polypeptide. In any one of the above embodiments, the TMAPP can include a single immunomodulatory polypeptide. In any one of the above embodiments, the TMAPP can include 2 copies of the immunomodulatory polypeptide; the 2 copies can be in tandem, or can be separated by a linker. In any one of the above embodiments, the TMAPP can include 3 copies of the immunomodulatory polypeptide; the 3 copies can be in tandem, or can be separated by a linker. In some cases, the TMAPP comprises a linker (an “L1”) between the MHC polypeptide and the Ig Fc polypeptide; where exemplary suitable linkers include (GGGGS)n (SEQ ID NO: 1), where n is 1, 2, 3, 4, 5, 6, 7, or 8. In some cases, the TMAPP comprises a linker (an “L2”) between the immunomodulatory polypeptide and the MHC polypeptide, where exemplary suitable linkers include (GGGGS)n (SEQ ID NO: 1), where n is 1, 2, 3, 4, 5, 6, 7, or 8. In some cases, where the TMAPP comprises two immunomodulatory polypeptides, in some cases, the two immunomodulatory polypeptides are separated by a linker (an “L3); where exemplary suitable linkers include (GGGGS)n (SEQ ID NO: 1), where n is 1, 2, 3, 4, 5, 6, 7, or 8. In some cases, the linker between any two components of the TMAPP comprises the amino acid sequence GGSAAAGG (SEQ ID NO: 2). In any of the above embodiments, in some cases, the Ig Fc is an IgG1 Fc polypeptide. In any of the above embodiments, in some cases, the Ig Fc is an IgG4 Fc polypeptide. In any of the above embodiments, in some cases, the immunomodulatory polypeptide is a PD-L1 polypeptide. In any of the above embodiments, in some cases, the immunomodulatory polypeptide is a TGF-β polypeptide. In any of the above embodiments, in some cases, the immunomodulatory polypeptide is a FasL polypeptide. In some cases, the epitope is an auto-epitope (an epitope of a self antigen).

In some cases, a TMAPP of the present disclosure comprises: a) a first polypeptide comprising, in order from N-terminus to C-terminus: i) a peptide antigen (an “epitope”) that is recognized (e.g., is capable of being recognized and bound) by a TCR; ii) an MHC Class II β1 polypeptide; iii) an MHC Class II α1 polypeptide; and iv) an MHC Class II α2 polypeptide; and b) a second polypeptide comprising, in order from N-terminus to C-terminus: i) an immunomodulatory polypeptide; and ii) an MHC Class II β2 polypeptide. In some cases, a TMAPP of the present disclosure comprises: a) a first polypeptide comprising, in order from N-terminus to C-terminus: i) a peptide antigen (an “epitope”) that is recognized (e.g., is capable of being recognized and bound) by a TCR; ii) an MHC Class II β1 polypeptide; iii) an MHC Class II α1 polypeptide; iv) an MHC Class II α2 polypeptide; and v) an immunoglobulin or non-immunoglobulin scaffold polypeptide; and b) a second polypeptide comprising, in order from N-terminus to C-terminus: i) an immunomodulatory polypeptide; and ii) an MHC Class II β2 polypeptide. In some cases, a TMAPP of the present disclosure comprises: a) a first polypeptide comprising, in order from N-terminus to C-terminus: i) a peptide antigen (an “epitope”) that is recognized (e.g., is capable of being recognized and bound) by a TCR; ii) an MHC Class II β1 polypeptide; iii) an MHC Class II α1 polypeptide; iv) an MHC Class II α2 polypeptide; and v) an Ig Fc polypeptide; and b) a second polypeptide comprising, in order from N-terminus to C-terminus: i) an immunomodulatory polypeptide; and ii) an MHC Class II β2 polypeptide. In some cases, a TMAPP of the present disclosure comprises: a) a first polypeptide comprising, in order from N-terminus to C-terminus: i) a peptide antigen (an “epitope”) that is recognized (e.g., is capable of being recognized and bound) by a TCR; ii) an MHC Class II β1 polypeptide; iii) an MHC Class II α1 polypeptide; iv) an MHC Class II α2 polypeptide; and v) a first member of a dimerizer pair; and b) a second polypeptide comprising, in order from N-terminus to C-terminus: i) an immunomodulatory polypeptide; ii) an MHC Class II β2 polypeptide; and iii) a second member of the dimerizer pair. In some cases, a TMAPP of the present disclosure comprises: a) a first polypeptide comprising, in order from N-terminus to C-terminus: i) a peptide antigen (an “epitope”) that is recognized (e.g., is capable of being recognized and bound) by a TCR; ii) an MHC Class II β1 polypeptide; iii) an MHC Class II α1 polypeptide; iv) an MHC Class II α2 polypeptide; and v) a first leucine zipper polypeptide; and b) a second polypeptide comprising, in order from N-terminus to C-terminus: i) an immunomodulatory polypeptide; ii) an MHC Class II β2 polypeptide; and iii) a second leucine zipper polypeptide. In any one of the above embodiments, the TMAPP can include a single immunomodulatory polypeptide. In any one of the above embodiments, the TMAPP can include 2 copies of the immunomodulatory polypeptide; the 2 copies can be in tandem, or can be separated by a linker. In any one of the above embodiments, the TMAPP can include 3 copies of the immunomodulatory polypeptide; the 3 copies can be in tandem, or can be separated by a linker. In some cases, the TMAPP comprises a linker (an “L1”) between the MHC polypeptide and the Ig Fc polypeptide; where exemplary suitable linkers include (GGGGS)n (SEQ ID NO: 1), where n is 1, 2, 3, 4, 5, 6, 7, or 8. In some cases, the TMAPP comprises a linker (an “L2”) between the immunomodulatory polypeptide and the MHC polypeptide, where exemplary suitable linkers include (GGGGS)n (SEQ ID NO: 1), where n is 1, 2, 3, 4, 5, 6, 7, or 8. In some cases, where the TMAPP comprises two immunomodulatory polypeptides, in some cases, the two immunomodulatory polypeptides are separated by a linker (an “L3); where exemplary suitable linkers include (GGGGS)n (SEQ ID NO: 1), where n is 1, 2, 3, 4, 5, 6, 7, or 8. In some cases, the linker between any two components of the TMAPP comprises the amino acid sequence GGSAAAGG (SEQ ID NO: 2). In any of the above embodiments, in some cases, the Ig Fc is an IgG1 Fc polypeptide. In any of the above embodiments, in some cases, the Ig Fc is an IgG4 Fc polypeptide. In any of the above embodiments, in some cases, the immunomodulatory polypeptide is a PD-L1 polypeptide. In any of the above embodiments, in some cases, the immunomodulatory polypeptide is a TGF-β polypeptide. In any of the above embodiments, in some cases, the immunomodulatory polypeptide is a FasL polypeptide. In some cases, the epitope is an auto-epitope (an epitope of a self antigen).

### Single-Chain T-Cell Modulatory Antigen-Presenting Polypeptides

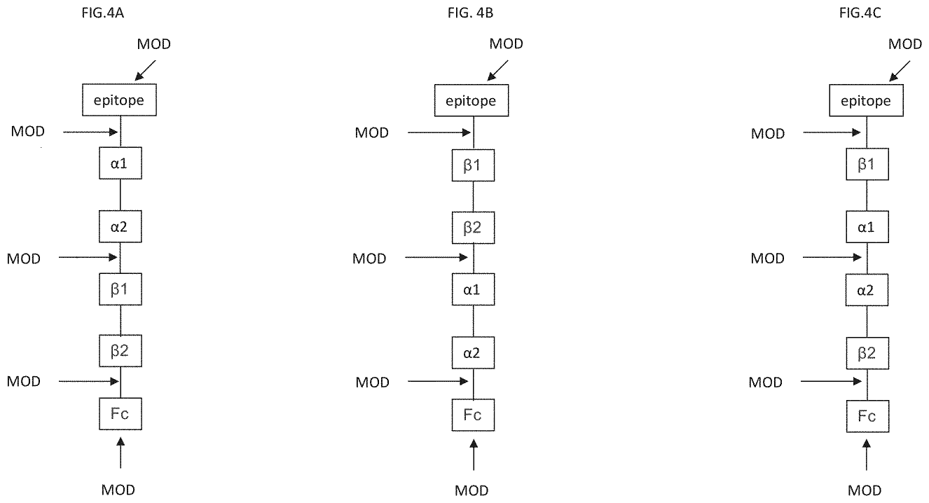

As noted above, in some cases, a TMAPP of the present disclosure is a single-chain (a single polypeptide chain) TMAPP. A single-chain TMAPP of the present disclosure comprises: i) a peptide antigen (an “epitope”) that is recognized (e.g., is capable of being recognized and bound) by a TCR; ii) an MHC Class II β1 polypeptide; iii) an MHC Class II β2 polypeptide; iv) an MHC Class II α1 polypeptide; v) an MHC Class II α2 polypeptide; and vi) at least one immunomodulatory polypeptide. A single-chain TMAPP of the present disclosure can also include an Ig Fc polypeptide. A single-chain TMAPP of the present disclosure can comprise two or more immunomodulatory polypeptides, where the two or more immunomodulatory polypeptides can have the same amino acid sequence or different amino acid sequences. The arrangement of the components, including the placement of the immunomodulatory polypeptide, of a single-chain TMAPP of the present disclosure can vary. Non-limiting examples are depicted in FIG. 4A-4C. For example, in some cases, a single-chain TMAPP of the present disclosure can comprise, in order from N-terminus to C-terminus: i) a peptide antigen (an “epitope”) that is recognized (e.g., is capable of being recognized and bound) by a TCR; ii) an MHC Class II α1 polypeptide; iii) an MHC Class II α2 polypeptide; iv) an MHC Class II β1 polypeptide; v) an MHC Class II β2 polypeptide; and vi) an Ig Fc polypeptide, where the immunomodulatory polypeptide of the single-chain TMAPP is located at one or more of: i) at the N-terminus (N-terminal to the peptide antigen); ii) between the peptide antigen (“epitope”) and the MHC Class II α1 polypeptide; iii) between the MHC Class II α2 polypeptide and the MHC Class II β1 polypeptide; iv) between the MHC Class II β2 polypeptide and the Ig Fc polypeptide; and v) C-terminal to the Ig Fc polypeptide. Such arrangements are depicted schematically in FIG. 4A. As another example, in some cases, a single-chain TMAPP of the present disclosure can comprise, in order from N-terminus to C-terminus: i) a peptide antigen (an “epitope”) that is recognized (e.g., is capable of being recognized and bound) by a TCR; ii) an MHC Class II β1 polypeptide; iii) an MHC Class II β2 polypeptide; iv) an MHC Class II α1 polypeptide; v) an MHC Class II α2 polypeptide; and vi) an Ig Fc polypeptide, where the immunomodulatory polypeptide of the single-chain TMAPP is located at one or more of: i) at the N-terminus (N-terminal to the peptide antigen); ii) between the peptide antigen (“epitope”) and the MHC Class II β1 polypeptide; iii) between the MHC Class II β2 polypeptide and the MHC Class II α1 polypeptide; iv) between the MHC Class II α2 polypeptide and the Ig Fc polypeptide; and v) C-terminal to the Ig Fc polypeptide. Such arrangements are depicted schematically in FIG. 4B. As another example, in some cases, a single-chain TMAPP of the present disclosure can comprise, in order from N-terminus to C-terminus: i) a peptide antigen (an “epitope”) that is recognized (e.g., is capable of being recognized and bound) by a TCR; ii) an MHC Class II β1 polypeptide; iii) an MHC Class II α1 polypeptide; iv) an MHC Class II α2 polypeptide; v) an MHC Class II β2 polypeptide; and vi) an Ig Fc polypeptide, where the immunomodulatory polypeptide of the single-chain TMAPP is located at one or more of: i) at the N-terminus (N-terminal to the peptide antigen); ii) between the peptide antigen (“epitope”) and the MHC Class II β1 polypeptide; iii) between the MHC Class II α1 polypeptide and the MHC Class II α2 polypeptide; iv) between the MHC Class II β2 polypeptide and the Ig Fc polypeptide; and v) v) C-terminal to the Ig Fc polypeptide. Such arrangements are depicted schematically in FIG. 4C. In any one of the above embodiments, the TMAPP can include a single immunomodulatory polypeptide. In any one of the above embodiments, the TMAPP can include 2 copies of the immunomodulatory polypeptide; the 2 copies can be in tandem, or can be separated by a linker. In any one of the above embodiments, the TMAPP can include 3 copies of the immunomodulatory polypeptide; the 3 copies can be in tandem, or can be separated by a linker. In some cases, the TMAPP comprises a linker (an “L1”) between the MHC polypeptide and the Ig Fc polypeptide; where exemplary suitable linkers include (GGGGS)n (SEQ ID NO: 1), where n is 1, 2, 3, 4, 5, 6, 7, or 8. In some cases, the TMAPP comprises a linker (an “L2”) between the immunomodulatory polypeptide and the MHC polypeptide, where exemplary suitable linkers include (GGGGS)n (SEQ ID NO: 1), where n is 1, 2, 3, 4, 5, 6, 7, or 8. In some cases, where the TMAPP comprises two immunomodulatory polypeptides, in some cases, the two immunomodulatory polypeptides are separated by a linker (an “L3); where exemplary suitable linkers include (GGGGS)n (SEQ ID NO: 1), where n is 1, 2, 3, 4, 5, 6, 7, or 8. In some cases, the linker between any two components of the TMAPP comprises the amino acid sequence GGSAAAGG (SEQ ID NO: 2). In any of the above embodiments, in some cases, the Ig Fc is an IgG1 Fc polypeptide. In any of the above embodiments, in some cases, the Ig Fc is an IgG4 Fc polypeptide. In any of the above embodiments, in some cases, the immunomodulatory polypeptide is a PD-L1 polypeptide. In any of the above embodiments, in some cases, the immunomodulatory polypeptide is a TGF-β polypeptide. In any of the above embodiments, in some cases, the immunomodulatory polypeptide is a FasL polypeptide. In some cases, the epitope is an auto-epitope (an epitope of a self antigen

### Class II MHC Polypeptides

As noted above, a TMAPP of the present disclosure comprises Class II MHC polypeptides.

Naturally occurring Class II MHC polypeptides comprise an α chain and a β chain. “Class II MHC polypeptides” include human leukocyte antigen (HLA) α- and β-chains. MHC Class II polypeptides include MHC Class II DP α and β polypeptides, DM α and β polypeptides, DOA α and β polypeptides, DOB α and β polypeptides, DQ α and β polypeptides, and DR α and β polypeptides. As used herein, a “Class II MHC polypeptide” can comprise a class II MHC α chain polypeptide, a class II MHC β chain polypeptide, or only a portion of a class II MHC α or β chain polypeptide. For example, a “Class II MHC polypeptide” can be a polypeptide that includes: i) only the α1 domain of a class II MHC α chain polypeptide; ii) only the α2 domain of a class II MHC α chain; iii) only the α1 domain and an α2 domain of a class II MHC α chain; iv) only the β1 domain of a class II MHC β chain; v) only the β2 domain of a class II MHC β chain; vi) only the β1 domain and the β2 domain of a class II MHC β chain; vii) the α1 domain of a class II MHC α chain, the β1 domain of a class II MHC β chain, and the β2 domain of a class II MHC; and the like.

Class II MHC polypeptides include allelic forms. The HLA locus is highly polymorphic in nature. As disclosed in the Nomenclature for Factors of the HLA System 2000 (Hum. Immunol.; 62(4):419-68, 2001) there are 221 HLA-DRB 1 alleles, 19 DRB3 alleles, 89 DRB4 alleles, 14 DRB5 alleles, 19 DQA1 alleles and 39 DQB1 alleles, with new alleles being discovered continuously. A 2007 update by the WHO nomenclature Committee for Factors of the HLA System (www.anthonynolan.com/HIG/) showed there are 3 DRA alleles, 494 DRB 1 alleles, 1 DRB2 alleles, 44 DRB3 alleles, 13 DRB4 alleles, 18 DRB5 alleles, 3 DRB6 alleles, 2 DRB7 alleles, 10 DRB8 alleles, 1 DRB9 alleles, 34 DQA1 alleles, 83 DQB1 alleles, 23 DPA1, 126 DPB1 alleles, 4 DMA alleles, 7 DMB alleles, 12 DOA alleles and 9 DOB alleles. As used herein, the term “Class II MHC polypeptide” includes allelic forms of any known Class II MHC polypeptide.

In some cases, a TMAPP of the present disclosure comprises a Class II MHC α chain, without the leader, transmembrane, and intracellular portions (e.g., cytoplasmic tails) that may be present in a naturally-occurring Class II MHC α chain. Thus, in some cases, a TMAPP of the present disclosure comprises only the α1 and α2 portions of a Class II MHC α chain; and does not include the leader, transmembrane, and intracellular portions (e.g., cytoplasmic tails) that may be present in a naturally-occurring Class II MHC α chain.

In some cases, a TMAPP of the present disclosure comprises a Class II MHC β chain, without the leader, transmembrane, and intracellular portions (e.g., cytoplasmic tails) that may be present in a naturally-occurring Class II MHC β chain. Thus, in some cases, a TMAPP of the present disclosure comprises only the β1 and β2 portions of a Class II MHC β chain; and does not include the leader, transmembrane, and intracellular portions (e.g., cytoplasmic tails) that may be present in a naturally-occurring Class II MHC β chain.

### MHC Class II Alpha Chains

MHC Class II alpha chains comprise an α1 domain and an α2 domain. In some cases, the α1 domain and the α2 domain present in an antigen-presenting cell are from the same MHC Class II α chain polypeptide. In some cases, the α1 domain and the α2 domain present in an antigen-presenting cell are from two different MHC Class II α chain polypeptides.

MHC Class II alpha chains suitable for inclusion in a TMAPP (e.g., a multimeric TMAPP; a single-chain TMAPP) of the present disclosure lack a signal peptide. An MHC Class II alpha chain suitable for inclusion in a multimeric polypeptide of the present disclosure can have a length of from about 60 amino acids to about 190 amino acids; for example, an MHC Class II alpha chain suitable for inclusion in a TMAPP of the present disclosure can have a length of from about 60 amino acids to about 80 amino acids, from about 80 amino acids to about 100 amino acids, from about 100 amino acids to about 120 amino acids, from about 120 amino acids to about 140 amino acids, from about 140 amino acids to about 160 amino acids, from about 160 amino acids to about 180 amino acids, or from about 180 amino acids to about 200 amino acids. An MHC Class II α1 domain suitable for inclusion in a TMAPP of the present disclosure can have a length of from about 30 amino acids to about 95 amino acids; for example, an MHC Class II α1 domain suitable for inclusion in a TMAPP of the present disclosure can have a length of from about 30 amino acids to about 40 amino acids, from about 40 amino acids to about 50 amino acids, from about 50 amino acids to about 60 amino acids, from about 60 amino acids to about 70 amino acids, from about 70 amino acids to about 80 amino acids, from about 80 amino acids to about 90 amino acids, or from about 90 amino acids to about 95 amino acids. An MHC Class II α2 domain suitable for inclusion in a TMAPP of the present disclosure can have a length of from about 30 amino acids to about 95 amino acids; for example, an MHC Class II α2 domain suitable for inclusion in a TMAPP of the present disclosure can have a length of from about 30 amino acids to about 40 amino acids, from about 40 amino acids to about 50 amino acids, from about 50 amino acids to about 60 amino acids, from about 60 amino acids to about 70 amino acids, from about 70 amino acids to about 80 amino acids, from about 80 amino acids to about 90 amino acids, or from about 90 amino acids to about 95 amino acids.

### DRA

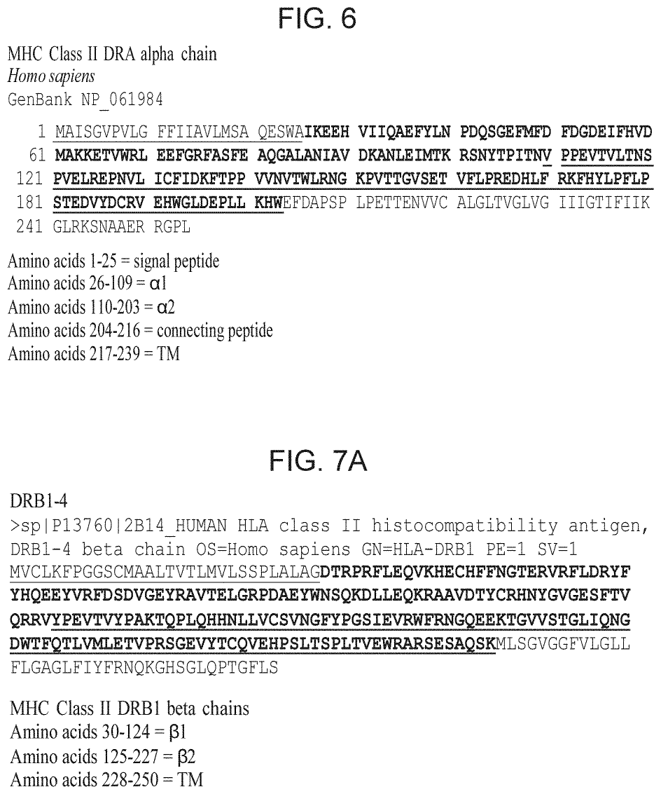

In some cases, a suitable MHC Class II α chain polypeptide is a DRA polypeptide. A DRA polypeptide can have at least 60%, at least 70%, at least 80%, at least 90%, at least 95%, at least 98%, at least 99%, or 100%, amino acid sequence identity with amino acids 26-203 of the DRA amino acid sequence depicted in FIG. 6. In some cases, the DRA polypeptide has a length of about 178 amino acids (e.g., 175, 176, 177, 178, 179, or 180 amino acids).

A “DRA polypeptide” includes allelic variants, e.g., naturally occurring allelic variants. Thus, in some cases, a suitable DRA polypeptide comprises the following amino acid sequence: IKEEH VIIQAEFYLN PDQSGEFMFD FDGDEIFHVD MAKKETVWRL EEFGRFASFE AQGALANIAV DKANLEIMTK RSNYTPITNV PPEVTVLTNSPVELREPNVL ICFIDKFTPP VVNVTWLRNG KPVTTGVSET VFLPREDHLF RKFHYLPFLPSTEDVYDCRV EHWGLDEPLL KHW (SEQ ID NO: 5, amino acids 26-203 of DRA*01:02:01, see FIG. 6), or an allelic variant thereof. In some cases, the allelic variant is the DRA*01:01:01:01 allelic variant that differs from DRA*01:02:01 by having a valine in place of the leucine at position 242 of the sequence in FIG. 6.

A suitable DRA α1 domain comprises an amino acid sequence having at least 60%, at least 70%, at least 80%, at least 90%, at least 95%, at least 98%, at least 99%, or 100%, amino acid sequence identity to the following amino acid sequence: VIIQAEFYLN PDQSGEFMFD FDGDEIFHVD MAKKETVWRL EEFGRFASFE AQGALANIAV DKANLEIMTK RSNYTPITN (SEQ ID NO: 6); and can have a length of about 84 amino acids (e.g., 80, 81, 82, 83, 84, 85, or 86 amino acids). A suitable DRA α1 domain can comprise the following amino acid sequence: VIIQAEFYLN PDQSGEFMFD FDGDEIFHVD MAKKETVWRL EEFGRFASFE AQGALANIAV DKANLEIMTK RSNYTPITN (SEQ ID NO: 6), or a naturally-occurring allelic variant.

A suitable DRA α2 domain comprises an amino acid sequence having at least 60%, at least 70%, at least 80%, at least 90%, at least 95%, at least 98%, at least 99%, or 100%, amino acid sequence identity to the following amino acid sequence: V PPEVTVLTNSPVELREPNVL ICFIDKFTPP VVNVTWLRNG KPVTTGVSET VFLPREDHLF RKFHYLPFLPSTEDVYDCRV EHWGLDEPLL KHW (SEQ ID NO: 7); and can have a length of about 94 amino acids (e.g., 90, 91, 92, 93, 94, 95, 96, 97, or 98 amino acids).

### DMA

In some cases, a suitable MHC Class II α chain polypeptide is a DMA polypeptide. A DMA polypeptide can have at least 60%, at least 70%, at least 80%, at least 90%, at least 95%, at least 98%, at least 99%, or 100%, amino acid sequence identity with amino acids 27-217 of the DMA amino acid sequence depicted in FIG. 11. In some cases, the DMA polypeptide has a length of about 191 amino acids (e.g., 188, 189, 190, 191, 192, or 193 amino acids).

A “DMAA polypeptide” includes allelic variants, e.g., naturally occurring allelic variants. Thus, in some cases, a suitable DMAA polypeptide comprises the following amino acid sequence: VPEA PTPMWPDDLQ NHTFLHTVYC QDGSPSVGLS EAYDEDQLFF FDFSQNTRVP RLPEFADWAQ EQGDAPAILF DKEFCEWMIQ QIGPKLDGKI PVSRGFPIAE VFTLKPLEFG KPNTLVCFVS NLFPPMLTVN WQHHSVPVEG FGPTFVSAVD GLSFQAFSYL NFTPEPSDIF SCIVTHEIDR YTAIAYW (SEQ ID NO: 8 amino acids 27-217 of DMA*01:01:01, see FIG. 11), or an allelic variant thereof.

A suitable DMA α1 domain comprises an amino acid sequence having at least 60%, at least 70%, at least 80%, at least 90%, at least 95%, at least 98%, at least 99%, or 100%, amino acid sequence identity to the following amino acid sequence: VPEA PTPMWPDDLQ NHTFLHTVYC QDGSPSVGLS EAYDEDQLFF FDFSQNTRVP RLPEFADWAQ EQGDAPAILF DKEFCEWMIQ QIGPKLDGKI PVSR (SEQ ID NO: 9); and can have a length of about 98 amino acids (e.g., 94, 95, 96, 97, 98, 99, 100, or 101 amino acids). A suitable DMA α1 domain can comprise the following amino acid sequence: VPEA PTPMWPDDLQ NHTFLHTVYC QDGSPSVGLS EAYDEDQLFF FDFSQNTRVP RLPEFADWAQ EQGDAPAILF DKEFCEWMIQ QIGPKLDGKI PVSR (SEQ ID NO: 9), or a naturally-occurring allelic variant thereof.

A suitable DMA α2 domain comprises an amino acid sequence having at least 60%, at least 70%, at least 80%, at least 90%, at least 95%, at least 98%, at least 99%, or 100%, amino acid sequence identity to the following amino acid sequence: GFPIAE VFTLKPLEFG KPNTLVCFVS NLFPPMLTVN WQHHSVPVEG FGPTFVSAVD GLSFQAFSYL NFTPEPSDIF SCIVTHEIDR YTAIAYW (SEQ ID NO: 10); and can have a length of about 93 amino acids (e.g., 90, 91, 92, 93, 94, 95, 96, or 97 amino acids). A suitable DMA α2 domain can comprise the following amino acid sequence: GFPIAE VFTLKPLEFG KPNTLVCFVS NLFPPMLTVN WQHHSVPVEG FGPTFVSAVD GLSFQAFSYL NFTPEPSDIF SCIVTHEIDR YTAIAYW (SEQ ID NO: 10), or a naturally-occurring allelic variant thereof.

### DOA

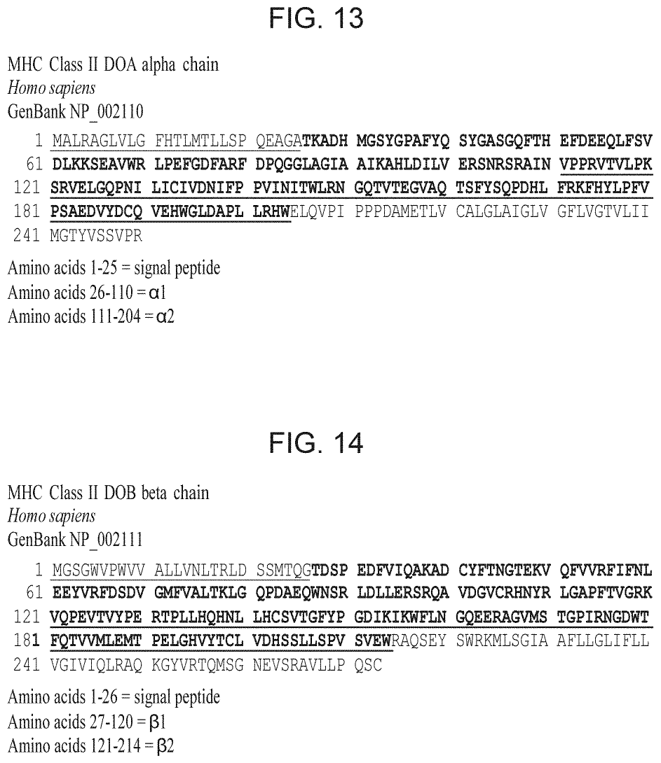

In some cases, a suitable MHC Class II α chain polypeptide is a DOA polypeptide. A DOA polypeptide can have at least 60%, at least 70%, at least 80%, at least 90%, at least 95%, at least 98%, at least 99%, or 100%, amino acid sequence identity with amino acids 26-204 of the DOA amino acid sequence depicted in FIG. 13. In some cases, the DOA polypeptide has a length of about 179 amino acids (e.g., 175, 176, 177, 178, 179, 180, 181, or 182 amino acids).

A “DOA polypeptide” includes allelic variants, e.g., naturally occurring allelic variants. Thus, in some cases, a suitable DOA polypeptide comprises the following amino acid sequence: TKADH MGSYGPAFYQ SYGASGQFTH EFDEEQLFSV DLKKSEAVWR LPEFGDFARF DPQGGLAGIA AIKAHLDILV ERSNRSRAIN VPPRVTVLPK SRVELGQPNI LICIVDNIFP PVINITWLRN GQTVTEGVAQ TSFYSQPDHL FRKFHYLPFV PSAEDVYDCQ VEHWGLDAPL LRHW (SEQ ID NO: 11; amino acids 26-204 of DOA*01:01:01:01, see FIG. 13), or an allelic variant thereof. In some cases, the allelic variant may be the DOA*01:02 by having an arginine in place of the cysteine (R80C) at position 80 or the DOA*01:03 variant having a valine in place of the leucine at position 74 (L74V) relative to DOA*01:01:01:01.

A suitable DOA α1 domain comprises an amino acid sequence having at least 60%, at least 70%, at least 80%, at least 90%, at least 95%, at least 98%, at least 99%, or 100%, amino acid sequence identity to the following amino acid sequence: TKADH MGSYGPAFYQ SYGASGQFTH EFDEEQLFSV DLKKSEAVWR LPEFGDFARF DPQGGLAGIA AIKAHLDILV ERSNRSRAIN (SEQ ID NO: 12); and can have a length of about 85 amino acids (e.g., 83, 84, 85, 86, 87, or 88 amino acids). Suitable α1 domain sequence may incorporate the L74V and/or R80C substitutions found in DOA*01:02 and DOA*01:03 (the amino acids corresponding to L74 and R 80 are shown italicized and bolded). A suitable DOA α1 domain can comprise the following amino acid sequence: TKADH MGSYGPAFYQ SYGASGQFTH EFDEEQLFSV DLKKSEAVWR LPEFGDFARF DPQGGLAGIA AIKAHLDILV ERSNRSRAIN (SEQ ID NO: 12), or a naturally-occurring allelic variant.

A suitable DOA α2 domain comprises an amino acid sequence having at least 60%, at least 70%, at least 80%, at least 90%, at least 95%, at least 98%, at least 99%, or 100%, amino acid sequence identity to the following amino acid sequence: VPPRVTVLPK SRVELGQPNI LICIVDNIFP PVINITWLRN GQTVTEGVAQ TSFYSQPDHL FRKFHYLPFV PSAEDVYDCQ VEHWGLDAPL LRHW (SEQ ID NO: 13); and can have a length of about 94 amino acids (e.g., 91, 92, 93, 94, 95, 96, or 97 amino acids). A suitable DOA α2 domain can comprise the following amino acid sequence: VPPRVTVLPK SRVELGQPNI LICIVDNIFP PVINITWLRN GQTVTEGVAQ TSFYSQPDHL FRKFHYLPFV PSAEDVYDCQ VEHWGLDAPL LRHW (SEQ ID NO: 13), or a naturally-occurring allelic variant thereof.

### DPA1

In some cases, a suitable MHC Class II α chain polypeptide is a DPA1 polypeptide. A DPA1 polypeptide can have at least 60%, at least 70%, at least 80%, at least 90%, at least 95%, at least 98%, at least 99%, or 100%, amino acid sequence identity with amino acids 29-209 of the DPA1 amino acid sequence depicted in FIG. 15. In some cases, the DPA1 polypeptide has a length of about 181 amino acids (e.g., 178, 179, 180, 181, 182, 183, or 184 amino acids).

A “DPA1 polypeptide” includes allelic variants, e.g., naturally occurring allelic variants. Thus, in some cases, a suitable DPA1 polypeptide comprises the following amino acid sequence: AG AIKADHVSTY AAFVQTHRPT GEFMFEFDED EMFYVDLDKK ETVWHLEEFG QAFSFEAQGG LANIAILNNN LNTLIQRSNH TQATNDPPEV TVFPKEPVEL GQPNTLICHI DKFFPPVLNV TWLCNGELVT EGVAESLFLP RTDYSFHKFH YLTFVPSAED FYDCRVEHWG LDQPLLKHW (SEQ ID NO: 14, amino acids 29-209 of DPA1*01:03:01:01, see FIG. 15), or an allelic variant thereof.

A suitable DPA1 α1 domain may comprise an amino acid sequence having at least 60%, at least 70%, at least 80%, at least 90%, at least 95%, at least 98%, at least 99%, or 100%, amino acid sequence identity to the following amino acid sequence: AIKADHVSTY AAFVQTHRPT GEFMFEFDED EMFYVDLDKK ETVWHLEEFG QAFSFEAQGG LANIAILNNN LNTLIQRSNH TQATN (SEQ ID NO: 15); and can have a length of about 87 amino acids (e.g., 84, 85, 86, 87, 88, or 89 amino acids). A suitable DPA1 α1 domain can comprise the following amino acid sequence: AIKADHVSTY AAFVQTHRPT GEFMFEFDED EMFYVDLDKK ETVWHLEEFG QAFSFEAQGG LANIAILNNN LNTLIQRSNH TQATN (SEQ ID NO: 15), or a naturally-occurring allelic variant.

A suitable DPA1 α2 domain may comprise an amino acid sequence having at least 60%, at least 70%, at least 80%, at least 90%, at least 95%, at least 98%, at least 99%, or 100%, amino acid sequence identity to the following amino acid sequence: DPPEV TVFPKEPVEL GQPNTLICHI DKFFPPVLNV TWLCNGELVT EGVAESLFLP RTDYSFHKFH YLTFVPSAED FYDCRVEHWG LDQPLLKHW (SEQ ID NO: 16); and can have a length of about 97 amino acids (e.g., 91, 92, 93, 94, 95, 96, or 97 amino acids). A suitable DPA1 α2 domain can comprise the following amino acid sequence: DPPEV TVFPKEPVEL GQPNTLICHI DKFFPPVLNV TWLCNGELVT EGVAESLFLP RTDYSFHKFH YLTFVPSAED FYDCRVEHWG LDQPLLKHW (SEQ ID NO: 16), or a naturally-occurring allelic variant thereof.

Other DPA1 polypeptides comprise the sequence: MRPEDRMFHIRAVILRALSLAFLLSLRGAGAIKADHVSTYAAFVQTHRPTGEFMFEFDE DEQFYVDLDKKETVWHLEEFGRAFSFEAQGGLANIAILNNNLNTLIQRSNHTQAANDPP EVTVFPKEPVELGQPNTLICHIDRFFPPVLNVTWLCNGEPVTEGVAESLFLPRTDYSFHKF HYLTFVPSAEDVYDCRVEHWGLDQPLLKHWEAQEPIQMPETTETVLCALGLVLGLVGII VGTVLIIKSLRSGHDPRAQGPL (SEQ ID NO: 17; amino acids 29-209 of DPA1*02:01:01:01, see FIG. 15), or variant thereof having at least 60%, at least 70%, at least 80%, at least 90%, at least 95%, at least 98%, at least 99%, or 100%, amino acid sequence identity.

A suitable DPA1 α1 domain may comprise an amino acid sequence having at least 60%, at least 70%, at least 80%, at least 90%, at least 95%, at least 98%, at least 99%, or 100%, amino acid sequence identity to the amino acids 29-115 of DPA1*02:01:01:01, SEQ ID NO: 17; and can have a length of about 87 amino acids (e.g., 84, 85, 86, 87, 88, or 89 amino acids. A suitable DPA1 α2 domain may comprise an amino acid sequence having at least 60%, at least 70%, at least 80%, at least 90%, at least 95%, at least 98%, at least 99%, or 100%, amino acid sequence identity to amino acids 116 to 209 of DPA1*02:01:01:01, SEQ ID NO: 17; and can have a length of about 97 amino acids (e.g., 91, 92, 93, 94, 95, 96, or 97 amino acids).

### DQA1

In some cases, a suitable MHC Class II α chain polypeptide is a DQA1 polypeptide. A DQA1 polypeptide can have at least 60%, at least 70%, at least 80%, at least 90%, at least 95%, at least 98%, at least 99%, or 100%, amino acid sequence identity with amino acids 24-204 of any of the DQA1 amino acid sequences depicted in FIG. 17. In some cases, the DQA1 polypeptide has a length of about 181 amino acids (e.g., 177, 178, 179, 180, 181, 182, or 183 amino acids). In an embodiment, a DQA1 α chain polypeptide can have at least 60%, at least 70%, at least 80%, at least 90%, at least 95%, at least 98%, at least 99%, or 100%, amino acid sequence identity with amino acids 30-227 of the DQA1*01:01 α chain amino acid in FIG. 17, ImMunoGeneTics (“IMGT”)/HLA Acc No:HLA00601. In an embodiment, a DQA1 α chain polypeptide can have at least 60%, at least 70%, at least 80%, at least 90%, at least 95%, at least 98%, at least 99%, or 100%, amino acid sequence identity with amino acids 30-227 of the DQA1*01:02 α chain amino acid in FIG. 17, IMGT/HLA Acc No:HLA00603, GenBank NP_002113. In an embodiment, a DQA1 α chain polypeptide can have at least 60%, at least 70%, at least 80%, at least 90%, at least 95%, at least 98%, at least 99%, or 100%, amino acid sequence identity with amino acids 30-227 of the DQA1*02:01 α chain amino acid in FIG. 17, IMGT/HLA Acc No:HLA00607. In an embodiment, a DQA1 α chain polypeptide can have at least 60%, at least 70%, at least 80%, at least 90%, at least 95%, at least 98%, at least 99%, or 100%, amino acid sequence identity with amino acids 30-227 of the DQA1*03:01: α chain amino acid in FIG. 17, IMGT/HLA Acc No:HLA00609. In an embodiment, a DQA1 α chain polypeptide can have at least 60%, at least 70%, at least 80%, at least 90%, at least 95%, at least 98%, at least 99%, or 100%, amino acid sequence identity with amino acids 30-227 of the DQA1*04:01 α chain amino acid in FIG. 17, IMGT/HLA Acc No:HLA00612. In an embodiment, a DQA1 α chain polypeptide can have at least 60%, at least 70%, at least 80%, at least 90%, at least 95%, at least 98%, at least 99%, or 100%, amino acid sequence identity with amino acids 30-227 of the DQA1*05:01 α chain amino acid in FIG. 17, IMGT/HLA Acc No:HLA00613. In an embodiment, a DQA1 α chain polypeptide can have at least 60%, at least 70%, at least 80%, at least 90%, at least 95%, at least 98%, at least 99%, or 100%, amino acid sequence identity with amino acids 30-227 of the DQA1*06:01 α chain amino acid in FIG. 17, IMGT/HLA Acc No:HLA00620.

A “DQA1 polypeptide” includes allelic variants, e.g., naturally occurring allelic variants. Thus, in some cases, a suitable DQA1 polypeptide comprises the following amino acid sequence: EDIVADH VASCGVNLYQ FYGPSGQYTH EFDGDEQFYV DLERKETAWR WPEFSKFGGF DPQGALRNMA VAKHNLNIMI KRYNSTAATN EVPEVTVFSK SPVTLGQPNT LICLVDNIFP PVVNITWLSN GQSVTEGVSE TSFLSKSDHS FFKISYLTFL PSADEIYDCK VEHWGLDQPL LKHW (SEQ ID NO: 18), or an allelic variant thereof.

A suitable DQA1 α1 domain comprises an amino acid sequence having at least 60%, at least 70%, at least 80%, at least 90%, at least 95%, at least 98%, at least 99%, or 100%, amino acid sequence identity to the following amino acid sequence: EDIVADH VASCGVNLYQ FYGPSGQYTH EFDGDEQFYV DLERKETAWR WPEFSKFGGF DPQGALRNMA VAKHNLNIMI KRYNSTAATN (SEQ ID NO: 19); and can have a length of about 87 amino acids (e.g., 84, 85, 86, 87, 88, or 89 amino acids). A suitable DQA1 α1 domain can comprise the following amino acid sequence: EDIVADH VASCGVNLYQ FYGPSGQYTH EFDGDEQFYV DLERKETAWR WPEFSKFGGF DPQGALRNMA VAKHNLNIMI KRYNSTAATN (SEQ ID NO: 19), or a naturally-occurring allelic variant.

A suitable DQA1 α2 domain comprises an amino acid sequence having at least 60%, at least 70%, at least 80%, at least 90%, at least 95%, at least 98%, at least 99%, or 100%, amino acid sequence identity to the following amino acid sequence: EVPEVTVFSK SPVTLGQPNT LICLVDNIFP PVVNITWLSN GQSVTEGVSE TSFLSKSDHS FFKISYLTFL PSADEIYDCK VEHWGLDQPL LKHW (SEQ ID NO: 20); and can have a length of about 94 amino acids (e.g., 91, 92, 93, 94, 95, 96, or 97 amino acids). A suitable DQA1 α2 domain can comprise the following amino acid sequence: EVPEVTVFSK SPVTLGQPNT LICLVDNIFP PVVNITWLSN GQSVTEGVSE TSFLSKSDHS FFKISYLTFL PSADEIYDCK VEHWGLDQPL LKHW (SEQ ID NO: 20), or a naturally-occurring allelic variant thereof.

### DQA2

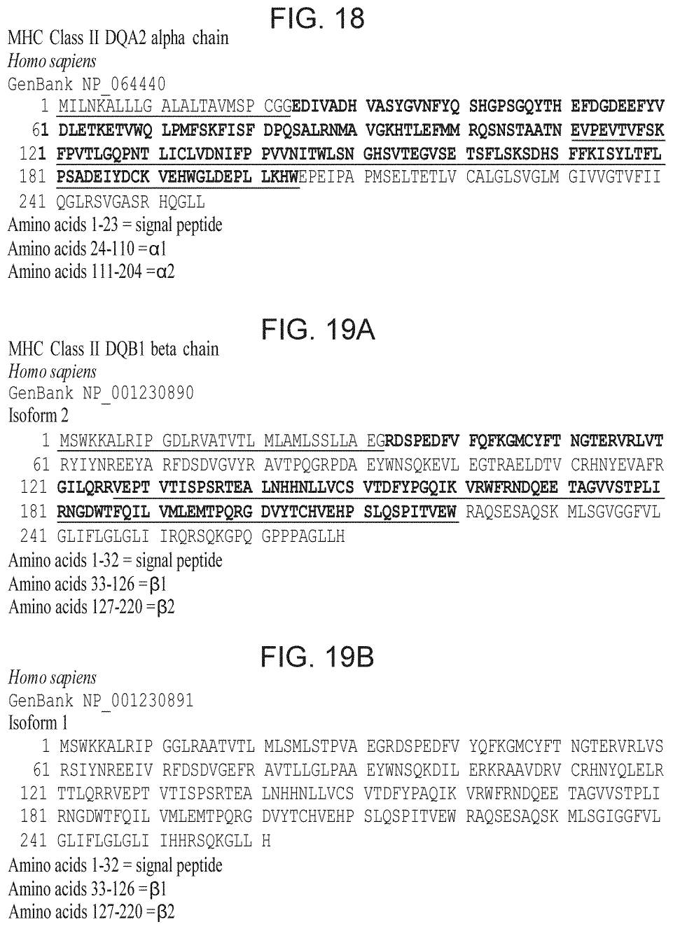

In some cases, a suitable MHC Class II α chain polypeptide is a DQA2 polypeptide. A DQA2 polypeptide can have at least 60%, at least 70%, at least 80%, at least 90%, at least 95%, at least 98%, at least 99%, or 100%, amino acid sequence identity with amino acids 24-204 of the DQA2 amino acid sequence depicted in FIG. 18. In some cases, the DQA2 polypeptide has a length of about 181 amino acids (e.g., 177, 178, 179, 180, 181, 182, or 183 amino acids).

A “DQA2 polypeptide” includes allelic variants, e.g., naturally occurring allelic variants. Thus, in some cases, a suitable DQA2 polypeptide comprises the following amino acid sequence: EDIVADH VASYGVNFYQ SHGPSGQYTH EFDGDEEFYV DLETKETVWQ LPMFSKFISF DPQSALRNMA VGKHTLEFMM RQSNSTAATN EVPEVTVFSK FPVTLGQPNT LICLVDNIFP PVVNITWLSN GHSVTEGVSE TSFLSKSDHS FFKISYLTFL PSADEIYDCK VEHWGLDEPL LKHW (SEQ ID NO: 21), or an allelic variant thereof.

A suitable DQA2 α1 domain comprises an amino acid sequence having at least 60%, at least 70%, at least 80%, at least 90%, at least 95%, at least 98%, at least 99%, or 100%, amino acid sequence identity to the following amino acid sequence: EDIVADH VASYGVNFYQ SHGPSGQYTH EFDGDEEFYV DLETKETVWQ LPMFSKFISF DPQSALRNMA VGKHTLEFMM RQSNSTAATN (SEQ ID NO: 22); and can have a length of about 87 amino acids (e.g., 84, 85, 86, 87, 88, or 89 amino acids). A suitable DQA2 α1 domain can comprise the following amino acid sequence: EDIVADH VASYGVNFYQ SHGPSGQYTH EFDGDEEFYV DLETKETVWQ LPMFSKFISF DPQSALRNMA VGKHTLEFMM RQSNSTAATN (SEQ ID NO: 22), or a naturally-occurring allelic variant.

A suitable DQA2 α2 domain comprises an amino acid sequence having at least 60%, at least 70%, at least 80%, at least 90%, at least 95%, at least 98%, at least 99%, or 100%, amino acid sequence identity to the following amino acid sequence: EVPEVTVFSK FPVTLGQPNT LICLVDNIFP PVVNITWLSN GHSVTEGVSE TSFLSKSDHS FFKISYLTFL PSADEIYDCK VEHWGLDEPL LKHW (SEQ ID NO: 23); and can have a length of about 94 amino acids (e.g., 91, 92, 93, 94, 95, 96, or 97 amino acids). A suitable DQA2 α2 domain can comprise the following amino acid sequence: EVPEVTVFSK FPVTLGQPNT LICLVDNIFP PVVNITWLSN GHSVTEGVSE TSFLSKSDHS FFKISYLTFL PSADEIYDCK VEHWGLDEPL LKHW (SEQ ID NO: 23), or a naturally-occurring allelic variant thereof.

### MHC Class II Beta Chains

MHC Class II beta chains comprise a β1 domain and a β2 domain. In some cases, the β1 domain and the β2 domain present in an antigen-presenting cell are from the same MHC Class II β chain polypeptide. In some cases, the β1 domain and the β2 domain present in an antigen-presenting cell are from two different MHC Class II β chain polypeptides.

MHC Class II beta chains suitable for inclusion in a TMAPP (e.g., a multimeric TMAPP; a single-chain TMAPP) of the present disclosure lack a signal peptide. An MHC Class II beta chain suitable for inclusion in a TMAPP of the present disclosure can have a length of from about 60 amino acids to about 210 amino acids; for example, an MHC Class II beta chain suitable for inclusion in a TMAPP of the present disclosure can have a length of from about 60 amino acids to about 80 amino acids, from about 80 amino acids to about 100 amino acids, from about 100 amino acids to about 120 amino acids, from about 120 amino acids to about 140 amino acids, from about 140 amino acids to about 160 amino acids, from about 160 amino acids to about 180 amino acids, from about 180 amino acids to about 200 amino acids, or from about 200 amino acids to about 210 amino acids. An MHC Class II β1 domain suitable for inclusion in a TMAPP of the present disclosure can have a length of from about 30 amino acids to about 105 amino acids; for example, an MHC Class II β1 domain suitable for inclusion in a TMAPP of the present disclosure can have a length of from about 30 amino acids to about 40 amino acids, from about 40 amino acids to about 50 amino acids, from about 50 amino acids to about 60 amino acids, from about 60 amino acids to about 70 amino acids, from about 70 amino acids to about 80 amino acids, from about 80 amino acids to about 90 amino acids, from about 90 amino acids to about 95 amino acids, from about 95 amino acids to about 100 amino acids, or from about 100 amino acids to about 105 amino acids. An MHC Class II β2 domain suitable for inclusion in a TMAPP of the present disclosure can have a length of from about 30 amino acids to about 105 amino acids; for example, an MHC Class II β2 domain suitable for inclusion in a TMAPP of the present disclosure can have a length of from about 30 amino acids to about 40 amino acids, from about 40 amino acids to about 50 amino acids, from about 50 amino acids to about 60 amino acids, from about 60 amino acids to about 70 amino acids, from about 70 amino acids to about 80 amino acids, from about 80 amino acids to about 90 amino acids, from about 90 amino acids to about 95 amino acids, from about 95 amino acids to about 100 amino acids, or from about 100 amino acids to about 105 amino acids.

### DRB1

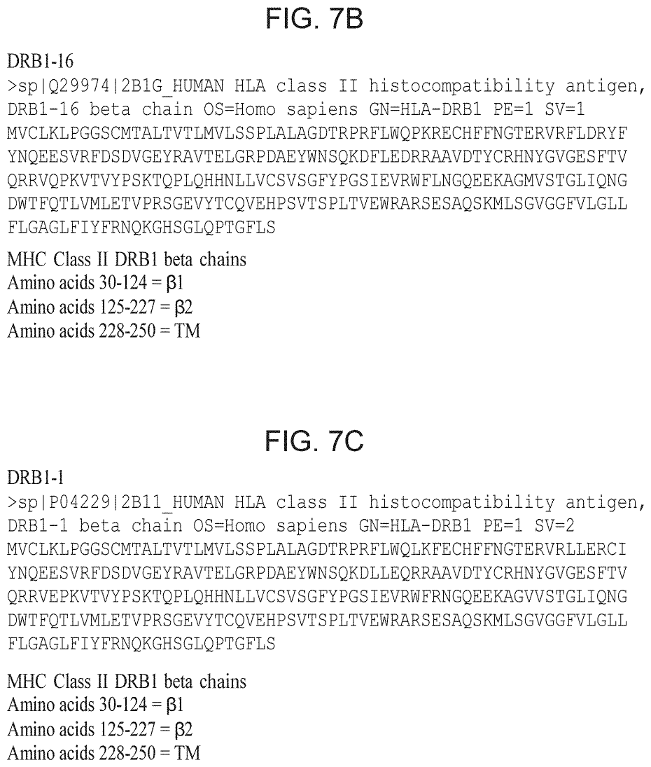

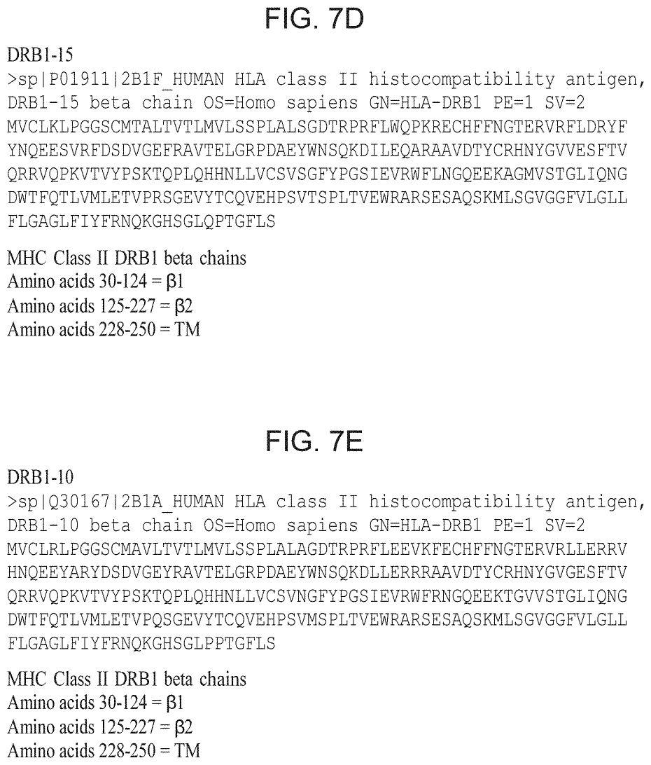

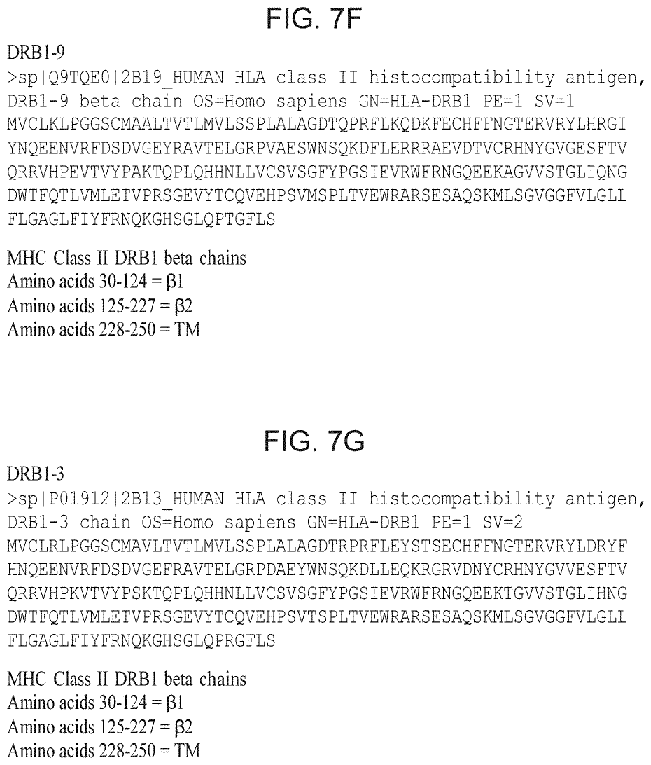

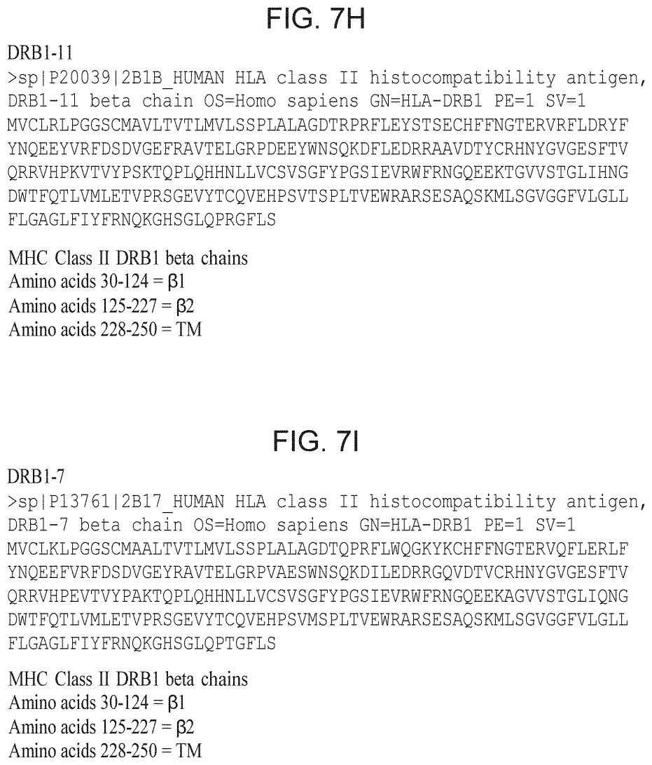

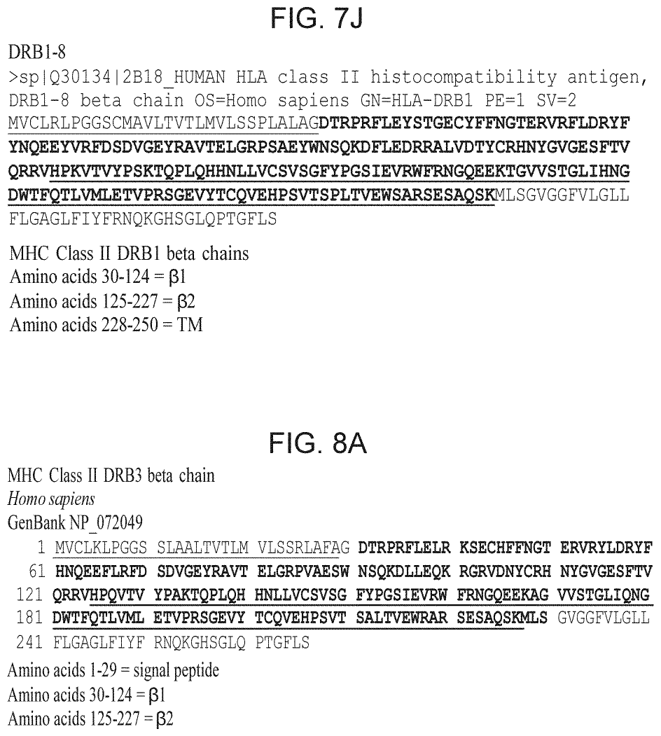

In some cases, a suitable MHC Class II β chain polypeptide is a DRB1 polypeptide. In an embodiment, a DRB1 polypeptide can have at least 60%, at least 70%, at least 80%, at least 90%, at least 95%, at least 98%, at least 99%, or 100%, amino acid sequence identity with amino acids 30-227 of any DRB1 amino acid sequence depicted in FIG. 7, which displays the DRB1 precursor proteins in which amino acids 1-29 are the signal sequence (underlined), 30-124 form the β1 region (bolded), 125-227 for the β2 region (bolded and underlined), and 228-250 the transmembrane region.

In an embodiment, a DRB1 β chain polypeptide can have at least 60%, at least 70%, at least 80%, at least 90%, at least 95%, at least 98%, at least 99%, or 100%, amino acid sequence identity with amino acids 30-227 of the DRB1-1 (DRB1*01:01) beta chain amino acid sequence Swiss-Prot/UniProt reference (“sp”) P04229.2 in FIG. 7. In an embodiment, a DRB1 β chain polypeptide can have at least 60%, at least 70%, at least 80%, at least 90%, at least 95%, at least 98%, at least 99%, or 100%, amino acid sequence identity with amino acids 30-227 of the DRB1-3 (DRB1*03:01) beta chain amino acid sequence sp P01912.2 in FIG. 7. In an embodiment, a DRB1 β chain polypeptide can have at least 60%, at least 70%, at least 80%, at least 90%, at least 95%, at least 98%, at least 99%, or 100%, amino acid sequence identity with amino acids 30-227 of the DRB1-4 (DRB1*04:01) beta chain amino acid sequence sp P13760.1 in FIG. 7. In an embodiment, a DRB1 β chain polypeptide can have at least 60%, at least 70%, at least 80%, at least 90%, at least 95%, at least 98%, at least 99%, or 100%, amino acid sequence identity with amino acids 30-227 of the DRB1-7 (DRB1*07:01) beta chain amino acid sequence sp P13761.1 in FIG. 7. In an embodiment, a DRB1 β chain polypeptide can have at least 60%, at least 70%, at least 80%, at least 90%, at least 95%, at least 98%, at least 99%, or 100%, amino acid sequence identity with amino acids 30-227 of the DRB1-8 (DRB1*08:01) beta chain amino acid sequence sp Q30134.2 in FIG. 7. In an embodiment, a DRB1 β chain polypeptide can have at least 60%, at least 70%, at least 80%, at least 90%, at least 95%, at least 98%, at least 99%, or 100%, amino acid sequence identity with amino acids 30-227 of the DRB1-9 (DRB1*09:01) beta chain amino acid sequence sp Q9TQE0.1 in FIG. 7. In an embodiment, a DRB1 β chain polypeptide can have at least 60%, at least 70%, at least 80%, at least 90%, at least 95%, at least 98%, at least 99%, or 100%, amino acid sequence identity with amino acids 30-227 of the DRB1-10 (DRB1*10:01) beta chain amino acid sequence sp Q30167.2 in FIG. 7. In an embodiment, a DRB1 β chain polypeptide can have at least 60%, at least 70%, at least 80%, at least 90%, at least 95%, at least 98%, at least 99%, or 100%, amino acid sequence identity with amino acids 30-227 of the DRB1-11 (DRB1*11:01) beta chain amino acid sequence sp P20039.1 in FIG. 7. In an embodiment, a DRB1 β chain polypeptide can have at least 60%, at least 70%, at least 80%, at least 90%, at least 95%, at least 98%, at least 99%, or 100%, amino acid sequence identity with amino acids 30-227 of the DRB1-12 (DRB1*12:01) beta chain amino acid sequence sp Q95IE3.1 in FIG. 7. In an embodiment, a DRB1 β chain polypeptide can have at least 60%, at least 70%, at least 80%, at least 90%, at least 95%, at least 98%, at least 99%, or 100%, amino acid sequence identity with amino acids 30-227 of the DRB1-13 (DRB1*13:01) beta chain amino acid sequence sp Q5Y7A7.1 in FIG. 7. In an embodiment, a DRB1 β chain polypeptide can have at least 60%, at least 70%, at least 80%, at least 90%, at least 95%, at least 98%, at least 99%, or 100%, amino acid sequence identity with amino acids 30-227 of the DRB1-14 (DRB1*14:01) beta chain amino acid sequence sp Q9GIY3.1 in FIG. 7. In an embodiment, a DRB1 β chain polypeptide can have at least 60%, at least 70%, at least 80%, at least 90%, at least 95%, at least 98%, at least 99%, or 100%, amino acid sequence identity with amino acids 30-227 of the DRB1-15 (DRB1*15:01) beta chain amino acid sequence sp P01911 in FIG. 7. In an embodiment, a DRB1 β chain polypeptide can have at least 60%, at least 70%, at least 80%, at least 90%, at least 95%, at least 98%, at least 99%, or 100%, amino acid sequence identity with amino acids 30-227 of the DRB1-16 (DRB1*16:01) beta chain amino acid sequence sp Q29974.1 in FIG. 7. In some cases, the DRB1 β chain polypeptide has a length of about 198 amino acids (e.g., 195, 196, 197, 198, 199, 200, 201, or 202 amino acids).

A “DRB1 polypeptide” includes allelic variants, e.g., naturally occurring allelic variants. Thus, in some cases, a suitable DRB1 polypeptide comprises the following amino acid sequence: DTRPRFLEQVKHECHFFNGTERVRFLDRYFYHQEEYVRFDSDVGEYRAVTELGRPDAE YWNSQKDLLEQKRAAVDTYCRHNYGVGESFTVQRRVYPEVTVYPAKTQPLQHHNLLV CSVNGFYPGSIEVRWFRNGQEEKTGVVSTGLIQNGDWTFQTLVMLETVPRSGEVYTCQ VEHPSLTSPLTVEWRARSESAQSK (SEQ ID NO: 24) (amino acids 31-227 of DRB1-4, see FIG. 7A), or an allelic variant thereof.

A suitable DRB1 β1 domain comprises an amino acid sequence having at least 60%, at least 70%, at least 80%, at least 90%, at least 95%, at least 98%, at least 99%, or 100%, amino acid sequence identity to the following amino acid sequence: DTRPRFLEQVKHECHFFNGTERVRFLDRYFYHQEEYVRFDSDVGEYRAVTELGRPDAE YWNSQKDLLEQKRAAVDTYCRHNYGVGESFTVQRRV (SEQ ID NO: 25); and can have a length of about 95 amino acids (e.g., 92, 93, 94, 95, 96, 97, or 98 amino acids). A suitable DRB1 β1 domain can comprise the following amino acid sequence: DTRPRFLEQVKHECHFFNGTERVRFLDRYFYHQEEYVRFDSDVGEYRAVTELGRPDAE YWNSQKDLLEQKRAAVDTYCRHNYGVGESFTVQRRV (SEQ ID NO: 25), or a naturally-occurring allelic variant.

A suitable DRB1 β2 domain comprises an amino acid sequence having at least 60%, at least 70%, at least 80%, at least 90%, at least 95%, at least 98%, at least 99%, or 100%, amino acid sequence identity to the following amino acid sequence: YPEVTVYPAKTQPLQHHNLLVCSVNGFYPGSIEVRWFRNGQEEKTGVVSTGLIQNGDW TFQTLVMLETVPRSGEVYTCQVEHPSLTSPLTVEWRARSESAQSK (SEQ ID NO: 26); and can have a length of about 103 amino acids (e.g., 100, 101, 102, 103, 104, 105, or 106 amino acids). A suitable DRB1 β2 domain can comprise the following amino acid sequence: YPEVTVYPAKTQPLQHHNLLVCSVNGFYPGSIEVRWFRNGQEEKTGVVSTGLIQNGDW TFQTLVMLETVPRSGEVYTCQVEHPSLTSPLTVEWRARSESAQSK (SEQ ID NO: 26), or a naturally-occurring allelic variant thereof.

### DRB3

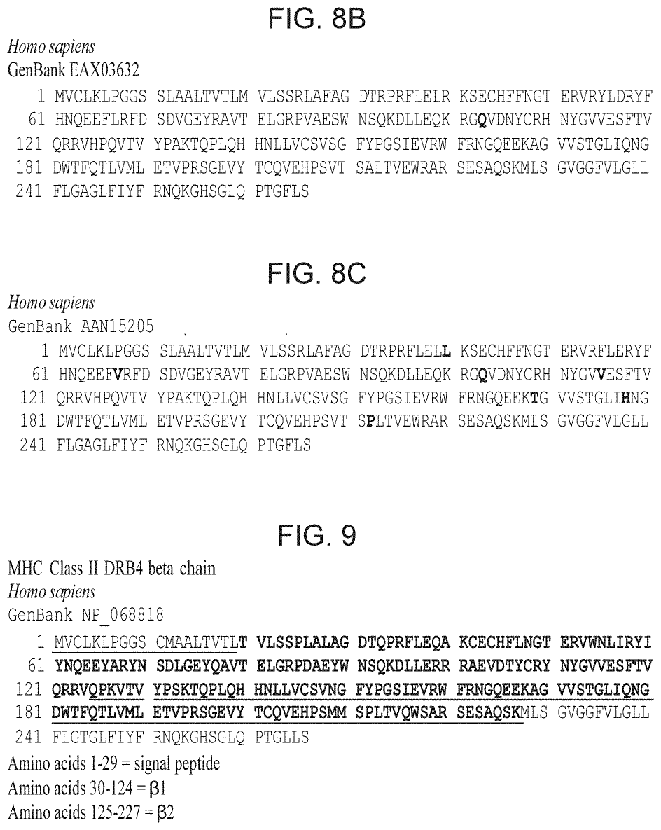

In some cases, a suitable MHC Class II β chain polypeptide is a DRB3 polypeptide. In an embodiment, a DRB3 polypeptide can have at least 60%, at least 70%, at least 80%, at least 90%, at least 95%, at least 98%, at least 99%, or 100%, amino acid sequence identity with amino acids 30-227 of any DRB3 amino acid sequence depicted in FIG. 8, which displays the DRB3 precursor proteins in which amino acids 1-29 are the signal sequence (underlined), 30-124 form the β1 region (shown bolded), 125-227 for the β2 region, and 228-250 the transmembrane region. In an embodiment, a DRB3 β chain polypeptide can have at least 60%, at least 70%, at least 80%, at least 90%, at least 95%, at least 98%, at least 99%, or 100%, amino acid sequence identity with amino acids 30-227 of the DRB1-3 (DRB3*01:01) beta chain amino acid sequence GenBank NP_072049.1 in FIG. 8. In an embodiment, a DRB3 β chain polypeptide can have at least 60%, at least 70%, at least 80%, at least 90%, at least 95%, at least 98%, at least 99%, or 100%, amino acid sequence identity with amino acids 30-227 of the DRB1-3 beta chain amino acid sequence in GenBank accession EAX03632.1 in FIG. 8. In an embodiment, a DRB3 β chain polypeptide can have at least 60%, at least 70%, at least 80%, at least 90%, at least 95%, at least 98%, at least 99%, or 100%, amino acid sequence identity with amino acids 30-227 of the DRB1-3 (DRB3*02:01) beta chain amino acid sequence GenBank CAA23781.1 in FIG. 8. In an embodiment, a DRB3 β chain polypeptide can have at least 60%, at least 70%, at least 80%, at least 90%, at least 95%, at least 98%, at least 99%, or 100%, amino acid sequence identity with amino acids 30-227 of the DRB1-3 (DRB3*03:01) beta chain amino acid sequence GenBank AAN15205.1 in FIG. 8.

A “DRB3 polypeptide” includes allelic variants, e.g., naturally occurring allelic variants. Thus, in some cases, a suitable DRB3 polypeptide comprises the following amino acid sequence: DTRPRFLELR KSECHFFNGT ERVRYLDRYF HNQEEFLRFD SDVGEYRAVT ELGRPVAESW NSQKDLLEQK RGRVDNYCRH NYGVGESFTV QRRVHPQVTV YPAKTQPLQH HNLLVCSVSG FYPGSIEVRW FRNGQEEKAG VVSTGLIQNG DWTFQTLVML ETVPRSGEVY TCQVEHPSVT SALTVEWRAR SESAQSK (SEQ ID NO: 27), or an allelic variant thereof.

A suitable DRB3 β1 domain comprises an amino acid sequence having at least 60%, at least 70%, at least 80%, at least 90%, at least 95%, at least 98%, at least 99%, or 100%, amino acid sequence identity to the following amino acid sequence: DTRPRFLELR KSECHFFNGT ERVRYLDRYF HNQEEFLRFD SDVGEYRAVT ELGRPVAESW NSQKDLLEQK RGRVDNYCRH NYGVGESFTV QRRV (SEQ ID NO: 28); and can have a length of about 95 amino acids (e.g., 93, 94, 95, 96, 97, or 98 amino acids). A suitable DRB3 β1 domain can comprise the following amino acid sequence: DTRPRFLELR KSECHFFNGT ERVRYLDRYF HNQEEFLRFD SDVGEYRAVT ELGRPVAESW NSQKDLLEQK RGRVDNYCRH NYGVGESFTV QRRV (SEQ ID NO: 28), or a naturally-occurring allelic variant.

A suitable DRB3 β2 domain comprises an amino acid sequence having at least 60%, at least 70%, at least 80%, at least 90%, at least 95%, at least 98%, at least 99%, or 100%, amino acid sequence identity to the following amino acid sequence: HPQVTV YPAKTQPLQH HNLLVCSVSG FYPGSIEVRW FRNGQEEKAG VVSTGLIQNG DWTFQTLVML ETVPRSGEVY TCQVEHPSVT SALTVEWRAR SESAQSK (SEQ ID NO: 29); and can have a length of about 103 amino acids (e.g., 100, 101, 102, 103, 104, or 105 amino acids). A suitable DRB3 β2 domain can comprise the following amino acid sequence: HPQVTV YPAKTQPLQH HNLLVCSVSG FYPGSIEVRW FRNGQEEKAG VVSTGLIQNG DWTFQTLVML ETVPRSGEVY TCQVEHPSVT SALTVEWRAR SESAQSK (SEQ ID NO: 29), or a naturally-occurring allelic variant thereof.

### DRB4

In some cases, a suitable MHC Class II β chain polypeptide is a DRB4 polypeptide. A DRB4 polypeptide can have at least 60%, at least 70%, at least 80%, at least 90%, at least 95%, at least 98%, at least 99%, or 100%, amino acid sequence identity with amino acids 30-227 of the DRB4 amino acid sequence depicted in FIG. 9. In some cases, the DRB4 polypeptide has a length of about 198 amino acids (e.g., 195, 196, 197, 198, 199, 200, 201, or 202 amino acids).

A “DRB4 polypeptide” includes allelic variants, e.g., naturally occurring allelic variants. Thus, in some cases, a suitable DRB4 polypeptide comprises the following amino acid sequence: T VLSSPLALAG DTQPRFLEQA KCECHFLNGT ERVWNLIRYI YNQEEYARYN SDLGEYQAVT ELGRPDAEYW NSQKDLLERR RAEVDTYCRY NYGVVESFTV QRRVQPKVTV YPSKTQPLQH HNLLVCSVNG FYPGSIEVRW FRNGQEEKAG VVSTGLIQNG DWTFQTLVML ETVPRSGEVY TCQVEHPSMM SPLTVQWSAR SESAQSK (SEQ ID NO: 30), or an allelic variant thereof.

A suitable DRB4 β1 domain comprises an amino acid sequence having at least 60%, at least 70%, at least 80%, at least 90%, at least 95%, at least 98%, at least 99%, or 100%, amino acid sequence identity to the following amino acid sequence: T VLSSPLALAG DTQPRFLEQA KCECHFLNGT ERVWNLIRYI YNQEEYARYN SDLGEYQAVT ELGRPDAEYW NSQKDLLERR RAEVDTYCRY NYGVVESFTV QRRV (SEQ ID NO: 31); and can have a length of about 95 amino acids (e.g., 93, 94, 95, 96, 97, or 98 amino acids). A suitable DRB4 β1 domain can comprise the following amino acid sequence: T VLSSPLALAG DTQPRFLEQA KCECHFLNGT ERVWNLIRYI YNQEEYARYN SDLGEYQAVT ELGRPDAEYW NSQKDLLERR RAEVDTYCRY NYGVVESFTV QRRV (SEQ ID NO: 31), or a naturally-occurring allelic variant.

A suitable DRB4 β2 domain comprises an amino acid sequence having at least 60%, at least 70%, at least 80%, at least 90%, at least 95%, at least 98%, at least 99%, or 100%, amino acid sequence identity to the following amino acid sequence: QPKVTV YPSKTQPLQH HNLLVCSVNG FYPGSIEVRW FRNGQEEKAG VVSTGLIQNG DWTFQTLVML ETVPRSGEVY TCQVEHPSMM SPLTVQWSAR SESAQSK (SEQ ID NO: 32); and can have a length of about 103 amino acids (e.g., 100, 101, 102, 103, 104, or 105 amino acids). A suitable DRB4 β2 domain can comprise the following amino acid sequence: QPKVTV YPSKTQPLQH HNLLVCSVNG FYPGSIEVRW FRNGQEEKAG VVSTGLIQNG DWTFQTLVML ETVPRSGEVY TCQVEHPSMM SPLTVQWSAR SESAQSK (SEQ ID NO: 32), or a naturally-occurring allelic variant thereof.

### DRB5

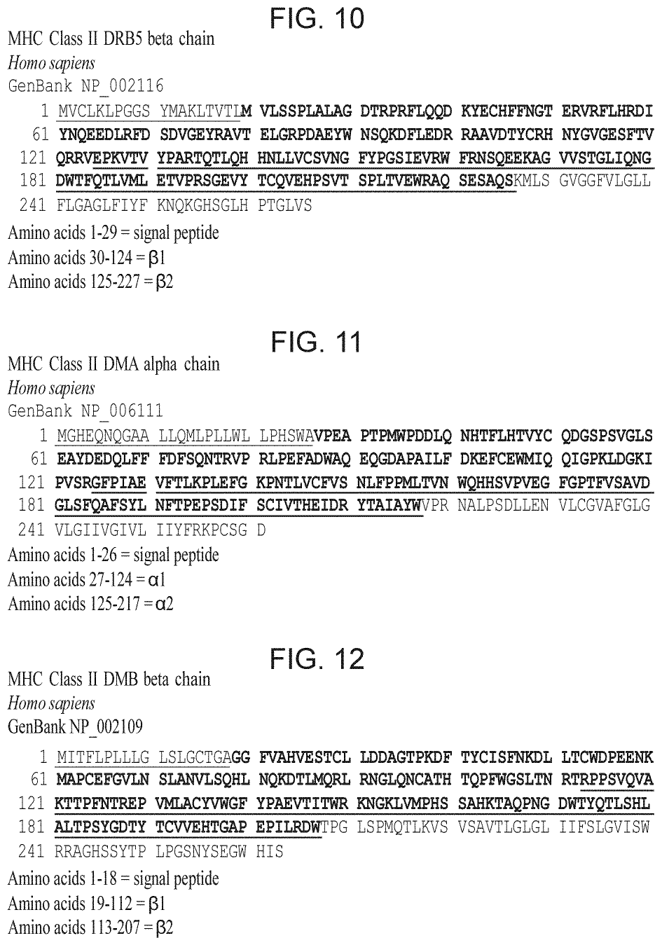

In some cases, a suitable MHC Class II β chain polypeptide is a DRB5 polypeptide. A DRB5 polypeptide can have at least 60%, at least 70%, at least 80%, at least 90%, at least 95%, at least 98%, at least 99%, or 100%, amino acid sequence identity with amino acids 30-227 of the DRB5 amino acid sequence depicted in FIG. 10. In some cases, the DRB5 polypeptide has a length of about 198 amino acids (e.g., 195, 196, 197, 198, 199, 200, 201, or 202 amino acids).

A “DRB5 polypeptide” includes allelic variants, e.g., naturally occurring allelic variants. Thus, in some cases, a suitable DRB5 polypeptide comprises the following amino acid sequence: M VLSSPLALAG DTRPRFLQQD KYECHFFNGT ERVRFLHRDI YNQEEDLRFD SDVGEYRAVT ELGRPDAEYW NSQKDFLEDR RAAVDTYCRH NYGVGESFTV QRRVEPKVTV YPARTQTLQH HNLLVCSVNG FYPGSIEVRW FRNSQEEKAG VVSTGLIQNG DWTFQTLVML ETVPRSGEVY TCQVEHPSVT SPLTVEWRAQ SESAQS (SEQ ID NO: 33), or an allelic variant thereof.

A suitable DRB5 β1 domain comprises an amino acid sequence having at least 60%, at least 70%, at least 80%, at least 90%, at least 95%, at least 98%, at least 99%, or 100%, amino acid sequence identity to the following amino acid sequence: M VLSSPLALAG DTRPRFLQQD KYECHFFNGT ERVRFLHRDI YNQEEDLRFD SDVGEYRAVT ELGRPDAEYW NSQKDFLEDR RAAVDTYCRH NYGVGESFTV QRRV (SEQ ID NO: 34); and can have a length of about 95 amino acids (e.g., 93, 94, 95, 96, 97, or 98 amino acids). A suitable DRB5 β1 domain can comprise the following amino acid sequence: M VLSSPLALAG DTRPRFLQQD KYECHFFNGT ERVRFLHRDI YNQEEDLRFD SDVGEYRAVT ELGRPDAEYW NSQKDFLEDR RAAVDTYCRH NYGVGESFTV QRRV (SEQ ID NO: 34), or a naturally-occurring allelic variant.

A suitable DRB5 β2 domain comprises an amino acid sequence having at least 60%, at least 70%, at least 80%, at least 90%, at least 95%, at least 98%, at least 99%, or 100%, amino acid sequence identity to the following amino acid sequence: EPKVTV YPARTQTLQH HNLLVCSVNG FYPGSIEVRW FRNSQEEKAG VVSTGLIQNG DWTFQTLVML ETVPRSGEVY TCQVEHPSVT SPLTVEWRAQ SESAQS (SEQ ID NO: 35); and can have a length of about 103 amino acids (e.g., 100, 101, 102, 103, 104, or 105 amino acids). A suitable DRB5 β2 domain can comprise the following amino acid sequence: EPKVTV YPARTQTLQH HNLLVCSVNG FYPGSIEVRW FRNSQEEKAG VVSTGLIQNG DWTFQTLVML ETVPRSGEVY TCQVEHPSVT SPLTVEWRAQ SESAQS (SEQ ID NO: 35), or a naturally-occurring allelic variant thereof.

### DMB

In some cases, a suitable MHC Class II β chain polypeptide is a DMB polypeptide. A DMB polypeptide can have at least 60%, at least 70%, at least 80%, at least 90%, at least 95%, at least 98%, at least 99%, or 100%, amino acid sequence identity with amino acids 19-207 of the DMB amino acid sequence depicted in FIG. 12. In some cases, the DMB polypeptide has a length of about 189 amino acids (e.g., 187, 188, 189, 190, or 191 amino acids).

A “DMB polypeptide” includes allelic variants, e.g., naturally occurring allelic variants. Thus, in some cases, a suitable DMB polypeptide comprises the following amino acid sequence: GG FVAHVESTCL LDDAGTPKDF TYCISFNKDL LTCWDPEENK MAPCEFGVLN SLANVLSQHL NQKDTLMQRL RNGLQNCATH TQPFWGSLTN RTRPPSVQVA KTTPFNTREP VMLACYVWGF YPAEVTITWR KNGKLVMPHS SAHKTAQPNG DWTYQTLSHL ALTPSYGDTY TCVVEHTGAP EPILRDW (SEQ ID NO: 36), or an allelic variant thereof.

A suitable DMB β1 domain comprises an amino acid sequence having at least 60%, at least 70%, at least 80%, at least 90%, at least 95%, at least 98%, at least 99%, or 100%, amino acid sequence identity to the following amino acid sequence: GG FVAHVESTCL LDDAGTPKDF TYCISFNKDL LTCWDPEENK MAPCEFGVLN SLANVLSQHL NQKDTLMQRL RNGLQNCATH TQPFWGSLTN RT (SEQ ID NO: 37); and can have a length of about 94 amino acids (e.g., 92, 93, 94, 95, 96, or 97 amino acids). A suitable DMB β1 domain can comprise the following amino acid sequence: GG FVAHVESTCL LDDAGTPKDF TYCISFNKDL LTCWDPEENK MAPCEFGVLN SLANVLSQHL NQKDTLMQRL RNGLQNCATH TQPFWGSLTN RT (SEQ ID NO: 37), or a naturally-occurring allelic variant.

A suitable DMB β2 domain comprises an amino acid sequence having at least 60%, at least 70%, at least 80%, at least 90%, at least 95%, at least 98%, at least 99%, or 100%, amino acid sequence identity to the following amino acid sequence: RPPSVQVA KTTPFNTREP VMLACYVWGF YPAEVTITWR KNGKLVMPHS SAHKTAQPNG DWTYQTLSHL ALTPSYGDTY TCVVEHTGAP EPILRDW (SEQ ID NO: 38); and can have a length of about 95 amino acids (e.g., 93, 94, 95, 96, 97, or 98 amino acids). A suitable DMB β2 domain can comprise the following amino acid sequence: RPPSVQVA KTTPFNTREP VMLACYVWGF YPAEVTITWR KNGKLVMPHS SAHKTAQPNG DWTYQTLSHL ALTPSYGDTY TCVVEHTGAP EPILRDW (SEQ ID NO: 38), or a naturally-occurring allelic variant thereof.

### DOB

In some cases, a suitable MHC Class II β chain polypeptide is a DOB polypeptide. A DOB polypeptide can have at least 60%, at least 70%, at least 80%, at least 90%, at least 95%, at least 98%, at least 99%, or 100%, amino acid sequence identity with amino acids 27-214 of the DOB amino acid sequence depicted in FIG. 14. In some cases, the DOB polypeptide has a length of about 188 amino acids (e.g., 186, 187, 188, 189, or 190 amino acids).

A “DOB polypeptide” includes allelic variants, e.g., naturally occurring allelic variants. Thus, in some cases, a suitable DOB polypeptide comprises the following amino acid sequence: TDSP EDFVIQAKAD CYFTNGTEKV QFVVRFIFNL EEYVRFDSDV GMFVALTKLG QPDAEQWNSR LDLLERSRQA VDGVCRHNYR LGAPFTVGRK VQPEVTVYPE RTPLLHQHNL LHCSVTGFYP GDIKIKWFLN GQEERAGVMS TGPIRNGDWT FQTVVMLEMT PELGHVYTCL VDHSSLLSPV SVEW (SEQ ID NO: 39), or an allelic variant thereof.

A suitable DOB β1 domain comprises an amino acid sequence having at least 60%, at least 70%, at least 80%, at least 90%, at least 95%, at least 98%, at least 99%, or 100%, amino acid sequence identity to the following amino acid sequence: TDSP EDFVIQAKAD CYFTNGTEKV QFVVRFIFNL EEYVRFDSDV GMFVALTKLG QPDAEQWNSR LDLLERSRQA VDGVCRHNYR LGAPFTVGRK (SEQ ID NO: 40); and can have a length of about 94 amino acids (e.g., 92, 93, 94, 95, 96, or 97 amino acids). A suitable DOB β1 domain can comprise the following amino acid sequence: TDSP EDFVIQAKAD CYFTNGTEKV QFVVRFIFNL EEYVRFDSDV GMFVALTKLG QPDAEQWNSR LDLLERSRQA VDGVCRHNYR LGAPFTVGRK (SEQ ID NO: 40), or a naturally-occurring allelic variant.

A suitable DOB β2 domain comprises an amino acid sequence having at least 60%, at least 70%, at least 80%, at least 90%, at least 95%, at least 98%, at least 99%, or 100%, amino acid sequence identity to the following amino acid sequence: VQPEVTVYPE RTPLLHQHNL LHCSVTGFYP GDIKIKWFLN GQEERAGVMS TGPIRNGDWT FQTVVMLEMT PELGHVYTCL VDHSSLLSPV SVEW (SEQ ID NO: 41); and can have a length of about 94 amino acids (e.g., 92, 93, 94, 95, 96, or 97 amino acids). A suitable DOB β2 domain can comprise the following amino acid sequence: VQPEVTVYPE RTPLLHQHNL LHCSVTGFYP GDIKIKWFLN GQEERAGVMS TGPIRNGDWT FQTVVMLEMT PELGHVYTCL VDHSSLLSPV SVEW (SEQ ID NO: 41), or a naturally-occurring allelic variant thereof.

### DPB1

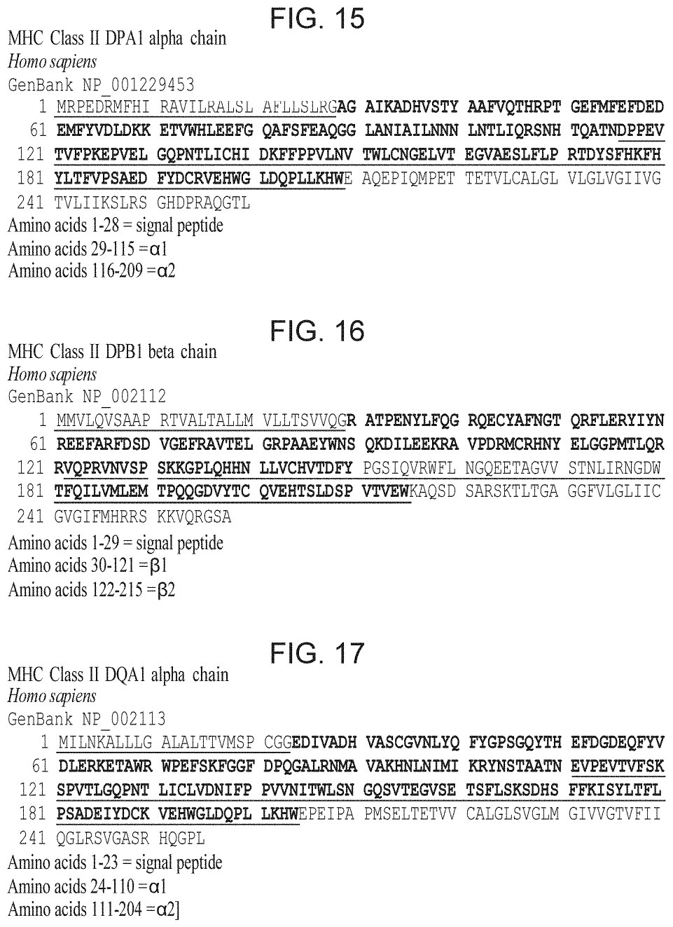

In some cases, a suitable MHC Class II β chain polypeptide is a DPB1 polypeptide. A DPB1 polypeptide can have at least 60%, at least 70%, at least 80%, at least 90%, at least 95%, at least 98%, at least 99%, or 100%, amino acid sequence identity with amino acids 30-215 of any of the DPB1 amino acid sequences depicted in FIG. 16. In some cases, the DPB1 polypeptide has a length of about 186 amino acids (e.g., 184, 185, 186, 187, or 188 amino acids). In an embodiment, a DRB3 β chain polypeptide can have at least 60%, at least 70%, at least 80%, at least 90%, at least 95%, at least 98%, at least 99%, or 100%, amino acid sequence identity with amino acids 30-227 of the DPB1*01:01 beta chain amino acid sequence in FIG. 16 IMGT/HLA Acc No: HLA00514. In an embodiment, a DRB3 β chain polypeptide can have at least 60%, at least 70%, at least 80%, at least 90%, at least 95%, at least 98%, at least 99%, or 100%, amino acid sequence identity with amino acids 30-227 of the DPB1*01:01 beta chain amino acid sequence in FIG. 16, IMGT/HLA Acc No: HLA00517. In an embodiment, a DRB3 β chain polypeptide can have at least 60%, at least 70%, at least 80%, at least 90%, at least 95%, at least 98%, at least 99%, or 100%, amino acid sequence identity with amino acids 30-227 of the DPB1*03:01 beta chain amino acid sequence in FIG. 16, IMGT/HLA Acc No: HLA00520. In an embodiment, a DRB3 β chain polypeptide can have at least 60%, at least 70%, at least 80%, at least 90%, at least 95%, at least 98%, at least 99%, or 100%, amino acid sequence identity with amino acids 30-227 of the DPB1*04:01 beta chain amino acid sequence in FIG. 16, IMGT/HLA Acc No: HLA00521, GenBank NP_002112.3. In an embodiment, a DRB3 β chain polypeptide can have at least 60%, at least 70%, at least 80%, at least 90%, at least 95%, at least 98%, at least 99%, or 100%, amino acid sequence identity with amino acids 30-227 of the DPB106:01 beta chain amino acid sequence in FIG. 16, IMGT/HLA Acc No: HLA00524. In an embodiment, a DRB3 β chain polypeptide can have at least 60%, at least 70%, at least 80%, at least 90%, at least 95%, at least 98%, at least 99%, or 100%, amino acid sequence identity with amino acids 30-227 of the DPB1*11:01 beta chain amino acid sequence in FIG. 16, IMGT/HLA Acc No: HLA00528. In an embodiment, a DRB3 β chain polypeptide can have at least 60%, at least 70%, at least 80%, at least 90%, at least 95%, at least 98%, at least 99%, or 100%, amino acid sequence identity with amino acids 30-227 of the DPB1*71:01 beta chain amino acid sequence in FIG. 16, IMGT/HLA Acc No:HLA00590. In an embodiment, a DRB3 β chain polypeptide can have at least 60%, at least 70%, at least 80%, at least 90%, at least 95%, at least 98%, at least 99%, or 100%, amino acid sequence identity with amino acids 30-227 of the DPB1*104:01 beta chain amino acid sequence in FIG. 16, IMGT/HLA Acc No: HLA02046. In an embodiment, a DRB3 β chain polypeptide can have at least 60%, at least 70%, at least 80%, at least 90%, at least 95%, at least 98%, at least 99%, or 100%, amino acid sequence identity with amino acids 30-227 of the DPB1*141:01 beta chain amino acid sequence in FIG. 16, IMGT/HLA Acc No: HLA10364.

A “DPB1 polypeptide” includes allelic variants, e.g., naturally occurring allelic variants. Thus, in some cases, a suitable DPB1 polypeptide comprises the following amino acid sequence: R ATPENYLFQG RQECYAFNGT QRFLERYIYN REEFARFDSD VGEFRAVTEL GRPAAEYWNS QKDILEEKRA VPDRMCRHNY ELGGPMTLQR RVQPRVNVSP SKKGPLQHHN LLVCHVTDFY PGSIQVRWFL NGQEETAGVV STNLIRNGDW TFQILVMLEM TPQQGDVYTC QVEHTSLDSP VTVEW (SEQ ID NO: 42), or an allelic variant thereof.

A suitable DPB1 β1 domain comprises an amino acid sequence having at least 60%, at least 70%, at least 80%, at least 90%, at least 95%, at least 98%, at least 99%, or 100%, amino acid sequence identity to the following amino acid sequence: R ATPENYLFQG RQECYAFNGT QRFLERYIYN REEFARFDSD VGEFRAVTEL GRPAAEYWNS QKDILEEKRA VPDRMCRHNY ELGGPMTLQR R (SEQ ID NO: 43); and can have a length of about 92 amino acids (e.g., 90, 91, 92, 93, or 94 amino acids). A suitable DPB1 β1 domain can comprise the following amino acid sequence: R ATPENYLFQG RQECYAFNGT QRFLERYIYN REEFARFDSD VGEFRAVTEL GRPAAEYWNS QKDILEEKRA VPDRMCRHNY ELGGPMTLQR R (SEQ ID NO: 43), or a naturally-occurring allelic variant.

A suitable DPB1 β2 domain comprises an amino acid sequence having at least 60%, at least 70%, at least 80%, at least 90%, at least 95%, at least 98%, at least 99%, or 100%, amino acid sequence identity to the following amino acid sequence: VQPRVNVSP SKKGPLQHHN LLVCHVTDFY PGSIQVRWFL NGQEETAGVV STNLIRNGDW TFQILVMLEM TPQQGDVYTC QVEHTSLDSP VTVEW (SEQ ID NO: 44); and can have a length of about 94 amino acids (e.g., 92, 93, 94, 95, 96, or 97 amino acids). A suitable DPB1 β2 domain can comprise the following amino acid sequence: VQPRVNVSP SKKGPLQHHN LLVCHVTDFY PGSIQVRWFL NGQEETAGVV STNLIRNGDW TFQILVMLEM TPQQGDVYTC QVEHTSLDSP VTVEW (SEQ ID NO: 44), or a naturally-occurring allelic variant thereof.

### DQB1

In some cases, a suitable MHC Class II β chain polypeptide is a DQB1 polypeptide. A DQB1 polypeptide can have at least 60%, at least 70%, at least 80%, at least 90%, at least 95%, at least 98%, at least 99%, or 100%, amino acid sequence identity with amino acids 33-220 of the DQB1 amino acid sequence depicted in FIG. 19A or FIG. 19B or FIG. 19C. In some cases, the DQB1 polypeptide has a length of about 188 amino acids (e.g., 186, 187, 188, 190, 191, or 192 amino acids).

A “DQB1 polypeptide” includes allelic variants, e.g., naturally occurring allelic variants. Thus, in some cases, a suitable DQB1 polypeptide comprises the following amino acid sequence: RDSPEDFV FQFKGMCYFT NGTERVRLVT RYIYNREEYA RFDSDVGVYR AVTPQGRPDA EYWNSQKEVL EGTRAELDTV CRHNYEVAFR GILQRRVEPT VTISPSRTEA LNHHNLLVCS VTDFYPGQIK VRWFRNDQEE TAGVVSTPLI RNGDWTFQIL VMLEMTPQRG DVYTCHVEHP SLQSPITVEW (SEQ ID NO: 45), or an allelic variant thereof.

A suitable DQB1 β1 domain comprises an amino acid sequence having at least 60%, at least 70%, at least 80%, at least 90%, at least 95%, at least 98%, at least 99%, or 100%, amino acid sequence identity to the following amino acid sequence: RDSPEDFV FQFKGMCYFT NGTERVRLVT RYIYNREEYA RFDSDVGVYR AVTPQGRPDA EYWNSQKEVL EGTRAELDTV CRHNYEVAFR GILQRR (SEQ ID NO: 46); and can have a length of about 94 amino acids (e.g., 92, 93, 94, 95, or 96 amino acids). A suitable DQB1 β1 domain can comprise the following amino acid sequence: RDSPEDFV FQFKGMCYFT NGTERVRLVT RYIYNREEYA RFDSDVGVYR AVTPQGRPDA EYWNSQKEVL EGTRAELDTV CRHNYEVAFR GILQRR (SEQ ID NO: 46), or a naturally-occurring allelic variant.

A suitable DQB1 β2 domain comprises an amino acid sequence having at least 60%, at least 70%, at least 80%, at least 90%, at least 95%, at least 98%, at least 99%, or 100%, amino acid sequence identity to the following amino acid sequence: VEPT VTISPSRTEA LNHHNLLVCS VTDFYPGQIK VRWFRNDQEE TAGVVSTPLI RNGDWTFQIL VMLEMTPQRG DVYTCHVEHP SLQSPITVEW (SEQ ID NO: 47); and can have a length of about 94 amino acids (e.g., 92, 93, 94, 95, or 96 amino acids). A suitable DQB1 β2 domain can comprise the following amino acid sequence: VEPT VTISPSRTEA LNHHNLLVCS VTDFYPGQIK VRWFRNDQEE TAGVVSTPLI RNGDWTFQIL VMLEMTPQRG DVYTCHVEHP SLQSPITVEW (SEQ ID NO: 47), or a naturally-occurring allelic variant thereof.

### DQB2

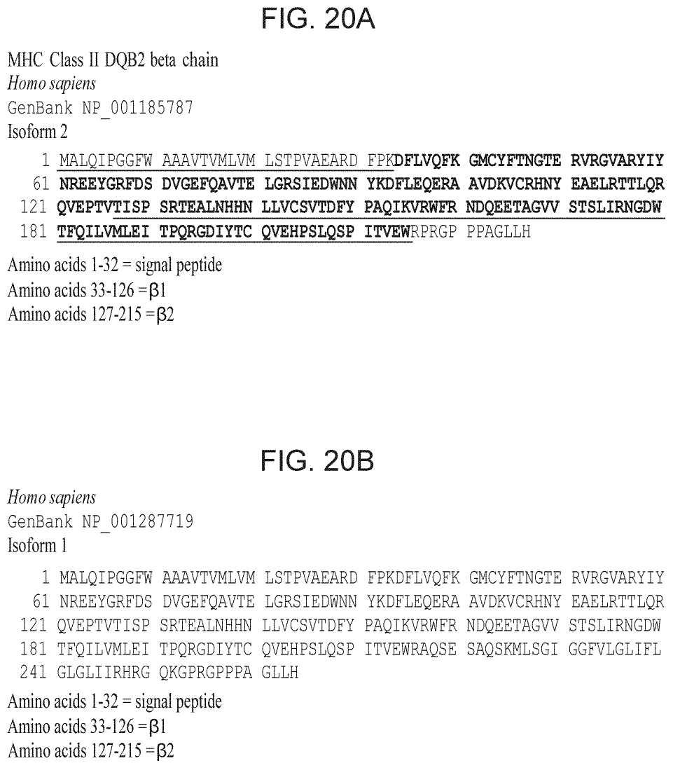

In some cases, a suitable MHC Class II β chain polypeptide is a DQB2 polypeptide. A DQB2 polypeptide can have at least 60%, at least 70%, at least 80%, at least 90%, at least 95%, at least 98%, at least 99%, or 100%, amino acid sequence identity with amino acids 33-215 of the DQB2 amino acid sequence depicted in FIG. 20A or FIG. 20. In some cases, the DQB2 polypeptide has a length of about 182 amino acids (e.g., 175, 176, 177, 178, 179, 180, 181, or 182 amino acids).

A “DQB2 polypeptide” includes allelic variants, e.g., naturally occurring allelic variants. Thus, in some cases, a suitable DQB2 polypeptide comprises the following amino acid sequence: DFLVQFK GMCYFTNGTE RVRGVARYIY NREEYGRFDS DVGEFQAVTE LGRSIEDWNN YKDFLEQERA AVDKVCRHNY EAELRTTLQR QVEPTVTISP SRTEALNHHN LLVCSVTDFY PAQIKVRWFR NDQEETAGVV STSLIRNGDW TFQILVMLEI TPQRGDIYTC QVEHPSLQSP ITVEW (SEQ ID NO: 48), or an allelic variant thereof.

A suitable DQB2 β1 domain comprises an amino acid sequence having at least 60%, at least 70%, at least 80%, at least 90%, at least 95%, at least 98%, at least 99%, or 100%, amino acid sequence identity to the following amino acid sequence: DFLVQFK GMCYFTNGTE RVRGVARYIY NREEYGRFDS DVGEFQAVTE LGRSIEDWNN YKDFLEQERA AVDKVCRHNY EAELRTTLQR QVEPTV (SEQ ID NO: 49); and can have a length of about 94 amino acids (e.g., 92 93, 94, 95, 96, or 97 amino acids). A suitable DQB2 β1 domain can comprise the following amino acid sequence: DFLVQFK GMCYFTNGTE RVRGVARYIY NREEYGRFDS DVGEFQAVTE LGRSIEDWNN YKDFLEQERA AVDKVCRHNY EAELRTTLQR QVEPTV (SEQ ID NO: 49), or a naturally-occurring allelic variant.

A suitable DQB2 β2 domain comprises an amino acid sequence having at least 60%, at least 70%, at least 80%, at least 90%, at least 95%, at least 98%, at least 99%, or 100%, amino acid sequence identity to the following amino acid sequence: TISP SRTEALNHHN LLVCSVTDFY PAQIKVRWFR NDQEETAGVV STSLIRNGDW TFQILVMLEI TPQRGDIYTC QVEHPSLQSP ITVEW (SEQ ID NO: 50); and can have a length of about 94 amino acids (e.g., 92 93, 94, 95, 96, or 97 amino acids). A suitable DQB2 β2 domain can comprise the following amino acid sequence: TISP SRTEALNHHN LLVCSVTDFY PAQIKVRWFR NDQEETAGVV STSLIRNGDW TFQILVMLEI TPQRGDIYTC QVEHPSLQSP ITVEW (SEQ ID NO: 50), or a naturally-occurring allelic variant thereof.

### Disease Risk-Associated Alleles and Haplotypes

Certain alleles and haplotypes of MHC Class II have been associated with disease, e.g., increased risk of developing a particular disease. See, e.g., Erlich et al. (2008) Diabetes 57:1084; Gough and Simmonds (2007) Curr. Genomics 8:453; Mitchell et al. (2007) Robbins Basic Pathology Philadelphia: Saunders, 8^{th} ed.; Margaritte-Jeannin et al. (2004) Tissue Antigens 63:562; and Kurko et al. (2013) Clin. Rev. Allergy Immunol. 45:170.

Further information regarding HLA-autoimmune disease associations and autoantigens can be found at, e.g., the following: 1) Fernando, et al., PLoS Genetics, Volume 4 (4) e1000024 (2008); 2) Jones, et al., Nature Reviews Immunology, 6: 271-282 (2006); 3) Gough, et al., Current Genomics, 2007, 8, 453-465 (2007); 4) Autoimmunity from Bench to Bedside, Anaya et al. Eds. Center for Autoimmune Diseases Research, CREA Texts Collection, School of Medicine and Health Sciences, El Rosario University (2013); see e.g., Cruz-Tapias et al., Chapter 17 HLA Association with Autoimmune Diseases, and Chapter 8 for immunogens; 5) Zanelli, et al., Rheumatology, Volume 39, Issue 10, 1 Oct. 2000, pages 1060-1066; 6) Santos et al., Expert Opin Drug Discov; 10(3): 269-292 (2015); 7) Conghua, et al., Medicine, 97(32): e11790 (2018); 8) Fallatah et al., Autoimmune Dis. 2012; 312817 (2012); 9) Ban et al., Genes and Immunity, 5, 203-208 (2004); 10) Menconi et al., Journal of Autoimmunity, 35(1), 45-51 (2010); 11) Sinkovits et al., Front. Immunol., 9: 1646 (2018); 12) Gladman et al., J. Rheumatology, 32 (8) 1481-1487 (2005); 13) Wang et al., PlosOne 9(1), e87363 (2014); 14) Zhou, et al., Int J. Immunopathol Pharmacol., 26(3): 747-751(2013); 15) Kemp et al. Autoimmunity in vitiligo in Autoimmune Disorders-Pathogenetic Aspects, Ed. C. P. Mavragani, in InTech Press: Rijeka, Croatia (2011); 16) Simmonds et al., Am. J. Hum. Genet. 76:157-163, (2005); 17) Ma & Qiu, J. Gastroenterol, 7(5):718-721 (2001); 18) Oka et al., PLoS One, 12(10):e0187325 (2017); 19) Miller et al., Genes Immun. 16(7): 470-480 (2015); 20) Tennebo et al., Rheumatology 54:528-535 (2015); 21) Greidinger et al. J. Immunol. 180:8444-8454 (2008); 22) Hollenbach et al., J. Autoimmun 65:13-25 (2015) and references cited therein; 23) Fogdell, et al., Tissue Antigens 1995: 46: 333-336; 24) Chen et al. J. Clin Endocrinol. Metab., 84: 3182-3186 (1999); 25) Sulzer et al., Nature, 546: 656-663 (2017); 26) Somma et al., Gastroenterol Res Pract. 2013: 683824 (2013); 27) Li et al., Mol Med Rep.; 17(5): 6533-6541 (2018); 28) Bodis, et al., Rheumatol Ther., 5(1):5-20 (2018); 29) Ooi, et al., Nature; 545(7653): 243-247(2017); 30) Yamamoto et al., J. Dermatol., 38(7) 711-716 (2011); 31) Hammers & Stanley, Annu. Rev. Pathol., 11: 175-197 (2016).

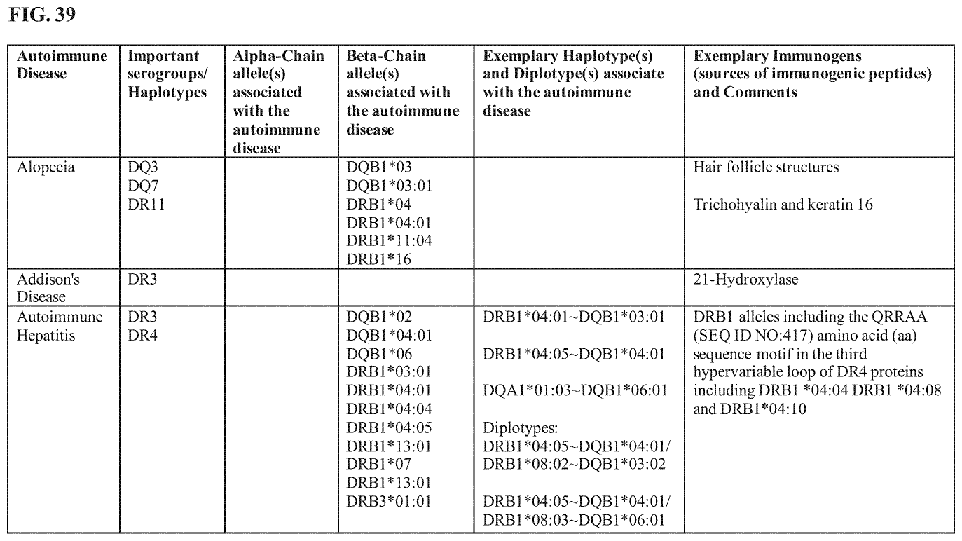

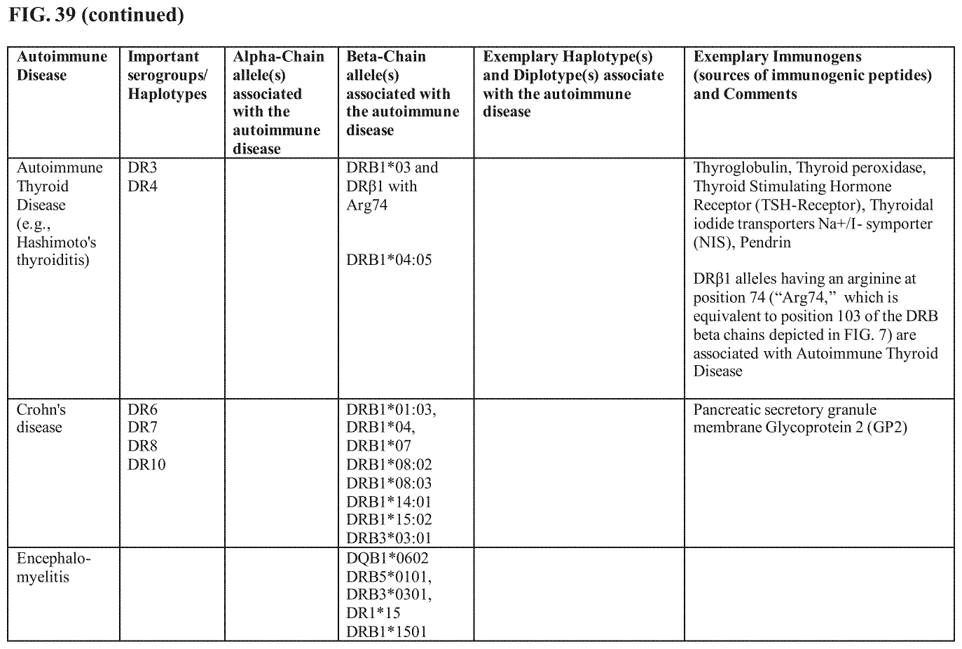

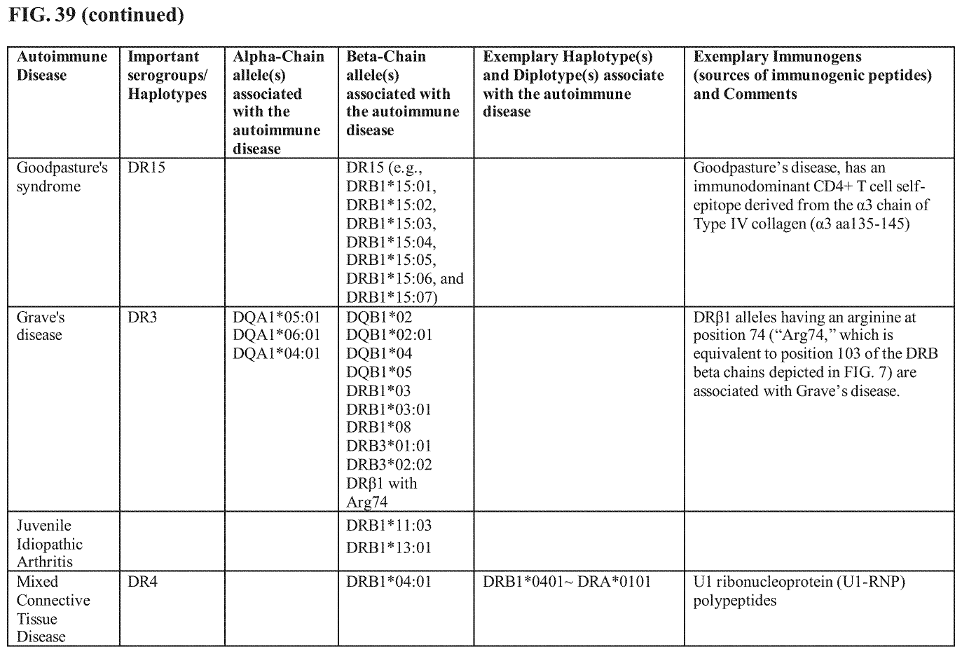

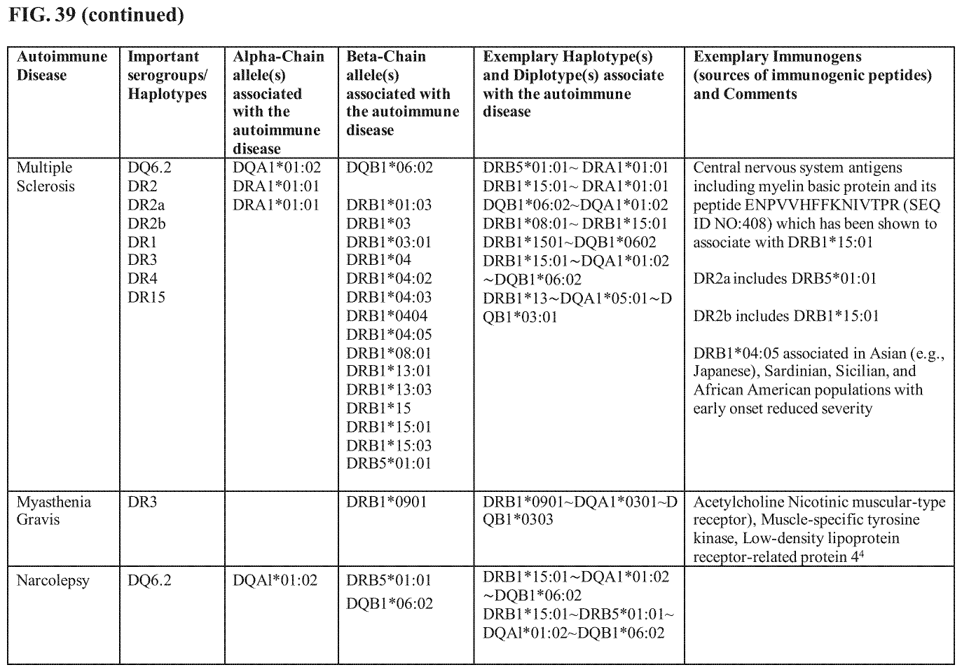

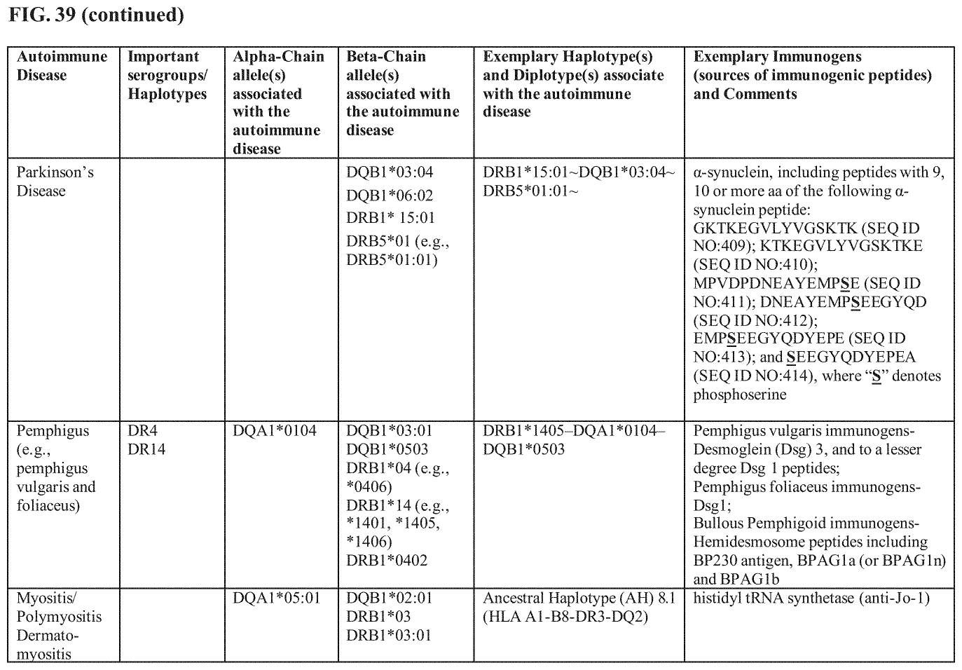

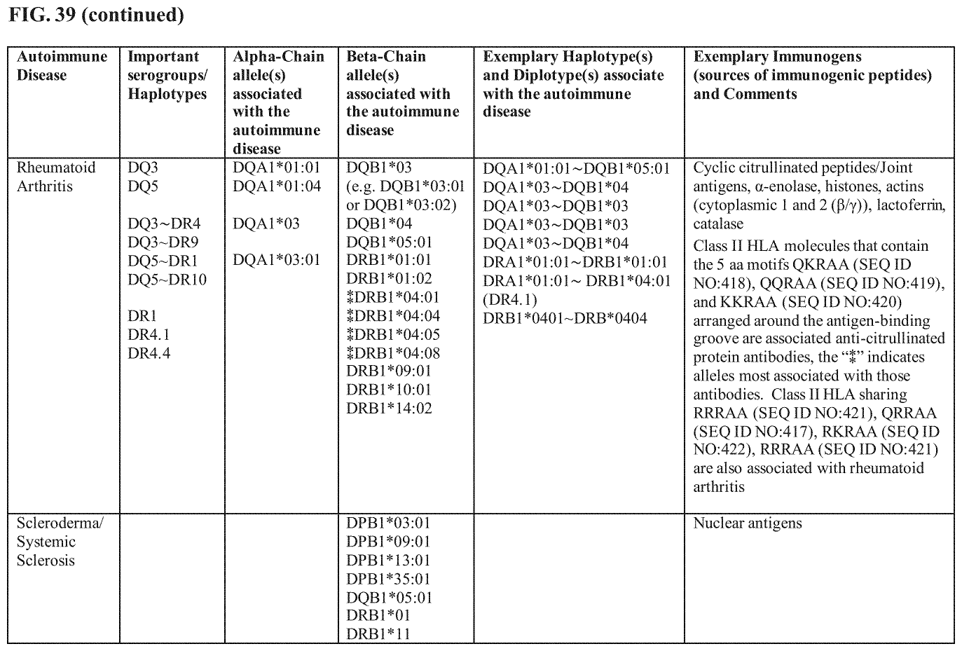

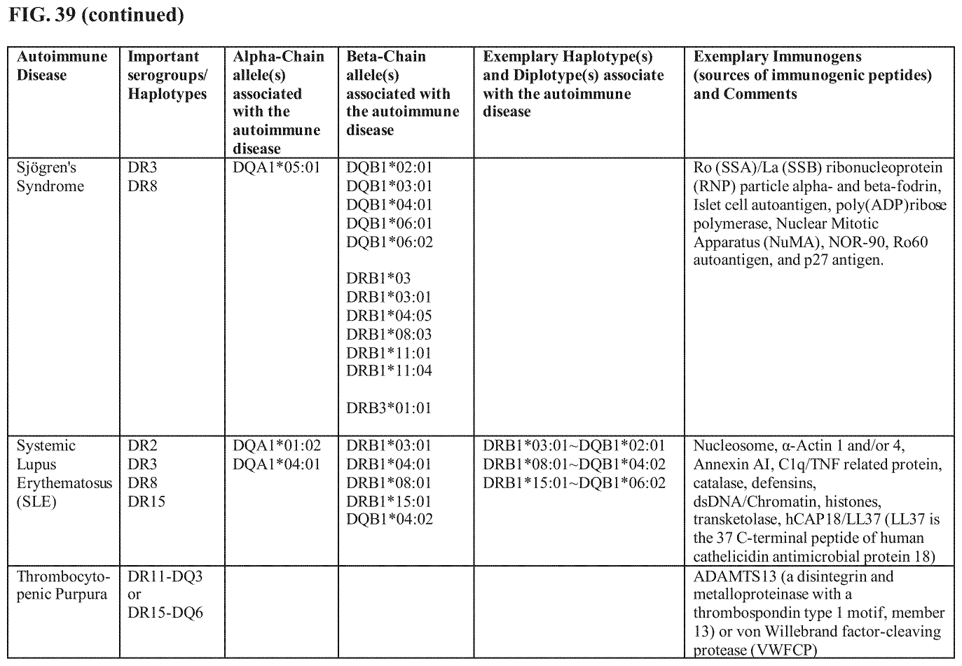

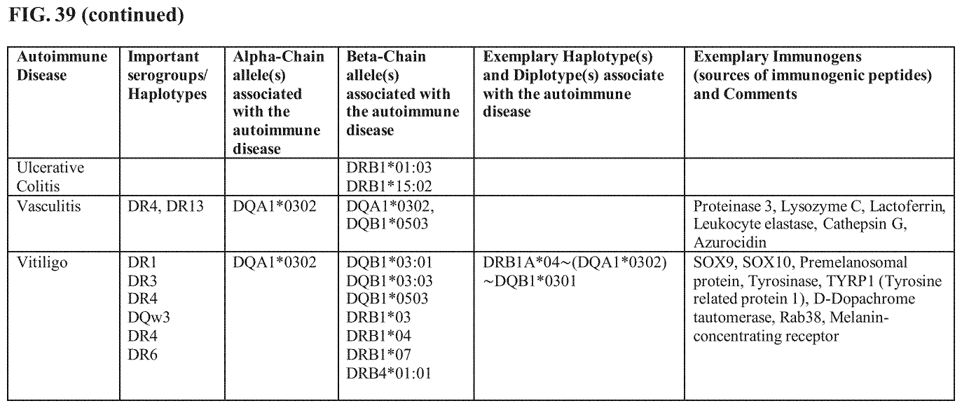

HLA haplotypes and alleles associated with increased risk that an individual expressing such HLA haplotypes and/or alleles will develop a given autoimmune disease are provided in the table provided in FIG. 39. A TMAPP of the present disclosure can include any of the HLA haplotypes and/or alleles set out in the table provided in FIG. 39. The table provided in FIG. 39 also provides examples of autoantigens associated with particular autoimmune diseases. A TMAPP of the present disclosure can include a peptide epitope (e.g., a peptide epitope of from 4 amino acids to about 25 amino acids in length) of any of the autoantigens set out in the table.

The following are notes to the table provided in FIG. 39. 1) AH8.1 (HLA A1-B8-DR3-DQ2 haplotype); 2) DQ3 alleles include DQB1*03 alleles such as DQB1*03:01 to DQB1*03:05 proteins; 3) DQ5 alleles include DQB1*05 alleles such as DQB1*05:01 to DQB1*05:04 and may be associated with DQA1*01:01; 4) DR2 alleles include DRB1*15:01-15:04 and DRB1*16:01-16:06; 5) DR3 haplotypes include: DRB1*03:01, DRB1*03:02, DRB1*03:03, and DRB1*03:04; 6) DR4 haplotypes include: DRB1*04:01 through DRB1*04:13; 7) AH=ancestral haplotype; 7) Simmonds et al., Am. J. Hum. Genet. 76:157-163, (2005), see Table 1, HLAs with odds ratios greater than 1.5 include the following DRB1, DAB1 and DQA1 alleles: DRB1* −03:01 to 05, −10:01, −08:01 to 11, −16:01 to 6, −11:01 to 21, −01:01 to 04, −04:01 to 22, and −15:01 to 05; DQB1* −02, −04, −03:01, 03:04, −05, −06:01 to 09, and −03:02; and HLA-DQA1* −05:01 to 02, −06:01, −04:01, −01:01, −01:04, −01:02, −01:03, −03:11, and −03:12; 8) Li et al., Mol Med Rep.; 17(5): 6533-6541 (2018) noting epitopes from auto antigens including: SMD1 (NCBI Accession: CAE11897.1); SMD2 (NCBI Accession: AAC13776.1); SMD3 (NCBI Accession: AAA57034.1); Proliferating cell nuclear antigen (PCNA) (NCBI Accession: NP_872590.1); Acidic ribosomal phosphoprotein (P1) (NCBI Accession: AAA36471.1); Acidic ribosomal phosphoprotein (P2) (NCBI Accession: AAA36472.1); snRNP-B/B′ (NCBI Accession: P14678.2); U1-snRNP-C(NCBI Accession: NP_003084.1); U1-snRNP-A (NCBI Accession: NP_004587.1); Nucleolin (NCBI Accession: AAA59954.1); Acidic ribosomal phosphoprotein (P0) (NCBI Accession: AAA36470.1); DNA topoisomerase1 (truncated) (NCBI Accession: NP_003277.1); DNA topoisomerase 1 (full length) (NCBI Accession: NP_003277.1); and U1-SnRNP 68/70 KDa (NCBI Accession: P08621.2).

### DRB1

HLA-DR3 serotype is associated with early-age onset myasthenia gravis, Hashimoto's thyroiditis, autoimmune hepatitis, primary Sjögren's syndrome, and SLE. Certain DRB1 alleles are associated with increased risk that an individual expressing such alleles will develop a particular autoimmune disease or diseases.

### DRB1*01:01

In some cases, a TMAPP of the present disclosure comprises a DRB1*01:01 polypeptide comprising an amino acid sequence having at least 90%, at least 95%, at least 98%, at least 99%, or 100%, amino acid sequence identity to amino acids 30-227 of the DRB1*01:01 amino acid sequence provided below. In some cases, a TMAPP of the present disclosure comprises a DRB1*01:01 polypeptide comprising an amino acid sequence having at least 90%, at least 95%, at least 98%, at least 99%, or 100%, amino acid sequence identity to amino acids 30-124 of the DRB1*01:01 amino acid sequence provided below. In some cases, a TMAPP of the present disclosure comprises a DRB1*01:01 polypeptide comprising an amino acid sequence having at least 90%, at least 95%, at least 98%, at least 99%, or 100%, amino acid sequence identity to amino acids 125-227 of the DRB1*01:01 amino acid sequence provided below.

DRB1*01:01:
(SEQ ID NO: 51)
MVCLKLPGGSCMTALTVTLMVLSSPLALAGDTRPRFLWQLKFECHFFNG
TERVRLLERCIYNQEESVRFDSDVGEYRAVTELGRPDAEYWNSQKDLLE
QRRAAVDTYCRHNYGVGESFTVQRRVEPKVTVYPSKTQPLQHHNLLVCS
VSGFYPGSIEVRWFRNGQEEKAGVVSTGLIQNGDWTFQTLVMLETVPRS
GEVYTCQVEHPSVTSPLTVEWRARSESAQSKMLSGVGGFVLGLLFLGAG
LFIYFRNQKGHSGLQPTGFLS.

### DRB1*01:02

In some cases, a TMAPP of the present disclosure comprises a DRB1*01:02 polypeptide comprising an amino acid sequence having at least 90%, at least 95%, at least 98%, at least 99%, or 100%, amino acid sequence identity to amino acids 30-227 of the DRB1*01:02 amino acid sequence provided below. In some cases, a TMAPP of the present disclosure comprises a DRB1*01:02 polypeptide comprising an amino acid sequence having at least 90%, at least 95%, at least 98%, at least 99%, or 100%, amino acid sequence identity to amino acids 30-124 of the DRB1*01:02 amino acid sequence provided below. In some cases, a TMAPP of the present disclosure comprises a DRB1*01:02 polypeptide comprising an amino acid sequence having at least 90%, at least 95%, at least 98%, at least 99%, or 100%, amino acid sequence identity to amino acids 125-227 of the DRB1*01:02 amino acid sequence provided below.

DRB1*01:02:
(SEQ ID NO: 52)
MVCLKLPGGSCMTALTVTLMVLSSPLALAGDTRPRFLWQLKFECHFFNG
TERVRLLERCIYNQEESVRFDSDVGEYRAVTELGRPDAEYWNSQKDLLE
QRRAAVDTYCRHNYGAVESFTVQRRVEPKVTVYPSKTQPLQHHNLLVCS
VSGFYPGSIEVRWFRNGQEEKAGVVSTGLIQNGDWTFQTLVMLETVPRS
GEVYTCQVEHPSVTSPLTVEWRARSESAQSKMLSGVGGFVLGLLFLGAG
LFIYFRNQKGHSGLQPTGFLS.

### DRB1*01:03

In some cases, a TMAPP of the present disclosure comprises a DRB1*01:03 polypeptide comprising an amino acid sequence having at least 90%, at least 95%, at least 98%, at least 99%, or 100%, amino acid sequence identity to amino acids 30-227 of the DRB1*01:03 amino acid sequence provided below. In some cases, a TMAPP of the present disclosure comprises a DRB1*01:03 polypeptide comprising an amino acid sequence having at least 90%, at least 95%, at least 98%, at least 99%, or 100%, amino acid sequence identity to amino acids 30-124 of the DRB1*01:03 amino acid sequence provided below. In some cases, a TMAPP of the present disclosure comprises a DRB1*01:03 polypeptide comprising an amino acid sequence having at least 90%, at least 95%, at least 98%, at least 99%, or 100%, amino acid sequence identity to amino acids 125-227 of the DRB1*01:03 amino acid sequence provided below.

DRB1*01:03:
(SEQ ID NO: 55)
MVCLKLPGGSCMTALTVTLMVLSSPLALAGDTRPRFLWQLKFECHFFNG
TERVRLLERCIYNQEESVRFDSDVGEYRAVTELGRPDAEYWNSQKDILE
DERAAVDTYCRHNYGVGESFTVQRRVEPKVTVYPSKTQPLQHHNLLVCS
VSGFYPGSIEVRWFRNGQEEKAGVVSTGLIQNGDWTFQTLVMLETVPRS
GEVYTCQVEHPSVTSPLTVEWRARSESAQSKMLSGVGGFVLGLLFLGAG
LFIYFRNQKGHSGLQPTGFLS.

### DRB1*03:01

DRB1*0301 (“DRB1*03:01” in FIG. 7) is associated with increased risk of developing early onset Grave's disease and/or type 1 autoimmune hepatitis. Thus, in some cases, a TMAPP of the present disclosure comprises a DRB1*03:01 polypeptide comprising an amino acid sequence having at least 90%, at least 95%, at least 98%, at least 99%, or 100%, amino acid sequence identity to amino acids 30-227 of the DRB1*03:01 amino acid sequence depicted in FIG. 7. In some cases, a TMAPP of the present disclosure comprises a DRB1*03:01 polypeptide comprising an amino acid sequence having at least 90%, at least 95%, at least 98%, at least 99%, or 100%, amino acid sequence identity to amino acids 30-124 of the DRB1*03:01 amino acid sequence depicted in FIG. 7. In some cases, a TMAPP of the present disclosure comprises a DRB1*03:01 polypeptide comprising an amino acid sequence having at least 90%, at least 95%, at least 98%, at least 99%, or 100%, amino acid sequence identity to amino acids 125-227 of the DRB1*03:01 amino acid sequence depicted in FIG. 7.

### DRB1*03:02

In some cases, a TMAPP of the present disclosure comprises a DRB1*03:02 polypeptide comprising an amino acid sequence having at least 90%, at least 95%, at least 98%, at least 99%, or 100%, amino acid sequence identity to amino acids 30-227 of the DRB1*03:02 amino acid sequence provided below. In some cases, a TMAPP of the present disclosure comprises a DRB1*03:02 polypeptide comprising an amino acid sequence having at least 90%, at least 95%, at least 98%, at least 99%, or 100%, amino acid sequence identity to amino acids 30-124 of the DRB1*03:02 amino acid sequence provided below. In some cases, a TMAPP of the present disclosure comprises a DRB1*03:02 polypeptide comprising an amino acid sequence having at least 90%, at least 95%, at least 98%, at least 99%, or 100%, amino acid sequence identity to amino acids 125-227 of the DRB1*03:02 amino acid sequence provided below.

DRB1*03:02:
(SEQ ID NO: 56)
MVCLRLPGGSCMAVLTVTLMVLSSPLALAGDTRPRFLEYSTSECHFFNG
TERVRFLERYFHNQEENVRFDSDVGEYRAVTELGRPDAEYWNSQKDLLE
QKRGRVDNYCRHNYGVGESFTVQRRVHPKVTVYPSKTQPLQHHNLLVCS
VSGFYPGSIEVRWFRNGQEEKTGVVSTGLIHNGDWTFQTLVMLETVPRS
GEVYTCQVEHPSVTSPLTVEWRARSESAQSKMLSGVGGFVLGLLFLGAG
LFIYFRNQKGHSGLQPRGFLS.

### DRB1*0304

DRB1*0304 is associated with Grave's disease. Thus, in some cases, a TMAPP of the present disclosure comprises a DRB1*03:04 polypeptide comprising an amino acid sequence having at least 90%, at least 95%, at least 98%, at least 99%, or 100%, amino acid sequence identity to amino acids 30-227 of the DRB1*03:04 amino acid sequence provided below. In some cases, a TMAPP of the present disclosure comprises a DRB1*03:04 polypeptide comprising an amino acid sequence having at least 90%, at least 95%, at least 98%, at least 99%, or 100%, amino acid sequence identity to amino acids 30-124 of the DRB1*03:04 amino acid sequence provided below. In some cases, a TMAPP of the present disclosure comprises a DRB1*03:04 polypeptide comprising an amino acid sequence having at least 90%, at least 95%, at least 98%, at least 99%, or 100%, amino acid sequence identity to amino acids 125-227 of the DRB1*03:04 amino acid sequence provided below.

DRB1*0304:
(SEQ ID NO: 57)
MVCLRLPGGSCMAVLTVTLMVLSSPLALAGDTRPRFLEYSTSECHFFNG
TERVRYLDRYFHNQEESVRFDSDVGEFRAVTELGRPDAEYWNSQKDLLE
QKRGRVDNYCRHNYGVVESFTVQRRVHPKVTVYPSKTQPLQHHNLLVCS
VSGFYPGSIEVRWFRNGQEEKTGVVSTGLIHNGDWTFQTLVMLETVPRS
GEVYTCQVEHPSVTSPLTVEWRARSESAQSKMLSGVGGFVLGLLFLGAG
LFIYFRNQKGHSGLQPRGFLS.

### DRB1*04:01

DRB1*04:01 is associated with increased risk of developing multiple sclerosis and/or rheumatoid arthritis. Thus, in some cases, a TMAPP of the present disclosure comprises a DRB1*04:01 polypeptide comprising an amino acid sequence having at least 90%, at least 95%, at least 98%, at least 99%, or 100%, amino acid sequence identity to amino acids 30-227 of the DRB1*04:01 amino acid sequence depicted in FIG. 7. In some cases, a TMAPP of the present disclosure comprises a DRB1*04:01 polypeptide comprising an amino acid sequence having at least 90%, at least 95%, at least 98%, at least 99%, or 100%, amino acid sequence identity to amino acids 30-124 of the DRB1*04:01 amino acid sequence depicted in FIG. 7. In some cases, a TMAPP of the present disclosure comprises a DRB1*04:01 polypeptide comprising an amino acid sequence having at least 90%, at least 95%, at least 98%, at least 99%, or 100%, amino acid sequence identity to amino acids 125-227 of the DRB1*04:01 amino acid sequence depicted in FIG. 7.

### DRB1*04:02

DRB1*04:02 is associated with increased risk of developing idiopathic pemphigus vulgaris, and/or SLE (e.g., SLE-associated anti-cardiolipin; SLE-associated anti-β2 glycoprotein I). Thus, in some cases, a TMAPP of the present disclosure comprises a DRB1*04:02 polypeptide comprising an amino acid sequence having at least 90%, at least 95%, at least 98%, at least 99%, or 100%, amino acid sequence identity to amino acids 30-227 of the DRB1*04:02 amino acid sequence provided below. In some cases, a TMAPP of the present disclosure comprises a DRB1*04:02 polypeptide comprising an amino acid sequence having at least 90%, at least 95%, at least 98%, at least 99%, or 100%, amino acid sequence identity to amino acids 30-124 of the DRB1*04:02 amino acid sequence provided below. In some cases, a TMAPP of the present disclosure comprises a DRB1*04:02 polypeptide comprising an amino acid sequence having at least 90%, at least 95%, at least 98%, at least 99%, or 100%, amino acid sequence identity to amino acids 125-227 of the DRB1*04:02 amino acid sequence provided below.

DRB1*04:02:
(SEQ ID NO: 53)
MVCLKFPGGSCMAALTVTLMVLSSPLALAGDTRPRFLEQVKHECHFFNG
TERVRFLDRYFYHQEEYVRFDSDVGEYRAVTELGRPDAEYWNSQKDILE
DERAAVDTYCRHNYGVVESFTVQRRVYPEVTVYPAKTQPLQHHNLLVCS
VNGFYPGSIEVRWFRNGQEEKTGVVSTGLIQNGDWTFQTLVMLETVPRS
GEVYTCQVEHPSLTSPLTVEWRARSESAQSKMLSGVGGFVLGLLFLGAG
LFIYFRNQKGHSGLQPTGFLS.

### DRB1*04:03

DRB1*0403 is associated with increased risk of developing SLE (e.g., increased risk of developing SLE-associated anti-cardiolipin antibodies and/or SLE-associated anti-β2 glycoprotein I antibodies). Thus, in some cases, a TMAPP of the present disclosure comprises a DRB1*04:03 polypeptide comprising an amino acid sequence having at least 90%, at least 95%, at least 98%, at least 99%, or 100%, amino acid sequence identity to amino acids 30 to 227 of the DRB1*04:03 amino acid sequence provided below. In some cases, a TMAPP of the present disclosure comprises a DRB1*04:03 polypeptide comprising an amino acid sequence having at least 90%, at least 95%, at least 98%, at least 99%, or 100%, amino acid sequence identity to amino acids 30-124 of the DRB1*04:03 amino acid sequence provided below. In some cases, a TMAPP of the present disclosure comprises a DRB1*04:03 polypeptide comprising an amino acid sequence having at least 90%, at least 95%, at least 98%, at least 99%, or 100%, amino acid sequence identity to amino acids 125-227 of the DRB1*04:03 amino acid sequence provided below.

DRB1*04:03:
(SEQ ID NO: 415)
MVCLKFPGGSCMAALTVTLMVLSSPLALAGDTRPRFLEQVKHECHFFNG
TERVRFLDRYFYHQEEYVRFDSDVGEYRAVTELGRPDAEYWNSQKDLLE
QRRAEVDTYCRHNYGVVESFTVQRRVYPEVTVYPAKTQPLQHHNLLVCS
VNGFYPGSIEVRWFRNGQEEKTGVVSTGLIQNGDWTFQTLVMLETVPRS
GEVYTCQVEHPSLTSPLTVEWRARSESAQSKMLSGVGGFVLGLLFLGAG
LFIYFRNQKGHSGLQPTGFLS

### DRB1*04:04

DRB1*04:04 is associated with increased risk of developing rheumatoid arthritis (e.g., increased risk of developing high titers of circulating anti-cyclic citrullinated peptide antibodies) and/or autoimmune hepatitis. Thus, in some cases, a TMAPP of the present disclosure comprises a DRB1*04:04 polypeptide comprising an amino acid sequence having at least 90%, at least 95%, at least 98%, at least 99%, or 100%, amino acid sequence identity to amino acids 30-227 of the DRB1*04:04 amino acid sequence provided below. In some cases, a TMAPP of the present disclosure comprises a DRB1*04:04 polypeptide comprising an amino acid sequence having at least 90%, at least 95%, at least 98%, at least 99%, or 100%, amino acid sequence identity to amino acids 30-124 of the DRB1*04:04 amino acid sequence provided below. In some cases, a TMAPP of the present disclosure comprises a DRB1*04:04 polypeptide comprising an amino acid sequence having at least 90%, at least 95%, at least 98%, at least 99%, or 100%, amino acid sequence identity to amino acids 125-227 of the DRB1*04:04 amino acid sequence provided below.

(SEQ ID NO: 416)
MVCLKFPGGSCMAALTVTLMVLSSPLALAGDTRPRFLEQVKHECHFFNG
TERVRFLDRYFYHQEEYVRFDSDVGEYRAVTELGRPDAEYWNSQKDLLE
QRRAAVDTYCRHNYGVVESFTVQRRVYPEVTVYPAKTQPLQHHNLLVCS
VNGFYPGSIEVRWFRNGQEEKTGVVSTGLIQNGDWTFQTLVMLETVPRS
GEVYTCQVEHPSLTSPLTVEWRARSESAQSKMLSGVGGFVLGLLFLGAG
LFIYFRNQKGHSGLQPTGFLS.

### DRB1*04:05

DRB1*04:05 is associated with increased risk of developing rheumatoid arthritis and/or autoimmune hepatitis. Thus, in some cases, a TMAPP of the present disclosure comprises a DRB1*04:05 polypeptide comprising an amino acid sequence having at least 90%, at least 95%, at least 98%, at least 99%, or 100%, amino acid sequence identity to amino acids 30-227 of the DRB1*04:05 amino acid sequence provided below. In some cases, a TMAPP of the present disclosure comprises a DRB1*04:05 polypeptide comprising an amino acid sequence having at least 90%, at least 95%, at least 98%, at least 99%, or 100%, amino acid sequence identity to amino acids 30-124 of the DRB1*04:05 amino acid sequence provided below. In some cases, a TMAPP of the present disclosure comprises a DRB1*04:05 polypeptide comprising an amino acid sequence having at least 90%, at least 95%, at least 98%, at least 99%, or 100%, amino acid sequence identity to amino acids 125-227 of the DRB1*04:05 amino acid sequence provided below.

DRB1*04:05:
(SEQ ID NO: 54)
MVCLKFPGGSCMAALTVTLMVLSSPLALAGDTRPRFLEQVKHECHFFNG
TERVRFLDRYFYHQEEYVRFDSDVGEYRAVTELGRPSAEYWNSQKDLLE
QRRAAVDTYCRHNYGVGESFTVQRRVYPEVTVYPAKTQPLQHHNLLVCS
VNGFYPGSIEVRWFRNGQEEKTGVVSTGLIQNGDWTFQTLVMLETVPRS
GEVYTCQVEHPSLTSPLTVEWRARSESAQSKMLSGVGGFVLGLLFLGAG
LFIYFRNQKGHSGLQPTGFLS.

### DRB1*04:06

DRB1*04:06 is associated with increased risk of developing anti-caspase-8 autoantibodies (e.g., in silicosis-systemic sclerosis (SSc)-systemic lupus erythematosus (SLE). Thus, in some cases, a TMAPP of the present disclosure comprises a DRB1*04:06 polypeptide comprising an amino acid sequence having at least 90%, at least 95%, at least 98%, at least 99%, or 100%, amino acid sequence identity to amino acids 30-227 of the DRB1*04:06 amino acid sequence provided below. In some cases, a TMAPP of the present disclosure comprises a DRB1*04:06 polypeptide comprising an amino acid sequence having at least 90%, at least 95%, at least 98%, at least 99%, or 100%, amino acid sequence identity to amino acids 30-124 of the DRB1*04:06 amino acid sequence provided below. In some cases, a TMAPP of the present disclosure comprises a DRB1*04:06 polypeptide comprising an amino acid sequence having at least 90%, at least 95%, at least 98%, at least 99%, or 100%, amino acid sequence identity to amino acids 125-227 of the DRB1*04:06 amino acid sequence provided below.

DRB1*04:06
(SEQ ID NO: 58)
MVCLKFPGGSCMAALTVTLMVLSSPLALAGDTRPRFLEQVKHECHFFNG
TERVRFLDRYFYHQEESVRFDSDVGEYRAVTELGRPDAEYWNSQKDLLE
QRRAEVDTYCRHNYGVVESFTVQRRVYPEVTVYPAKTQPLQHHNLLVCS
VNGFYPGSIEVRWFRNGQEEKTGVVSTGLIQNGDWTFQTLVMLETVPRS
GEVYTCQVEHPSLTSPLTVEWRARSESAQSKMLSGVGGFVLGLLFLGAG
LFIYFRNQKGHSGLQPTGFLS.

### DRB1*04:08

In some cases, a TMAPP of the present disclosure comprises a DRB1*04:08 polypeptide comprising an amino acid sequence having at least 90%, at least 95%, at least 98%, at least 99%, or 100%, amino acid sequence identity to amino acids 30-227 of the DRB1*04:08 amino acid sequence provided below. In some cases, a TMAPP of the present disclosure comprises a DRB1*04:08 polypeptide comprising an amino acid sequence having at least 90%, at least 95%, at least 98%, at least 99%, or 100%, amino acid sequence identity to amino acids 30-124 of the DRB1*04:08 amino acid sequence provided below. In some cases, a TMAPP of the present disclosure comprises a DRB1*04:08 polypeptide comprising an amino acid sequence having at least 90%, at least 95%, at least 98%, at least 99%, or 100%, amino acid sequence identity to amino acids 125-227 of the DRB1*04:08 amino acid sequence provided below.

DRB1*04:08:
(SEQ ID NO: 72)
MVCLKFPGGSCMAALTVTLMVLSSPLALAGDTRPRFLEQVKHECHFFNG
TERVRFLDRYFYHQEEYVRFDSDVGEYRAVTELGRPDAEYWNSQKDLLE
QRRAAVDTYCRHNYGVGESFTVQRRVYPEVTVYPAKTQPLQHHNLLVCS
VNGFYPGSIEVRWFRNGQEEKTGVVSTGLIQNGDWTFQTLVMLETVPRS
GEVYTCQVEHPSLTSPLTVEWRARSESAQSKMLSGVGGFVLGLLFLGAG
LFIYFRNQKGHSGLQPTGFLS.

### DRB1*08:01

In some cases, a TMAPP of the present disclosure comprises a DRB1*08:01 polypeptide comprising an amino acid sequence having at least 90%, at least 95%, at least 98%, at least 99%, or 100%, amino acid sequence identity to amino acids 30-227 of the DRB1*08:01 amino acid sequence provided below. In some cases, a TMAPP of the present disclosure comprises a DRB1*08:01 polypeptide comprising an amino acid sequence having at least 90%, at least 95%, at least 98%, at least 99%, or 100%, amino acid sequence identity to amino acids 30-124 of the DRB1*08:01 amino acid sequence provided below. In some cases, a TMAPP of the present disclosure comprises a DRB1*08:01 polypeptide comprising an amino acid sequence having at least 90%, at least 95%, at least 98%, at least 99%, or 100%, amino acid sequence identity to amino acids 125-227 of the DRB1*08:01 amino acid sequence provided below.

DRB1*08:01:
(SEQ ID NO: 73)
MVCLRLPGGSCMAVLTVTLMVLSSPLALAGDTRPRFLEYSTGECYFFNG
TERVRFLDRYFYNQEEYVRFDSDVGEYRAVTELGRPSAEYWNSQKDFLE
DRRALVDTYCRHNYGVGESFTVQRRVHPKVTVYPSKTQPLQHHNLLVCS
VSGFYPGSIEVRWFRNGQEEKTGVVSTGLIHNGDWTFQTLVMLETVPRS
GEVYTCQVEHPSVTSPLTVEWSARSESAQSKMLSGVGGFVLGLLFLGAG
LFIYFRNQKGHSGLQPTGFLS.

### DRB1*08:02

In some cases, a TMAPP of the present disclosure comprises a DRB1*08:02 polypeptide comprising an amino acid sequence having at least 90%, at least 95%, at least 98%, at least 99%, or 100%, amino acid sequence identity to amino acids 30-227 of the DRB1*08:02 amino acid sequence provided below. In some cases, a TMAPP of the present disclosure comprises a DRB1*08:02 polypeptide comprising an amino acid sequence having at least 90%, at least 95%, at least 98%, at least 99%, or 100%, amino acid sequence identity to amino acids 30-124 of the DRB1*08:02 amino acid sequence provided below. In some cases, a TMAPP of the present disclosure comprises a DRB1*08:02 polypeptide comprising an amino acid sequence having at least 90%, at least 95%, at least 98%, at least 99%, or 100%, amino acid sequence identity to amino acids 125-227 of the DRB1*08:02 amino acid sequence provided below.

DRB1*08:02:
(SEQ ID NO: 74)
MVCLRLPGGSCMAVLTVTLMVLSSPLALAGDTRPRFLEYSTGECYFFNG
TERVRFLDRYFYNQEEYVRFDSDVGEYRAVTELGRPDAEYWNSQKDFLE
DRRALVDTYCRHNYGVGESFTVQRRVHPKVTVYPSKTQPLQHHNLLVCS
VSGFYPGSIEVRWFRNGQEEKTGVVSTGLIHNGDWTFQTLVMLETVPRS
GEVYTCQVEHPSVTSPLTVEWSARSESAQSKMLSGVGGFVLGLLFLGAG
LFIYFRNQKGHSGLQPTGFLS.

### DRB1*08:03

In some cases, a TMAPP of the present disclosure comprises a DRB1*08:03 polypeptide comprising an amino acid sequence having at least 90%, at least 95%, at least 98%, at least 99%, or 100%, amino acid sequence identity to amino acids 30-227 of the DRB1*08:03 amino acid sequence provided below. In some cases, a TMAPP of the present disclosure comprises a DRB1*08:03 polypeptide comprising an amino acid sequence having at least 90%, at least 95%, at least 98%, at least 99%, or 100%, amino acid sequence identity to amino acids 30-124 of the DRB1*08:03 amino acid sequence provided below. In some cases, a TMAPP of the present disclosure comprises a DRB1*08:03 polypeptide comprising an amino acid sequence having at least 90%, at least 95%, at least 98%, at least 99%, or 100%, amino acid sequence identity to amino acids 125-227 of the DRB1*08:03 amino acid sequence provided below.

DRB1*08:03:
(SEQ ID NO: 75)
MVCLRLPGGSCMAVLTVTLMVLSSPLALAGDTRPRFLEYSTGECYFFNG
TERVRFLDRYFYNQEEYVRFDSDVGEYRAVTELGRPSAEYWNSQKDILE
DRRALVDTYCRHNYGVGESFTVQRRVHPKVTVYPSKTQPLQHHNLLVCS
VSGFYPGSIEVRWFRNGQEEKTGVVSTGLIHNGDWTFQTLVMLETVPRS
GEVYTCQVEHPSVTSPLTVEWSARSESAQSKMLSGVGGFVLGLLFLGAG
LFIYFRNQKGHSGLQPTGFLS.

### DRB1*09:01

In some cases, a TMAPP of the present disclosure comprises a DRB1*09:01 polypeptide comprising an amino acid sequence having at least 90%, at least 95%, at least 98%, at least 99%, or 100%, amino acid sequence identity to amino acids 30-227 of the DRB1*09:01 amino acid sequence provided below. In some cases, a TMAPP of the present disclosure comprises a DRB1*09:01 polypeptide comprising an amino acid sequence having at least 90%, at least 95%, at least 98%, at least 99%, or 100%, amino acid sequence identity to amino acids 30-124 of the DRB1*09:01 amino acid sequence provided below. In some cases, a TMAPP of the present disclosure comprises a DRB1*09:01 polypeptide comprising an amino acid sequence having at least 90%, at least 95%, at least 98%, at least 99%, or 100%, amino acid sequence identity to amino acids 125-227 of the DRB1*09:01 amino acid sequence provided below.

DRB1*09:01:
(SEQ ID NO: 76)
MVCLKLPGGSCMAALTVTLMVLSSPLALAGDTQPRFLKQDKFECHFFNG
TERVRYLHRGIYNQEENVRFDSDVGEYRAVTELGRPVAESWNSQKDFLE
RRRAEVDTVCRHNYGVGESFTVQRRVHPEVTVYPAKTQPLQHHNLLVCS
VSGFYPGSIEVRWFRNGQEEKAGVVSTGLIQNGDWTFQTLVMLETVPRS
GEVYTCQVEHPSVMSPLTVEWRARSESAQSKMLSGVGGFVLGLLFLGAG
LFIYFRNQKGHSGLQPTGFLS.

### DRB1*10:01

In some cases, a TMAPP of the present disclosure comprises a DRB1*10:01 polypeptide comprising an amino acid sequence having at least 90%, at least 95%, at least 98%, at least 99%, or 100%, amino acid sequence identity to amino acids 30-227 of the DRB1*10:01 amino acid sequence provided below. In some cases, a TMAPP of the present disclosure comprises a DRB1*10:01 polypeptide comprising an amino acid sequence having at least 90%, at least 95%, at least 98%, at least 99%, or 100%, amino acid sequence identity to amino acids 30-124 of the DRB1*10:01 amino acid sequence provided below. In some cases, a TMAPP of the present disclosure comprises a DRB1*10:01 polypeptide comprising an amino acid sequence having at least 90%, at least 95%, at least 98%, at least 99%, or 100%, amino acid sequence identity to amino acids 125-227 of the DRB1*10:01 amino acid sequence provided below.

DRB1*10:01:
(SEQ ID NO: 77)
MVCLRLPGGSCMAVLTVTLMVLSSPLALAGDTRPRFLEEVKFECHFFNG
TERVRLLERRVHNQEEYARYDSDVGEYRAVTELGRPDAEYWNSQKDLLE
RRRAAVDTYCRHNYGVGESFTVQRRVQPKVTVYPSKTQPLQHHNLLVCS
VNGFYPGSIEVRWFRNGQEEKTGVVSTGLIQNGDWTFQTLVMLETVPQS
GEVYTCQVEHPSVMSPLTVEWRARSESAQSKMLSGVGGFVLGLLFLGAG
LFIYFRNQKGHSGLPPTGFLS.

### DRB1*11:01

In some cases, a TMAPP of the present disclosure comprises a DRB1*11:01 polypeptide comprising an amino acid sequence having at least 90%, at least 95%, at least 98%, at least 99%, or 100%, amino acid sequence identity to amino acids 30-227 of the DRB1*11:01 amino acid sequence provided below. In some cases, a TMAPP of the present disclosure comprises a DRB1*11:01 polypeptide comprising an amino acid sequence having at least 90%, at least 95%, at least 98%, at least 99%, or 100%, amino acid sequence identity to amino acids 30-124 of the DRB1*11:01 amino acid sequence provided below. In some cases, a TMAPP of the present disclosure comprises a DRB1*11:01 polypeptide comprising an amino acid sequence having at least 90%, at least 95%, at least 98%, at least 99%, or 100%, amino acid sequence identity to amino acids 125-227 of the DRB1*11:01 amino acid sequence provided below.

DRB1*11:01:
(SEQ ID NO: 78)
MVCLRLPGGSCMAVLTVTLMVLSSPLALAGDTRPRFLEYSTSECHFFNG
TERVRFLDRYFYNQEEYVRFDSDVGEFRAVTELGRPDEEYWNSQKDFLE
DRRAAVDTYCRHNYGVGESFTVQRRVHPKVTVYPSKTQPLQHHNLLVCS
VSGFYPGSIEVRWFRNGQEEKTGVVSTGLIHNGDWTFQTLVMLETVPRS
GEVYTCQVEHPSVTSPLTVEWRARSESAQSKMLSGVGGFVLGLLFLGAG
LFIYFRNQKGHSGLQPRGFLS.

### DRB1*11:03

In some cases, a TMAPP of the present disclosure comprises a DRB1*11:03 polypeptide comprising an amino acid sequence having at least 90%, at least 95%, at least 98%, at least 99%, or 100%, amino acid sequence identity to amino acids 30-227 of the DRB1*11:03 amino acid sequence provided below. In some cases, a TMAPP of the present disclosure comprises a DRB1*11:03 polypeptide comprising an amino acid sequence having at least 90%, at least 95%, at least 98%, at least 99%, or 100%, amino acid sequence identity to amino acids 30-124 of the DRB1*11:03 amino acid sequence provided below. In some cases, a TMAPP of the present disclosure comprises a DRB1*11:03 polypeptide comprising an amino acid sequence having at least 90%, at least 95%, at least 98%, at least 99%, or 100%, amino acid sequence identity to amino acids 125-227 of the DRB1*11:03 amino acid sequence provided below.

DRB1*11:03:
(SEQ ID NO: 79)
MVCLRLPGGSCMAVLTVTLMVLSSPLALAGDTRPRFLEYSTSECHFFNG
TERVRFLDRYFYNQEEYVRFDSDVGEFRAVTELGRPDEEYWNSQKDFLE
DERAAVDTYCRHNYGVVESFTVQRRVHPKVTVYPSKTQPLQHHNLLVCS
VSGFYPGSIEVRWFRNGQEEKTGVVSTGLIHNGDWTFQTLVMLETVPRS
GEVYTCQVEHPSVTSPLTVEWRARSESAQSKMLSGVGGFVLGLLFLGAG
LFIYFRNQKGHSGLQPRGFLS.

### DRB1*11:04

In some cases, a TMAPP of the present disclosure comprises a DRB1*11:04 polypeptide comprising an amino acid sequence having at least 90%, at least 95%, at least 98%, at least 99%, or 100%, amino acid sequence identity to amino acids 30-227 of the DRB1*11:04 amino acid sequence provided below. In some cases, a TMAPP of the present disclosure comprises a DRB1*11:04 polypeptide comprising an amino acid sequence having at least 90%, at least 95%, at least 98%, at least 99%, or 100%, amino acid sequence identity to amino acids 30-124 of the DRB1*11:04 amino acid sequence provided below. In some cases, a TMAPP of the present disclosure comprises a DRB1*11:04 polypeptide comprising an amino acid sequence having at least 90%, at least 95%, at least 98%, at least 99%, or 100%, amino acid sequence identity to amino acids 125-227 of the DRB1*11:04 amino acid sequence provided below.

DRB1*11:04:
(SEQ ID NO: 80)
MVCLRLPGGSCMAVLTVTLMVLSSPLALAGDTRPRFLEYSTSECHFFNGT
ERVRFLDRYFYNQEEYVRFDSDVGEFRAVTELGRPDEEYWNSQKDFLEDR
RAAVDTYCRHNYGVVESFTVQRRVHPKVTVYPSKTQPLQHHNLLVCSVSG
FYPGSIEVRWFRNGQEEKTGVVSTGLIHNGDWTFQTLVMLETVPRSGEVY
TCQVEHPSVTSPLTVEWRARSESAQSKMLSGVGGFVLGLLFLGAGLFIYF
RNQKGHSGLQPRGFLS.

### DRB1*13:01

In some cases, a TMAPP of the present disclosure comprises a DRB1*13:01 polypeptide comprising an amino acid sequence having at least 90%, at least 95%, at least 98%, at least 99%, or 100%, amino acid sequence identity to amino acids 30-227 of the DRB1*13:01 amino acid sequence provided below. In some cases, a TMAPP of the present disclosure comprises a DRB1*13:01 polypeptide comprising an amino acid sequence having at least 90%, at least 95%, at least 98%, at least 99%, or 100%, amino acid sequence identity to amino acids 30-124 of the DRB1*13:01 amino acid sequence provided below. In some cases, a TMAPP of the present disclosure comprises a DRB1*13:01 polypeptide comprising an amino acid sequence having at least 90%, at least 95%, at least 98%, at least 99%, or 100%, amino acid sequence identity to amino acids 125-227 of the DRB1*13:01 amino acid sequence provided below.

DRB1*13:01:
(SEQ ID NO: 81)
MVCLRLPGGSCMAVLTVTLMVLSSPLALAGDTRPRFLEYSTSECHFFNGT
ERVRFLDRYFHNQEENVRFDSDVGEFRAVTELGRPDAEYWNSQKDILEDE
RAAVDTYCRHNYGVVESFTVQRRVHPKVTVYPSKTQPLQHHNLLVCSVSG
FYPGSIEVRWFRNGQEEKTGVVSTGLIHNGDWTFQTLVMLETVPRSGEVY
TCQVEHPSVTSPLTVEWRARSESAQSKMLSGVGGFVLGLLFLGAGLFIYF
RNQKGHSGLQPRGFLS.

### DRB1*13:03

In some cases, a TMAPP of the present disclosure comprises a DRB1*13:03 polypeptide comprising an amino acid sequence having at least 90%, at least 95%, at least 98%, at least 99%, or 100%, amino acid sequence identity to amino acids 30-227 of the DRB1*13:03 amino acid sequence provided below. In some cases, a TMAPP of the present disclosure comprises a DRB1*13:03 polypeptide comprising an amino acid sequence having at least 90%, at least 95%, at least 98%, at least 99%, or 100%, amino acid sequence identity to amino acids 30-124 of the DRB1*13:03 amino acid sequence provided below. In some cases, a TMAPP of the present disclosure comprises a DRB1*13:03 polypeptide comprising an amino acid sequence having at least 90%, at least 95%, at least 98%, at least 99%, or 100%, amino acid sequence identity to amino acids 125-227 of the DRB1*13:031 amino acid sequence provided below.

DRB1*13:03:
(SEQ ID NO: 82)
MVCLRLPGGSCMAVLTVTLMVLSSPLALAGDTRPRFLEYSTSECHFFNG
TERVRFLDRYFYNQEEYVRFDSDVGEYRAVTELGRPSAEYWNSQKDILE
DKRAAVDTYCRHNYGVGESFTVQRRVHPKVTVYPSKTQPLQHHNLLVCS
VSGFYPGSIEVRWFRNGQEEKTGVVSTGLIHNGDWTFQTLVMLETVPRS
GEVYTCQVEHPSVTSPLTVEWRARSESAQSKMLSGVGGFVLGLLFLGAG
LFIYFRNQKGHSGLQPRGFLS.

### DRB1*14:01

In some cases, a TMAPP of the present disclosure comprises a DRB1*14:01 polypeptide comprising an amino acid sequence having at least 90%, at least 95%, at least 98%, at least 99%, or 100%, amino acid sequence identity to amino acids 30-227 of the DRB1*14:01 amino acid sequence provided below. In some cases, a TMAPP of the present disclosure comprises a DRB1*14:01 polypeptide comprising an amino acid sequence having at least 90%, at least 95%, at least 98%, at least 99%, or 100%, amino acid sequence identity to amino acids 30-124 of the DRB1*14:01 amino acid sequence provided below. In some cases, a TMAPP of the present disclosure comprises a DRB1*14:01 polypeptide comprising an amino acid sequence having at least 90%, at least 95%, at least 98%, at least 99%, or 100%, amino acid sequence identity to amino acids 125-227 of the DRB1*14:01 amino acid sequence provided below.

DRB1*14:01:
(SEQ ID NO: 83)
MVCLRLPGGSCMAVLTVTLMVLSSPLALAGDTRPRFLEYSTSECHFFNG
TERVRFLDRYFHNQEEFVRFDSDVGEYRAVTELGRPAAEHWNSQKDLLE
RRRAEVDTYCRHNYGVVESFTVQRRVHPKVTVYPSKTQPLQHYNLLVCS
VSGFYPGSIEVRWFRNGQEEKTGVVSTGLIHNGDWTFQTLVMLETVPRS
GEVYTCQVEHPSVTSPLTVEWRARSESAQSKMLSGVGGFVLGLLFLGAG
LFIYFRNQKGHSGLQPRGFLS.

### DRB1*14:02

In some cases, a TMAPP of the present disclosure comprises a DRB1*14:02 polypeptide comprising an amino acid sequence having at least 90%, at least 95%, at least 98%, at least 99%, or 100%, amino acid sequence identity to amino acids 30-227 of the DRB1*14:02 amino acid sequence provided below. In some cases, a TMAPP of the present disclosure comprises a DRB1*14:02 polypeptide comprising an amino acid sequence having at least 90%, at least 95%, at least 98%, at least 99%, or 100%, amino acid sequence identity to amino acids 30-124 of the DRB1*14:02 amino acid sequence provided below. In some cases, a TMAPP of the present disclosure comprises a DRB1*14:02 polypeptide comprising an amino acid sequence having at least 90%, at least 95%, at least 98%, at least 99%, or 100%, amino acid sequence identity to amino acids 125-227 of the DRB1*14:02 amino acid sequence provided below.

DRB1*14:02:
(SEQ ID NO: 84)
MVCLRLPGGSCMAVLTVTLMVLSSPLALAGDTRPRFLEYSTSECHFFNG
TERVRFLERYFHNQEENVRFDSDVGEYRAVTELGRPDAEYWNSQKDLLE
QRRAAVDTYCRHNYGVGESFTVQRRVHPKVTVYPSKTQPLQHHNLLVCS
VSGFYPGSIEVRWFRNGQEEKTGVVSTGLIHNGDWTFQTLVMLETVPRS
GEVYTCQVEHPSVTSPLTVEWRARSESAQSKMLSGVGGFVLGLLFLGAG
LFIYFRNQKGHSGLQPRGFLS.

### DRB1*14:05

In some cases, a TMAPP of the present disclosure comprises a DRB1*14:05 polypeptide comprising an amino acid sequence having at least 90%, at least 95%, at least 98%, at least 99%, or 100%, amino acid sequence identity to amino acids 30-227 of the DRB1*14:05 amino acid sequence provided below. In some cases, a TMAPP of the present disclosure comprises a DRB1*14:05 polypeptide comprising an amino acid sequence having at least 90%, at least 95%, at least 98%, at least 99%, or 100%, amino acid sequence identity to amino acids 30-124 of the DRB1*14:05 amino acid sequence provided below. In some cases, a TMAPP of the present disclosure comprises a DRB1*14:05 polypeptide comprising an amino acid sequence having at least 90%, at least 95%, at least 98%, at least 99%, or 100%, amino acid sequence identity to amino acids 125-227 of the DRB1*14:05 amino acid sequence provided below.

DRB1*14:05:
(SEQ ID NO: 85)
MVCLRLPGGSCMAVLTVTLMVLSSPLALAGDTRPRFLEYSTSECQFFNG
TERVRFLDRYFHNQEEFVRFDSDVGEYRAVTELGRPDAEYWNSQKDLLE
RRRAEVDTYCRHNYGVVESFTVQRRVHPKVTVYPSKTQPLQHHNLLVCS
VSGFYPGSIEVRWFRNGQEEKTGVVSTGLIHNGDWTFQTLVMLETVPRS
GEVYTCQVEHPSVTSPLTVEWRARSESAQSKMLSGVGGFVLGLLFLGAGL
FIYFRNQKGHSGLQPRGFLS.

### DRB1*14:06

DRB1*14:06:

In some cases, a TMAPP of the present disclosure comprises a DRB1*14:06 polypeptide comprising an amino acid sequence having at least 90%, at least 95%, at least 98%, at least 99%, or 100%, amino acid sequence identity to amino acids 30-227 of the DRB1*14:06 amino acid sequence provided below. In some cases, a TMAPP of the present disclosure comprises a DRB1*14:06 polypeptide comprising an amino acid sequence having at least 90%, at least 95%, at least 98%, at least 99%, or 100%, amino acid sequence identity to amino acids 30-124 of the DRB1*14:06 amino acid sequence provided below. In some cases, a TMAPP of the present disclosure comprises a DRB1*14:06 polypeptide comprising an amino acid sequence having at least 90%, at least 95%, at least 98%, at least 99%, or 100%, amino acid sequence identity to amino acids 125-227 of the DRB1*14:06 amino acid sequence provided below.

(SEQ ID NO: 86)
MVCLRLPGGSCMAVLTVTLMVLSSPLALAGDTRPRFLEYSTSECHFFNG
TERVRFLERYFHNQEENVRFDSDVGEYRAVTELGRPDAEYWNSQKDLLE
QRRAAVDTYCRHNYGVVESFTVQRRVHPKVTVYPSKTQPLQHHNLLVCS
VSGFYPGSIEVRWFRNGQEEKTGVVSTGLIHNGDWTFQTLVMLETVPRS
GEVYTCQVEHPSVTSPLTVEWRARSESAQSKMLSGVGGFVLGLLFLGAG
LFIYFRNQKGHSGLQPRGFLS.

### DRB1*1501, DRB1*1502, and DRB1*1503

DR2 serotypes are associated with increased risk of SLE and/or MS. HLA alleles associated with increased risk of SLE and/or MS include DRB1*1501, DRB1*1502, and DRB1*1503.

Thus, in some cases, a TMAPP of the present disclosure comprises a DRB1*1501 polypeptide comprising an amino acid sequence having at least 90%, at least 95%, at least 98%, at least 99%, or 100%, amino acid sequence identity to amino acids 30-227 of the DRB1*1501 amino acid sequence depicted in FIG. 7. In some cases, a TMAPP of the present disclosure comprises a DRB1*1501 polypeptide comprising an amino acid sequence having at least 90%, at least 95%, at least 98%, at least 99%, or 100%, amino acid sequence identity to amino acids 30-124 of the DRB1*1501 amino acid sequence depicted in FIG. 7. In some cases, a TMAPP of the present disclosure comprises a DRB1*1501 polypeptide comprising an amino acid sequence having at least 90%, at least 95%, at least 98%, at least 99%, or 100%, amino acid sequence identity to amino acids 125-227 of the DRB1*1501 amino acid sequence depicted in FIG. 7.

In some cases, a TMAPP of the present disclosure comprises a DRB1*1502 polypeptide comprising an amino acid sequence having at least 90%, at least 95%, at least 98%, at least 99%, or 100%, amino acid sequence identity to amino acids 30-227 of the DRB1*1502 amino acid sequence provided below. In some cases, a TMAPP of the present disclosure comprises a DRB1*1502 polypeptide comprising an amino acid sequence having at least 90%, at least 95%, at least 98%, at least 99%, or 100%, amino acid sequence identity to amino acids 30-124 of the DRB1*1502 amino acid sequence provided below. In some cases, a TMAPP of the present disclosure comprises a DRB1*1502 polypeptide comprising an amino acid sequence having at least 90%, at least 95%, at least 98%, at least 99%, or 100%, amino acid sequence identity to amino acids 125-227 of the DRB1*1502 amino acid sequence provided below.

DRB1*1502:
(SEQ ID NO: 87)
MVCLKLPGGSCMTALTVTLMVLSSPLALSGDTRPRFLWQPKRECHFFNGT
ERVRFLDRYFYNQEESVRFDSDVGEFRAVTELGRPDAEYWNSQKDILEQA
RAAVDTYCRHNYGVGESFTVQRRVQPKVTVYPSKTQPLQHHNLLVCSVSG
FYPGSIEVRWFLNGQEEKAGMVSTGLIQNGDWTFQTLVMLETVPRSGEVY
TCQVEHPSVTSPLTVEWRARSESAQSKMLSGVGGFVLGLLFLGAGLFIYF
RNQKGHSGLQPTGFLS.

In some cases, a TMAPP of the present disclosure comprises a DRB1*1503 polypeptide comprising an amino acid sequence having at least 90%, at least 95%, at least 98%, at least 99%, or 100%, amino acid sequence identity to amino acids 30-227 of the DRB1*1503 amino acid sequence provided below. In some cases, a TMAPP of the present disclosure comprises a DRB1*1503 polypeptide comprising an amino acid sequence having at least 90%, at least 95%, at least 98%, at least 99%, or 100%, amino acid sequence identity to amino acids 30-124 of the DRB1*1503 amino acid sequence provided below. In some cases, a TMAPP of the present disclosure comprises a DRB1*1503 polypeptide comprising an amino acid sequence having at least 90%, at least 95%, at least 98%, at least 99%, or 100%, amino acid sequence identity to amino acids 125-227 of the DRB1*1503 amino acid sequence provided below.

DRB1*1503:
(SEQ ID NO: 88)
MVCLKLPGGSCMTALTVTLMVLSSPLALSGDTRPRFLWQPKRECHFFNGT
ERVRFLDRHFYNQEESVRFDSDVGEFRAVTELGRPDAEYWNSQKDILEQA
RAAVDTYCRHNYGVVESFTVQRRVQPKVTVYPSKTQPLQHHNLLVCSVSG
FYPGSIEVRWFLNGQEEKAGMVSTGLIQNGDWTFQTLVMLETVPRSGEVY
TCQVEHPSVTSPLTVEWRARSESAQSKMLSGVGGFVLGLLFLGAGLFIYF
RNQKGHSGLQPTGFLS.

### DRB1*15:04

In some cases, a TMAPP of the present disclosure comprises a DRB1*1504 polypeptide comprising an amino acid sequence having at least 90%, at least 95%, at least 98%, at least 99%, or 100%, amino acid sequence identity to amino acids 30-227 of the DRB1*1504 amino acid sequence provided below. In some cases, a TMAPP of the present disclosure comprises a DRB1*1504 polypeptide comprising an amino acid sequence having at least 90%, at least 95%, at least 98%, at least 99%, or 100%, amino acid sequence identity to amino acids 30-124 of the DRB1*1504 amino acid sequence provided below. In some cases, a TMAPP of the present disclosure comprises a DRB1*1504 polypeptide comprising an amino acid sequence having at least 90%, at least 95%, at least 98%, at least 99%, or 100%, amino acid sequence identity to amino acids 125-227 of the DRB1*1504 amino acid sequence provided below.

DRB1*15:04:
(SEQ ID NO: 89)
MVCLKLPGGSCMTALTVTLMVLSSPLALSGDTRPRFLWQPKRECHFFNGT
ERVRFLDRYFYNQEESVRFDSDVGEFRAVTELGRPDAEYWNSQKDFLEQA
RAAVDTYCRHNYGVVESFTVQRRVQPKVTVYPSKTQPLQHHNLLVCSVSG
FYPGSIEVRWFLNGQEEKAGMVSTGLIQNGDWTFQTLVMLETVPRSGEVY
TCQVEHPSVTSPLTVEWRARSESAQSKMLSGVGGFVLGLLFLGAGLFIYF
RNQKGHSGLQPTGFLS

### DRB1*15:05

In some cases, a TMAPP of the present disclosure comprises a DRB1*15:05 polypeptide comprising an amino acid sequence having at least 90%, at least 95%, at least 98%, at least 99%, or 100%, amino acid sequence identity to amino acids 30-227 of the DRB1*15:05 amino acid sequence provided below. In some cases, a TMAPP of the present disclosure comprises a DRB1*15:05 polypeptide comprising an amino acid sequence having at least 90%, at least 95%, at least 98%, at least 99%, or 100%, amino acid sequence identity to amino acids 30-227 of the DRB1*15:05 amino acid sequence provided below. In some cases, a TMAPP of the present disclosure comprises a DRB1*15:05 polypeptide comprising an amino acid sequence having at least 90%, at least 95%, at least 98%, at least 99%, or 100%, amino acid sequence identity to amino acids 125-227 of the DRB1*15:05 amino acid sequence provided below.

DRB1*15:05:
(SEQ ID NO: 90)
MVCLKLPGGS CMTALTVTLM VLSSPLALSG DTRPRFLWQP
KRECHFFNGT ERVRFLDRYF YNQEESVRFD SDVGEFRAVT
ELGRPDAEYW NSQKDILEQA RAAVDTYCRH NYGVVESFTV
QRRVQPKVTV YPSKTQPLQH HNLLVCSVSG FYPGSIEVRW
FLNGQEEKAG MVSTGLIQNG DWTFQTLVML ETVPRSGEVY
TCQVEHPSVT SPLTVEWRAR SESAQSKMLS GVGGFVLGLL
FLGAGLFIYF RNQKGHSGLQ PTGFLS.

### DRB1*15:06

In some cases, a TMAPP of the present disclosure comprises a DRB1*15:06 polypeptide comprising an amino acid sequence having at least 90%, at least 95%, at least 98%, at least 99%, or 100%, amino acid sequence identity to amino acids 30-227 of the DRB1*15:06 amino acid sequence provided below. In some cases, a TMAPP of the present disclosure comprises a DRB1*15:06 polypeptide comprising an amino acid sequence having at least 90%, at least 95%, at least 98%, at least 99%, or 100%, amino acid sequence identity to amino acids 30-124 of the DRB1*15:06 amino acid sequence provided below. In some cases, a TMAPP of the present disclosure comprises a DRB1*15:06 polypeptide comprising an amino acid sequence having at least 90%, at least 95%, at least 98%, at least 99%, or 100%, amino acid sequence identity to amino acids 125-227 of the DRB1*15:06 amino acid sequence provided below.

DRB1*15:06:
(SEQ ID NO: 90)
MVCLKLPGGS CMTALTVTLM VLSSPLALSG DTRPRFLWQP
KRECHFFNGT ERVRFLDRYF YNQEESVRFD SDVGEFRAVT
ELGRPDAEYW NSQKDILEQA RAAVDTYCRH NYGVVESFTV
QRRVQPKVTV YPSKTQPLQH HNLLVCSVSG FYPGSIEVRW
FLNGQEEKAG MVSTGLIQNG DWTFQTLVML ETVPRSGEVY
TCQVEHPSVT SPLTVEWRAR SESAQSKMLS GVGGFVLGLL
FLGAGLFIYF RNQKGHSGLQ PTGFLS.

### DRB1*15:07

In some cases, a TMAPP of the present disclosure comprises a DRB1*15:07 polypeptide comprising an amino acid sequence having at least 90%, at least 95%, at least 98%, at least 99%, or 100%, amino acid sequence identity to amino acids 30-227 of the DRB1*15:07 amino acid sequence provided below. In some cases, a TMAPP of the present disclosure comprises a DRB1*15:07 polypeptide comprising an amino acid sequence having at least 90%, at least 95%, at least 98%, at least 99%, or 100%, amino acid sequence identity to amino acids 30-124 of the DRB1*15:07 amino acid sequence provided below. In some cases, a TMAPP of the present disclosure comprises a DRB1*15:07 polypeptide comprising an amino acid sequence having at least 90%, at least 95%, at least 98%, at least 99%, or 100%, amino acid sequence identity to amino acids 125-227 of the DRB1*15:07 amino acid sequence provided below.

DRB1*15:07:
(SEQ ID NO: 91)
MVCLKLPGGSCMTALTVTLMVLSSPLALSGDTRPRFLWQPKRECHFFNGT
ERVRFLDRYFYNQEESVRFDSDVGEYRAVTELGRPDAEYWNSQKDILEQA
RAAVDTYCRHNYGVVESFTVQRRVQPKVTVYPSKTQPLQHHNLLVCSVSG
FYPGSIEVRWFLNGQEEKAGMVSTGLIQNGDWTFQTLVMLETVPRSGEVY
TCQVEHPSVTSPLTVEWRARSESAQSKMLSGVGGFVLGLLFLGAGLFIYF
RNQKGHSGLQPTGFLS.

### DRB3

In some cases, a TMAPP of the present disclosure comprises an MHC Class II β chain polypeptide of a DRB3 allele.

### DRB3*01:01

In some cases, a TMAPP of the present disclosure comprises a DRB3*01:01 polypeptide comprising an amino acid sequence having at least 90%, at least 95%, at least 98%, at least 99%, or 100%, amino acid sequence identity to amino acids 30-227 of the DRB3*01:01 amino acid sequence provided below. In some cases, a TMAPP of the present disclosure comprises a DRB3*01:01 polypeptide comprising an amino acid sequence having at least 90%, at least 95%, at least 98%, at least 99%, or 100%, amino acid sequence identity to amino acids 30-124 of the DRB3*01:01 amino acid sequence provided below. In some cases, a TMAPP of the present disclosure comprises a DRB3*01:01 polypeptide comprising an amino acid sequence having at least 90%, at least 95%, at least 98%, at least 99%, or 100%, amino acid sequence identity to amino acids 125-227 of the DRB3*01:01 amino acid sequence provided below.

DRB3*01:01
(SEQ ID NO: 92)
MVCLKLPGGSSLAALTVTLMVLSSRLAFAGDTRPRFLELRKSECHFFNGT
ERVRYLDRYFHNQEEFLRFDSDVGEYRAVTELGRPVAESWNSQKDLLEQK
RGRVDNYCRHNYGVGESFTVQRRVHPQVTVYPAKTQPLQHHNLLVCSVSG
FYPGSIEVRWFRNGQEEKAGVVSTGLIQNGDWTFQTLVMLETVPRSGEVY
TCQVEHPSVTSALTVEWRARSESAQSKMLSGVGGFVLGLLFLGAGLFIYF
RNQKGHSGLQPTGFLS.

### DRB3*03:01

In some cases, a TMAPP of the present disclosure comprises a DRB3*03:01 polypeptide comprising an amino acid sequence having at least 90%, at least 95%, at least 98%, at least 99%, or 100%, amino acid sequence identity to amino acids 30-227 of the DRB3*03:01 amino acid sequence provided below. In some cases, a TMAPP of the present disclosure comprises a DRB3*03:01 polypeptide comprising an amino acid sequence having at least 90%, at least 95%, at least 98%, at least 99%, or 100%, amino acid sequence identity to amino acids 30-124 of the DRB3*03:01 amino acid sequence provided below. In some cases, a TMAPP of the present disclosure comprises a DRB3*03:01 polypeptide comprising an amino acid sequence having at least 90%, at least 95%, at least 98%, at least 99%, or 100%, amino acid sequence identity to amino acids 125-227 of the DRB3*03:01 amino acid sequence provided below.

DRB3*03:01:
(SEQ ID NO: 93)
MVCLKLPGGSSLAALTVTLMVLSSRLAFAGDTRPRFLELLKSECHFFNGT
ERVRFLERYFHNQEEFVRFDSDVGEYRAVTELGRPVAESWNSQKDLLEQK
RGQVDNYCRHNYGVVESFTVQRRVHPQVTVYPAKTQPLQHHNLLVCSVSG
FYPGSIEVRWFRNGQEEKTGVVSTGLIHNGDWTFQTLVMLETVPRSGEVY
TCQVEHPSVTSPLTVEWRARSESAQSKMLSGVGGFVLGLLFLGAGLFIYF
RNQKGHSGLQPTGFLS.

### DRB4

In some cases, a TMAPP of the present disclosure comprises an MHC Class II β chain polypeptide of a DRB4 allele.

For example, in some cases, a TMAPP of the present disclosure comprises a DRB4*01:01 polypeptide comprising an amino acid sequence having at least 90%, at least 95%, at least 98%, at least 99%, or 100%, amino acid sequence identity to amino acids 30-227 of the DRB4*01:01 amino acid sequence provided below. In some cases, a TMAPP of the present disclosure comprises a DRB4*01:01 polypeptide comprising an amino acid sequence having at least 90%, at least 95%, at least 98%, at least 99%, or 100%, amino acid sequence identity to amino acids 30-124 of the DRB4*01:01 amino acid sequence provided below. In some cases, a TMAPP of the present disclosure comprises a DRB3*03:01 polypeptide comprising an amino acid sequence having at least 90%, at least 95%, at least 98%, at least 99%, or 100%, amino acid sequence identity to amino acids 125-227 of the DRB4*01:01 amino acid sequence provided below.

DRB4*01:01:
(SEQ ID NO: 94)
MVCLKLPGGSCMAALTVTLTVLSSPLALAGDTQPRFLEQAKCECHFLNGT
ERVWNLIRYIYNQEEYARYNSDLGEYQAVTELGRPDAEYWNSQKDLLERR
RAEVDTYCRYNYGVVESFTVQRRVQPKVTVYPSKTQPLQHHNLLVCSVNG
FYPGSIEVRWFRNSQEEKAGVVSTGLIQNGDWTFQTLVMLETVPRSGEVY
TCQVEHPSMMSPLTVQWSARSESAQSKMLSGVGGFVLGLLFLGTGLFIYF
RNQKGHSGLQPTGLLS.

### DRB5

In some cases, a TMAPP of the present disclosure comprises an MHC Class II β chain polypeptide of a DRB5 allele.

### DRB5*01:01

In some cases, a TMAPP of the present disclosure comprises a DRB5*01:01 polypeptide comprising an amino acid sequence having at least 90%, at least 95%, at least 98%, at least 99%, or 100%, amino acid sequence identity to amino acids 30-227 of the DRB5*01:01 amino acid sequence provided below. In some cases, a TMAPP of the present disclosure comprises a DRB5*01:01 polypeptide comprising an amino acid sequence having at least 90%, at least 95%, at least 98%, at least 99%, or 100%, amino acid sequence identity to amino acids 30-124 of the DRB5*01:01 amino acid sequence provided below. In some cases, a TMAPP of the present disclosure comprises a DRB5*01:01 polypeptide comprising an amino acid sequence having at least 90%, at least 95%, at least 98%, at least 99%, or 100%, amino acid sequence identity to amino acids 125-227 of the DRB5*01:01 amino acid sequence provided below.

DRB5*01:01:
(SEQ ID NO: 95)
MVLSSPLALAGDTRPRFLQQDKYECHFFNGTERVRFLHRDIYNQEEDLRF
DSDVGEYRAVTELGRPDAEYWNSQKDFLEDRRAAVDTYCRHNYGVGESFT
VQRRVEPKVTVYPARTQTLQHHNLLVCSVNGFYPGSIEVRWFRNSQEEKA
GVVSTGLIQNGDWTFQTLVMLETVPRSGEVYTCQVEHPSVTSPLTVEWRA
QSESAQSKMLSGVGGFVLGLLFLGAGLFIYFKNQKGHSGLHPTGLVS.

### DQB1

Certain DQB1 alleles are associated with increased risk that an individual expressing such an allele will develop an autoimmune disease such as multiple sclerosis. For example, DQB1*0301, and DQB1*0602 are associated with an increased risk of developing MS and/or a more severe MS phenotype (e.g., more severe inflammatory and neurodegenerative damage).

### DQB1*02:01

In some cases, a TMAPP of the present disclosure comprises a DQB1*02:01 polypeptide comprising an amino acid sequence having at least 90%, at least 95%, at least 98%, at least 99%, or 100%, amino acid sequence identity to amino acids 33-220 of the DQB1*02:01 amino acid sequence provided below. In some cases, a TMAPP of the present disclosure comprises a DQB1*02:01 polypeptide comprising an amino acid sequence having at least 90%, at least 95%, at least 98%, at least 99%, or 100%, amino acid sequence identity to amino acids 33-126 of the DQB1*02:01 amino acid sequence provided below. In some cases, a TMAPP of the present disclosure comprises a DQB1*02:01 polypeptide comprising an amino acid sequence having at least 90%, at least 95%, at least 98%, at least 99%, or 100%, amino acid sequence identity to amino acids 127-220 of the DQB1*02:01 amino acid sequence provided below.

DQB1*02:01:
(SEQ ID NO: 96)
MSWKKALRIPGGLRAATVTLMLSMLSTPVAEGRDSPEDFVYQFKGMCYFT
NGTERVRLVSRSIYNREEIVRFDSDVGEFRAVTLLGLPAAEYWNSQKDIL
ERKRAAVDRVCRHNYQLELRTTLQRRVEPTVTISPSRTEALNHHNLLVCS
VTDFYPAQIKVRWFRNDQEETAGVVSTPLIRNGDWTFQILVMLEMTPQRG
DVYTCHVEHPSLQSPITVEWRAQSESAQSKMLSGIGGFVLGLIFLGLGLI
IHHRSQKGLLH.

### DQB1*02:02

In some cases, a TMAPP of the present disclosure comprises a DQB1*02:02 polypeptide comprising an amino acid sequence having at least 90%, at least 95%, at least 98%, at least 99%, or 100%, amino acid sequence identity to amino acids 33-220 of the DQB1*02:02 amino acid sequence provided below. In some cases, a TMAPP of the present disclosure comprises a DQB1*02:02 polypeptide comprising an amino acid sequence having at least 90%, at least 95%, at least 98%, at least 99%, or 100%, amino acid sequence identity to amino acids 33-126 of the DQB1*02:02 amino acid sequence provided below. In some cases, a TMAPP of the present disclosure comprises a DQB1*02:02 polypeptide comprising an amino acid sequence having at least 90%, at least 95%, at least 98%, at least 99%, or 100%, amino acid sequence identity to amino acids 127-220 of the DQB1*02:02 amino acid sequence provided below.

DQB1*02:02:
(SEQ ID NO: 97)
MSWKKALRIPGGLRAATVTLMLSMLSTPVAEGRDSPEDFVYQFKGMCYFT
NGTERVRLVSRSIYNREEIVRFDSDVGEFRAVTLLGLPAAEYWNSQKDIL
ERKRAAVDRVCRHNYQLELRTTLQRRVEPTVTISPSRTEALNHHNLLVCS
VTDFYPAQIKVRWFRNGQEETAGVVSTPLIRNGDWTFQILVMLEMTPQRG
DVYTCHVEHPSLQSPITVEWRAQSESAQSKMLSGIGGFVLGLIFLGLGLI
IHHRSQKGLLH.

### DQB1*03:01

In some cases, a TMAPP of the present disclosure comprises a DQB1*0301 polypeptide comprising an amino acid sequence having at least 90%, at least 95%, at least 98%, at least 99%, or 100%, amino acid sequence identity to amino acids 33-220 of the DQB1*0301 amino acid sequence provided below. In some cases, a TMAPP of the present disclosure comprises a DQB1*0301 polypeptide comprising an amino acid sequence having at least 90%, at least 95%, at least 98%, at least 99%, or 100%, amino acid sequence identity to amino acids 33-126 of the DQB1*0301 amino acid sequence provided below. In some cases, a TMAPP of the present disclosure comprises a DQB1*0301 polypeptide comprising an amino acid sequence having at least 90%, at least 95%, at least 98%, at least 99%, or 100%, amino acid sequence identity to amino acids 127-220 of the DQB1*0301 amino acid sequence provided below.

DQB1*03:01:
(SEQ ID NO: 98)
MSWKKALRIPGGLRAATVTLMLAMLSTPVAEGRDSPEDFVYQFKAMCYFT
NGTERVRYVTRYIYNREEYARFDSDVEVYRAVTPLGPPDAEYWNSQKEVL
ERTRAELDTVCRHNYQLELRTTLQRRVEPTVTISPSRTEALNHHNLLVCS
VTDFYPAQIKVRWFRNDQEETTGVVSTPLIRNGDWTFQILVMLEMTPQHG
DVYTCHVEHPSLQNPITVEWRAQSESAQSKMLSGIGGFVLGLIFLGLGLI
IHHRSQKGLLH.

### DQB1*03:02

In some cases, a TMAPP of the present disclosure comprises a DQB1*0302 polypeptide comprising an amino acid sequence having at least 90%, at least 95%, at least 98%, at least 99%, or 100%, amino acid sequence identity to amino acids 33-220 of the DQB1*0302 amino acid sequence provided below. In some cases, a TMAPP of the present disclosure comprises a DQB1*0302 polypeptide comprising an amino acid sequence having at least 90%, at least 95%, at least 98%, at least 99%, or 100%, amino acid sequence identity to amino acids 33-126 of the DQB1*0302 amino acid sequence provided below. In some cases, a TMAPP of the present disclosure comprises a DQB1*0302 polypeptide comprising an amino acid sequence having at least 90%, at least 95%, at least 98%, at least 99%, or 100%, amino acid sequence identity to amino acids 127-220 of the DQB1*0302 amino acid sequence provided below.

DQB1*0302:
(SEQ ID NO: 99)
MSWKKALRIPGGLRVATVTLMLAMLSTPVAEGRDSPEDFVYQFKGMCYFT
NGTERVRLVTRYIYNREEYARFDSDVGVYRAVTPLGPPAAEYWNSQKEVL
ERTRAELDTVCRHNYQLELRTTLQRRVEPTVTISPSRTEALNHHNLLVCS
VTDFYPAQIKVRWFRNDQEETTGVVSTPLIRNGDWTFQILVMLEMTPQRG
DVYTCHVEHPSLQNPIIVEWRAQSESAQSKMLSGIGGFVLGLIFLGLGLI
IHHRSQKGLLH.

### DQB1*03:03

In some cases, a TMAPP of the present disclosure comprises a DQB1*03:03 polypeptide comprising an amino acid sequence having at least 90%, at least 95%, at least 98%, at least 99%, or 100%, amino acid sequence identity to amino acids 33-220 of the DQB1*03:03 amino acid sequence provided below. In some cases, a TMAPP of the present disclosure comprises a DQB1*03:03 polypeptide comprising an amino acid sequence having at least 90%, at least 95%, at least 98%, at least 99%, or 100%, amino acid sequence identity to amino acids 33-126 of the DQB1*03:03 amino acid sequence provided below. In some cases, a TMAPP of the present disclosure comprises a DQB1*03:03 polypeptide comprising an amino acid sequence having at least 90%, at least 95%, at least 98%, at least 99%, or 100%, amino acid sequence identity to amino acids 127-220 of the DQB1*03:03 amino acid sequence provided below.

DQB1*03:03:
(SEQ ID NO: 100)
MSWKKALRIPGGLRVATVTLMLAMLSTPVAEGRDSPEDFVYQFKGMCYFT
NGTERVRLVTRYIYNREEYARFDSDVGVYRAVTPLGPPDAEYWNSQKEVL
ERTRAELDTVCRHNYQLELRTTLQRRVEPTVTISPSRTEALNHHNLLVCS
VTDFYPAQIKVRWFRNDQEETTGVVSTPLIRNGDWTFQILVMLEMTPQRG
DVYTCHVEHPSLQNPIIVEWRAQSESAQSKMLSGIGGFVLGLIFLGLGLI
IHHRSQKGLLH.

### DQB1*03:04

In some cases, a TMAPP of the present disclosure comprises a DQB1*03:04 polypeptide comprising an amino acid sequence having at least 90%, at least 95%, at least 98%, at least 99%, or 100%, amino acid sequence identity to amino acids 33-220 of the DQB1*03:04 amino acid sequence provided below. In some cases, a TMAPP of the present disclosure comprises a DQB1*03:04 polypeptide comprising an amino acid sequence having at least 90%, at least 95%, at least 98%, at least 99%, or 100%, amino acid sequence identity to amino acids 33-126 of the DQB1*03:04 amino acid sequence provided below. In some cases, a TMAPP of the present disclosure comprises a DQB1*03:04 polypeptide comprising an amino acid sequence having at least 90%, at least 95%, at least 98%, at least 99%, or 100%, amino acid sequence identity to amino acids 127-220 of the DQB1*03:04 amino acid sequence provided below.

DQB1*03:04:
(SEQ ID NO: 101)
MSWKKALRIPGGLRAATVTLMLAMLSTPVAEGRDS
PEDFVYQFKAMCYFTNGTERVRYVTRYIYNREEYA
RFDSDVEVYRAVTPLGPPAAEYWNSQKEVLERTRA
ELDTVCRHNYQLELRTTLQRRVEPTVTISPSRTEA
LNHHNLLVCSVTDFYPAQIKVRWFRNDQEETTGVV
STPLIRNGDWTFQILVMLEMTPQHGDVYTCHVEHP
SLQNPITVEWRAQSESAQSKMLSGIGGFVLGLIFL
GLGLIIHHRSQKGLLH.

### DQB1*04:01

In some cases, a TMAPP of the present disclosure comprises a DQB1*04:01 polypeptide comprising an amino acid sequence having at least 90%, at least 95%, at least 98%, at least 99%, or 100%, amino acid sequence identity to amino acids 33-220 of the DQB1*04:01 amino acid sequence provided below. In some cases, a TMAPP of the present disclosure comprises a DQB1*04:01 polypeptide comprising an amino acid sequence having at least 90%, at least 95%, at least 98%, at least 99%, or 100%, amino acid sequence identity to amino acids 33-126 of the DQB1*04:01 amino acid sequence provided below. In some cases, a TMAPP of the present disclosure comprises a DQB1*04:01 polypeptide comprising an amino acid sequence having at least 90%, at least 95%, at least 98%, at least 99%, or 100%, amino acid sequence identity to amino acids 127-220 of the DQB1*04:01 amino acid sequence provided below.

DQB1*04:01:
(SEQ ID NO: 102)
MSWKKALRIPGGLRVATVTLMLAMLSTPVAEGRDS
PEDFVFQFKGMCYFTNGTELVRGVTRYIYNREEYA
RFDSDVGVYRAVTPLGRLDAEYWNSQKDILEEDRA
SVDTVCRHNYQLELRTTLQRRVEPTVTISPSRTEA
LNHHNLLVCSVTDFYPAQIKVRWFRNDQEETTGVV
STPLIRNGDWTFQILVMLEMTPQRGDVYTCHVEHP
SLQNPIIVEWRAQSESAQSKMLSGIGGFVLGLIFL
GLGLIIHHRSQKGLLH.

### DQB1*04:02

In some cases, a TMAPP of the present disclosure comprises a DQB1*04:02 polypeptide comprising an amino acid sequence having at least 90%, at least 95%, at least 98%, at least 99%, or 100%, amino acid sequence identity to amino acids 33-220 of the DQB1*04:02 amino acid sequence provided below. In some cases, a TMAPP of the present disclosure comprises a DQB1*04:02 polypeptide comprising an amino acid sequence having at least 90%, at least 95%, at least 98%, at least 99%, or 100%, amino acid sequence identity to amino acids 33-126 of the DQB1*04:02 amino acid sequence provided below. In some cases, a TMAPP of the present disclosure comprises a DQB1*04:02 polypeptide comprising an amino acid sequence having at least 90%, at least 95%, at least 98%, at least 99%, or 100%, amino acid sequence identity to amino acids 127-220 of the DQB1*04:02 amino acid sequence provided below.

DQB1*04:02:
(SEQ ID NO: 386)
MSWKKALRIPGGLRVATVTLMLAMLSTPVAEGRDS
PEDFVFQFKGMCYFTNGTERVRGVTRYIYNREEYA
RFDSDVGVYRAVTPLGRLDAEYWNSQKDILEEDRA
SVDTVCRHNYQLELRTTLQRRVEPTVTISPSRTEA
LNHHNLLVCSVTDFYPAQIKVRWFRNDQEETTGVV
STPLIRNGDWTFQILVMLEMTPQRGDVYTCHVEHP
SLQNPIIVEWRAQSESAQSKMLSGIGGFVLGLIFL
GLGLIIHHRSQKGLLH.

### DQB1*05:01

In some cases, a TMAPP of the present disclosure comprises a DQB1*04:01 polypeptide comprising an amino acid sequence having at least 90%, at least 95%, at least 98%, at least 99%, or 100%, amino acid sequence identity to amino acids 33-220 of the DQB1*05:01 amino acid sequence provided below. In some cases, a TMAPP of the present disclosure comprises a DQB1*05:01 polypeptide comprising an amino acid sequence having at least 90%, at least 95%, at least 98%, at least 99%, or 100%, amino acid sequence identity to amino acids 33-126 of the DQB1*05:01 amino acid sequence provided below. In some cases, a TMAPP of the present disclosure comprises a DQB1*05:01 polypeptide comprising an amino acid sequence having at least 90%, at least 95%, at least 98%, at least 99%, or 100%, amino acid sequence identity to amino acids 127-220 of the DQB1*05:01 amino acid sequence provided below.

DQB1*05:01:
(SEQ ID NO: 387)
MSWKKSLRIPGDLRVATVTLMLAILSSSLAEGRDS
PEDFVYQFKGLCYFTNGTERVRGVTRHIYNREEYV
RFDSDVGVYRAVTPQGRPVAEYWNSQKEVLEGARA
SVDRVCRHNYEVAYRGILQRRVEPTVTISPSRTEA
LNHHNLLICSVTDFYPSQIKVRWFRNDQEETAGVV
STPLIRNGDWTFQILVMLEMTPQRGDVYTCHVEHP
SLQSPITVEWRAQSESAQSKMLSGVGGFVLGLIFL
GLGLIIRQRSRKGLLH.

### DQB1*05:03

In some cases, a TMAPP of the present disclosure comprises a DQB1*05:03 polypeptide comprising an amino acid sequence having at least 90%, at least 95%, at least 98%, at least 99%, or 100%, amino acid sequence identity to amino acids 33-220 of the DQB1*05:03 amino acid sequence provided below. In some cases, a TMAPP of the present disclosure comprises a DQB1*05:03 polypeptide comprising an amino acid sequence having at least 90%, at least 95%, at least 98%, at least 99%, or 100%, amino acid sequence identity to amino acids 33-126 of the DQB1*05:03 amino acid sequence provided below. In some cases, a TMAPP of the present disclosure comprises a DQB1*05:03 polypeptide comprising an amino acid sequence having at least 90%, at least 95%, at least 98%, at least 99%, or 100%, amino acid sequence identity to amino acids 127-220 of the DQB1*05:03 amino acid sequence provided below.

DQB1*05:03:
(SEQ ID NO: 388)
MSWKKSLRIPGDLRVATVTLMLAILSSSLAEGRDS
PEDFVYQFKGLCYFTNGTERVRGVTRHIYNREEYV
RFDSDVGVYRAVTPQGRPDAEYWNSQKEVLEGARA
SVDRVCRHNYEVAYRGILQRRVEPTVTISPSRTEA
LNHHNLLICSVTDFYPSQIKVRWFRNDQEETAGVV
STPLIRNGDWTFQILVMLEMTPQRGDVYTCHVEHP
SLQSPITVEWRAQSESAQSKMLSGVGGFVLGLIFL
GLGLIIRQRSRKGPQGPPPAGLLH.

### DQB1*06:01

In some cases, a TMAPP of the present disclosure comprises a DQB1*06:01 polypeptide comprising an amino acid sequence having at least 90%, at least 95%, at least 98%, at least 99%, or 100%, amino acid sequence identity to amino acids 33-220 of the DQB1*06:01 amino acid sequence provided below. In some cases, a TMAPP of the present disclosure comprises a DQB1*06:01 polypeptide comprising an amino acid sequence having at least 90%, at least 95%, at least 98%, at least 99%, or 100%, amino acid sequence identity to amino acids 33-126 of the DQB1*06:01 amino acid sequence provided below. In some cases, a TMAPP of the present disclosure comprises a DQB1*06:01 polypeptide comprising an amino acid sequence having at least 90%, at least 95%, at least 98%, at least 99%, or 100%, amino acid sequence identity to amino acids 127-220 of the DQB1*06:01 amino acid sequence provided below.

DQB1*06:01:
(SEQ ID NO: 389)
MSWKKALRIPGGLRAPTVTLMLAMLSTPVAEGRDP
PEDFVLQFKAMCYFTNGTERVRYVTRYIYNREEDV
RFDSDVGVYRAVTPQGRPDAEYWNSQKDILERTRA
ELDTVCRHNYEVAFRGILQRRVEPTVTISPSRTEA
LNHHNLLVCSVTDFYPGQIKVRWFRNDQEETAGVV
STPLIRNGDWTFQILVMLEMTPQHGDVYTCHVEHP
SLQSPITVEWRAQSESAQNKMLSGIGGFVLGLIFL
GLGLIIRQRSQKGPQGPPPAGLLH.

### DQB1*06:02

In some cases, a TMAPP of the present disclosure comprises a DQB1*0602 polypeptide comprising an amino acid sequence having at least 90%, at least 95%, at least 98%, at least 99%, or 100%, amino acid sequence identity to amino acids 33-220 of the DQB1*0602 amino acid sequence depicted in FIG. 19A. In some cases, a TMAPP of the present disclosure comprises a DQB1*0602 polypeptide comprising an amino acid sequence having at least 90%, at least 95%, at least 98%, at least 99%, or 100%, amino acid sequence identity to amino acids 33-126 of the DQB1*0602 amino acid sequence depicted in FIG. 19A. In some cases, a TMAPP of the present disclosure comprises a DQB1*0602 polypeptide comprising an amino acid sequence having at least 90%, at least 95%, at least 98%, at least 99%, or 100%, amino acid sequence identity to amino acids 127-220 of the DQB1*0602 amino acid sequence depicted in FIG. 19A.

### DPB1

A TMAPP of the present disclosure can include an MHC Class II β chain of a DPB1 allele.

### DPB1*03:01

For example, in some cases, a TMAPP of the present disclosure comprises a DPB1*03:01 polypeptide comprising an amino acid sequence having at least 90%, at least 95%, at least 98%, at least 99%, or 100%, amino acid sequence identity to amino acids 30-215 of the DPB1*03:01 amino acid sequence provided below. In some cases, a TMAPP of the present disclosure comprises a DPB1*03:01 polypeptide comprising an amino acid sequence having at least 90%, at least 95%, at least 98%, at least 99%, or 100%, amino acid sequence identity to amino acids 30-121 of the DPB1*03:01 amino acid sequence provided below. In some cases, a TMAPP of the present disclosure comprises a DPB1*03:01 polypeptide comprising an amino acid sequence having at least 90%, at least 95%, at least 98%, at least 99%, or 100%, amino acid sequence identity to amino acids 122-215 of the DPB1*03:01 amino acid sequence provided below.

DPB1*03:01:
(SEQ ID NO: 390)
MMVLQVSAAPRTVALTALLMVLLTSVVQGRATPEN
YVYQLRQECYAFNGTQRFLERYIYNREEFVRFDSD
VGEFRAVTELGRPDEDYWNSQKDLLEEKRAVPDRV
CRHNYELDEAVTLQRRVQPKVNVSPSKKGPLQHHN
LLVCHVTDFYPGSIQVRWFLNGQEETAGVVSTNLI
RNGDWTFQILVMLEMTPQQGDVYICQVEHTSLDSP
VTVEWKAQSDSARSKTLTGAGGFVLGLIICGVGIF
MHRRSKKVQRGSA.

### DPB1*09:01

As another example, in some cases, a TMAPP of the present disclosure comprises a DPB1*09:01 polypeptide comprising an amino acid sequence having at least 90%, at least 95%, at least 98%, at least 99%, or 100%, amino acid sequence identity to amino acids 30-215 of the DPB1*09:01 amino acid sequence provided below. In some cases, a TMAPP of the present disclosure comprises a DPB1*09:01 polypeptide comprising an amino acid sequence having at least 90%, at least 95%, at least 98%, at least 99%, or 100%, amino acid sequence identity to amino acids 30-121 of the DPB1*09:01 amino acid sequence provided below. In some cases, a TMAPP of the present disclosure comprises a DPB1*09:01 polypeptide comprising an amino acid sequence having at least 90%, at least 95%, at least 98%, at least 99%, or 100%, amino acid sequence identity to amino acids 122-215 of the DPB1*09:01 amino acid sequence provided below.

DPB1*09:01:
(SEQ ID NO: 391)
MMVLQVSAAPRTVALTALLMVLLTSVVQGRATPEN
YVHQLRQECYAFNGTQRFLERYIYNREEFVRFDSD
VGEFRAVTELGRPDEDYWNSQKDILEEERAVPDRV
CRHNYELDEAVTLQRRVQPKVNVSPSKKGPLQHHN
LLVCHVTDFYPGSIQVRWFLNGQEETAGVVSTNLI
RNGDWTFQILVMLEMTPQQGDVYICQVEHTSLDSP
VTVEWKAQSDSARSKTLTGAGGFVLGLIICGVGIF
MHRRSKKVQRGSA.

### DPB1*13:01

As another example, in some cases, a TMAPP of the present disclosure comprises a DPB1*13:01 polypeptide comprising an amino acid sequence having at least 90%, at least 95%, at least 98%, at least 99%, or 100%, amino acid sequence identity to amino acids 30-215 of the DPB1*13:01 amino acid sequence provided below. In some cases, a TMAPP of the present disclosure comprises a DPB1*13:01 polypeptide comprising an amino acid sequence having at least 90%, at least 95%, at least 98%, at least 99%, or 100%, amino acid sequence identity to amino acids 30-121 of the DPB1*13:01 amino acid sequence provided below. In some cases, a TMAPP of the present disclosure comprises a DPB1*13:01 polypeptide comprising an amino acid sequence having at least 90%, at least 95%, at least 98%, at least 99%, or 100%, amino acid sequence identity to amino acids 122-215 of the DPB1*13:01 amino acid sequence provided below.

DPB1*13:01:
(SEQ ID NO: 392)
MMVLQVSAAPRTVALTALLMVLLTSVVQGRATPEN
YVYQLRQECYAFNGTQRFLERYIYNREEYARFDSD
VGEFRAVTELGRPAAEYWNSQKDILEEERAVPDRI
CRHNYELDEAVTLQRRVQPKVNVSPSKKGPLQHHN
LLVCHVTDFYPGSIQVRWFLNGQEETAGVVSTNLI
RNGDWTFQILVMLEMTPQQGDVYICQVEHTSLDSP
VTVEWKAQSDSARSKTLTGAGGFVLGLIICGVGIF
MHRRSKKVQRGSA.

### DPB1*35:01

As another example, in some cases, a TMAPP of the present disclosure comprises a DPB1*35:01 polypeptide comprising an amino acid sequence having at least 90%, at least 95%, at least 98%, at least 99%, or 100%, amino acid sequence identity to amino acids 30-215 of the DPB1*35:01 amino acid sequence provided below. In some cases, a TMAPP of the present disclosure comprises a DPB1*35:01 polypeptide comprising an amino acid sequence having at least 90%, at least 95%, at least 98%, at least 99%, or 100%, amino acid sequence identity to amino acids 30-121 of the DPB1*35:01 amino acid sequence provided below. In some cases, a TMAPP of the present disclosure comprises a DPB1*35:01 polypeptide comprising an amino acid sequence having at least 90%, at least 95%, at least 98%, at least 99%, or 100%, amino acid sequence identity to amino acids 122-215 of the DPB1*35:01 amino acid sequence provided below.

DPB1*35:01:
(SEQ ID NO: 393)
MMVLQVSAAPRTVALTALLMVLLTSVVQGRATPEN
YVHQLRQECYAFNGTQRFLERYIYNREEFVRFDSD
VGEFRAVTELGRPDEDYWNSQKDILEEKRAVPDRV
CRHNYELDEAVTLQRRVQPKVNVSPSKKGPLQHHN
LLVCHVTDFYPGSIQVRWFLNGQEETAGVVSTNLI
RNGDWTFQILVMLEMTPQQGDVYICQVEHTSLDSP
VTVEWKAQSDSARSKTLTGAGGFVLGLIICGVGIF
MHRRSKKVQRGSA.

### DQA1

In some cases, a TMAPP of the present disclosure comprises an MHC Class II α chain polypeptide of a DQA1 allele.

### DQA1*01:01

For example, in some cases, a TMAPP of the present disclosure comprises a DQA1*01:01 polypeptide comprising an amino acid sequence having at least 90%, at least 95%, at least 98%, at least 99%, or 100%, amino acid sequence identity to amino acids 24-204 of the DQA1*01:01 amino acid sequence provided below. In some cases, a TMAPP of the present disclosure comprises a DQA1*01:01 polypeptide comprising an amino acid sequence having at least 90%, at least 95%, at least 98%, at least 99%, or 100%, amino acid sequence identity to amino acids 24-110 of the DQA1*01:01 amino acid sequence provided below. In some cases, a TMAPP of the present disclosure comprises a DQA1*01:01 polypeptide comprising an amino acid sequence having at least 90%, at least 95%, at least 98%, at least 99%, or 100%, amino acid sequence identity to amino acids 111-204 of the DQA1*01:01 amino acid sequence provided below.

DQA1*01:01:
(SEQ ID NO: 394)
MILNKALLLGALALTTVMSPCGGEDIVADHVASCG
VNLYQFYGPSGQYTHEFDGDEEFYVDLERKETAWR
WPEFSKFGGFDPQGALRNMAVAKHNLNIMIKRYNS
TAATNEVPEVTVFSKSPVTLGQPNTLICLVDNIFP
PVVNITWLSNGQSVTEGVSETSFLSKSDHSFFKIS
YLTFLPSADEIYDCKVEHWGLDQPLLKHWEPEIPA
PMSELTETVVCALGLSVGLVGIVVGTVFIIQGLRS
VGASRHQGPL.

### DQA1*01:02

In some cases, a TMAPP of the present disclosure comprises a DQA1*01:02 polypeptide comprising an amino acid sequence having at least 90%, at least 95%, at least 98%, at least 99%, or 100%, amino acid sequence identity to amino acids 24-204 of the DQA*01:02 amino acid sequence provided below. In some cases, a TMAPP of the present disclosure comprises a DQA*01:02 polypeptide comprising an amino acid sequence having at least 90%, at least 95%, at least 98%, at least 99%, or 100%, amino acid sequence identity to amino acids 24-110 of the DQA*01:02 amino acid sequence provided below. In some cases, a TMAPP of the present disclosure comprises a DQA*01:02 polypeptide comprising an amino acid sequence having at least 90%, at least 95%, at least 98%, at least 99%, or 100%, amino acid sequence identity to amino acids 111-204 of the DQA*01:02 amino acid sequence provided below.

DQA1*01:02:
(SEQ ID NO: 395)
MILNKALLLGALALTTVMSPCGGEDIVADHVASCG
VNLYQFYGPSGQYTHEFDGDEQFYVDLERKETAWR
WPEFSKFGGFDPQGALRNMAVAKHNLNIMIKRYNS
TAATNEVPEVTVFSKSPVTLGQPNTLICLVDNIFP
PVVNITWLSNGQSVTEGVSETSFLSKSDHSFFKIS
YLTFLPSADEIYDCKVEHWGLDQPLLKHWEPEIPA
PMSELTETVVCALGLSVGLMGIVVGTVFIIQGLRS
VGASRHQGPL.

### DQA1*01:03

In some cases, a TMAPP of the present disclosure comprises a DQA1*01:03 polypeptide comprising an amino acid sequence having at least 90%, at least 95%, at least 98%, at least 99%, or 100%, amino acid sequence identity to amino acids 24-204 of the DQA*01:03 amino acid sequence provided below. In some cases, a TMAPP of the present disclosure comprises a DQA*01:03 polypeptide comprising an amino acid sequence having at least 90%, at least 95%, at least 98%, at least 99%, or 100%, amino acid sequence identity to amino acids 24-110 of the DQA*01:03 amino acid sequence provided below. In some cases, a TMAPP of the present disclosure comprises a DQA*01:03 polypeptide comprising an amino acid sequence having at least 90%, at least 95%, at least 98%, at least 99%, or 100%, amino acid sequence identity to amino acids 111-204 of the DQA*01:03 amino acid sequence provided below.

DQA1*01:03:
(SEQ ID NO: 396)
MILNKALLLGALALTTVMSPCGGEDIVADHVASCG
VNLYQFYGPSGQFTHEFDGDEQFYVDLEKKETAWR
WPEFSKFGGFDPQGALRNMAVAKHNLNIMIKRYNS
TAATNEVPEVTVFSKSPVTLGQPNTLICLVDNIFP
PVVNITWLSNGHAVTEGVSETSFLSKSDHSFFKIS
YLTFLPSADEIYDCKVEHWGLDQPLLKHWEPEIPA
PMSELTETVVCALGLSVGLVGIVVGTVFIIQGLRS
VGASRHQGPL.

### DQA1*01:04

As another example, in some cases, a TMAPP of the present disclosure comprises a DQA1*01:04 polypeptide comprising an amino acid sequence having at least 90%, at least 95%, at least 98%, at least 99%, or 100%, amino acid sequence identity to amino acids 24-204 of the DQA*01:04 amino acid sequence provided below. In some cases, a TMAPP of the present disclosure comprises a DQA*01:04 polypeptide comprising an amino acid sequence having at least 90%, at least 95%, at least 98%, at least 99%, or 100%, amino acid sequence identity to amino acids 24-110 of the DQA*01:04 amino acid sequence provided below. In some cases, a TMAPP of the present disclosure comprises a DQA*01:04 polypeptide comprising an amino acid sequence having at least 90%, at least 95%, at least 98%, at least 99%, or 100%, amino acid sequence identity to amino acids 111-204 of the DQA*01:04 amino acid sequence provided below.

DQA1*01:04:
(SEQ ID NO: 397)
MILNKALLLGALALTTMMSPCGGEGIVADHVASCG
VNLYQFYGPSGQYTHEFDGDEEFYVDLERKETAWR
WPEFSKFGGFDPQGALRNMAVAKHNLNIMIKRYNS
TAATNEVPEVTVFSKSPVTLGQPNTLICLVDNIFP
PVVNITWLSNGQSVTEGVSETSFLSKSDHSFFKIS
YLTFLPSADEIYDCKVEHWGLDQPLLKHWEPEIPA
PMSELTETVVCTLGLSVGLVGIVVGTVFIIQGLRS
VGASRHQGPL.

### DQA1*03:01

As another example, in some cases, a TMAPP of the present disclosure comprises a DQA1*03:01 polypeptide comprising an amino acid sequence having at least 90%, at least 95%, at least 98%, at least 99%, or 100%, amino acid sequence identity to amino acids 24-204 of the DQA1*03:01 amino acid sequence provided below. In some cases, a TMAPP of the present disclosure comprises a DQA1*03:01 polypeptide comprising an amino acid sequence having at least 90%, at least 95%, at least 98%, at least 99%, or 100%, amino acid sequence identity to amino acids 24-110 of the DQA1*03:01 amino acid sequence provided below. In some cases, a TMAPP of the present disclosure comprises a DQA1*03:01 polypeptide comprising an amino acid sequence having at least 90%, at least 95%, at least 98%, at least 99%, or 100%, amino acid sequence identity to amino acids 111-204 of the DQA1*03:01 amino acid sequence provided below.

DQA1*03:01:
(SEQ ID NO: 398)
MILNKALMLGALALTTVMSPCGGEDIVADHVASYG
VNLYQSYGPSGQYSHEFDGDEEFYVDLERKETVWQ
LPLFRRFRRFDPQFALTNIAVLKHNLNIVIKRSNS
TAATNEVPEVTVFSKSPVTLGQPNTLICLVDNIFP
PVVNITWLSNGHSVTEGVSETSFLSKSDHSFFKIS
YLTFLPSADEIYDCKVEHWGLDEPLLKHWEPEIPT
PMSELTETVVCALGLSVGLVGIVVGTVLIIRGLRS
VGASRHQGPL.

### DQA1*03:02

As another example, in some cases, a TMAPP of the present disclosure comprises a DQA1*03:02 polypeptide comprising an amino acid sequence having at least 90%, at least 95%, at least 98%, at least 99%, or 100%, amino acid sequence identity to amino acids 24-204 of the DQA1*03:02 amino acid sequence provided below. In some cases, a TMAPP of the present disclosure comprises a DQA1*03:02 polypeptide comprising an amino acid sequence having at least 90%, at least 95%, at least 98%, at least 99%, or 100%, amino acid sequence identity to amino acids 24-110 of the DQA1*03:02 amino acid sequence provided below. In some cases, a TMAPP of the present disclosure comprises a DQA1*03:02 polypeptide comprising an amino acid sequence having at least 90%, at least 95%, at least 98%, at least 99%, or 100%, amino acid sequence identity to amino acids 111-204 of the DQA1*03:02 amino acid sequence provided below.

DQA1*03:02:
(SEQ ID NO: 399)
MILNKALMLGALALTTVTSPCGGEDIVADHVASYGVNLYQSYGPSGQYSHE
FDGDEEFYVDLERKETVWQLPLFRRFRRFDPQFALTNIAVLKHNLNIVIKR
SNSTAATNEVPEVTVFSKSPVTLGQPNTLICLVDNIFPPVVNITWLSNGHS
VTEGVSETSFLSKSDHSFFKISYLTFLPSDDEIYDCKVEHWGLDEPLLKHW
EPEIPTPMSELTETVVCALGLSVGLVGIVVGTVLIIRGLRSVGASRHQGP
L.

### DQA1*04:01

As another example, in some cases, a TMAPP of the present disclosure comprises a DQA1*04:01 polypeptide comprising an amino acid sequence having at least 90%, at least 95%, at least 98%, at least 99%, or 100%, amino acid sequence identity to amino acids 24-204 of the DQA1*04:01 amino acid sequence provided below. In some cases, a TMAPP of the present disclosure comprises a DQA1*04:01 polypeptide comprising an amino acid sequence having at least 90%, at least 95%, at least 98%, at least 99%, or 100%, amino acid sequence identity to amino acids 24-110 of the DQA1*04:01 amino acid sequence provided below. In some cases, a TMAPP of the present disclosure comprises a DQA1*04:01 polypeptide comprising an amino acid sequence having at least 90%, at least 95%, at least 98%, at least 99%, or 100%, amino acid sequence identity to amino acids 111-204 of the DQA1*04:01 amino acid sequence provided below.

DQA1*04:01:
(SEQ ID NO: 400)
MILNKALLLGALALTTVMSPCGGEDIVADHVASYGVNLYQSYGPSGQYTHE
FDGDEQFYVDLGRKETVWCLPVLRQFRFDPQFALTNIAVTKHNLNILIKRS
NSTAATNEVPEVTVFSKSPVTLGQPNTLICLVDNIFPPVVNITWLSNGHSV
TEGVSETSFLSKSDHSFFKISYLTFLPSADEIYDCKVEHWGLDEPLLKHWE
PEIPAPMSELTETVVCALGLSVGLVGIVVGTVFIIRGLRSVGASRHQGPL.

### DQA1*05:01

As another example, in some cases, a TMAPP of the present disclosure comprises a DQA1*05:01 polypeptide comprising an amino acid sequence having at least 90%, at least 95%, at least 98%, at least 99%, or 100%, amino acid sequence identity to amino acids 24-204 of the DQA1*05:01 amino acid sequence provided below. In some cases, a TMAPP of the present disclosure comprises a DQA1*05:01 polypeptide comprising an amino acid sequence having at least 90%, at least 95%, at least 98%, at least 99%, or 100%, amino acid sequence identity to amino acids 24-110 of the DQA1*05:01 amino acid sequence provided below. In some cases, a TMAPP of the present disclosure comprises a DQA1*05:01 polypeptide comprising an amino acid sequence having at least 90%, at least 95%, at least 98%, at least 99%, or 100%, amino acid sequence identity to amino acids 111-204 of the DQA1*05:01 amino acid sequence provided below.

DQA1*05:01:
(SEQ ID NO: 401)
MILNKALMLGALALTTVMSPCGGEDIVADHVASYGVNLYQSYGPSGQYTHE
FDGDEQFYVDLGRKETVWCLPVLRQFRFDPQFALTNIAVLKHNLNSLIKRS
NSTAATNEVPEVTVFSKSPVTLGQPNILICLVDNIFPPVVNITWLSNGHSV
TEGVSETSFLSKSDHSFFKISYLTLLPSAEESYDCKVEHWGLDKPLLKHWE
PEIPAPMSELTETVVCALGLSVGLVGIVVGTVFIIRGLRSVGASRHQGPL.

### DQA1*06:01

As another example, in some cases, a TMAPP of the present disclosure comprises a DQA1*06:01 polypeptide comprising an amino acid sequence having at least 90%, at least 95%, at least 98%, at least 99%, or 100%, amino acid sequence identity to amino acids 24-204 of the DQA1*06:01 amino acid sequence provided below. In some cases, a TMAPP of the present disclosure comprises a DQA1*06:01 polypeptide comprising an amino acid sequence having at least 90%, at least 95%, at least 98%, at least 99%, or 100%, amino acid sequence identity to amino acids 24-110 of the DQA1*06:01 amino acid sequence provided below. In some cases, a TMAPP of the present disclosure comprises a DQA1*06:01 polypeptide comprising an amino acid sequence having at least 90%, at least 95%, at least 98%, at least 99%, or 100%, amino acid sequence identity to amino acids 111-204 of the DQA1*06:01 amino acid sequence provided below.

DQA1*06:01:
(SEQ ID NO: 402)
MILNKALLLGALALTTVMSPCGGEDIVADHVASYGVNLYQSYGPSGQFTHE
FDGDEQFYVDLGRKETVWCLPVLRQFRFDPQFALTNIAVTKHNLNILIKRS
NSTAATNEVPEVTVFSKSPVTLGQPNTLICLVDNIFPPVVNITWLSNGHSV
TEGVSETSFLSKSDHSFFKISYLTFLPSADEIYDCKVEHWGLDEPLLKHWE
PEIPAPMSELTETVVCALGLSVGLVGIVVGTVFIIRGLRSVGASRHQGPL.

### DRA1

In some cases, a TMAPP of the present disclosure comprises an MHC Class II α chain polypeptide of a DRA1 allele.

### DRA1*01:01

For example, in some cases, a TMAPP of the present disclosure comprises a DRA1*01:01 (also referred to as “DRA*01:01”; referred to in FIG. 39 as “DRA1*01:01) polypeptide comprising an amino acid sequence having at least 90%, at least 95%, at least 98%, at least 99%, or 100%, amino acid sequence identity to amino acids 26-203 of the DRA1*01:01 amino acid sequence provided below. In some cases, a TMAPP of the present disclosure comprises a DRA1*01:01 polypeptide comprising an amino acid sequence having at least 90%, at least 95%, at least 98%, at least 99%, or 100%, amino acid sequence identity to amino acids 26-109 of the DRA1*01:01 amino acid sequence provided below. In some cases, a TMAPP of the present disclosure comprises a DRA1*01:01 polypeptide comprising an amino acid sequence having at least 90%, at least 95%, at least 98%, at least 99%, or 100%, amino acid sequence identity to amino acids 110-203 of the DRA1*01:01 amino acid sequence provided below.

DRA1*01:01:
(SEQ ID NO: 403)
MAISGVPVLGFFIIAVLMSAQESWAIKEEHVIIQAEFYLNPDQSGEFMFDF
DGDEIFHVDMAKKETVWRLEEFGRFASFEAQGALANIAVDKANLEIMTKRS
NYTPITNVPPEVTVLTNSPVELREPNVLICFIDKFTPPVVNVTWLRNGKPV
TTGVSETVFLPREDHLFRKFHYLPFLPSTEDVYDCRVEHWGLDEPLLKHWE
FDAPSPLPETTENVVCALGLTVGLVGIIIGTIFIIKGVRKSNAAERRGPL.

### Combinations (Haplotypes)

A TMAPP of the present disclosure can comprise any combination of MHC α chains and MHC β chains, e.g., where the α chain is a DRA α chain and the β chain is a DRB β chain; where the α chain is a DQA α chain and the β chain is a DQB β chain, etc. The following are examples of possible combinations (haplotypes).

### DQA1*01:01 and DQB1*05:01

In some cases, a TMAPP of the present disclosure comprises: i) an MHC α chain polypeptide comprising an amino acid sequence having at least 60%, at least 70%, at least 80%, at least 90%, at least 95%, at least 98%, at least 99%, or 100%, amino acid sequence identity to a contiguous stretch of from about 70 amino acids to about 200 amino acids (e.g., from about 70 aa to about 100 aa, or from about 100 aa to about 150 aa, or from about 150 aa to about 200 aa) of a DQA1*01:01 α chain polypeptide; and ii) an MHC β chain polypeptide comprising an amino acid sequence having at least 60%, at least 70%, at least 80%, at least 90%, at least 95%, at least 98%, at least 99%, or 100%, amino acid sequence identity to a contiguous stretch of from about 70 amino acids to about 200 amino acids (e.g., from about 70 aa to about 100 aa, or from about 100 aa to about 150 aa, or from about 150 aa to about 200 aa) of a DQB1*05:01 β chain polypeptide. In some cases, a TMAPP of the present disclosure comprises: i) a DQA1*01:01 α chain polypeptide; and ii) a DQB1*05:01 β chain polypeptide.

### DRA1*01:01 and DRB1*04:01

In some cases, a TMAPP of the present disclosure comprises: i) an MHC α chain polypeptide comprising an amino acid sequence having at least 60%, at least 70%, at least 80%, at least 90%, at least 95%, at least 98%, at least 99%, or 100%, amino acid sequence identity to a contiguous stretch of from about 70 amino acids to about 200 amino acids (e.g., from about 70 aa to about 100 aa, or from about 100 aa to about 150 aa, or from about 150 aa to about 200 aa) of a DRA1*01:01 α chain polypeptide; and ii) an MHC β chain polypeptide comprising an amino acid sequence having at least 60%, at least 70%, at least 80%, at least 90%, at least 95%, at least 98%, at least 99%, or 100%, amino acid sequence identity to a contiguous stretch of from about 70 amino acids to about 200 amino acids (e.g., from about 70 aa to about 100 aa, or from about 100 aa to about 150 aa, or from about 150 aa to about 200 aa) of a DRB1*04:01 β chain polypeptide. In some cases, a TMAPP of the present disclosure comprises: i) a DRA1*01:01 α chain polypeptide; and ii) a DRB1*04:01 β chain polypeptide.

### DRA1*01:01 (DRA*01:01) and DRB5*01:01

In some cases, a TMAPP of the present disclosure comprises: i) an MHC α chain polypeptide comprising an amino acid sequence having at least 60%, at least 70%, at least 80%, at least 90%, at least 95%, at least 98%, at least 99%, or 100%, amino acid sequence identity to a contiguous stretch of from about 70 amino acids to about 200 amino acids (e.g., from about 70 aa to about 100 aa, or from about 100 aa to about 150 aa, or from about 150 aa to about 200 aa) of a DRA1*01:01 α chain polypeptide; and ii) an MHC β chain polypeptide comprising an amino acid sequence having at least 60%, at least 70%, at least 80%, at least 90%, at least 95%, at least 98%, at least 99%, or 100%, amino acid sequence identity to a contiguous stretch of from about 70 amino acids to about 200 amino acids (e.g., from about 70 aa to about 100 aa, or from about 100 aa to about 150 aa, or from about 150 aa to about 200 aa) of a DRB5*01:01 β chain polypeptide. In some cases, a TMAPP of the present disclosure comprises: i) a DRA1*01:01 α chain polypeptide; and ii) a DRB5*01:01 β chain polypeptide.

### DRA1*01:01 and DRB1*15:01

In some cases, a TMAPP of the present disclosure comprises: i) an MHC α chain polypeptide comprising an amino acid sequence having at least 60%, at least 70%, at least 80%, at least 90%, at least 95%, at least 98%, at least 99%, or 100%, amino acid sequence identity to a contiguous stretch of from about 70 amino acids to about 200 amino acids (e.g., from about 70 aa to about 100 aa, or from about 100 aa to about 150 aa, or from about 150 aa to about 200 aa) of a DRA1*01:01 α chain polypeptide; and ii) an MHC β chain polypeptide comprising an amino acid sequence having at least 60%, at least 70%, at least 80%, at least 90%, at least 95%, at least 98%, at least 99%, or 100%, amino acid sequence identity to a contiguous stretch of from about 70 amino acids to about 200 amino acids (e.g., from about 70 aa to about 100 aa, or from about 100 aa to about 150 aa, or from about 150 aa to about 200 aa) of a DRB1*15:01 β chain polypeptide. In some cases, a TMAPP of the present disclosure comprises: i) a DRA1*01:01 α chain polypeptide; and ii) a DRB1*15:01 β chain polypeptide.

### DQA1*01:02 and DQB1*06:02

In some cases, a TMAPP of the present disclosure comprises: i) an MHC α chain polypeptide comprising an amino acid sequence having at least 60%, at least 70%, at least 80%, at least 90%, at least 95%, at least 98%, at least 99%, or 100%, amino acid sequence identity to a contiguous stretch of from about 70 amino acids to about 200 amino acids (e.g., from about 70 aa to about 100 aa, or from about 100 aa to about 150 aa, or from about 150 aa to about 200 aa) of a DQA1*01:02 α chain polypeptide; and ii) an MHC β chain polypeptide comprising an amino acid sequence having at least 60%, at least 70%, at least 80%, at least 90%, at least 95%, at least 98%, at least 99%, or 100%, amino acid sequence identity to a contiguous stretch of from about 70 amino acids to about 200 amino acids (e.g., from about 70 aa to about 100 aa, or from about 100 aa to about 150 aa, or from about 150 aa to about 200 aa) of a DQB1*06:02 β chain polypeptide. In some cases, a TMAPP of the present disclosure comprises: i) a DQA1*01:02 α chain polypeptide; and ii) a DQB1*06:02 β chain polypeptide.

### DQA1*03:01 and DQB1*03:03

In some cases, a TMAPP of the present disclosure comprises: i) an MHC α chain polypeptide comprising an amino acid sequence having at least 60%, at least 70%, at least 80%, at least 90%, at least 95%, at least 98%, at least 99%, or 100%, amino acid sequence identity to a contiguous stretch of from about 70 amino acids to about 200 amino acids (e.g., from about 70 aa to about 100 aa, or from about 100 aa to about 150 aa, or from about 150 aa to about 200 aa) of a DQA1*03:01 α chain polypeptide; and ii) an MHC β chain polypeptide comprising an amino acid sequence having at least 60%, at least 70%, at least 80%, at least 90%, at least 95%, at least 98%, at least 99%, or 100%, amino acid sequence identity to a contiguous stretch of from about 70 amino acids to about 200 amino acids (e.g., from about 70 aa to about 100 aa, or from about 100 aa to about 150 aa, or from about 150 aa to about 200 aa) of a DQB1*03:02 β chain polypeptide. In some cases, a TMAPP of the present disclosure comprises: i) a DQA1*03:01 α chain polypeptide; and ii) a DQB1*03:02 β chain polypeptide.

### DQA1*01:04 and DQB1*05:01

In some cases, a TMAPP of the present disclosure comprises: i) an MHC α chain polypeptide comprising an amino acid sequence having at least 60%, at least 70%, at least 80%, at least 90%, at least 95%, at least 98%, at least 99%, or 100%, amino acid sequence identity to a contiguous stretch of from about 70 amino acids to about 200 amino acids (e.g., from about 70 aa to about 100 aa, or from about 100 aa to about 150 aa, or from about 150 aa to about 200 aa) of a DQA1*01:04 α chain polypeptide; and ii) an MHC β chain polypeptide comprising an amino acid sequence having at least 60%, at least 70%, at least 80%, at least 90%, at least 95%, at least 98%, at least 99%, or 100%, amino acid sequence identity to a contiguous stretch of from about 70 amino acids to about 200 amino acids (e.g., from about 70 aa to about 100 aa, or from about 100 aa to about 150 aa, or from about 150 aa to about 200 aa) of a DQB1*05:01 β chain polypeptide. In some cases, a TMAPP of the present disclosure comprises: i) a DQA1*01:04 α chain polypeptide; and ii) a DQB1*05:01 β chain polypeptide.

### DRA1*01:01 and DRB1*01:01

In some cases, a TMAPP of the present disclosure comprises: i) an MHC α chain polypeptide comprising an amino acid sequence having at least 60%, at least 70%, at least 80%, at least 90%, at least 95%, at least 98%, at least 99%, or 100%, amino acid sequence identity to a contiguous stretch of from about 70 amino acids to about 200 amino acids (e.g., from about 70 aa to about 100 aa, or from about 100 aa to about 150 aa, or from about 150 aa to about 200 aa) of a DRA1*01:01 α chain polypeptide; and ii) an MHC β chain polypeptide comprising an amino acid sequence having at least 60%, at least 70%, at least 80%, at least 90%, at least 95%, at least 98%, at least 99%, or 100%, amino acid sequence identity to a contiguous stretch of from about 70 amino acids to about 200 amino acids (e.g., from about 70 aa to about 100 aa, or from about 100 aa to about 150 aa, or from about 150 aa to about 200 aa) of a DRB1*01:01 β chain polypeptide. In some cases, a TMAPP of the present disclosure comprises: i) a DRA1*01:01 α chain polypeptide; and ii) a DRB1*01:01 β chain polypeptide.

### DQA1*04:01 and DQB1*04:02

In some cases, a TMAPP of the present disclosure comprises: i) an MHC α chain polypeptide comprising an amino acid sequence having at least 60%, at least 70%, at least 80%, at least 90%, at least 95%, at least 98%, at least 99%, or 100%, amino acid sequence identity to a contiguous stretch of from about 70 amino acids to about 200 amino acids (e.g., from about 70 aa to about 100 aa, or from about 100 aa to about 150 aa, or from about 150 aa to about 200 aa) of a DQA1*04:01 α chain polypeptide; and ii) an MHC β chain polypeptide comprising an amino acid sequence having at least 60%, at least 70%, at least 80%, at least 90%, at least 95%, at least 98%, at least 99%, or 100%, amino acid sequence identity to a contiguous stretch of from about 70 amino acids to about 200 amino acids (e.g., from about 70 aa to about 100 aa, or from about 100 aa to about 150 aa, or from about 150 aa to about 200 aa) of a DQB1*04:02 β chain polypeptide. In some cases, a TMAPP of the present disclosure comprises: i) a DQA1*04:01 α chain polypeptide; and ii) a DQB1*04:02 β chain polypeptide.

### DQA1*03:02 and DQB1*03:01

In some cases, a TMAPP of the present disclosure comprises: i) an MHC α chain polypeptide comprising an amino acid sequence having at least 60%, at least 70%, at least 80%, at least 90%, at least 95%, at least 98%, at least 99%, or 100%, amino acid sequence identity to a contiguous stretch of from about 70 amino acids to about 200 amino acids (e.g., from about 70 aa to about 100 aa, or from about 100 aa to about 150 aa, or from about 150 aa to about 200 aa) of a DQA1*03:02 α chain polypeptide; and ii) an MHC β chain polypeptide comprising an amino acid sequence having at least 60%, at least 70%, at least 80%, at least 90%, at least 95%, at least 98%, at least 99%, or 100%, amino acid sequence identity to a contiguous stretch of from about 70 amino acids to about 200 amino acids (e.g., from about 70 aa to about 100 aa, or from about 100 aa to about 150 aa, or from about 150 aa to about 200 aa) of a DQB1*03:01 β chain polypeptide. In some cases, a TMAPP of the present disclosure comprises: i) a DQA1*03:02 α chain polypeptide; and ii) a DQB1*03:01 β chain polypeptide.

### DQA1*01:03 and DQB1*06:01

In some cases, a TMAPP of the present disclosure comprises: i) an MHC α chain polypeptide comprising an amino acid sequence having at least 60%, at least 70%, at least 80%, at least 90%, at least 95%, at least 98%, at least 99%, or 100%, amino acid sequence identity to a contiguous stretch of from about 70 amino acids to about 200 amino acids (e.g., from about 70 aa to about 100 aa, or from about 100 aa to about 150 aa, or from about 150 aa to about 200 aa) of a DQA1*01:03 α chain polypeptide; and ii) an MHC β chain polypeptide comprising an amino acid sequence having at least 60%, at least 70%, at least 80%, at least 90%, at least 95%, at least 98%, at least 99%, or 100%, amino acid sequence identity to a contiguous stretch of from about 70 amino acids to about 200 amino acids (e.g., from about 70 aa to about 100 aa, or from about 100 aa to about 150 aa, or from about 150 aa to about 200 aa) of a DQB1*06:01 β chain polypeptide. In some cases, a TMAPP of the present disclosure comprises: i) a DQA1*01:03 α chain polypeptide; and ii) a DQB1*06:01 β chain polypeptide.

In some cases, a TMAPP of the present disclosure comprises an MHC Class II α- and/or β-chain allele that is associated with increased risk of developing a disease, e.g., where the individual to be treated with the TMAPP expresses the MHC Class II α- and/or β-chain allele.

### Scaffold Polypeptides

A TMAPP of the present disclosure, whether multimeric or monomeric, can comprise an immunoglobulin or non-immunoglobulin scaffold. A TMAPP polypeptide of the present disclosure, whether multimeric or monomeric, can comprise an Fc polypeptide, or can comprise another suitable scaffold polypeptide.

Suitable scaffold polypeptides include antibody-based scaffold polypeptides and non-antibody-based scaffolds. Non-antibody-based scaffolds include, e.g., albumin, an XTEN (extended recombinant) polypeptide, transferrin, an Fc receptor polypeptide, an elastin-like polypeptide (see, e.g., Hassouneh et al. (2012) Methods Enzymol. 502:215; e.g., a polypeptide comprising a pentapeptide repeat unit of (Val-Pro-Gly-X-Gly; SEQ ID NO: 59), where X is any amino acid other than proline), an albumin-binding polypeptide, a silk-like polypeptide (see, e.g., Valluzzi et al. (2002) Philos Trans R Soc Loud B Biol Sci. 357:165), a silk-elastin-like polypeptide (SELP; see, e.g., Megeed et al. (2002) Adv Drug Deliv Rev. 54:1075), and the like. Suitable XTEN polypeptides include, e.g., those disclosed in WO 2009/023270, WO 2010/091122, WO 2007/103515, US 2010/0189682, and US 2009/0092582; see also Schellenberger et al. (2009) Nat Biotechnol. 27:1186). Suitable albumin polypeptides include, e.g., human serum albumin.

Suitable scaffold polypeptides will in some cases be a half-life extending polypeptides. Thus, in some cases, a suitable scaffold polypeptide increases the in vivo half-life (e.g., the serum half-life) of the multimeric polypeptide, compared to a control multimeric polypeptide lacking the scaffold polypeptide. For example, in some cases, a scaffold polypeptide increases the in vivo half-life (e.g., the serum half-life) of the multimeric polypeptide, compared to a control multimeric polypeptide lacking the scaffold polypeptide, by at least about 10%, at least about 15%, at least about 20%, at least about 25%, at least about 50%, at least about 2-fold, at least about 2.5-fold, at least about 5-fold, at least about 10-fold, at least about 25-fold, at least about 50-fold, at least about 100-fold, or more than 100-fold. As an example, in some cases, an Fc polypeptide increases the in vivo half-life (e.g., the serum half-life) of the multimeric polypeptide, compared to a control multimeric polypeptide lacking the Fc polypeptide, by at least about 10%, at least about 15%, at least about 20%, at least about 25%, at least about 50%, at least about 2-fold, at least about 2.5-fold, at least about 5-fold, at least about 10-fold, at least about 25-fold, at least about 50-fold, at least about 100-fold, or more than 100-fold.

### Fc Polypeptides

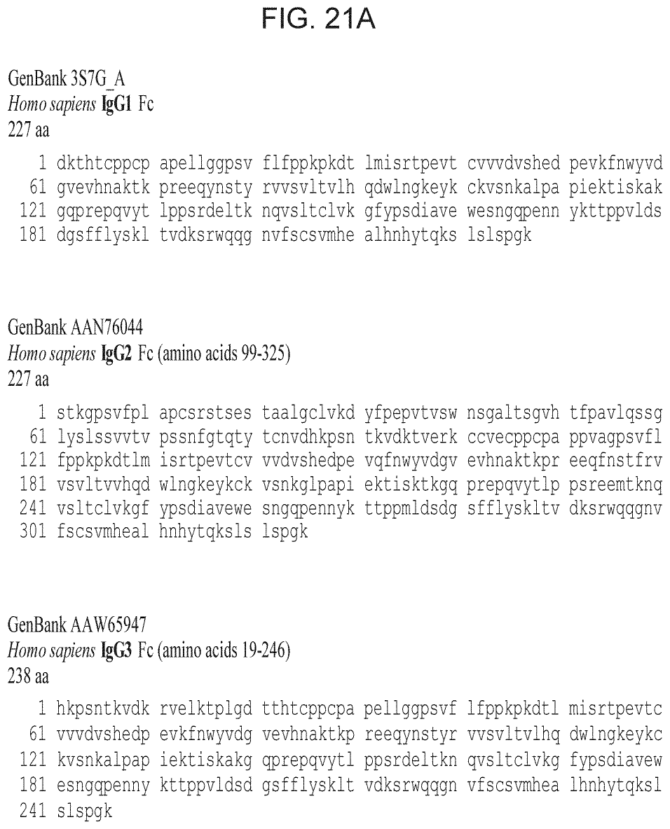

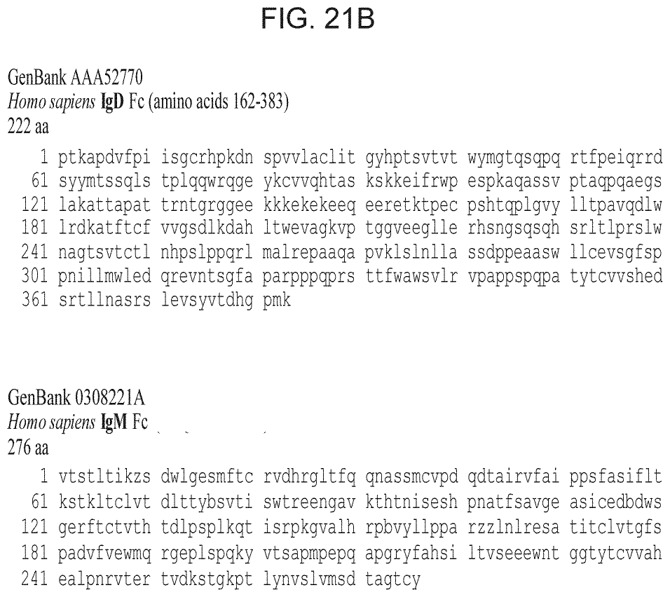

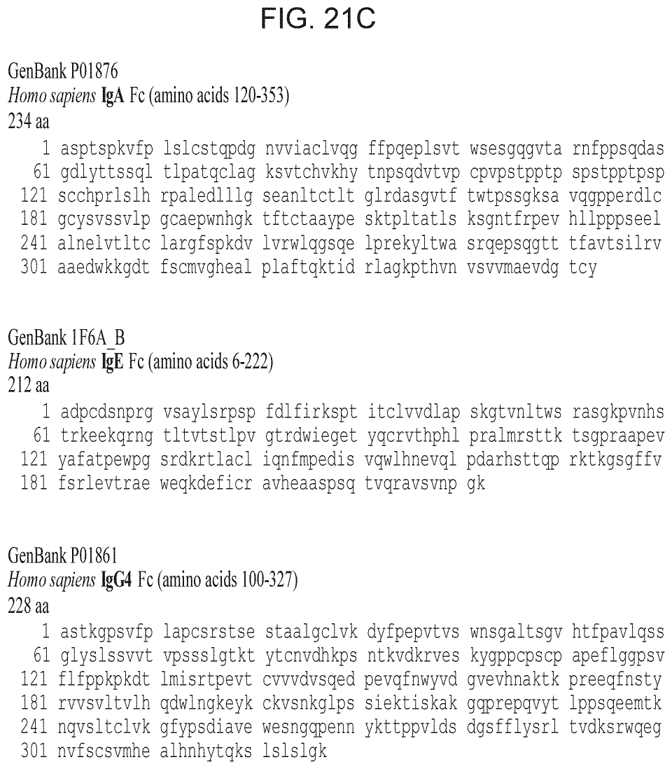

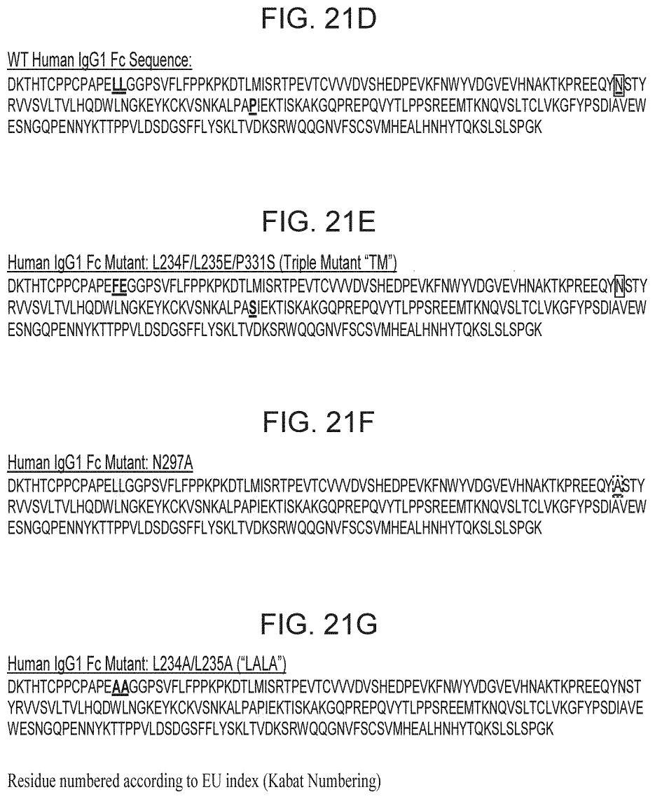

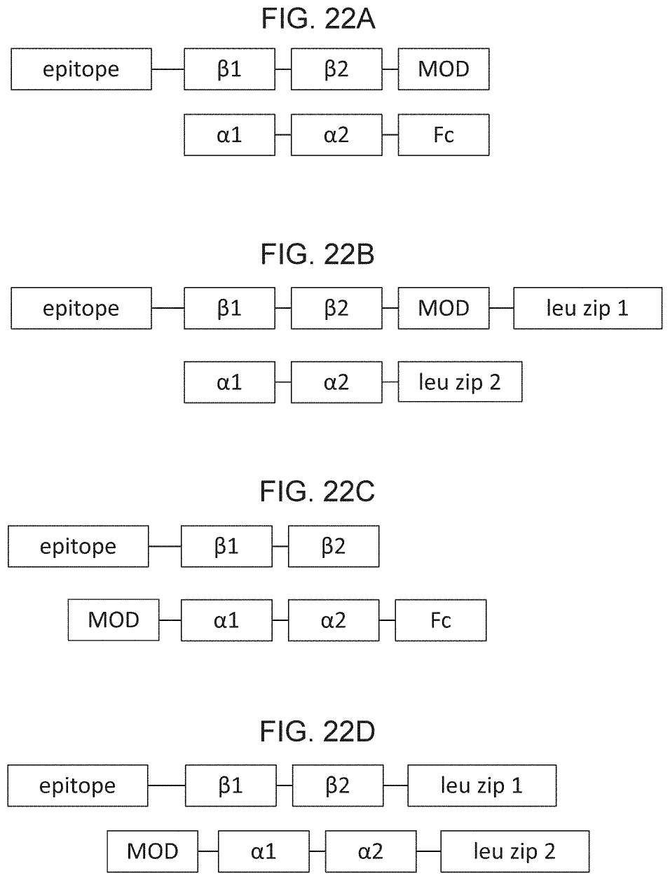

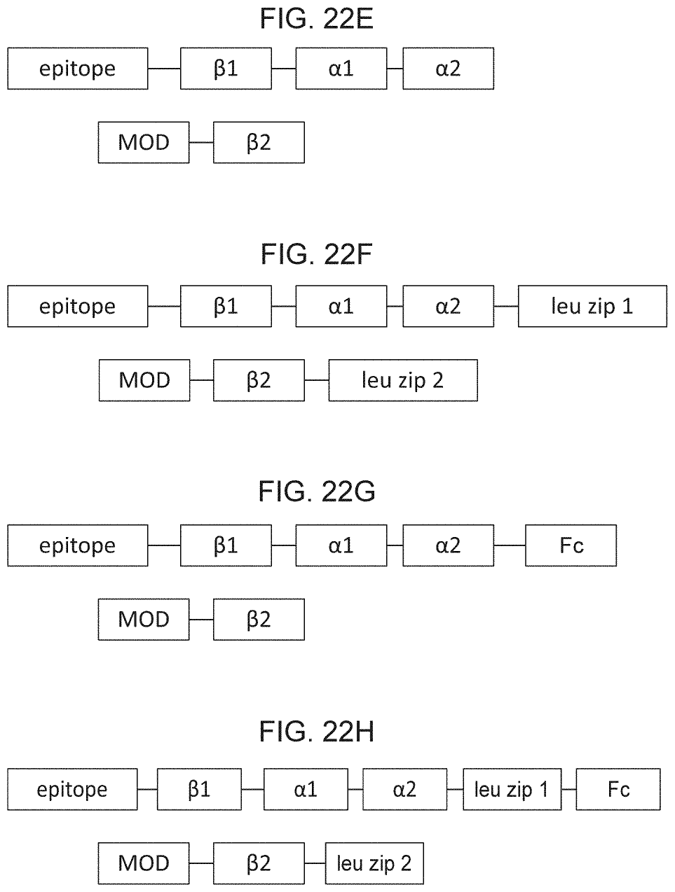

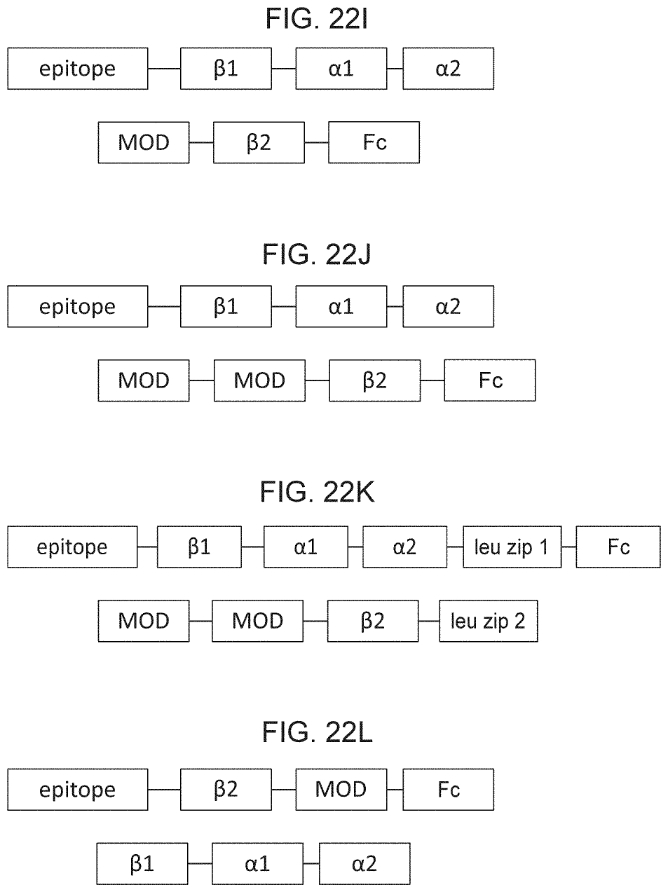

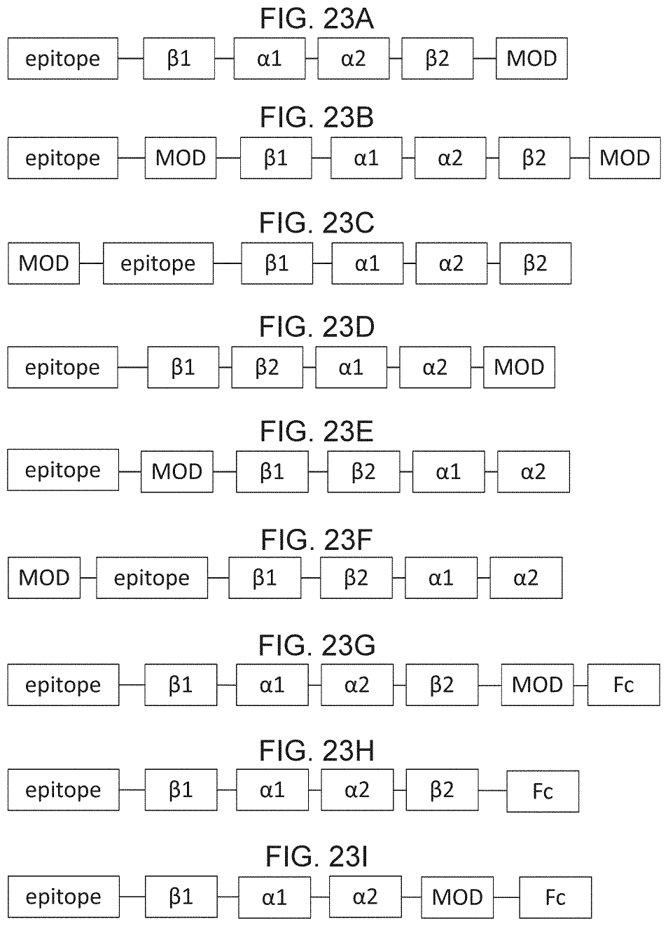

In some cases, the first and/or the second polypeptide chain of a TMMP of the present disclosure comprises an Fc polypeptide. The Fc polypeptide of a TMMP of the present disclosure can be a human IgG1 Fc, a human IgG2 Fc, a human IgG3 Fc, a human IgG4 Fc, etc. In some cases, the Fc polypeptide comprises an amino acid sequence having at least about 70%, at least about 75%, at least about 80%, at least about 85%, at least about 90%, at least about 95%, at least about 98%, at least about 99%, or 100%, amino acid sequence identity to an amino acid sequence of an Fc region depicted in FIG. 21A-21G. In some cases, the Fc region comprises an amino acid sequence having at least about 70%, at least about 75%, at least about 80%, at least about 85%, at least about 90%, at least about 95%, at least about 98%, at least about 99%, or 100%, amino acid sequence identity to the human IgG1 Fc polypeptide depicted in FIG. 21A. In some cases, the Fc region comprises an amino acid sequence having at least about 70%, at least about 75%, at least about 80%, at least about 85%, at least about 90%, at least about 95%, at least about 98%, at least about 99%, or 100%, amino acid sequence identity to the human IgG1 Fc polypeptide depicted in FIG. 21A; and comprises a substitution of N77; e.g., the Fc polypeptide comprises an N77A substitution. In some cases, the Fc polypeptide comprises an amino acid sequence having at least about 70%, at least about 75%, at least about 80%, at least about 85%, at least about 90%, at least about 95%, at least about 98%, at least about 99%, or 100%, amino acid sequence identity to the human IgG2 Fc polypeptide depicted in FIG. 21A; e.g., the Fc polypeptide comprises an amino acid sequence having at least about 70%, at least about 75%, at least about 80%, at least about 85%, at least about 90%, at least about 95%, at least about 98%, at least about 99%, or 100%, amino acid sequence identity to amino acids 99-325 of the human IgG2 Fc polypeptide depicted in FIG. 21A. In some cases, the Fc polypeptide comprises an amino acid sequence having at least about 70%, at least about 75%, at least about 80%, at least about 85%, at least about 90%, at least about 95%, at least about 98%, at least about 99%, or 100%, amino acid sequence identity to the human IgG3 Fc polypeptide depicted in FIG. 21A; e.g., the Fc polypeptide comprises an amino acid sequence having at least about 70%, at least about 75%, at least about 80%, at least about 85%, at least about 90%, at least about 95%, at least about 98%, at least about 99%, or 100%, amino acid sequence identity to amino acids 19-246 of the human IgG3 Fc polypeptide depicted in FIG. 21A. In some cases, the Fc polypeptide comprises an amino acid sequence having at least about 70%, at least about 75%, at least about 80%, at least about 85%, at least about 90%, at least about 95%, at least about 98%, at least about 99%, or 100%, amino acid sequence identity to the human IgM Fc polypeptide depicted in FIG. 21B; e.g., the Fc polypeptide comprises an amino acid sequence having at least about 70%, at least about 75%, at least about 80%, at least about 85%, at least about 90%, at least about 95%, at least about 98%, at least about 99%, or 100%, amino acid sequence identity to amino acids 1-276 to the human IgM Fc polypeptide depicted in FIG. 21B. In some cases, the Fc polypeptide comprises an amino acid sequence having at least about 70%, at least about 75%, at least about 80%, at least about 85%, at least about 90%, at least about 95%, at least about 98%, at least about 99%, or 100%, amino acid sequence identity to the human IgA Fc polypeptide depicted in FIG. 21C; e.g., the Fc polypeptide comprises an amino acid sequence having at least about 70%, at least about 75%, at least about 80%, at least about 85%, at least about 90%, at least about 95%, at least about 98%, at least about 99%, or 100%, amino acid sequence identity to amino acids 1-234 to the human IgA Fc polypeptide depicted in FIG. 21C.

In some cases, the Fc polypeptide comprises an amino acid sequence having at least about 70%, at least about 75%, at least about 80%, at least about 85%, at least about 90%, at least about 95%, at least about 98%, at least about 99%, or 100%, amino acid sequence identity to the human IgG4 Fc polypeptide depicted in FIG. 21C. In some cases, the Fc polypeptide comprises an amino acid sequence having at least about 70%, at least about 75%, at least about 80%, at least about 85%, at least about 90%, at least about 95%, at least about 98%, at least about 99%, or 100%, amino acid sequence identity to amino acids 100 to 327 of the human IgG4 Fc polypeptide depicted in FIG. 21C.

In some cases, the IgG4 Fc polypeptide comprises the following amino acid sequence:

(SEQ ID NO: 60)
PPCPSCPAPEFLGGPSVFLFPPKPKDTLMISRTPEVTCVVVDVSQEDPEVQ
FNWYVDGVEVHNAKTKPREEQFNSTYRVVSVLTVLHQDWLNGKEYKCKVSN
KGLPSSIEKTISKAKGQPREPQVYTLPPSQEEMTKNQVSLTCLVKGFYPSD
IAVEWESNGQPENNYKTTPPVLDSDGSFFLYSRLTVDKSRWQEGNVFSCSV
MHEALHNHYTQKSLSLSPG.

In some cases, the Fc polypeptide present in a TMMP comprises the amino acid sequence depicted in FIG. 21A (human IgG1 Fc). In some cases, the Fc polypeptide present in a TMMP comprises the amino acid sequence depicted in FIG. 21A (human IgG1 Fc), except for a substitution of N297 (N77 of the amino acid sequence depicted in FIG. 21A) with an amino acid other than asparagine. In some cases, the Fc polypeptide present in a TMMP comprises the amino acid sequence depicted in FIG. 21C (human IgG1 Fc comprising an N297A substitution, which is N77 of the amino acid sequence depicted in FIG. 21A). In some cases, the Fc polypeptide present in a TMMP comprises the amino acid sequence depicted in FIG. 21A (human IgG1 Fc), except for a substitution of L234 (L14 of the amino acid sequence depicted in FIG. 21A) with an amino acid other than leucine. In some cases, the Fc polypeptide present in a TMMP comprises the amino acid sequence depicted in FIG. 21A (human IgG1 Fc), except for a substitution of L235 (L15 of the amino acid sequence depicted in FIG. 21A) with an amino acid other than leucine.

In some cases, the Fc polypeptide present in a TMMP comprises the amino acid sequence depicted in FIG. 21E. In some cases, the Fc polypeptide present in a TMMP comprises the amino acid sequence depicted in FIG. 21F. In some cases, the Fc polypeptide present in a TMMP comprises the amino acid sequence depicted in FIG. 21G (human IgG1 Fc comprising an L234A substitution and an L235A substitution, corresponding to positions 14 and 15 of the amino acid sequence depicted in FIG. 21G). In some cases, the Fc polypeptide present in a TMMP comprises the amino acid sequence depicted in FIG. 21A (human IgG1 Fc), except for a substitution of P331 (P111 of the amino acid sequence depicted in FIG. 21A) with an amino acid other than proline; in some cases, the substitution is a P331S substitution. In some cases, the Fc polypeptide present in a TMMP comprises the amino acid sequence depicted in FIG. 21A (human IgG1 Fc), except for substitutions at L234 and L235 (L14 and L15 of the amino acid sequence depicted in FIG. 21A) with amino acids other than leucine. In some cases, the Fc polypeptide present in a TMMP comprises the amino acid sequence depicted in FIG. 21A (human IgG1 Fc), except for substitutions at L234 and L235 (L14 and L15 of the amino acid sequence depicted in FIG. 21A) with amino acids other than leucine, and a substitution of P331 (P111 of the amino acid sequence depicted in FIG. 21A) with an amino acid other than proline. In some cases, the Fc polypeptide present in a TMMP comprises the amino acid sequence depicted in FIG. 21E (human IgG1 Fc comprising L234F, L235E, and P331S substitutions (corresponding to amino acid positions 14, 15, and 111 of the amino acid sequence depicted in FIG. 21E). In some cases, the Fc polypeptide present in a TMMP is an IgG1 Fc polypeptide that comprises L234A and L235A substitutions (substitutions of L14 and L15 of the amino acid sequence depicted in FIG. 21A with Ala), as depicted in FIG. 21G.

### Linkers

As noted above, a TMAPP of the present disclosure can include a linker peptide interposed between, e.g., an epitope and an MHC polypeptide; between an MHC polypeptide and an Ig Fc polypeptide; between a first MHC polypeptide and a second MHC polypeptide; etc.

Suitable linkers (also referred to as “spacers”) can be readily selected and can be of any of a number of suitable lengths, such as from 1 amino acid to 25 amino acids, from 3 amino acids to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids. A suitable linker can be 1, 2, 3, 4, 5, 6, 7, 8, 9, 10, 11, 12, 13, 14, 15, 16, 17, 18, 19, 20, 21, 22, 23, 24, or 25 amino acids in length. A suitable linker can be from 25 to 35 amino acids in length. A suitable linker can be 25, 26, 27, 28, 29, 30, 31, 32, 33, 34, or 35 amino acids in length. A suitable linker can be from 35 to 45 amino acids in length. A suitable linker can be 35, 36, 37, 38, 39, 40, 41, 42, 43, 44, or 45 amino acids in length. A suitable linker can be from 45 to 50 amino acids in length. A suitable linker can be 45, 46, 47, 48, 49, or 50 amino acids in length.

Exemplary linkers include glycine polymers (G)ₙ, glycine-serine polymers (including, for example, (GS)ₙ, (GSGGS)ₙ (SEQ ID NO: 61) and (GGGS)ₙ (SEQ ID NO: 62), where n is an integer of at least one), glycine-alanine polymers, alanine-serine polymers, and other flexible linkers known in the art. Glycine and glycine-serine polymers can be used; both Gly and Ser are relatively unstructured, and therefore can serve as a neutral tether between components. Glycine polymers can be used; glycine accesses significantly more phi-psi space than even alanine, and is much less restricted than residues with longer side chains (see Scheraga, Rev. Computational Chem. 11173-142 (1992)). Exemplary linkers can comprise amino acid sequences including, but not limited to, GGSG (SEQ ID NO: 63), GGSGG (SEQ ID NO: 64), GSGSG (SEQ ID NO: 65), GSGGG (SEQ ID NO: 66), GGGSG (SEQ ID NO: 67), GSSSG (SEQ ID NO: 68), and the like. Exemplary linkers can include, e.g., Gly(Ser₄)n, (SEQ ID NO: 69) where n is 1, 2, 3, 4, 5, 6, 7, 8, 9, or 10. In some cases, a linker comprises the amino acid sequence (GSSSS)n (SEQ ID NO: 435), where n is 4. In some cases, a linker comprises the amino acid sequence (GSSSS)n (SEQ ID NO: 436), where n is 5. Exemplary linkers can include, e.g., (GlyGlyGlyGlySer)n (SEQ ID NO: 437), where n is 1, 2, 3, 4, 5, 6, 7, 8, 9, or 10. In some cases, a linker comprises the amino acid sequence (GGGGS)n (SEQ ID NO: 423), where n is 1. In some cases, a linker comprises the amino acid sequence (GGGGS)n (SEQ ID NO: 424), where n is 2. In some cases, a linker comprises the amino acid sequence (GGGGS)n (SEQ ID NO: 301), where n is 3. In some cases, a linker comprises the amino acid sequence (GGGGS)n (SEQ ID NO: 302), where n is 4. In some cases, a linker comprises the amino acid sequence (GGGGS)n (SEQ ID NO: 425), where n is 5. In some cases, a linker comprises the amino acid sequence (GGGGS)n (SEQ ID NO: 426), where n is 6. In some cases, a linker comprises the amino acid sequence (GGGGS)n (SEQ ID NO: 427), where n is 7. In some cases, a linker comprises the amino acid sequence (GGGGS)n (SEQ ID NO: 428), where n is 8. In some cases, a linker comprises the amino acid sequence (GGGGS)n (SEQ ID NO: 429), where n is 9. In some cases, a linker comprises the amino acid sequence (GGGGS)n (SEQ ID NO: 430), where n is 10. In some cases, a linker comprises the amino acid sequence AAAGG (SEQ ID NO: 70).

In some cases, a linker polypeptide present in a TMAPP of the present disclosure includes a cysteine residue that can form a disulfide bond with a cysteine residue present in a second polypeptide of the TMAPP. In some cases, for example, a suitable linker comprises the amino acid sequence GCGASGGGGSGGGGS (SEQ ID NO: 71).

### Epitope-Presenting Peptides

A peptide epitope (also referred to herein as a “peptide antigen” or “epitope-presenting peptide” or “epitope”) present in a TMAPP of the present disclosure presents an epitope to a TCR on the surface of a T cell. An epitope-presenting peptide can have a length of from about 4 amino acids to about 25 amino acids, e.g., the epitope can have a length of from 4 amino acids (aa) to 10 aa, from 10 aa to 15 aa, from 15 aa to 20 aa, or from 20 aa to 25 aa. For example, an epitope present in a TMAPP of the present disclosure can have a length of 4 amino acids (aa), 5 aa, 6 aa, 7, aa, 8 aa, 9 aa, 10 aa, 11 aa, 12 aa, 13 aa, 14 aa, 15 aa, 16 aa, 17 aa, 18 aa, 19 aa, 20 aa, 21 aa, 22 aa, 23 aa, 24 aa, or 25 aa. In some cases, an epitope-presenting peptide present in a TMAPP of the present disclosure has a length of from 5 amino acids to 10 amino acids, e.g., 5 aa, 6 aa, 7 aa, 8 aa, 9 aa, or 10 aa.

An epitope-presenting peptide present in a TMAPP of the present disclosure is specifically bound by a T-cell, i.e., the epitope is specifically bound by an epitope-specific T cell. An epitope-specific T cell binds an epitope-presenting peptide having a reference amino acid sequence, but does not substantially bind an epitope that differs from the reference amino acid sequence. For example, an epitope-specific T cell binds an epitope-presenting peptide having a reference amino acid sequence, and binds an epitope that differs from the reference amino acid sequence, if at all, with an affinity that is less than 10⁻⁶ M, less than 10⁻⁵ M, or less than 10⁻⁴ M. An epitope-specific T cell can bind an epitope-presenting peptide for which it is specific with an affinity of at least 10⁻⁷ M, at least 10⁻⁸ M, at least 10⁻⁹ M, or at least 10⁻¹⁰ M.

### Cancer Epitopes

Suitable epitope-presenting peptides include, but are not limited to, epitope-presenting peptides present in a cancer-associated antigen. Cancer-associated antigens include, but are not limited to, α-folate receptor; carbonic anhydrase IX (CAIX); CD19; CD20; CD22; CD30; CD33; CD44v7/8; carcinoembryonic antigen (CEA); epithelial glycoprotein-2 (EGP-2); epithelial glycoprotein-40 (EGP-40); folate binding protein (FBP); fetal acetylcholine receptor; ganglioside antigen GD2; Her2/neu; IL-13R-a2; kappa light chain; LeY; L1 cell adhesion molecule; melanoma-associated antigen (MAGE); MAGE-A1; mesothelin; MUC1; NKG2D ligands; oncofetal antigen (h5T4); prostate stem cell antigen (PSCA); prostate-specific membrane antigen (PSMA); tumor-associate glycoprotein-72 (TAG-72); and vascular endothelial growth factor receptor-2 (VEGF-R2). See, e.g., Vigneron et al. (2013) Cancer Immunity 13:15; and Vigneron (2015) BioMed Res. Int'l Article ID 948501. In some cases, the epitope is a human papilloma virus E7 antigen epitope; see, e.g., Ramos et al. (2013) J. Immunother. 36:66.

In some cases, a suitable peptide epitope is a peptide fragment of from about 4 amino acids to about 20 amino acids (e.g., 4 amino acids (aa), 5 aa, 6 aa, 7 aa, 8 aa, 9 aa, 10 aa, 11 aa, 12 aa, 13 aa, 14 aa, 15 aa, 16 aa, 17 aa, 18 aa, 19 aa, or 20 aa) in length of a MUC1 polypeptide, a human papillomavirus (HPV) E6 polypeptide, an LMP2 polypeptide, an HPV E7 polypeptide, an epidermal growth factor receptor (EGFR) VIII polypeptide, a HER-2/neu polypeptide, a melanoma antigen family A, 3 (MAGE A3) polypeptide, a p53 polypeptide, a mutant p53 polypeptide, an NY-ESO-1 polypeptide, a folate hydrolase (prostate-specific membrane antigen; PSMA) polypeptide, a carcinoembryonic antigen (CEA) polypeptide, a melanoma antigen recognized by T-cells (melanA/MART1) polypeptide, a Ras polypeptide, a gp100 polypeptide, a proteinase3 (PR1) polypeptide, a bcr-abl polypeptide, a tyrosinase polypeptide, a survivin polypeptide, a prostate specific antigen (PSA) polypeptide, an hTERT polypeptide, a sarcoma translocation breakpoints polypeptide, a synovial sarcoma X (SSX) breakpoint polypeptide, an EphA2 polypeptide, an acid phosphatase, prostate (PAP) polypeptide, a melanoma inhibitor of apoptosis (ML-IAP) polypeptide, an alpha-fetoprotein (AFP) polypeptide, an epithelial cell adhesion molecule (EpCAM) polypeptide, an ERG (TMPRSS2 ETS fusion) polypeptide, a NA17 polypeptide, a paired-box-3 (PAX3) polypeptide, an anaplastic lymphoma kinase (ALK) polypeptide, an androgen receptor polypeptide, a cyclin B1 polypeptide, an N-myc proto-oncogene (MYCN) polypeptide, a Ras homolog gene family member C (RhoC) polypeptide, a tyrosinase-related protein-2 (TRP-2) polypeptide, a mesothelin polypeptide, a prostate stem cell antigen (PSCA) polypeptide, a melanoma associated antigen-1 (MAGE A1) polypeptide, a cytochrome P450 1B1 (CYP1B1) polypeptide, a placenta-specific protein 1 (PLAC1) polypeptide, a BORIS polypeptide (also known as CCCTC-binding factor or CTCF), an ETV6-AML polypeptide, a breast cancer antigen NY-BR-1 polypeptide (also referred to as ankyrin repeat domain-containing protein 30A), a regulator of G-protein signaling (RGS5) polypeptide, a squamous cell carcinoma antigen recognized by T-cells (SART3) polypeptide, a carbonic anhydrase IX polypeptide, a paired box-5 (PAX5) polypeptide, an OY-TES1 (testis antigen; also known as acrosin binding protein) polypeptide, a sperm protein 17 polypeptide, a lymphocyte cell-specific protein-tyrosin kinase (LCK) polypeptide, a high molecular weight melanoma associated antigen (HMW-MAA), an A-kinase anchoring protein-4 (AKAP-4), a synovial sarcoma X breakpoint 2 (SSX2) polypeptide, an X antigen family member 1 (XAGE1) polypeptide, a B7 homolog 3 (B7H3; also known as CD276) polypeptide, a legumain polypeptide (LGMN1; also known as asparaginyl endopeptidase), a tyrosine kinase with Ig and EGF homology domains-2 (Tie-2; also known as angiopoietin-1 receptor) polypeptide, a P antigen family member 4 (PAGE4) polypeptide, a vascular endothelial growth factor receptor 2 (VEGF2) polypeptide, a MAD-CT-1 polypeptide, a fibroblast activation protein (FAP) polypeptide, a platelet derived growth factor receptor beta (PDGFβ) polypeptide, a MAD-CT-2 polypeptide, a Fos-related antigen-1 (FOSL) polypeptide, and a Wilms tumor-1 (WT1) polypeptide.

Amino acid sequences of cancer-associated antigens are known in the art; see, e.g., MUC1 (GenBank CAA56734); LMP2 (GenBank CAA47024); HPV E6 (GenBank AAD33252); HPV E7 (GenBank AHG99480); EGFRvIII (GenBank NP_001333870); HER-2/neu (GenBank AAI67147); MAGE-A3 (GenBank AAH11744); p53 (GenBank BAC16799); NY-ESO-1 (GenBank CAA05908); PSMA (GenBank AAH25672); CEA (GenBank AAA51967); melan/MART1 (GenBank NP_005502); Ras (GenBank NP_001123914); gp100 (GenBank AAC60634); bcr-abl (GenBank AAB60388); tyrosinase (GenBank AAB60319); survivin (GenBank AAC51660); PSA (GenBank CAD54617); hTERT (GenBank BAC11010); SSX (GenBank NP_001265620); Eph2A (GenBank NP_004422); PAP (GenBank AAH16344); ML-IAP (GenBank AAH14475); AFP (GenBank NP_001125); EpCAM (GenBank NP_002345); ERG (TMPRSS2 ETS fusion) (GenBank ACA81385); PAX3 (GenBank AAI01301); ALK (GenBank NP_004295); androgen receptor (GenBank NP_000035); cyclin B1 (GenBank CA099273); MYCN (GenBank NP_001280157); RhoC (GenBank AAH52808); TRP-2 (GenBank AAC60627); mesothelin (GenBank AAH09272); PSCA (GenBank AAH65183); MAGE A1 (GenBank NP_004979); CYP1B1 (GenBank AAM50512); PLAC1 (GenBank AAG22596); BORIS (GenBank NP_001255969); ETV6 (GenBank NP_001978); NY-BR1 (GenBank NP_443723); SART3 (GenBank NP_055521); carbonic anhydrase IX (GenBank EAW58359); PAX5 (GenBank NP_057953); OY-TES1 (GenBank NP_115878); sperm protein 17 (GenBank AAK20878); LCK (GenBank NP_001036236); HMW-MAA (GenBank NP_001888); AKAP-4 (GenBank NP_003877); SSX2 (GenBank CAA60111); XAGE1 (GenBank NP_001091073; XP_001125834; XP_001125856; and XP_001125872); B7H3 (GenBank NP_001019907; XP_947368; XP_950958; XP_950960; XP_950962; XP_950963; XP_950965; and XP_950967); LGMN1 (GenBank NP_001008530); TIE-2 (GenBank NP_000450); PAGE4 (GenBank NP_001305806); VEGFR2 (GenBank NP_002244); MAD-CT-1 (GenBank NP_005893 NP_056215); FAP (GenBank NP_004451); PDGFβ (GenBank NP_002600); MAD-CT-2 (GenBank NP_001138574); FOSL (GenBank NP_005429); and WT-1 (GenBank NP_000369). These polypeptides are also discussed in, e.g., Cheever et al. (2009) Clin. Cancer Res. 15:5323, and references cited therein; Wagner et al. (2003) J. Cell. Sci. 116:1653; Matsui et al. (1990) Oncogene 5:249; Zhang et al. (1996) Nature 383:168.

In some cases, the epitope is HPV16E7/82-90 (LLMGTLGIV; SEQ ID NO: 404). In some cases, the epitope is HPV16E7/86-93 (TLGIVCPI; SEQ ID NO: 405). In some cases, the epitope is HPV16E7/11-20 (YMLDLQPETT; SEQ ID NO: 406). In some cases, the epitope is HPV16E7/11-19 (YMLDLQPET; SEQ ID NO: 407). See, e.g., Ressing et al. ((1995) J. Immunol. 154:5934) for additional suitable HPV epitopes.

### Self Epitopes

In some cases, the peptide epitope is an epitope associated with or present in a “self” antigen (an autoantigen). Antigens associated with autoimmune disease can be autoantigens associated with autoimmune diseases such as Addison disease (autoimmune adrenalitis, Morbus Addison), alopecia areata, Addison's anemia (Morbus Biermer), autoimmune hemolytic anemia (AIHA), autoimmune hemolytic anemia (AIHA) of the cold type (cold hemagglutinin disease, cold autoimmune hemolytic anemia (AIHA) (cold agglutinin disease), (CHAD)), autoimmune hemolytic anemia (AIHA) of the warm type (warm AIHA, warm autoimmune hemolytic anemia (AIHA)), autoimmune hemolytic Donath-Landsteiner anemia (paroxysmal cold hemoglobinuria), antiphospholipid syndrome (APS), atherosclerosis, autoimmune arthritis, arteriitis temporalis, Takayasu arteriitis (Takayasu's disease, aortic arch disease), temporal arteriitis/giant cell arteriitis, autoimmune chronic gastritis, autoimmune infertility, autoimmune inner ear disease (AIED), Basedow's disease (Morbus Basedow), Bechterew's disease (Morbus Bechterew, ankylosing spondylitis, spondylitis ankylosans), Behcet's syndrome (Morbus Behcet), bowel disease including autoimmune inflammatory bowel disease (including colitis ulcerosa (Morbus Crohn, Crohn's disease), autoimmune cardiomyopathy, idiopathic dilated cardiomyopathy (DCM), chronic fatigue immune dysfunction syndrome (CFIDS), chronic inflammatory demyelinating polyneuropathy (CIDP), chronic polyarthritis, Churg-Strauss syndrome, cicatricial pemphigoid, Cogan syndrome, CREST syndrome (syndrom with Calcinosis cutis, Raynaud phenomenon, motility disorders of the esophagus, sklerodaktylia and teleangiectasia), Crohn's disease (Morbus Crohn, colitis ulcerosa), dermatitis herpetiformis during, dermatologic autoimmune diseases, dermatomyositis, essential mixed cryoglobulinemia, essential mixed cryoglobulinemia, fibromyalgia, fibromyositis, Goodpasture syndrome (anti-GBM mediated glomerulonephritis), graft versus host disease, Guillain-Barre syndrome (GBM, Polyradikuloneuritis), hematologic autoimmune diseases, Hashimoto thyroiditis, hemophilia, acquired hemophilia, autoimmune hepatitis, idiopathic pulmonary fibrosis (IPF), idiopathic thrombocytopenic purpura, Immuno-thrombocytopenic purpura (Morbus Werlhof, ITP), IgA nephropathy, autoimmune infertility, juvenile rheumatoid arthritis (Morbus Still, Still syndrome), Lambert-Eaton syndrome, systemic lupus erythematosus (SLE), lupus erythematosus (discoid form), Lyme arthritis (Lyme disease, borrelia arthritis), Meniere's disease (Morbus Meniere); mixed connective tissue disease (MCTD), multiple sclerosis (MS, encephalomyelitis disseminate, Charcot's disease), myasthenia gravis (myasthenia, MG), myositis, polymyositis, neural autoimmune diseases, pemphigus vulgaris, bullous pemphigoid, polyglandular (autoimmune) syndrome (PGA syndrome, Schmidt's syndrome), polymyalgia rheumatica, primary agammaglobulinemia, primary autoimmune cholangitis, progressive systemic sclerosis (PSS), rheumatoid arthritis (RA, chronic polyarthritis, rheumatic disease of the joints, rheumatic fever), sarcoidosis (Morbus Boeck, Besnier-Boeck-Schaumann disease), stiff-man syndrome, Sclerodermia, Scleroderma, Sjögren's syndrome, autoimmune uveiitis, and Wegner's disease (Morbus Wegner, Wegner's granulomatosis).

In some cases, a peptide epitope present in a TMMP of the present disclosure is a peptide associated with Addison's disease, alopecia areata, ankylosing spondylitis, autoimmune encephalomyelitis, autoimmune hemolytic anemia, autoimmune hepatitis, autoimmune-associated infertility, autoimmune thrombocytopenic purpura, bullous pemphigoid, Crohn's disease, Goodpasture's syndrome, glomerulonephritis (e.g., crescentic glomerulonephritis, proliferative glomerulonephritis), Grave's disease, Hashimoto's thyroiditis, mixed connective tissue disease, multiple sclerosis, myasthenia gravis (MG), pemphigus (e.g., pemphigus vulgaris), pernicious anemia, polymyositis, psoriasis, psoriatic arthritis, rheumatoid arthritis, scleroderma, Sjögren's syndrome, systemic lupus erythematosus (SLE), vasculitis, or vitiligo.

Autoantigens include, e.g., aggrecan, alanyl-tRNA syntetase (PL-12), alpha beta crystallin, alpha fodrin (Sptan 1), alpha-actinin, α1 antichymotrypsin, α1 antitripsin, α1 microglobulin, aldolase, aminoacyl-tRNA synthetase, an amyloid, an annexin, an apolipoprotein, aquaporin, bactericidal/permeability-increasing protein (BPI), β-globin precursor BP1, β-actin, β-lactoglobulin A, β-2-glycoprotein I, β2-microglobulin, a blood group antigen, C reactive protein (CRP), calmodulin, calreticulin, cardiolipin, catalase, cathepsin B, a centromere protein, chondroitin sulfate, chromatin, collagen, a complement component, cytochrome C, cytochrome P450 2D6, cytokeratins, decorin, dermatan sulfate, DNA topoisomerase I, elastin, Epstein-Barr nuclear antigen 1 (EBNA1), elastin, entaktin, an extractable nuclear antigen, Factor I, Factor P, Factor B, Factor D, Factor H, Factor X, fibrinogen, fibronectin, formiminotransferase cyclodeaminase (LC-1), gp210 nuclear envelope protein, GP2 (major zymogen granule membrane glycoprotein), a glutenin, glycoprotein gpIIb/IIIa, glial fibrillary acidic protein (GFAP), glycated albumin, glyceraldehyde 3-phosphate dehydrogenase (GAPDH), haptoglobin A2, heat shock proteins, hemocyanin, heparin, a histone, histidyl-tRNA synthetase (Jo-1), a hordein, hyaluronidase, immunoglobulins, an integrin, interstitial retinol-binding protein 3, intrinsic factor, Ku (p70/p80), lactate dehydrogenase, laminin, liver cytosol antigen type 1 (LC1), liver/kidney microsomal antigen 1 (LKM1), lysozyme, melanoma differentiation-associated protein 5 (MDAS), Mi-2 (chromodomain helicase DNA binding protein 4), a mitochondrial protein, muscarinic receptors, myelin-associated glycoprotein, myosin, myelin basic protein, myelin proteolipid protein, myelin oligodendrocyte glycoprotein, myeloperoxidase (MPO), rheumatoid factor (IgM anti-IgG), neuron-specific enolase, nicotinic acetylcholine receptor A chain, nucleolin, a nucleoporin, nucleosome antigen, PM/Sc1100, PM/Scl 75, pancreatic β-cell antigen, pepsinogen, peroxiredoxin 1, phosphoglucose isomerase, phospholipids, phosphatidyl inositol, platelet derived growth factors, polymerase beta (POLB), potassium channel KIR4.1, proliferating cell nuclear antigen (PCNA), proteinase-3, proteolipid protein, proteoglycan, prothrombin, recoverin, rhodopsin, ribonuclease, a ribonucleoprotein, ribosomes, a ribosomal phosphoprotein, RNA, an Sm protein, Sp100 nuclear protein, SRP54 (signal recognition particle 54 kDa), a selectin, smooth muscle proteins, sphingomyelin, streptococcal antigens, superoxide dismutase, synovial joint proteins, T1F1 gamma collagen, threonyl-tRNA synthetase (PL-7), tissue transglutaminase, thyroid peroxidase, thyroglobulin, thyroid stimulating hormone receptor, transferrin, triosephosphate isomerase, tubulin, tumor necrosis factor-alpha, topoisomerase, U1-dnRNP 68/70 kDa, U1-snRNP A, U1-snRNP C, U-snRNP B/B′, ubiquitin, vascular endothelial growth factor, vimentin, and vitronectin.

The antigens/epitopes included in a TMAPP of the present disclosure are those that are useful for treating an autoimmune disease other than (or in addition to) T1D and celiac disease. Accordingly, the present disclosure encompasses only protein constructs (e.g., TMAPPs) and methods of preparing protein constructs (e.g., TMAPPs) (as well as compositions comprising such protein constructs) comprising antigens/epitopes useful for treating an autoimmune disease other than (or in addition to) celiac disease or T1D. Likewise, the present disclosure encompasses only methods of treating, and the treatment of, an autoimmune disease other than (or in addition to) T1D or celiac disease. For clarity, an TMAPP comprising an antigen/epitope that is useful for treating an autoimmune disease other than T1D and/or celiac disease is not excluded from the scope of this disclosure if it also may provide some therapeutic benefit for the treatment of T1D and/or celiac disease. Likewise, a method of treating, or treatment of, an autoimmune disease other than T1D and/or celiac disease is not excluded from the scope of this disclosure if it also may have a use as a method of treating or treatment of T1D or celiac disease.

Autoantigens associated with alopecia areata (autoimmune alopecia) include, e.g., hair follicle keratinocyte polypeptides, melanogenesis-associated autoantigens, and melanocyte polypeptides. An example of a melanocyte autoantigen is tyrosinase. Autoantigens associated with autoimmune alopecia also include trichohyalin (Leung et al. (2010) J. Proteome Res. 9:5153) and keratin 16. A suitable epitope-presenting peptide for inclusion in a TMAPP of the present disclosure can be an epitope-presenting peptide of from 4 amino acids to about 25 amino acids in length of a hair follicle keratinocyte polypeptide, a melanocyte polypeptide, a melanogenesis-associated polypeptide, tyrosinase, trichohyalin, or keratin 16.

Autoantigens associated with Addison's disease include, e.g., 21-hydroxylase. A suitable epitope-presenting peptide for inclusion in a TMAPP of the present disclosure can be an epitope-presenting peptide of from 4 amino acids to about 25 amino acids in length of 21-hydroxylase.

Autoantigens associated with autoimmune thyroiditis (Hashimoto's thyroiditis) include, e.g., thyroglobulin, thyroid peroxidase, thyroid Stimulating Hormone Receptor (TSH-Receptor), thyroidal iodide transporters Na⁺/I-symporter (NIS), pendrin, and the like. A suitable epitope-presenting peptide for inclusion in a TMAPP of the present disclosure can be an epitope-presenting peptide of from 4 amino acids to about 25 amino acids in length of any one of the aforementioned Hashimoto's thyroiditis-associated polypeptides.

Autoantigens associated with Crohn's disease include, e.g., pancreatic secretory granule membrane glycoprotein-2 (GP2). A suitable epitope-presenting peptide for inclusion in a TMAPP of the present disclosure can be an epitope-presenting peptide of from 4 amino acids to about 25 amino acids in length of GP2.

Autoantigens associated with Goodpasture's disease include, e.g., the α3 chain of type IV collagen, e.g., amino acids 135-145 of the α3 chain of type IV collagen. Penades et al. (1995) Eur. J. Biochem. 229:754; Kalluri et al. (1994) Proc. Natl. Acad. Sci. USA 91:6201. A suitable epitope-presenting peptide for inclusion in a TMAPP of the present disclosure can be an epitope-presenting peptide of from 4 amino acids to about 25 amino acids in length of the α3 chain of type IV collagen.

Autoantigens associated with Grave's disease include, for example, thyroglobulin, thyroid peroxidase, and thyrotropin receptor (TSH-R). A suitable epitope-presenting peptide for inclusion in a TMAPP of the present disclosure can be an epitope-presenting peptide of from 4 amino acids to about 25 amino acids in length of any one of the aforementioned Grave's disease-associated antigens.

Autoantigens associated with mixed connective tissue disease include, e.g., U1 ribonucleoprotein (U1-RNP) polypeptide (also known as snRNP70). Sato et al. (2010) Mol. Cell. Biochem. 106:55. A suitable epitope-presenting peptide for inclusion in a TMAPP of the present disclosure can be an epitope-presenting peptide of from 4 amino acids to about 25 amino acids in length of U1-RNP polypeptide.

Autoantigens associated with multiple sclerosis include, e.g., myelin basic protein, myelin oligodendrocyte glycoprotein, and myelin proteolipid protein. A suitable epitope-presenting peptide for inclusion in a TMAPP of the present disclosure can be an epitope-presenting peptide of from 4 amino acids to about 25 amino acids in length of any one of the aforementioned multiple sclerosis-associated antigens. As one non-limiting example, the peptide epitope can comprise the amino acid sequence ENPVVHFFKNIVTPR (SEQ ID NO: 408). In some cases, a TMAPP of the present disclosure comprises a DRB1*15:01 MHC class II β chain; and a peptide epitope of the amino acid sequence ENPVVHFFKNIVTPR (SEQ ID NO: 408).

Autoantigens associated with myasthenia gravis include, e.g., acetylcholine receptor (AchR; see, e.g., Lindstrom (2000) Muscle & Nerve 23:453), muscle-specific tyrosine kinase, and low-density lipoprotein receptor-related protein-4. A suitable epitope-presenting peptide for inclusion in a TMAPP of the present disclosure can be an epitope-presenting peptide of from 4 amino acids to about 25 amino acids in length of any one of the aforementioned myasthenia gravis-associated antigens. In some cases, a suitable epitope-presenting peptide for inclusion in a TMAPP of the present disclosure is an epitope-presenting peptide of from 4 amino acids to about 25 amino acids in length of an AchR.

Autoantigens associated with Parkinson's disease include, e.g., α-synuclein. A suitable epitope-presenting peptide for inclusion in a TMAPP of the present disclosure can be an epitope-presenting peptide of from 4 amino acids to about 25 amino acids in length of α-synuclein. For example, a suitable epitope-presenting peptide for inclusion in a TMAPP of the present disclosure includes a peptide of from 5 amino acids to the entire length of any one of the following: GKTKEGVLYVGSKTK (SEQ ID NO: 409); KTKEGVLYVGSKTKE (SEQ ID NO: 410); MPVDPDNEAYEMPSE (SEQ ID NO: 411); DNEAYEMPSEEGYQD (SEQ ID NO: 412); EMPSEEGYQDYEPE (SEQ ID NO: 413); and SEEGYQDYEPEA (SEQ ID NO: 414), where “S” denotes phosphoserine.

Autoantigens associated with pemphigus (e.g., pemphigus vulgaris, pemphigus foliaceus, bullous pemphigoid) include pemphigus vulgaris immunogens such as desmosomal cadherin desmoglein 3 (Dsg3); pemphigus foliaceus immunogens such as Dsg1; bullous pemphigoid immunogens such as hemidesmosome peptides including BP230 antigen, GPAG1a, and BPAG1b. See, e.g., Cirillo et al. (2007) Immunology 121:377. Autoantigens associated with bullous pemphigoid include bullous pemphigoid antigen 1 (BPAG1; also known as BP230 or dystonin), bullous pemphigoid antigen 2 (BPAG2; also known as BP180 or type XVII collagen), and subunits of human integrins α-5 and β-4. A suitable epitope-presenting peptide for inclusion in a TMAPP of the present disclosure can be an epitope-presenting peptide of from 4 amino acids to about 25 amino acids in length of any of the aforementioned pemphigus-associated antigens.

Autoantigens associated with myositis (e.g., polymyositis; dermatomyositis) include, e.g., histidyl tRNA synthetase. A suitable epitope-presenting peptide for inclusion in a TMAPP of the present disclosure can be an epitope-presenting peptide of from 4 amino acids to about 25 amino acids in length of histidyl tRNA synthetase.

Autoantigens associated with rheumatoid arthritis include, e.g., collagen, vimentin, aggregan, fibrinogen, cyclic citrullinated peptides, α-enolase, histone polypeptides, lactoferrin, catalase, actinin, and actins (cytoplasmic 1 and 2(β/γ). A suitable epitope-presenting peptide for inclusion in a TMAPP of the present disclosure can be an epitope-presenting peptide of from 4 amino acids to about 25 amino acids in length of any one of the aforementioned rheumatoid arthritis-associated antigens.

Autoantigens associated with scleroderma include nuclear antigens. A suitable epitope-presenting peptide for inclusion in a TMAPP of the present disclosure can be an epitope-presenting peptide of from 4 amino acids to about 25 amino acids in length of a nuclear antigen associated with scleroderma.

Autoantigens associated with Sjögren's syndrome include, e.g., Ro/La ribonucleoprotein (RNP) complex, alpha-fodrin, beta-fodrin, islet cell autoantigen, poly(ADP)ribose polymerase (PARP), nuclear mitotic apparatus (NuMA), NOR-90, Ro60 kD autoantigen, Ro52 antigen, La antigen (see, e.g., GenBank Accession No. NP_001281074.1), and p27 antigen. A suitable epitope-presenting peptide for inclusion in a TMAPP of the present disclosure can be an epitope-presenting peptide of from 4 amino acids to about 25 amino acids in length of any one of the aforementioned Sjögren's syndrome-associated antigens.

Autoantigens associated with systemic lupus erythematosus (SLE) include, e.g., Ro60 autoantigen, low-density lipoproteins, Sm antigens of the U-1 small nuclear ribonucleoprotein complex (B/B′, D1, D2, D3, E, F, G), α-actin 1, α-actin 4, annexin AI, C1q/tumor necrosis factor-related protein, catalase, defensins, chromatin, histone proteins, transketolase, hCAP18/LL37, and ribonucleoproteins (RNPs). A suitable epitope-presenting peptide for inclusion in a TMAPP of the present disclosure can be an epitope-presenting peptide of from 4 amino acids to about 25 amino acids in length of any one of the aforementioned SLE-associated antigens.

Autoantigens associated with thrombocytopenia purpura include ADAMTS13 (a disintegrin and metalloproteinase with a thrombospondin type 1 motif, member 13), and von Willebrand factor-cleaving protease (VWFCP). A suitable epitope-presenting peptide for inclusion in a TMAPP of the present disclosure can be an epitope-presenting peptide of from 4 amino acids to about 25 amino acids in length of an ADAMTS13 polypeptide or a VWFCP polypeptide.

Autoantigens associated with vasculitis include proteinase-3, lysozyme C, lactoferrin, leukocyte elastase, cathepsin G, and azurocidin. A suitable epitope-presenting peptide for inclusion in a TMAPP of the present disclosure can be an epitope-presenting peptide of from 4 amino acids to about 25 amino acids in length of any of the aforementioned vasculitis-associated antigens.

Autoantigens associated with vitiligo include SOX9, SOX10, PMEL (Premelanosomal protein), tyrosinase, TYRP1 (Tyrosine related protein 1), DDT (D-Dopachrome tautomerase), Rab38, and MCHR1 (Melanin-concentrating receptor. A suitable epitope-presenting peptide for inclusion in a TMAPP of the present disclosure can be an epitope-presenting peptide of from 4 amino acids to about 25 amino acids in length of any one of the aforementioned vitiligo-associated polypeptides.

Autoantigens associated with autoimmune uveitis include, for example, interphotoreceptor retinoid-binding protein (IRBP). A suitable epitope-presenting peptide for inclusion in a TMAPP of the present disclosure can be an epitope-presenting peptide of from 4 amino acids to about 25 amino acids in length IRBP. A suitable epitope-presenting peptide for inclusion in a TMAPP of the present disclosure can be an epitope-presenting peptide of from 4 amino acids to about 25 amino acids in length of any one of the aforementioned antigens.

Autoantigens associated with autoimmune polyendocrine syndrome include, e.g., 17-alpha hydroxylase, histidine decarboxylase, tryptophan hydroxylase, and tyrosine hydroxylase. A suitable epitope-presenting peptide for inclusion in a TMAPP of the present disclosure can be an epitope-presenting peptide of from 4 amino acids to about 25 amino acids in length of any one of the aforementioned autoimmune polyendocrine syndrome-associated antigens.

Autoantigens associated with psoriasis include ADAMTS15. See, e.g., Prinz (2017) Autoimmunity Reviews 16:970. A suitable epitope-presenting peptide for inclusion in a TMAPP of the present disclosure can be an epitope-presenting peptide of from 4 amino acids to about 25 amino acids in length of an ADAMTS15 polypeptide.

### Immunomodulatory Polypeptides (“MODs”)

Immunomodulatory polypeptides that are suitable for inclusion in a TMAPP of the present disclosure include, but are not limited to, IL-2, CD7, B7-1 (CD80), B7-2 (CD86), PD-L1, PD-L2, 4-1BBL, OX40L, Fas ligand (FasL), inducible costimulatory ligand (ICOS-L), intercellular adhesion molecule (ICAM), CD30L, CD40, CD70, CD83, HLA-G, MICA, MICB, HVEM, lymphotoxin beta receptor, 3/TR6, ILT3, and ILT4.

Examples of immunomodulatory polypeptides (MOD) suitable for inclusion in a TMAPP of the present disclosure, and their cognate costimulatory receptors (also referred to herein as “co-immunomodulatory polypeptides” or “coMODs”) include the following:

a) 4-1BBL (MOD) and 4-1BB (Co-MOD);

b) PD-L1 (MOD) and PD1 (Co-MOD);

c) IL-2 (MOD) and IL-2 receptor (Co-MOD);

d) CD80 (MOD) and CD28 (Co-MOD);

e) CD86 (MOD) and CD28 (Co-MOD);

f) OX40L (CD252) (MOD) and OX40 (CD134) (Co-MOD);

g) Fas ligand (FasL) (MOD) and Fas (Co-MOD);

h) ICOS-L (MOD) and ICOS (Co-MOD);

i) ICAM (MOD) and LFA-1 (Co-MOD);

j) CD30L (MOD) and CD30 (Co-MOD);

k) CD40 (MOD) and CD40L (Co-MOD);

l) CD83 (MOD) and CD83L (Co-MOD);

m) HVEM (CD270) (MOD) and CD160 (Co-MOD);

n) JAG1 (CD339) (MOD) and Notch (Co-MOD);

o) JAG1 (CD339) (MOD) and CD46 (Co-MOD);

p) CD70 (MOD) and CD27 (Co-MOD);

q) CD80 (MOD) and CTLA4 (Co-MOD);

r) CD86 (MOD) and CTLA4 (Co-MOD); and

s) TGF-β1, TGF-β2, and/or TGF-β3 (MODs) and TGF-β Receptor (e.g., TGFBR1 and/or TGFBR2) (Co-MOD).

In some cases, the immunomodulatory polypeptide is selected from a 4-1BBL polypeptide, a B7-1 polypeptide; a B7-2 polypeptide, an ICOS-L polypeptide, an OX-40L polypeptide, a CD80 polypeptide, a CD86 polypeptide, a PD-L1 polypeptide, a FasL polypeptide, a TGFβ polypeptide, and a PD-L2 polypeptide. The immunomodulatory polypeptide can comprise only the extracellular portion of a full-length immunomodulatory polypeptide. Thus, for example, the immunomodulatory polypeptide can in some cases exclude one or more of a signal peptide, a transmembrane domain, and an intracellular domain normally found in a naturally-occurring immunomodulatory polypeptide.

In some cases, an immunomodulatory polypeptide suitable for inclusion in a TMAPP of the present disclosure comprises all or a portion of (e.g., an extracellular portion of) the amino acid sequence of a naturally-occurring immunomodulatory polypeptide. In other instances, an immunomodulatory polypeptide suitable for inclusion in a TMAPP of the present disclosure is a variant immunomodulatory polypeptide that comprises at least one amino acid substitution compared to the amino acid sequence of a naturally-occurring immunomodulatory polypeptide. In some instances, a variant immunomodulatory polypeptide exhibits a binding affinity for a co-immunomodulatory polypeptide that is lower than the affinity of a corresponding naturally-occurring immunomodulatory polypeptide (e.g., an immunomodulatory polypeptide not comprising the amino acid substitution(s) present in the variant) for the co-immunomodulatory polypeptide.

Variant Immunomodulatory Polypeptides with Reduced Affinity

Suitable immunomodulatory domains that exhibit reduced affinity for a co-immunomodulatory domain can have from 1 amino acid (aa) to 20 aa differences from a wild-type immunomodulatory domain For example, in some cases, a variant immunomodulatory polypeptide present in a TMAPP of the present disclosure differs in amino acid sequence by 1 aa, 2 aa, 3 aa, 4 aa, 5 aa, 6 aa, 7 aa, 8 aa, 9 aa, or 10 aa, from a corresponding wild-type immunomodulatory polypeptide. As another example, in some cases, a variant immunomodulatory polypeptide present in a TMAPP of the present disclosure differs in amino acid sequence by 11 aa, 12 aa, 13 aa, 14 aa, 15 aa, 16 aa, 17 aa, 18 aa, 19 aa, or 20 aa, from a corresponding wild-type immunomodulatory polypeptide. As an example, in some cases, a variant immunomodulatory polypeptide present in a TMAPP of the present disclosure includes 1, 2, 3, 4, 5, 6, 7, 8, 9, or 10 amino acid substitutions, compared to a corresponding reference (e.g., wild-type) immunomodulatory polypeptide. In some cases, a variant immunomodulatory polypeptide present in a TMAPP of the present disclosure includes a single amino acid substitution compared to a corresponding reference (e.g., wild-type) immunomodulatory polypeptide. In some cases, a variant immunomodulatory polypeptide present in a TMAPP of the present disclosure includes 2 amino acid substitutions (e.g., no more than 2 amino acid substitutions) compared to a corresponding reference (e.g., wild-type) immunomodulatory polypeptide. In some cases, a variant immunomodulatory polypeptide present in a TMAPP of the present disclosure includes 3 amino acid substitutions (e.g., no more than 3 amino acid substitutions) compared to a corresponding reference (e.g., wild-type) immunomodulatory polypeptide. In some cases, a variant immunomodulatory polypeptide present in a TMAPP of the present disclosure includes 4 amino acid substitutions (e.g., no more than 4 amino acid substitutions) compared to a corresponding reference (e.g., wild-type) immunomodulatory polypeptide. In some cases, a variant immunomodulatory polypeptide present in a TMAPP of the present disclosure includes 5 amino acid substitutions (e.g., no more than 5 amino acid substitutions) compared to a corresponding reference (e.g., wild-type) immunomodulatory polypeptide. In some cases, a variant immunomodulatory polypeptide present in a TMAPP of the present disclosure includes 6 amino acid substitutions (e.g., no more than 6 amino acid substitutions) compared to a corresponding reference (e.g., wild-type) immunomodulatory polypeptide. In some cases, a variant immunomodulatory polypeptide present in a TMAPP of the present disclosure includes 7 amino acid substitutions (e.g., no more than 7 amino acid substitutions) compared to a corresponding reference (e.g., wild-type) immunomodulatory polypeptide. In some cases, a variant immunomodulatory polypeptide present in a TMAPP of the present disclosure includes 8 amino acid substitutions (e.g., no more than 8 amino acid substitutions) compared to a corresponding reference (e.g., wild-type) immunomodulatory polypeptide. In some cases, a variant immunomodulatory polypeptide present in a TMAPP of the present disclosure includes 9 amino acid substitutions (e.g., no more than 9 amino acid substitutions) compared to a corresponding reference (e.g., wild-type) immunomodulatory polypeptide. In some cases, a variant immunomodulatory polypeptide present in a TMAPP of the present disclosure includes 10 amino acid substitutions (e.g., no more than 10 amino acid substitutions) compared to a corresponding reference (e.g., wild-type) immunomodulatory polypeptide.

In some cases, a variant immunomodulatory polypeptide present in a TMAPP of the present disclosure includes 11 amino acid substitutions (e.g., no more than 11 amino acid substitutions) compared to a corresponding reference (e.g., wild-type) immunomodulatory polypeptide. In some cases, a variant immunomodulatory polypeptide present in a TMAPP of the present disclosure includes 12 amino acid substitutions (e.g., no more than 12 amino acid substitutions) compared to a corresponding reference (e.g., wild-type) immunomodulatory polypeptide. In some cases, a variant immunomodulatory polypeptide present in a TMAPP of the present disclosure includes 13 amino acid substitutions (e.g., no more than 13 amino acid substitutions) compared to a corresponding reference (e.g., wild-type) immunomodulatory polypeptide. In some cases, a variant immunomodulatory polypeptide present in a TMAPP of the present disclosure includes 14 amino acid substitutions (e.g., no more than 14 amino acid substitutions) compared to a corresponding reference (e.g., wild-type) immunomodulatory polypeptide. In some cases, a variant immunomodulatory polypeptide present in a TMAPP of the present disclosure includes 15 amino acid substitutions (e.g., no more than 15 amino acid substitutions) compared to a corresponding reference (e.g., wild-type) immunomodulatory polypeptide. In some cases, a variant immunomodulatory polypeptide present in a TMAPP of the present disclosure includes 16 amino acid substitutions (e.g., no more than 16 amino acid substitutions) compared to a corresponding reference (e.g., wild-type) immunomodulatory polypeptide. In some cases, a variant immunomodulatory polypeptide present in a TMAPP of the present disclosure includes 17 amino acid substitutions (e.g., no more than 17 amino acid substitutions) compared to a corresponding reference (e.g., wild-type) immunomodulatory polypeptide. In some cases, a variant immunomodulatory polypeptide present in a TMAPP of the present disclosure includes 18 amino acid substitutions (e.g., no more than 18 amino acid substitutions) compared to a corresponding reference (e.g., wild-type) immunomodulatory polypeptide. In some cases, a variant immunomodulatory polypeptide present in a TMAPP of the present disclosure includes 19 amino acid substitutions (e.g., no more than 19 amino acid substitutions) compared to a corresponding reference (e.g., wild-type) immunomodulatory polypeptide. In some cases, a variant immunomodulatory polypeptide present in a TMAPP of the present disclosure includes 20 amino acid substitutions (e.g., no more than 20 amino acid substitutions) compared to a corresponding reference (e.g., wild-type) immunomodulatory polypeptide.

As discussed above, a variant immunomodulatory polypeptide suitable for inclusion in a TMAPP of the present disclosure exhibits reduced affinity for a cognate co-immunomodulatory polypeptide, compared to the affinity of a corresponding wild-type immunomodulatory polypeptide for the cognate co-immunomodulatory polypeptide.

Exemplary pairs of immunomodulatory polypeptide and cognate co-immunomodulatory polypeptide include, but are not limited to:

a) 4-1BBL (immunomodulatory polypeptide) and 4-1BB (cognate co-immunomodulatory polypeptide);

b) PD-L1 (immunomodulatory polypeptide) and PD1 (cognate co-immunomodulatory polypeptide);

c) IL-2 (immunomodulatory polypeptide) and IL-2 receptor (cognate co-immunomodulatory polypeptide);

d) CD80 (immunomodulatory polypeptide) and CD28 (cognate co-immunomodulatory polypeptide);

e) CD86 (immunomodulatory polypeptide) and CD28 (cognate co-immunomodulatory polypeptide);

f) OX40L (CD252) (immunomodulatory polypeptide) and OX40 (CD134) (cognate co-immunomodulatory polypeptide);

g) Fas ligand (immunomodulatory polypeptide) and Fas (cognate co-immunomodulatory polypeptide);

h) ICOS-L (immunomodulatory polypeptide) and ICOS (cognate co-immunomodulatory polypeptide);

i) ICAM (immunomodulatory polypeptide) and LFA-1 (cognate co-immunomodulatory polypeptide);

j) CD30L (immunomodulatory polypeptide) and CD30 (cognate co-immunomodulatory polypeptide);

k) CD40 (immunomodulatory polypeptide) and CD40L (cognate co-immunomodulatory polypeptide);

l) CD83 (immunomodulatory polypeptide) and CD83L (cognate co-immunomodulatory polypeptide);

m) HVEM (CD270) (immunomodulatory polypeptide) and CD160 (cognate co-immunomodulatory polypeptide);

n) JAG1 (CD339) (immunomodulatory polypeptide) and Notch (cognate co-immunomodulatory polypeptide);

o) JAG1 (immunomodulatory polypeptide) and CD46 (cognate co-immunomodulatory polypeptide);

p) CD80 (immunomodulatory polypeptide) and CTLA4 (cognate co-immunomodulatory polypeptide);

q) CD86 (immunomodulatory polypeptide) and CTLA4 (cognate co-immunomodulatory polypeptide); and

r) CD70 (immunomodulatory polypeptide) and CD27 (cognate co-immunomodulatory polypeptide).

In some cases, a variant immunomodulatory polypeptide present in a TMAPP of the present disclosure has a binding affinity for a cognate co-immunomodulatory polypeptide that is from 100 nM to 100 μM. For example, in some cases, a variant immunomodulatory polypeptide present in a TMAPP of the present disclosure has a binding affinity for a cognate co-immunomodulatory polypeptide that is from about 100 nM to 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

### Determining Binding Affinity

Binding affinity between an immunomodulatory polypeptide and its cognate co-immunomodulatory polypeptide can be determined by bio-layer interferometry (BLI) using purified immunomodulatory polypeptide and purified cognate co-immunomodulatory polypeptide. Binding affinity between a TMAPP and its cognate co-immunomodulatory polypeptide can also be determined by BLI using purified TMAPP and the cognate co-immunomodulatory polypeptide. BLI methods are well known to those skilled in the art. See, e.g., Lad et al. (2015) J. Biomol. Screen. 20(4):498-507; and Shah and Duncan (2014) J. Vis. Exp. 18:e51383. The specific and relative binding affinities described in this disclosure between an immunomodulatory polypeptide and its cognate co-immunomodulatory polypeptide, or between a synTac and its cognate co-immunomodulatory polypeptide, can be determined using the following procedures.

To determine binding affinity between a TMAPP and its cognate co-immunomodulatory polypeptide, a BLI assay can be carried out using an Octet RED 96 (Pal FortéBio) instrument, or a similar instrument, as follows. A TMAPP (e.g., a TMAPP of the present disclosure; a control TMAPP (where a control TMAPP comprises a wild-type immunomodulatory polypeptide)) is immobilized onto an insoluble support (a “biosensor”). The immobilized TMAPP is the “target.” Immobilization can be effected by immobilizing a capture antibody onto the insoluble support, where the capture antibody immobilizes the TMAPP. For example, immobilization can be effected by immobilizing anti-Fc (e.g., anti-human IgG Fc) antibodies onto the insoluble support, where the immobilized anti-Fc antibodies bind to and immobilize the TMAPP (where the TMAPP comprises an IgFc polypeptide). A co-immunomodulatory polypeptide is applied, at several different concentrations, to the immobilized TMAPP, and the instrument's response recorded. Assays are conducted in a liquid medium comprising 25 mM HEPES pH 6.8, 5% poly(ethylene glycol) 6000, 50 mM KCl, 0.1% bovine serum albumin, and 0.02% Tween 20 nonionic detergent. Binding of the co-immunomodulatory polypeptide to the immobilized TMAPP is conducted at 30° C. As a positive control for binding affinity, an anti-MHC Class II monoclonal antibody can be used. For example, an anti-HLD-DR3 monoclonal antibody such as the 16-23 antibody (Sigma; also referred to as “16.23”; see, e.g., Pious et al. (1985) J. Exp. Med. 162:1193; Mellins et al. (1991) J. Exp. Med. 174:1607; ECACC hybridoma collection 16-23, ECACC 99043001) can be used as a positive control for binding affinity. As another example, a pan-HLA Class II antibody, such as the HKB1 antibody (Immunotools; Holte et al. (1989) Eur. J. Immunol. 19:1221) can be used as a positive control for binding affinity. A standard curve can be generated using serial dilutions of the anti-MHC Class II monoclonal antibody. The co-immunomodulatory polypeptide, or the anti-MHC Class II mAb, is the “analyte.” BLI analyzes the interference pattern of white light reflected from two surfaces: i) from the immobilized polypeptide (“target”); and ii) an internal reference layer. A change in the number of molecules (“analyte”; e.g., co-immunomodulatory polypeptide; anti-HLA antibody) bound to the biosensor tip causes a shift in the interference pattern; this shift in interference pattern can be measured in real time. The two kinetic terms that describe the affinity of the target/analyte interaction are the association constant (kₐ) and dissociation constant (k_{d}). The ratio of these two terms (k_{d/a}) gives rise to the affinity constant K_{D}.

As noted above, determining binding affinity between an immunomodulatory polypeptide (e.g., IL-2 or an IL-2 variant) and its cognate co-immunomodulatory polypeptide (e.g., IL-2R) also can be determined by BLI. The assay is similar to that described above for the TMAPP. A BLI assay can be carried out using an Octet RED 96 (Pal FortéBio) instrument, or a similar instrument, as follows. A component immunomodulatory polypeptide of a TMAPP of the present disclosure (e.g., a variant IL-2 polypeptide of the present disclosure); and a control immunomodulatory polypeptide (where a control immunomodulatory polypeptide comprises a wild-type immunomodulatory polypeptide, e.g. wild-type IL-2)) are immobilized onto an insoluble support (a “biosensor”). The immunomodulatory polypeptide is the “target.” Immobilization can be effected by immobilizing a capture antibody onto the insoluble support, where the capture antibody immobilizes the immunomodulatory polypeptide. For example, if the target is fused to an immuno-affinity tag (e.g. FLAG, human IgG Fc) immobilization can be effected by immobilizing with the appropriate antibody to the immuno-affinity tag (e.g. anti-human IgG Fc) onto the insoluble support, where the immobilized antibodies bind to and immobilize the immunomodulatory polypeptide (where the immunomodulatory polypeptide comprises an IgFc polypeptide). A co-immunomodulatory polypeptide (or polypeptides) is applied, at several different concentrations, to the immobilized immunomodulatory polypeptide, and the instrument's response recorded. Alternatively, a co-immunomodulatory polypeptide (or polypeptides) is immobilized to the biosensor (e.g., for the IL-2 receptor heterotrimer, as a monomeric subunit, heterodimeric subcomplex, or the complete heterotrimer) and the immunomodulatory polypeptide is applied, at several different concentrations, to the immobilized coimmunomodulatory polypeptide(s), and the instrument's response is recorded. Assays are conducted in a liquid medium comprising 25 mM HEPES pH 6.8, 5% poly(ethylene glycol) 6000, 50 mM KCl, 0.1% bovine serum albumin, and 0.02% Tween 20 nonionic detergent. Binding of the co-immunomodulatory polypeptide to the immobilized immunomodulatory polypeptide is conducted at 30° C. BLI analyzes the interference pattern of white light reflected from two surfaces: i) from the immobilized polypeptide (“target”); and ii) an internal reference layer. A change in the number of molecules (“analyte”; e.g., co-immunomodulatory polypeptide) bound to the biosensor tip causes a shift in the interference pattern; this shift in interference pattern can be measured in real time. The two kinetic terms that describe the affinity of the target/analyte interaction are the association constant (kₐ) and dissociation constant (k_{d}). The ratio of these two terms (k_{d/a}) gives rise to the affinity constant K_{D}. Determining the binding affinity of both a wild-type immunomodulatory polypeptide (e.g., IL-2) for its receptor (e.g., IL-2R) and a variant immunomodulatory polypeptide (e.g., an IL-2 variant as disclosed herein) for its cognate co-immunomodulatory polypeptide (e.g., its receptor) (e.g., IL-2R) thus allows one to determine the relative binding affinity of the variant co-immunomodulatory polypeptide, as compared to the wild-type co-immunomodulatory polypeptide, for the cognate co-immunomodulatory polypeptide. That is, one can determine whether the binding affinity of a variant immunomodulatory polypeptide for its receptor (its cognate co-immunomodulatory polypeptide) is reduced as compared to the binding affinity of the wild-type immunomodulatory polypeptide for the same cognate co-immunomodulatory polypeptide, and, if so, what is the percentage reduction from the binding affinity of the wild-type co-immunomodulatory polypeptide.

The BLI assay is carried out in a multi-well plate. To run the assay, the plate layout is defined, the assay steps are defined, and biosensors are assigned in Octet Data Acquisition software. The biosensor assembly is hydrated. The hydrated biosensor assembly and the assay plate are equilibrated for 10 minutes on the Octet instrument. Once the data are acquired, the acquired data are loaded into the Octet Data Analysis software. The data are processed in the Processing window by specifying method for reference subtraction, y-axis alignment, inter-step correction, and Savitzky-Golay filtering. Data are analyzed in the Analysis window by specifying steps to analyze (Association and Dissociation), selecting curve fit model (1:1), fitting method (global), and window of interest (in seconds). The quality of fit is evaluated. K_{D} values for each data trace (analyte concentration) can be averaged if within a 3-fold range. K_{D} error values should be within one order of magnitude of the affinity constant values; R² values should be above 0.95. See, e.g., Abdiche et al. (2008) J. Anal. Biochem. 377:209.

In some cases, the ratio of: i) the binding affinity of a control TMAPP (where the control TMAPP comprises a wild-type immunomodulatory polypeptide) to a cognate co-immunomodulatory polypeptide to ii) the binding affinity of a TMAPP of the present disclosure comprising a variant of the wild-type immunomodulatory polypeptide to the cognate co-immunomodulatory polypeptide, when measured by BLI (as described above), is at least 1.5:1, at least 2:1, at least 5:1, at least 10:1, at least 15:1, at least 20:1, at least 25:1, at least 50:1, at least 100:1, at least 500:1, at least 10²:1, at least 5×10²:1, at least 10³:1, at least 5×10³:1, at least 10⁴:1, at least 10⁵:1, or at least 10⁶:1. In some cases, the ratio of: i) the binding affinity of a control TMAPP (where the control TMAPP comprises a wild-type immunomodulatory polypeptide) to a cognate co-immunomodulatory polypeptide to ii) the binding affinity of a TMAPP of the present disclosure comprising a variant of the wild-type immunomodulatory polypeptide to the cognate co-immunomodulatory polypeptide, when measured by BLI, is in a range of from 1.5:1 to 10⁶:1, e.g., from 1.5:1 to 10:1, from 10:1 to 50:1, from 50:1 to 10²:1, from 10²:1 to 10³:1, from 10³:1 to 10⁴:1, from 10⁴:1 to 10⁵:1, or from 10⁵:1 to 10⁶:1.

The epitope present in a TMAPP of the present disclosure binds to a T-cell receptor (TCR) on a T cell with an affinity of at least 100 μM (e.g., at least 10 μM, at least 1 μM, at least 100 nM, at least 10 nM, or at least 1 nM). In some cases, the epitope present in a TMAPP of the present disclosure binds to a TCR on a T cell with an affinity of from about 10⁻⁴M to about 5×10⁻⁴ M, from about 5×10⁻⁴M to about 10⁻⁵ M, from about 10⁻⁵ M to 5×10⁻⁵ M, from about 5×10⁻⁵ M to 10⁻⁶ M, from about 10⁻⁶ M to about 5×10⁻⁶ M, from about 5×10⁻⁶ M to about 10⁻⁷ M, from about 10⁻⁷ M to about 5×10⁻⁷M, from about 5×10⁻⁷ M to about 10⁻⁸M, or from about 10⁻⁸ M to about 10⁻⁹ M. Expressed another way, in some cases, the epitope present in a TMAPP of the present disclosure binds to a TCR on a T cell with an affinity of from about 1 nM to about 5 nM, from about 5 nM to about 10 nM, from about 10 nM to about 50 nM, from about 50 nM to about 100 nM, from about 0.1 μM to about 0.5 μM, from about 0.5 μM to about 1 μM, from about 1 μM to about 5 μM, from about 5 μM to about 10 μM, from about 10 μM to about 25 μM, from about 25 μM to about 50 μM, from about 50 μM to about 75 μM, from about 75 μM to about 100 μM.

In some cases, a variant immunomodulatory polypeptide present in a TMAPP of the present disclosure has a binding affinity for a cognate co-immunomodulatory polypeptide that is from 1 nM to 100 nM, or from 100 nM to 100 μM. For example, in some cases, a variant immunomodulatory polypeptide present in a TMAPP of the present disclosure has a binding affinity for a cognate co-immunomodulatory polypeptide that is from about 100 nM to 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM. In some cases, a variant immunomodulatory polypeptide present in a TMAPP of the present disclosure has a binding affinity for a cognate co-immunomodulatory polypeptide that is from about 1 nM to about 5 nM, from about 5 nM to about 10 nM, from about 10 nM to about 50 nM, from about 50 nM to about 100 nM.

### PD-L1 Variants

As one non-limiting example, in some cases, a variant immunomodulatory polypeptide present in a TMAPP of the present disclosure is a variant PD-L1 polypeptide. Wild-type PD-L1 binds to PD1.

A wild-type human PD-L1 polypeptide can comprise the following amino acid sequence:

(SEQ ID NO: 103)
MRIFAVFIFM TYWHLLNAFT VTVPKDLYVV EYGSNMTIEC
KFPVEKQLDL AALIVYWEME DKNIIQFVHG EEDLKVQHSS
YRQRARLLKD QLSLGNAALQ ITDVKLQDAG VYRCMISYGG
ADYKRITVKV NAPYNKINQR ILVVDPVTSE HELTCQAEGY
PKAEVIWTSS DHQVLSGKTT TTNSKREEKL FNVTSTLRIN
TTTNEIEYCT ERRLDPEENH TAELVIPGNI LNVSIKICLT
LSPST.

A wild-type human PD-L1 ectodomain can comprise the following amino acid sequence:

(SEQ ID NO: 104)
FT VTVPKDLYVV EYGSNMTIEC KFPVEKQLDL AALIVYWEME
DKNIIQFVHG EEDLKVQHSS YRQRARLLKD QLSLGNAALQ
ITDVKLQDAG VYRCMISYGG ADYKRITVKV NAPYNKINQR
ILVVDPVTSE HELTCQAEGY PKAEVIWTSS DHQVLSGKTT
TTNSKREEKL FNVTSTLRIN TTTNEIFYCT FRRLDPEENH
TAELVIPGNI LNVSIKI.

A wild-type PD-1 polypeptide can comprise the following amino acid sequence:

(SEQ ID NO: 105)
PGWFLDSPDR PWNPPTFSPA LLVVTEGDNA TFTCSFSNTS
ESFVLNWYRM SPSNQTDKLA AFPEDRSQPG QDCRFRVTQL
PNGRDFHMSV VRARRNDSGT YLCGAISLAP KAQIKESLRA
ELRVTERRAE VPTAHPSPSP RPAGQFQTLV VGVVGGLLGS
LVLLVWVLAV ICSRAARGTI GARRTGQPLK EDPSAVPVFS
VDYGELDFQW REKTPEPPVP CVPEQTEYAT IVFPSGMGTS
SPARRGSADG PRSAQPLRPE DGHCSWPL.

In some cases, a variant PD-L1 polypeptide exhibits reduced binding affinity to PD-1 (e.g., a PD-1 polypeptide comprising the amino acid sequence set forth in SEQ ID NO: 105), compared to the binding affinity of a PD-L1 polypeptide comprising the amino acid sequence set forth in SEQ ID NO: 103 or SEQ ID NO: 104. For example, in some cases, a variant PD-L1 polypeptide of the present disclosure binds PD-1 (e.g., a PD-1 polypeptide comprising the amino acid sequence set forth in SEQ ID NO: 105) with a binding affinity that is at least 10% less, at least 15% less, at least 20% less, at least 25% less, at least 30% less, at least 35% less, at least 40% less, at least 45% less, at least 50% less, at least 55% less, at least 60% less, at least 65% less, at least 70% less, at least 75% less, at least 80% less, at least 85% less, at least 90% less, at least 95% less, or more than 95% less, than the binding affinity of a PD-L1 polypeptide comprising the amino acid sequence set forth in SEQ ID NO: 103 or SEQ ID NO: 104.

In some cases, a variant PD-L1 polypeptide has a binding affinity to PD-1 that is from 1 nM to 1 mM. In some cases, a variant PD-L1 polypeptide of the present disclosure has a binding affinity to PD-1 that is from 100 nM to 100 μM. As another example, in some cases, a variant PD-L1 polypeptide has a binding affinity for PD1 (e.g., a PD1 polypeptide comprising the amino acid sequence set forth in SEQ ID NO: 105) that is from about 100 nM to 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

In some cases, a variant PD-L1 polypeptide has a single amino acid substitution compared to the PD-L1 amino acid sequence set forth in SEQ ID NO: 103 or SEQ ID NO: 104. In some cases, a variant PD-L1 polypeptide has from 2 to 10 amino acid substitutions compared to the PD-L1 amino acid sequence set forth in SEQ ID NO: 103 or SEQ ID NO: 104. In some cases, a variant PD-L1 polypeptide has 2 amino acid substitutions compared to the PD-L1 amino acid sequence set forth in SEQ ID NO: 103 or SEQ ID NO: 104. In some cases, a variant PD-L1 polypeptide has 3 amino acid substitutions compared to the PD-L1 amino acid sequence set forth in SEQ ID NO: 103 or SEQ ID NO: 104. In some cases, a variant PD-L1 polypeptide has 4 amino acid substitutions compared to the PD-L1 amino acid sequence set forth in SEQ ID NO: 103 or SEQ ID NO: 104. In some cases, a variant PD-L1 polypeptide has 5 amino acid substitutions compared to the PD-L1 amino acid sequence set forth in SEQ ID NO: 103 or SEQ ID NO: 104. In some cases, a variant PD-L1 polypeptide has 6 amino acid substitutions compared to the PD-L1 amino acid sequence set forth in SEQ ID NO: 103 or SEQ ID NO: 104. In some cases, a variant PD-L1 polypeptide has 7 amino acid substitutions compared to the PD-L1 amino acid sequence set forth in SEQ ID NO: 103 or SEQ ID NO: 104. In some cases, a variant PD-L1 polypeptide has 8 amino acid substitutions compared to the PD-L1 amino acid sequence set forth in SEQ ID NO: 103 or SEQ ID NO: 104. In some cases, a variant PD-L1 polypeptide has 9 amino acid substitutions compared to the PD-L1 amino acid sequence set forth in SEQ ID NO: 103 or SEQ ID NO: 104. In some cases, a variant PD-L1 polypeptide has 10 amino acid substitutions compared to the PD-L1 amino acid sequence set forth in SEQ ID NO: 103 or SEQ ID NO: 104.

A suitable PD-L1 variant includes a polypeptide that comprises an amino acid sequence having at least 90%, at least 95%, at least 98%, at least 99%, or 100%, amino acid sequence identity to the following amino acid sequence:

FT VTVPKXLYVV EYGSNMTIEC KFPVEKQLDL AALIVYWEME DKNIIQFVHG EEDLKVQHSS YRQRARLLKD QLSLGNAALQ ITDVKLQDAG VYRCMISYGG ADYKRITVKV NAPYNKINQR ILVVDPVTSE HELTCQAEGY PKAEVIWTSS DHQVLSGKTT TTNSKREEKL FNVTSTLRIN TTTNEIFYCT FRRLDPEENH TAELVIPGNI LNVSIKI (SEQ ID NO: 106), where X is any amino acid other than Asp. In some cases, X is Ala. In some cases, X is Arg.

A suitable PD-L1 variant includes a polypeptide that comprises an amino acid sequence having at least 90%, at least 95%, at least 98%, at least 99%, or 100%, amino acid sequence identity to the following amino acid sequence:

FT VTVPKDLYVV EYGSNMTIEC KFPVEKQLDL AALXVYWEME DKNIIQFVHG EEDLKVQHSS YRQRARLLKD QLSLGNAALQ ITDVKLQDAG VYRCMISYGG ADYKRITVKV NAPYNKINQR ILVVDPVTSE HELTCQAEGY PKAEVIWTSS DHQVLSGKTT TTNSKREEKL FNVTSTLRIN TTTNEIFYCT FRRLDPEENH TAELVIPGNI LNVSIKI (SEQ ID NO: 107), where X is any amino acid other than Ile. In some cases, X is Asp.

A suitable PD-L1 variant includes a polypeptide that comprises an amino acid sequence having at least 90%, at least 95%, at least 98%, at least 99%, or 100%, amino acid sequence identity to the following amino acid sequence:

FT VTVPKDLYVV EYGSNMTIEC KFPVEKQLDL AALIVYWEME DKNIIQFVHG EXDLKVQHSS YRQRARLLKD QLSLGNAALQ ITDVKLQDAG VYRCMISYGG ADYKRITVKV NAPYNKINQR ILVVDPVTSE HELTCQAEGY PKAEVIWTSS DHQVLSGKTT TTNSKREEKL FNVTSTLRIN TTTNEIFYCT FRRLDPEENH TAELVIPGNI LNVSIKI (SEQ ID NO: 108), where X is any amino acid other than Glu. In some cases, X is Arg.

### CD80 Variants

In some cases, a variant immunomodulatory polypeptide present in a TMAPP of the present disclosure is a variant CD80 polypeptide. Wild-type CD80 binds to CD28.

A wild-type amino acid sequence of the ectodomain of human CD80 can be as follows:

(SEQ ID NO: 109)
VIHVTK EVKEVATLSC GHNVSVEELA QTRIYWQKEK KMVLTMMSGD
MNIWPEYKNR TIFDITNNLS IVILALRPSD EGTYECVVLK
YEKDAFKREH LAEVTLSVKA DFPTPSISDF EIPTSNIRRI
ICSTSGGFPE PHLSWLENGE ELNAINTTVS QDPETELYAV
SSKLDFNMTT NHSFMCLIKY GHLRVNQTFN WNTTKQEHFP DN.

A wild-type CD28 amino acid sequence can be as follows: MLRLLLALNL FPSIQVTGNK ILVKQSPMLV AYDNAVNLSC KYSYNLFSRE FRASLHKGLD SAVEVCVVYG NYSQQLQVYS KTGFNCDGKL GNESVTFYLQ NLYVNQTDIY FCKIEVMYPP PYLDNEKSNG TIIHVKGKHL CPSPLFPGPS KPFWVLVVVG GVLACYSLLV TVAFIIFWVR SKRSRLLHSD YMNMTPRRPG PTRKHYQPYA PPRDFAAYRS (SEQ ID NO: 110).

A wild-type CD28 amino acid sequence can be as follows: MLRLLLALNL FPSIQVTGNK ILVKQSPMLV AYDNAVNLSW KHLCPSPLFP GPSKPFWVLV VVGGVLACYS LLVTVAFIIF WVRSKRSRLL HSDYMNMTPR RPGPTRKHYQ PYAPPRDFAA YRS (SEQ ID NO: 111)

A wild-type CD28 amino acid sequence can be as follows: MLRLLLALNL FPSIQVTGKH LCPSPLFPGP SKPFWVLVVV GGVLACYSLL VTVAFIIFWV RSKRSRLLHS DYMNMTPRRP GPTRKHYQPY APPRDFAAYR S (SEQ ID NO: 112).

In some cases, a variant CD80 polypeptide exhibits reduced binding affinity to CD28, compared to the binding affinity of a CD80 polypeptide comprising the amino acid sequence set forth in SEQ ID NO: 109 for CD28. For example, in some cases, a variant CD80 polypeptide binds CD28 with a binding affinity that is at least 10% less, at least 15% less, at least 20% less, at least 25% less, at least 30% less, at least 35% less, at least 40% less, at least 45% less, at least 50% less, at least 55% less, at least 60% less, at least 65% less, at least 70% less, at least 75% less, at least 80% less, at least 85% less, at least 90% less, at least 95% less, or more than 95% less, than the binding affinity of a CD80 polypeptide comprising the amino acid sequence set forth in SEQ ID NO: 109 for CD28 (e.g., a CD28 polypeptide comprising the amino acid sequence set forth in one of SEQ ID NO: 110, 111, or 112).

In some cases, a variant CD80 polypeptide has a binding affinity to CD28 that is from 100 nM to 100 μM. As another example, in some cases, a variant CD80 polypeptide of the present disclosure has a binding affinity for CD28 (e.g., a CD28 polypeptide comprising the amino acid sequence set forth in SEQ ID NO: 110, SEQ ID NO: 111, or SEQ ID NO: 112) that is from about 100 nM to 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

In some cases, a variant CD80 polypeptide has a single amino acid substitution compared to the CD80 amino acid sequence set forth in SEQ ID NO: 109. In some cases, a variant CD80 polypeptide has from 2 to 10 amino acid substitutions compared to the CD80 amino acid sequence set forth in SEQ ID NO: 109. In some cases, a variant CD80 polypeptide has 2 amino acid substitutions compared to the CD80 amino acid sequence set forth in SEQ ID NO: 109. In some cases, a variant CD80 polypeptide has 3 amino acid substitutions compared to the CD80 amino acid sequence set forth in SEQ ID NO: 109. In some cases, a variant CD80 polypeptide has 4 amino acid substitutions compared to the CD80 amino acid sequence set forth in SEQ ID NO: 109. In some cases, a variant CD80 polypeptide has 5 amino acid substitutions compared to the CD80 amino acid sequence set forth in SEQ ID NO: 109. In some cases, a variant CD80 polypeptide has 6 amino acid substitutions compared to the CD80 amino acid sequence set forth in SEQ ID NO: 109. In some cases, a variant CD80 polypeptide has 7 amino acid substitutions compared to the CD80 amino acid sequence set forth in SEQ ID NO: 109. In some cases, a variant CD80 polypeptide has 8 amino acid substitutions compared to the CD80 amino acid sequence set forth in SEQ ID NO: 109. In some cases, a variant CD80 polypeptide has 9 amino acid substitutions compared to the CD80 amino acid sequence set forth in SEQ ID NO: 109. In some cases, a variant CD80 polypeptide has 10 amino acid substitutions compared to the CD80 amino acid sequence set forth in SEQ ID NO: 109.

Suitable CD80 variants include a polypeptide that comprises an amino acid sequence having at least 90%, at least 95%, at least 98%, at least 99%, or 100%, amino acid sequence identity to any one of the following amino acid sequences:

VIHVTK EVKEVATLSC GHXVSVEELA QTRIYWQKEK KMVLTMMSGD MNIWPEYKNR TIFDITNNLS IVILALRPSD EGTYECVVLK YEKDAFKREH LAEVTLSVKA DFPTPSISDF EIPTSNIRRI ICSTSGGFPE PHLSWLENGE ELNAINTTVS QDPETELYAV SSKLDFNMTT NHSFMCLIKY GHLRVNQTFN WNTTKQEHFP DN (SEQ ID NO: 113), where X is any amino acid other than Asn. In some cases, X is Ala;

VIHVTK EVKEVATLSC GHNVSVEELA QTRIYWQKEK KMVLTMMSGD MNIWPEYKNR TIFDITXNLS IVILALRPSD EGTYECVVLK YEKDAFKREH LAEVTLSVKA DFPTPSISDF EIPTSNIRRI ICSTSGGFPE PHLSWLENGE ELNAINTTVS QDPETELYAV SSKLDFNMTT NHSFMCLIKY GHLRVNQTFN WNTTKQEHFP DN (SEQ ID NO: 114), where X is any amino acid other than Asn. In some cases, X is Ala;

VIHVTK EVKEVATLSC GHNVSVEELA QTRIYWQKEK KMVLTMMSGD MNIWPEYKNR TIFDITNNLS XVILALRPSD EGTYECVVLK YEKDAFKREH LAEVTLSVKA DFPTPSISDF EIPTSNIRRI ICSTSGGFPE PHLSWLENGE ELNAINTTVS QDPETELYAV SSKLDFNMTT NHSFMCLIKY GHLRVNQTFN WNTTKQEHFP DN (SEQ ID NO: 115), where X is any amino acid other than Ile. In some cases, X is Ala;

VIHVTK EVKEVATLSC GHNVSVEELA QTRIYWQKEK KMVLTMMSGD MNIWPEYKNR TIFDITNNLS IVILALRPSD EGTYECVVLX YEKDAFKREH LAEVTLSVKA DFPTPSISDF EIPTSNIRRI ICSTSGGFPE PHLSWLENGE ELNAINTTVS QDPETELYAV SSKLDFNMTT NHSFMCLIKY GHLRVNQTFN WNTTKQEHFP DN (SEQ ID NO: 116), where X is any amino acid other than Lys. In some cases, X is Ala;

VIHVTK EVKEVATLSC GHNVSVEELA QTRIYWQKEK KMVLTMMSGD MNIWPEYKNR TIFDITNNLS IVILALRPSD EGTYECVVLK YEKDAFKREH LAEVTLSVKA DFPTPSISDF EIPTSNIRRI ICSTSGGFPE PHLSWLENGE ELNAINTTVS XDPETELYAV SSKLDFNMTT NHSFMCLIKY GHLRVNQTFN WNTTKQEHFP DN (SEQ ID NO: 117), where X is any amino acid other than Gln. In some cases, X is Ala;

VIHVTK EVKEVATLSC GHNVSVEELA QTRIYWQKEK KMVLTMMSGD MNIWPEYKNR TIFDITNNLS IVILALRPSD EGTYECVVLK YEKDAFKREH LAEVTLSVKA DFPTPSISDF EIPTSNIRRI ICSTSGGFPE PHLSWLENGE ELNAINTTVS QXPETELYAV SSKLDFNMTT NHSFMCLIKY GHLRVNQTFN WNTTKQEHFP DN (SEQ ID NO: 118), where X is any amino acid other than Asp. In some cases, X is Ala;

VIHVTK EVKEVATLSC GHNVSVEEXA QTRIYWQKEK KMVLTMMSGD MNIWPEYKNR TIFDITNNLS IVILALRPSD EGTYECVVLK YEKDAFKREH LAEVTLSVKA DFPTPSISDF EIPTSNIRRI ICSTSGGFPE PHLSWLENGE ELNAINTTVS QDPETELYAV SSKLDFNMTT NHSFMCLIKY GHLRVNQTFN WNTTKQEHFP DN (SEQ ID NO: 119), where X is any amino acid other than Leu. In some cases, X is Ala;

VIHVTK EVKEVATLSC GHNVSVEELA QTRIXWQKEK KMVLTMMSGD MNIWPEYKNR TIFDITNNLS IVILALRPSD EGTYECVVLK YEKDAFKREH LAEVTLSVKA DFPTPSISDF EIPTSNIRRI ICSTSGGFPE PHLSWLENGE ELNAINTTVS QDPETELYAV SSKLDFNMTT NHSFMCLIKY GHLRVNQTFN WNTTKQEHFP DN (SEQ ID NO: 120), where X is any amino acid other than Tyr. In some cases, X is Ala;

VIHVTK EVKEVATLSC GHNVSVEELA QTRIYWXKEK KMVLTMMSGD MNIWPEYKNR TIFDITNNLS IVILALRPSD EGTYECVVLK YEKDAFKREH LAEVTLSVKA DFPTPSISDF EIPTSNIRRI ICSTSGGFPE PHLSWLENGE ELNAINTTVS QDPETELYAV SSKLDFNMTT NHSFMCLIKY GHLRVNQTFN WNTTKQEHFP DN (SEQ ID NO: 121), where X is any amino acid other than Gln. In some cases, X is Ala;

VIHVTK EVKEVATLSC GHNVSVEELA QTRIYWQKEK KXVLTMMSGD MNIWPEYKNR TIFDITNNLS IVILALRPSD EGTYECVVLK YEKDAFKREH LAEVTLSVKA DFPTPSISDF EIPTSNIRRI ICSTSGGFPE PHLSWLENGE ELNAINTTVS QDPETELYAV SSKLDFNMTT NHSFMCLIKY GHLRVNQTFN WNTTKQEHFP DN (SEQ ID NO: 122), where X is any amino acid other than Met. In some cases, X is Ala;

VIHVTK EVKEVATLSC GHNVSVEELA QTRIYWQKEK KMXLTMMSGD MNIWPEYKNR TIFDITNNLS IVILALRPSD EGTYECVVLK YEKDAFKREH LAEVTLSVKA DFPTPSISDF EIPTSNIRRI ICSTSGGFPE PHLSWLENGE ELNAINTTVS QDPETELYAV SSKLDFNMTT NHSFMCLIKY GHLRVNQTFN WNTTKQEHFP DN (SEQ ID NO: 123), where X is any amino acid other than Val. In some cases, X is Ala;

VIHVTK EVKEVATLSC GHNVSVEELA QTRIYWQKEK KMVLTMMSGD MNXWPEYKNR TIFDITNNLS IVILALRPSD EGTYECVVLK YEKDAFKREH LAEVTLSVKA DFPTPSISDF EIPTSNIRRI ICSTSGGFPE PHLSWLENGE ELNAINTTVS QDPETELYAV SSKLDFNMTT NHSFMCLIKY GHLRVNQTFN WNTTKQEHFP DN (SEQ ID NO: 124), where X is any amino acid other than Ile. In some cases, X is Ala;

VIHVTK EVKEVATLSC GHNVSVEELA QTRIYWQKEK KMVLTMMSGD MNIWPEXKNR TIFDITNNLS IVILALRPSD EGTYECVVLK YEKDAFKREH LAEVTLSVKA DFPTPSISDF EIPTSNIRRI ICSTSGGFPE PHLSWLENGE ELNAINTTVS QDPETELYAV SSKLDFNMTT NHSFMCLIKY GHLRVNQTFN WNTTKQEHFP DN (SEQ ID NO: 125), where X is any amino acid other than Tyr. In some cases, X is Ala;

VIHVTK EVKEVATLSC GHNVSVEELA QTRIYWQKEK KMVLTMMSGD MNIWPEYKNR TIFXITNNLS IVILALRPSD EGTYECVVLK YEKDAFKREH LAEVTLSVKA DFPTPSISDF EIPTSNIRRI ICSTSGGFPE PHLSWLENGE ELNAINTTVS QDPETELYAV SSKLDFNMTT NHSFMCLIKY GHLRVNQTFN WNTTKQEHFP DN (SEQ ID NO: 126), where X is any amino acid other than Asp. In some cases, X is Ala;

VIHVTK EVKEVATLSC GHNVSVEELA QTRIYWQKEK KMVLTMMSGD MNIWPEYKNR TIFDITNNLS IVILALRPSD EGTYECVVLK YEKDAFKREH LAEVTLSVKA DXPTPSISDF EIPTSNIRRI ICSTSGGFPE PHLSWLENGE ELNAINTTVS QDPETELYAV SSKLDFNMTT NHSFMCLIKY GHLRVNQTFN WNTTKQEHFP DN (SEQ ID NO: 127), where X is any amino acid other than Phe. In some cases, X is Ala;

VIHVTK EVKEVATLSC GHNVSVEELA QTRIYWQKEK KMVLTMMSGD MNIWPEYKNR TIFDITNNLS IVILALRPSD EGTYECVVLK YEKDAFKREH LAEVTLSVKA DFPTPSISDF EIPTSNIRRI ICSTSGGFPE PHLSWLENGE ELNAINTTVX QDPETELYAV SSKLDFNMTT NHSFMCLIKY GHLRVNQTFN WNTTKQEHFP DN (SEQ ID NO: 128), where X is any amino acid other than Ser. In some cases, X is Ala; and

VIHVTK EVKEVATLSC GHNVSVEELA QTRIYWQKEK KMVLTMMSGD MNIWPEYKNR TIFDITNNLS IVILALRPSD EGTYECVVLK YEKDAFKREH LAEVTLSVKA DFPTXSISDF EIPTSNIRRI ICSTSGGFPE PHLSWLENGE ELNAINTTVS QDPETELYAV SSKLDFNMTT NHSFMCLIKY GHLRVNQTFN WNTTKQEHFP DN (SEQ ID NO: 129), where X is any amino acid other than Pro. In some cases, X is Ala.

### CD86 Variants

In some cases, a variant immunomodulatory polypeptide present in a T TMAPP of the present disclosure is a variant CD86 polypeptide. Wild-type CD86 binds to CD28.

The amino acid sequence of the full ectodomain of a wild-type human CD86 can be as follows:

(SEQ ID NO: 130)
APLKIQAYFNETADLPCQFANSQNQSLSELVVFWQDQENLVLNEVYLGKEK
FDSVHSKYMNRTSFDSDSWTLRLHNLQIKDKGLYQCIIHHKKPTGMIRIHQ
MNSELSVLANFSQPEIVPISNITENVYINLTCSSIHGYPEPKKMSVLLRTK
NSTIEYDGIMQKSQDNVTELYDVSISLSVSFPDVTSNMTIFCILETDKTRL
LSSPFSIELEDPQPPPDHIP.

The amino acid sequence of the IgV domain of a wild-type human CD86 can be as follows:

(SEQ ID NO: 131)
APLKIQAYFNETADLPCQFANSQNQSLSELVVFWQDQENLVLNEVYLGKEK
FDSVHSKYMNRTSFDSDSWTLRLHNLQIKDKGLYQCIIHHKKPTGMIRIHQ
MNSELSVL.

In some cases, a variant CD86 polypeptide exhibits reduced binding affinity to CD28, compared to the binding affinity of a CD86 polypeptide comprising the amino acid sequence set forth in SEQ ID NO: 130 or SEQ ID NO: 131 for CD28. For example, in some cases, a variant CD86 polypeptide binds CD28 with a binding affinity that is at least 10% less, at least 15% less, at least 20% less, at least 25% less, at least 30% less, at least 35% less, at least 40% less, at least 45% less, at least 50% less, at least 55% less, at least 60% less, at least 65% less, at least 70% less, at least 75% less, at least 80% less, at least 85% less, at least 90% less, at least 95% less, or more than 95% less, than the binding affinity of a CD86 polypeptide comprising the amino acid sequence set forth in SEQ ID NO: 130 or SEQ ID NO: 131 for CD28 (e.g., a CD28 polypeptide comprising the amino acid sequence set forth in one of SEQ ID NO: 110, 111, or 112).

In some cases, a variant CD86 polypeptide has a binding affinity to CD28 that is from 100 nM to 100 μM. As another example, in some cases, a variant CD86 polypeptide of the present disclosure has a binding affinity for CD28 (e.g., a CD28 polypeptide comprising the amino acid sequence set forth in one of SEQ ID NOs: 110, 111, or 112) that is from about 100 nM to 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

In some cases, a variant CD86 polypeptide has a single amino acid substitution compared to the CD86 amino acid sequence set forth in SEQ ID NO: 130. In some cases, a variant CD86 polypeptide has from 2 to 10 amino acid substitutions compared to the CD86 amino acid sequence set forth in SEQ ID NO: 130. In some cases, a variant CD86 polypeptide has 2 amino acid substitutions compared to the CD86 amino acid sequence set forth in SEQ ID NO: 130. In some cases, a variant CD86 polypeptide has 3 amino acid substitutions compared to the CD86 amino acid sequence set forth in SEQ ID NO: 130. In some cases, a variant CD86 polypeptide has 4 amino acid substitutions compared to the CD86 amino acid sequence set forth in SEQ ID NO: 130. In some cases, a variant CD86 polypeptide has 5 amino acid substitutions compared to the CD86 amino acid sequence set forth in SEQ ID NO: 130. In some cases, a variant CD86 polypeptide has 6 amino acid substitutions compared to the CD86 amino acid sequence set forth in SEQ ID NO: 130. In some cases, a variant CD86 polypeptide has 7 amino acid substitutions compared to the CD86 amino acid sequence set forth in SEQ ID NO: 130. In some cases, a variant CD86 polypeptide has 8 amino acid substitutions compared to the CD86 amino acid sequence set forth in SEQ ID NO: 130. In some cases, a variant CD86 polypeptide has 9 amino acid substitutions compared to the CD86 amino acid sequence set forth in SEQ ID NO: 130. In some cases, a variant CD86 polypeptide has 10 amino acid substitutions compared to the CD86 amino acid sequence set forth in SEQ ID NO: 130.

In some cases, a variant CD86 polypeptide has a single amino acid substitution compared to the CD86 amino acid sequence set forth in SEQ ID NO: 131. In some cases, a variant CD86 polypeptide has from 2 to 10 amino acid substitutions compared to the CD86 amino acid sequence set forth in SEQ ID NO: 131. In some cases, a variant CD86 polypeptide has 2 amino acid substitutions compared to the CD86 amino acid sequence set forth in SEQ ID NO: β1. In some cases, a variant CD86 polypeptide has 3 amino acid substitutions compared to the CD86 amino acid sequence set forth in SEQ ID NO: 131. In some cases, a variant CD86 polypeptide has 4 amino acid substitutions compared to the CD86 amino acid sequence set forth in SEQ ID NO: 131. In some cases, a variant CD86 polypeptide has 5 amino acid substitutions compared to the CD86 amino acid sequence set forth in SEQ ID NO: 131. In some cases, a variant CD86 polypeptide has 6 amino acid substitutions compared to the CD86 amino acid sequence set forth in SEQ ID NO: 131. In some cases, a variant CD86 polypeptide has 7 amino acid substitutions compared to the CD86 amino acid sequence set forth in SEQ ID NO: 131. In some cases, a variant CD86 polypeptide has 8 amino acid substitutions compared to the CD86 amino acid sequence set forth in SEQ ID NO: 131. In some cases, a variant CD86 polypeptide has 9 amino acid substitutions compared to the CD86 amino acid sequence set forth in SEQ ID NO: β1. In some cases, a variant CD86 polypeptide has 10 amino acid substitutions compared to the CD86 amino acid sequence set forth in SEQ ID NO: 131.

Suitable CD86 variants include a polypeptide that comprises an amino acid sequence having at least 90%, at least 95%, at least 98%, at least 99%, or 100%, amino acid sequence identity to any one of the following amino acid sequences:

APLKIQAYFNETADLPCQFANSQNQSLSELVVFWQDQENLVLNEVYLGKEKFDSVHSKY MXRTSFDSDSWTLRLHNLQIKDKGLYQCIIHHKKPTGMIRIHQMNSELSVLANFSQPEIVPISN ITENVYINLTCSSIHGYPEPKKMSVLLRTKNSTIEYDGIMQKSQDNVTELYDVSISLSVSFPDV TSNMTIFCILETDKTRLLSSPFSIELEDPQPPPDHIP (SEQ ID NO: β2), where X is any amino acid other than Asn. In some cases, X is Ala;

APLKIQAYFNETADLPCQFANSQNQSLSELVVFWQDQENLVLNEVYLGKEKFDSVHSKY MNRTSFXSDSWTLRLHNLQIKDKGLYQCIIHHKKPTGMIRIHQMNSELSVLANFSQPEIVPISN ITENVYINLTCSSIHGYPEPKKMSVLLRTKNSTIEYDGIMQKSQDNVTELYDVSISLSVSFPDV TSNMTIFCILETDKTRLLSSPFSIELEDPQPPPDHIP (SEQ ID NO: 133), where X is any amino acid other than Asp. In some cases, X is Ala;

APLKIQAYFNETADLPCQFANSQNQSLSELVVFWQDQENLVLNEVYLGKEKFDSVHSKY MNRTSFDSDSXTLRLHNLQIKDKGLYQCIIHHKKPTGMIRIHQMNSELSVLANFSQPEIVPISN ITENVYINLTCSSIHGYPEPKKMSVLLRTKNSTIEYDGIMQKSQDNVTELYDVSISLSVSFPDV TSNMTIFCILETDKTRLLSSPFSIELEDPQPPPDHIP (SEQ ID NO:134), where X is any amino acid other than Trp. In some cases, X is Ala;

APLKIQAYFNETADLPCQFANSQNQSLSELVVFWQDQENLVLNEVYLGKEKFDSVHSKY MNRTSFDSDSWTLRLHNLQIKDKGLYQCIIHXKKPTGMIRIHQMNSELSVLANFSQPEIVPISN ITENVYINLTCSSIHGYPEPKKMSVLLRTKNSTIEYDGIMQKSQDNVTELYDVSISLSVSFPDV TSNMTIFCILETDKTRLLSSPFSIELEDPQPPPDHIP (SEQ ID NO: 135), where X is any amino acid other than His. In some cases, X is Ala;

APLKIQAYFNETADLPCQFANSQNQSLSELVVFWQDQENLVLNEVYLGKEKFDSVHSKY MXRTSFDSDSWTLRLHNLQIKDKGLYQCIIHHKKPTGMIRIHQMNSELSVL (SEQ ID NO: 136), where X is any amino acid other than Asn. In some cases, X is Ala;

APLKIQAYFNETADLPCQFANSQNQSLSELVVFWQDQENLVLNEVYLGKEKFDSVHSKY MNRTSFXSDSWTLRLHNLQIKDKGLYQCIIHHKKPTGMIRIHQMNSELSVL (SEQ ID NO: 137), where X is any amino acid other than Asp. In some cases, X is Ala;

APLKIQAYFNETADLPCQFANSQNQSLSELVVFWQDQENLVLNEVYLGKEKFDSVHSKY MNRTSFDSDSXTLRLHNLQIKDKGLYQCIIHHKKPTGMIRIHQMNSELSVL (SEQ ID NO: 138), where X is any amino acid other than Trp. In some cases, X is Ala;

APLKIQAYFNETADLPCQFANSQNQSLSELVVFWQDQENLVLNEVYLGKEKFDSVHSKY MNRTSFDSDSWTLRLHNLQIKDKGLYQCIIHXKKPTGMIRIHQMNSELSVL (SEQ ID NO: 139), where X is any amino acid other than His. In some cases, X is Ala;

APLKIQAYFNETADLPCQFANSQNQSLSELVVFWQDQENLXLNEVYLGKEKFDSVHSKY MNRTSFDSDSWTLRLHNLQIKDKGLYQCIIHHKKPTGMIRIHQMNSELSVLANFSQPEIVPISN ITENVYINLTCSSIHGYPEPKKMSVLLRTKNSTIEYDGIMQKSQDNVTELYDVSISLSVSFPDV TSNMTIFCILETDKTRLLSSPFSIELEDPQPPPDHIP (SEQ ID NO: 140), where X is any amino acid other than Val. In some cases, X is Ala;

APLKIQAYFNETADLPCQFANSQNQSLSELVVFWQDQENLXLNEVYLGKEKFDSVHSKY MNRTSFDSDSWTLRLHNLQIKDKGLYQCIIHHKKPTGMIRIHQMNSELSVL (SEQ ID NO: 141), where X is any amino acid other than Val. In some cases, X is Ala;

APLKIQAYFNETADLPCQFANSQNQSLSELVVFWXDQENLVLNEVYLGKEKFDSVHSKY MNRTSFDSDSWTLRLHNLQIKDKGLYQCIIHHKKPTGMIRIHQMNSELSVLANFSQPEIVPISN ITENVYINLTCSSIHGYPEPKKMSVLLRTKNSTIEYDGIMQKSQDNVTELYDVSISLSVSFPDV TSNMTIFCILETDKTRLLSSPFSIELEDPQPPPDHIP (SEQ ID NO: 142), where X is any amino acid other than Gln. In some cases, X is Ala;

APLKIQAYFNETADLPCQFANSQNQSLSELVVFWXDQENLVLNEVYLGKEKFDSVHSKY MNRTSFDSDSWTLRLHNLQIKDKGLYQCIIHHKKPTGMIRIHQMNSELSVL (SEQ ID NO: 143), where X is any amino acid other than Gln. In some cases, X is Ala;

APLKIQAYFNETADLPCQFANSQNQSLSELVVXWQDQENLVLNEVYLGKEKFDSVHSKY MNRTSFDSDSWTLRLHNLQIKDKGLYQCIIHHKKPTGMIRIHQMNSELSVLANFSQPEIVPISN ITENVYINLTCSSIHGYPEPKKMSVLLRTKNSTIEYDGIMQKSQDNVTELYDVSISLSVSFPDV TSNMTIFCILETDKTRLLSSPFSIELEDPQPPPDHIP (SEQ ID NO: 144), where X is any amino acid other than Phe. In some cases, X is Ala;

APLKIQAYFNETADLPCQFANSQNQSLSELVVXWQDQENLVLNEVYLGKEKFDSVHSKY MNRTSFDSDSWTLRLHNLQIKDKGLYQCIIHHKKPTGMIRIHQMNSELSVL (SEQ ID NO: 145), where X is any amino acid other than Phe. In some cases, X is Ala;

APLKIQAYFNETADLPCQFANSQNQSLSELVVFWQDQENLVLNEVYLGKEKFDSVHSKY MNRTSFDSDSWTXRLHNLQIKDKGLYQCIIHHKKPTGMIRIHQMNSELSVLANFSQPEIVPISN ITENVYINLTCSSIHGYPEPKKMSVLLRTKNSTIEYDGIMQKSQDNVTELYDVSISLSVSFPDV TSNMTIFCILETDKTRLLSSPFSIELEDPQPPPDHIP (SEQ ID NO: 146), where X is any amino acid other than Leu. In some cases, X is Ala;

APLKIQAYFNETADLPCQFANSQNQSLSELVVFWQDQENLVLNEVYLGKEKFDSVHSKY MNRTSFDSDSWTXRLHNLQIKDKGLYQCIIHHKKPTGMIRIHQMNSELSVL (SEQ ID NO: 147), where X is any amino acid other than Leu. In some cases, X is Ala;

APLKIQAYFNETADLPCQFANSQNQSLSELVVFWQDQENLVLNEVYLGKEKFDSVHSKX MNRTSFDSDSWTLRLHNLQIKDKGLYQCIIHHKKPTGMIRIHQMNSELSVLANFSQPEIVPISN ITENVYINLTCSSIHGYPEPKKMSVLLRTKNSTIEYDGIMQKSQDNVTELYDVSISLSVSFPDV TSNMTIFCILETDKTRLLSSPFSIELEDPQPPPDHIP (SEQ ID NO: 148), where X is any amino acid other than Tyr. In some cases, X is Ala;

APLKIQAYFNETADLPCQFANSQNQSLSELVVFWQDQENLVLNEVYLGKEKFDSVHSKX MNRTSFDSDSWTLRLHNLQIKDKGLYQCIIHHKKPTGMIRIHQMNSELSVL (SEQ ID NO: 149), where X is any amino acid other than Tyr. In some cases, X is Ala;

APLKIQAYFNETADLPCQFANSQNQSLSELVVFWQDQENLVLNEVYLGKEKFDSVHSKY MXRTSFDSDSWTLRLHNLQIKDKGLYQCIIHXKKPTGMIRIHQMNSELSVLANFSQPEIVPISN ITENVYINLTCSSIHGYPEPKKMSVLLRTKNSTIEYDGIMQKSQDNVTELYDVSISLSVSFPDV TSNMTIFCILETDKTRLLSSPFSIELEDPQPPPDHIP (SEQ ID NO: 150), where the first X is any amino acid other than Asn and the second X is any amino acid other than His. In some cases, the first and the second X are both Ala;

APLKIQAYFNETADLPCQFANSQNQSLSELVVFWQDQENLVLNEVYLGKEKFDSVHSKY MXRTSFDSDSWTLRLHNLQIKDKGLYQCIIHXKKPTGMIRIHQMNSELSVL (SEQ ID NO: 151), where the first X is any amino acid other than Asn and the second X is any amino acid other than His. In some cases, the first and the second X are both Ala;

APLKIQAYFNETADLPCQFANSQNQSLSELVVFWQDQENLVLNEVYLGKEKFDSVHSKY MNRTSFX₁SDSWTLRLHNLQIKDKGLYQCIIHX₂KKPTGMIRIHQMNSELSVLANFSQPEIVPIS NITENVYINLTCSSIHGYPEPKKMSVLLRTKNSTIEYDGIMQKSQDNVTELYDVSISLSVSFPD VTSNMTIFCILETDKTRLLSSPFSIELEDPQPPPDHIP (SEQ ID NO: 152), where X₁ is any amino acid other than Asp, and X₂ is any amino acid other than His. In some cases, X₁ is Ala and X₂ is Ala;

APLKIQAYFNETADLPCQFANSQNQSLSELVVFWQDQENLVLNEVYLGKEKFDSVHSKY MNRTSFX₁SDSWILRLHNLQIKDKGLYQCIIHX₂KKPTGMIRIHQMNSELSVL (SEQ ID NO: 153), where the first X is any amino acid other than Asn and the second X is any amino acid other than His. In some cases, the first and the second X are both Ala;

APLKIQAYFNETADLPCQFANSQNQSLSELVVFWQDQENLVLNEVYLGKEKFDSVHSKY MX₁RTSFX₂SDSWITRLHNLQIKDKGLYQCIIHX₃KKPTGMIRIHQMNSELSVLANFSQPEIVPI SNITENVYINLTCSSIHGYPEPKKMSVLLRTKNSTIEYDGIMQKSQDNVTELYDVSISLSVSFP DVTSNMTIFCILETDKTRLLSSPFSIELEDPQPPPDHIP (SEQ ID NO: 154), where X₁ is any amino acid other than Asn, X₂ is any amino acid other than Asp, and X₃ is any amino acid other than His. In some cases, X₁ is Ala, X₂ is Ala, and X₃ is Ala; and

APLKIQAYFNETADLPCQFANSQNQSLSELVVFWQDQENLVLNEVYLGKEKFDSVHSKY MX₁RTSFX₂SDSWITRLHNLQIKDKGLYQCIIHX₃KKPTGMIRIHQMNSELSVL (SEQ ID NO: 155), where X₁ is any amino acid other than Asn, X₂ is any amino acid other than Asp, and X₃ is any amino acid other than His In some cases, X₁ is Ala, X₂ is Ala, and X₃ is Ala.

### 4-1BBL Variants

In some cases, a variant immunomodulatory polypeptide present in a TMAPP of the present disclosure is a variant 4-1BBL polypeptide. Wild-type 4-1BBL binds to 4-1BB (CD137).

A wild-type 4-1BBL amino acid sequence can be as follows: MEYASDASLD PEAPWPPAPR ARACRVLPWA LVAGLLLLLL LAAACAVFLA CPWAVSGARA SPGSAASPRL REGPELSPDD PAGLLDLRQG MFAQLVAQNV LLIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO: 156).

In some cases, a variant 4-1BBL polypeptide is a variant of the tumor necrosis factor (TNF) homology domain (THD) of human 4-1BBL.

A wild-type amino acid sequence of the THD of human 4-1BBL can be, e.g., one of SEQ ID NOs: 157-159, as follows:

(SEQ ID NO: 157)
PAGLLDLRQG MFAQLVAQNV LLIDGPLSWY SDPGLAGVSL
TGGLSYKEDT KELVVAKAGV YYVFFQLELR RVVAGEGSGS
VSLALHLQPL RSAAGAAALA LTVDLPPASS EARNSAFGFQ
GRLLHLSAGQ RLGVHLHTEA RARHAWQLTQ GATVLGLFRV
TPEIPAGLPS PRSE.
(SEQ ID NO: 158)
D PAGLLDLRQG MFAQLVAQNV LLIDGPLSWY SDPGLAGVSL
TGGLSYKEDT KELVVAKAGV YYVFFQLELR RVVAGEGSGS
VSLALHLQPL RSAAGAAALA LTVDLPPASS EARNSAFGFQ
GRLLHLSAGQ RLGVHLHTEA RARHAWQLTQ GATVLGLFRV
TPEIPAGLPS PRSE.
(SEQ ID NO: 159)
D PAGLLDLRQG MFAQLVAQNV LLIDGPLSWY SDPGLAGVSL
TGGLSYKEDT KELVVAKAGV YYVFFQLELR RVVAGEGSGS
VSLALHLQPL RSAAGAAALA LTVDLPPASS EARNSAFGFQ
GRLLHLSAGQ RLGVHLHTEA RARHAWQLTQ GATVLGLFRV TPEIPA.

A wild-type 4-1BB amino acid sequence can be as follows: MGNSCYNIVA TLLLVLNFER TRSLQDPCSN CPAGTFCDNN RNQICSPCPP NSFSSAGGQR TCDICRQCKG VFRTRKECSS TSNAECDCTP GFHCLGAGCS MCEQDCKQGQ ELTKKGCKDC CFGTFNDQKR GICRPWTNCS LDGKSVLVNG TKERDVVCGP SPADLSPGAS SVTPPAPARE PGHSPQIISF FLALTSTALL FLLFFLTLRF SVVKRGRKKL LYIFKQPFMR PVQTTQEEDG CSCRFPEEEE GGCEL (SEQ ID NO: 160).

In some cases, a variant 4-1BBL polypeptide exhibits reduced binding affinity to 4-1BB, compared to the binding affinity of a 4-1BBL polypeptide comprising the amino acid sequence set forth in one of SEQ ID NOs: 156-159. For example, in some cases, a variant 4-1BBL polypeptide of the present disclosure binds 4-1BB with a binding affinity that is at least 10% less, at least 15% less, at least 20% less, at least 25%, at least 30% less, at least 35% less, at least 40% less, at least 45% less, at least 50% less, at least 55% less, at least 60% less, at least 65% less, at least 70% less, at least 75% less, at least 80% less, at least 85% less, at least 90% less, at least 95% less, or more than 95% less, than the binding affinity of a 4-1BBL polypeptide comprising the amino acid sequence set forth in one of SEQ ID NOs: 156-159 for a 4-1BB polypeptide (e.g., a 4-1BB polypeptide comprising the amino acid sequence set forth in SEQ ID NO: 160), when assayed under the same conditions.

In some cases, a variant 4-1BBL polypeptide has a binding affinity to 4-1BB that is from 100 nM to 100 μM. As another example, in some cases, a variant 4-1BBL polypeptide has a binding affinity for 4-1BB (e.g., a 4-1BB polypeptide comprising the amino acid sequence set forth in SEQ ID NO: 160) that is from about 100 nM to 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

In some cases, a variant 4-1BBL polypeptide has a single amino acid substitution compared to the 4-1BBL amino acid sequence set forth in one of SEQ ID NOs: 156-159. In some cases, a variant 4-1BBL polypeptide has from 2 to 10 amino acid substitutions compared to the 4-1BBL amino acid sequence set forth in one of SEQ ID NOs: 156-159. In some cases, a variant 4-1BBL polypeptide has 2 amino acid substitutions compared to the 4-1BBL amino acid sequence set forth in one of SEQ ID NOs: 156-159. In some cases, a variant 4-1BBL polypeptide has 3 amino acid substitutions compared to the 4-1BBL amino acid sequence set forth in one of SEQ ID NOs: 156-159. In some cases, a variant 4-1BBL polypeptide has 4 amino acid substitutions compared to the 4-1BBL amino acid sequence set forth in one of SEQ ID NOs: 156-159. In some cases, a variant 4-1BBL polypeptide has 5 amino acid substitutions compared to the 4-1BBL amino acid sequence set forth in one of SEQ ID NOs: 156-159. In some cases, a variant 4-1BBL polypeptide has 6 amino acid substitutions compared to the 4-1BBL amino acid sequence set forth in one of SEQ ID NOs: 156-159. In some cases, a variant 4-1BBL polypeptide has 7 amino acid substitutions compared to the 4-1BBL amino acid sequence set forth in one of SEQ ID NOs: 156-159. In some cases, a variant 4-1BBL polypeptide has 8 amino acid substitutions compared to the 4-1BBL amino acid sequence set forth in one of SEQ ID NOs: 156-159. In some cases, a variant 4-1BBL polypeptide has 9 amino acid substitutions compared to the 4-1BBL amino acid sequence set forth in one of SEQ ID NOs: 156-159. In some cases, a variant 4-1BBL polypeptide has 10 amino acid substitutions compared to the 4-1BBL amino acid sequence set forth in one of SEQ ID NOs: 156-159.

Suitable 4-1BBL variants include a polypeptide that comprises an amino acid sequence having at least 90%, at least 95%, at least 98%, at least 99%, or 100%, amino acid sequence identity to any one of the following amino acid sequences:

PAGLLDLRQG MFAQLVAQNV LLIDGPLSWY SDPGLAGVSL TGGLSYXEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO: 161), where X is any amino acid other than Lys. In some cases, X is Ala;

PAGLLDLRQG MFAQLVAQNV LLIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWXLTQ GATVLGLFRV TPEIPAGLPS PRSE (SEQ ID NO: 162), where X is any amino acid other than Gln. In some cases, X is Ala;

PAGLLDLRQG XFAQLVAQNV LLIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO: 163), where X is any amino acid other than Met. In some cases, X is Ala;

PAGLLDLRQG MXAQLVAQNV LLIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO: 164), where X is any amino acid other than Phe. In some cases, X is Ala;

PAGLLDLRQG MFAXLVAQNV LLIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO: 165), where X is any amino acid other than Gln. In some cases, X is Ala;

PAGLLDLRQG MFAQXVAQNV LLIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:166), where X is any amino acid other than Leu. In some cases, X is Ala;

PAGLLDLRQG MFAQLXAQNV LLIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO: 167), where X is any amino acid other than Val. In some cases, X is Ala;

PAGLLDLRQG MFAQLVAXNV LLIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO: 168), where X is any amino acid other than Gln. In some cases, X is Ala;

PAGLLDLRQG MFAQLVAQXV LLIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO: 169), where X is any amino acid other than Asn. In some cases, X is Ala;

PAGLLDLRQG MFAQLVAQNX LLIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO: 170), where X is any amino acid other than Val. In some cases, X is Ala;

PAGLLDLRQG MFAQLVAQNV XLIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO: 171), where X is any amino acid other than Leu. In some cases, X is Ala;

PAGLLDLRQG MFAQLVAQNV LXIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO: 172), where X is any amino acid other than Leu. In some cases, X is Ala;

PAGLLDLRQG MFAQLVAQNV LLXDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO: 173), where X is any amino acid other than Ile. In some cases, X is Ala;

PAGLLDLRQG MFAQLVAQNV LLIXGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO: 174), where X is any amino acid other than Asp. In some cases, X is Ala;

PAGLLDLRQG MFAQLVAQNV LLIDXPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO: 175), where X is any amino acid other than Gly. In some cases, X is Ala;

PAGLLDLRQG MFAQLVAQNV LLIGGXLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO: 176), where X is any amino acid other than Pro. In some cases, X is Ala;

PAGLLDLRQG MFAQLVAQNV LLIGGPXSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO: 177), where X is any amino acid other than Leu. In some cases, X is Ala;

PAGLLDLRQG MFAQLVAQNV LLIGGPLXWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO: 178), where X is any amino acid other than Ser. In some cases, X is Ala;

PAGLLDLRQG MFAQLVAQNV LLIGGPLSXY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO: 179), where X is any amino acid other than Trp. In some cases, X is Ala;

PAGLLDLRQG MFAQLVAQNV LLIGGPLSWX SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO: 180), where X is any amino acid other than Tyr. In some cases, X is Ala;

PAGLLDLRQG MFAQLVAQNV LLIGGPLSWY XDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO: 181), where X is any amino acid other than Ser. In some cases, X is Ala;

PAGLLDLRQG MFAQLVAQNV LLIGGPLSWY SXPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO: 182), where X is any amino acid other than Asp. In some cases, X is Ala;

PAGLLDLRQG MFAQLVAQNV LLIGGPLSWY SDXGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO: 183), where X is any amino acid other than Pro. In some cases, X is Ala;

PAGLLDLRQG MFAQLVAQNV LLIGGPLSWY SDPXLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO: 184), where X is any amino acid other than Gly. In some cases, X is Ala;

PAGLLDLRQG MFAQLVAQNV LLIGGPLSWY SDPGXAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO: 185), where X is any amino acid other than Leu. In some cases, X is Ala;

PAGLLDLRQG MFAQLVAQNV LLIGGPLSWY SDPGLAXVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO: 186), where X is any amino acid other than Gly. In some cases, X is Ala;

PAGLLDLRQG MFAQLVAQNV LLIGGPLSWY SDPGLAGXSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO: 187), where X is any amino acid other than Val. In some cases, X is Ala;

PAGLLDLRQG MFAQLVAQNV LLIGGPLSWY SDPGLAGVXL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO: 188), where X is any amino acid other than Ser. In some cases, X is Ala;

PAGLLDLRQG MFAQLVAQNV LLIGGPLSWY SDPGLAGVSX TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO: 189), where X is any amino acid other than Leu. In some cases, X is Ala;

PAGLLDLRQG MFAQLVAQNV LLIGGPLSWY SDPGLAGVSL XGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO: 190), where X is any amino acid other than Thr. In some cases, X is Ala;

PAGLLDLRQG MFAQLVAQNV LLIGGPLSWY SDPGLAGVSL TXGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:191), where X is any amino acid other than Gly. In some cases, X is Ala;

PAGLLDLRQG MFAQLVAQNV LLIGGPLSWY SDPGLAGVSL TGXLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO: 192), where X is any amino acid other than Gly. In some cases, X is Ala;

PAGLLDLRQG MFAQLVAQNV LLIGGPLSWY SDPGLAGVSL TGGXSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO: 193), where X is any amino acid other than Leu. In some cases, X is Ala;

PAGLLDLRQG MFAQLVAQNV LLIGGPLSWY SDPGLAGVSL TGGLXYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO: 194), where X is any amino acid other than Ser. In some cases, X is Ala;

PAGLLDLRQG MFAQLVAQNV LLIGGPLSWY SDPGLAGVSL TGGLSXKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO: 195), where X is any amino acid other than Tyr. In some cases, X is Ala;

PAGLLDLRQG MFAQLVAQNV LLIGGPLSWY SDPGLAGVSL TGGLSYKXDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO: 196), where X is any amino acid other than Glu. In some cases, X is Ala;

PAGLLDLRQG MFAQLVAQNV LLIGGPLSWY SDPGLAGVSL TGGLSYKEXT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO: 197), where X is any amino acid other than Asp. In some cases, X is Ala;

PAGLLDLRQG MFAQLVAQNV LLIGGPLSWY SDPGLAGVSL TGGLSYKEDX KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO: 198), where X is any amino acid other than Thr. In some cases, X is Ala;

PAGLLDLRQG MFAQLVAQNV LLIGGPLSWY SDPGLAGVSL TGGLSYKEDT XELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO: 199), where X is any amino acid other than Lys. In some cases, X is Ala;

PAGLLDLRQG MFAQLVAQNV LLIGGPLSWY SDPGLAGVSL TGGLSYKEDT KXLVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO: 200), where X is any amino acid other than Glu. In some cases, X is Ala;

PAGLLDLRQG MFAQLVAQNV LLIGGPLSWY SDPGLAGVSL TGGLSYKEDT KELVVAKAGV YYVXFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO: 201), where X is any amino acid other than Phe. In some cases, X is Ala;

PAGLLDLRQG MFAQLVAQNV LLIGGPLSWY SDPGLAGVSL TGGLSYKEDT KELVVAKAGV YYVFXQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO: 202), where X is any amino acid other than Phe. In some cases, X is Ala;

PAGLLDLRQG MFAQLVAQNV LLIGGPLSWY SDPGLAGVSL TGGLSYKEDT KELVVAKAGV YYVFFXLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO: 203), where X is any amino acid other than Gln. In some cases, X is Ala;

PAGLLDLRQG MFAQLVAQNV LLIGGPLSWY SDPGLAGVSL TGGLSYKEDT KELVVAKAGV YYVFFQXELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO: 204), where X is any amino acid other than Leu. In some cases, X is Ala;

PAGLLDLRQG MFAQLVAQNV LLIGGPLSWY SDPGLAGVSL TGGLSYKEDT KELVVAKAGV YYVFFQLXLR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO: 205), where X is any amino acid other than Glu. In some cases, X is Ala;

PAGLLDLRQG MFAQLVAQNV LLIGGPLSWY SDPGLAGVSL TGGLSYKEDT KELVVAKAGV YYVFFQLEXR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO: 206), where X is any amino acid other than Leu. In some cases, X is Ala;

PAGLLDLRQG MFAQLVAQNV LLIGGPLSWY SDPGLAGVSL TGGLSYKEDT KELVVAKAGV YYVFFQLELX RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO: 207), where X is any amino acid other than Arg. In some cases, X is Ala;

PAGLLDLRQG MFAQLVAQNV LLIGGPLSWY SDPGLAGVSL TGGLSYKEDT KELVVAKAGV YYVFFQLELR XVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO: 208), where X is any amino acid other than Arg. In some cases, X is Ala;

PAGLLDLRQG MFAQLVAQNV LLIGGPLSWY SDPGLAGVSL TGGLSYKEDT KELVVAKAGV YYVFFQLELR RXVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO: 209), where X is any amino acid other than Val. In some cases, X is Ala;

PAGLLDLRQG MFAQLVAQNV LLIGGPLSWY SDPGLAGVSL TGGLSYKEDT KELVVAKAGV YYVFFQLELR RVXAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO: 210), where X is any amino acid other than Val. In some cases, X is Ala;

PAGLLDLRQG MFAQLVAQNV LLIGGPLSWY SDPGLAGVSL TGGLSYKEDT KELVVAKAGV YYVFFQLELR RVVAXEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO: 211), where X is any amino acid other than Gly. In some cases, X is Ala;

PAGLLDLRQG MFAQLVAQNV LLIGGPLSWY SDPGLAGVSL TGGLSYKEDT KELVVAKAGV YYVFFQLELR RVVAGXGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO: 212), where X is any amino acid other than Glu. In some cases, X is Ala;

PAGLLDLRQG MFAQLVAQNV LLIGGPLSWY SDPGLAGVSL TGGLSYKEDT KELVVAKAGV YYVFFQLELR RVVAGEXSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO: 213), where X is any amino acid other than Gly. In some cases, X is Ala;

PAGLLDLRQG MFAQLVAQNV LLIGGPLSWY SDPGLAGVSL TGGLSYKEDT KELVVAKAGV YYVFFQLELR RVVAGEGXGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO: 214), where X is any amino acid other than Ser. In some cases, X is Ala;

PAGLLDLRQG MFAQLVAQNV LLIGGPLSWY SDPGLAGVSL TGGLSYKEDT KELVVAKAGV YYVFFQLELR RVVAGEGSGS VSLALHLQPL RSAAGAAALA LTVXLPPASS EARNSAFGFQ GRLLHLSAGQ RLGVHLHTEA RARHAWQLTQ GATVLGLFRV TPEIPAGLPS PRSE (SEQ ID NO: 215), where X is any amino acid other than Asp. In some cases, X is Ala;

PAGLLDLRQG MFAQLVAQNV LLIGGPLSWY SDPGLAGVSL TGGLSYKEDT KELVVAKAGV YYVFFQLELR RVVAGEGSGS VSLALHLQPL RSAAGAAALA LTVDXPPASS EARNSAFGFQ GRLLHLSAGQ RLGVHLHTEA RARHAWQLTQ GATVLGLFRV TPEIPAGLPS PRSE (SEQ ID NO: 216), where X is any amino acid other than Leu. In some cases, X is Ala;

PAGLLDLRQG MFAQLVAQNV LLIGGPLSWY SDPGLAGVSL TGGLSYKEDT KELVVAKAGV YYVFFQLELR RVVAGEGSGS VSLALHLQPL RSAAGAAALA LTVDLXPASS EARNSAFGFQ GRLLHLSAGQ RLGVHLHTEA RARHAWQLTQ GATVLGLFRV TPEIPAGLPS PRSE (SEQ ID NO: 217), where X is any amino acid other than Pro. In some cases, X is Ala;

PAGLLDLRQG MFAQLVAQNV LLIGGPLSWY SDPGLAGVSL TGGLSYKEDT KELVVAKAGV YYVFFQLELR RVVAGEGSGS VSLALHLQPL RSAAGAAALA LTVDLPPAXS EARNSAFGFQ GRLLHLSAGQ RLGVHLHTEA RARHAWQLTQ GATVLGLFRV TPEIPAGLPS PRSE (SEQ ID NO: 218), where X is any amino acid other than Ser. In some cases, X is Ala;

PAGLLDLRQG MFAQLVAQNV LLIGGPLSWY SDPGLAGVSL TGGLSYKEDT KELVVAKAGV YYVFFQLELR RVVAGEGSGS VSLALHLQPL RSAAGAAALA LTVDLPPASX EARNSAFGFQ GRLLHLSAGQ RLGVHLHTEA RARHAWQLTQ GATVLGLFRV TPEIPAGLPS PRSE (SEQ ID NO: 219), where X is any amino acid other than Ser. In some cases, X is Ala;

PAGLLDLRQG MFAQLVAQNV LLIGGPLSWY SDPGLAGVSL TGGLSYKEDT KELVVAKAGV YYVFFQLELR RVVAGEGSGS VSLALHLQPL RSAAGAAALA LTVDLPPASS XARNSAFGFQ GRLLHLSAGQ RLGVHLHTEA RARHAWQLTQ GATVLGLFRV TPEIPAGLPS PRSE (SEQ ID NO: 220), where X is any amino acid other than Glu. In some cases, X is Ala;

PAGLLDLRQG MFAQLVAQNV LLIGGPLSWY SDPGLAGVSL TGGLSYKEDT KELVVAKAGV YYVFFQLELR RVVAGEGSGS VSLALHLQPL RSAAGAAALA LTVDLPPASS EAXNSAFGFQ GRLLHLSAGQ RLGVHLHTEA RARHAWQLTQ GATVLGLFRV TPEIPAGLPS PRSE (SEQ ID NO: 221), where X is any amino acid other than Arg. In some cases, X is Ala;

PAGLLDLRQG MFAQLVAQNV LLIGGPLSWY SDPGLAGVSL TGGLSYKEDT KELVVAKAGV YYVFFQLELR RVVAGEGSGS VSLALHLQPL RSAAGAAALA LTVDLPPASS EARXSAFGFQ GRLLHLSAGQ RLGVHLHTEA RARHAWQLTQ GATVLGLFRV TPEIPAGLPS PRSE (SEQ ID NO: 222), where X is any amino acid other than Asn. In some cases, X is Ala;

PAGLLDLRQG MFAQLVAQNV LLIGGPLSWY SDPGLAGVSL TGGLSYKEDT KELVVAKAGV YYVFFQLELR RVVAGEGSGS VSLALHLQPL RSAAGAAALA LTVDLPPASS EARNXAFGFQ GRLLHLSAGQ RLGVHLHTEA RARHAWQLTQ GATVLGLFRV TPEIPAGLPS PRSE (SEQ ID NO: 223), where X is any amino acid other than Ser. In some cases, X is Ala;

PAGLLDLRQG MFAQLVAQNV LLIGGPLSWY SDPGLAGVSL TGGLSYKEDT KELVVAKAGV YYVFFQLELR RVVAGEGSGS VSLALHLQPL RSAAGAAALA LTVDLPPASS EARNSAXGFQ GRLLHLSAGQ RLGVHLHTEA RARHAWQLTQ GATVLGLFRV TPEIPAGLPS PRSE (SEQ ID NO: 224), where X is any amino acid other than Phe. In some cases, X is Ala;

PAGLLDLRQG MFAQLVAQNV LLIGGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGX RLGVHLHTEA RARHAWQLTQ GATVLGLFRV TPEIPAGLPS PRSE (SEQ ID NO: 225), where X is any amino acid other than Gln. In some cases, X is Ala;

PAGLLDLRQG MFAQLVAQNV LLIGGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ XLGVHLHTEA RARHAWQLTQ GATVLGLFRV TPEIPAGLPS PRSE (SEQ ID NO: 226), where X is any amino acid other than Arg. In some cases, X is Ala;

PAGLLDLRQG MFAQLVAQNV LLIGGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RXGVHLHTEA RARHAWQLTQ GATVLGLFRV TPEIPAGLPS PRSE (SEQ ID NO: 227), where X is any amino acid other than Leu. In some cases, X is Ala;

PAGLLDLRQG MFAQLVAQNV LLIGGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLXVHLHTEA RARHAWQLTQ GATVLGLFRV TPEIPAGLPS PRSE (SEQ ID NO: 228), where X is any amino acid other than Gly. In some cases, X is Ala;

PAGLLDLRQG MFAQLVAQNV LLIGGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGXHLHTEA RARHAWQLTQ GATVLGLFRV TPEIPAGLPS PRSE (SEQ ID NO: 229), where X is any amino acid other than Val. In some cases, X is Ala;

PAGLLDLRQG MFAQLVAQNV LLIGGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVXLHTEA RARHAWQLTQ GATVLGLFRV TPEIPAGLPS PRSE (SEQ ID NO: 230), where X is any amino acid other than His. In some cases, X is Ala;

PAGLLDLRQG MFAQLVAQNV LLIGGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHXHTEA RARHAWQLTQ GATVLGLFRV TPEIPAGLPS PRSE (SEQ ID NO: 231), where X is any amino acid other than Leu. In some cases, X is Ala;

PAGLLDLRQG MFAQLVAQNV LLIGGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLXTEA RARHAWQLTQ GATVLGLFRV TPEIPAGLPS PRSE (SEQ ID NO: 232), where X is any amino acid other than His. In some cases, X is Ala;

PAGLLDLRQG MFAQLVAQNV LLIGGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHXEA RARHAWQLTQ GATVLGLFRV TPEIPAGLPS PRSE (SEQ ID NO: 233), where X is any amino acid other than Thr. In some cases, X is Ala;

PAGLLDLRQG MFAQLVAQNV LLIGGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTXA RARHAWQLTQ GATVLGLFRV TPEIPAGLPS PRSE (SEQ ID NO: 234), where X is any amino acid other than Glu. In some cases, X is Ala;

PAGLLDLRQG MFAQLVAQNV LLIGGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA XARHAWQLTQ GATVLGLFRV TPEIPAGLPS PRSE (SEQ ID NO: 235), where X is any amino acid other than Arg. In some cases, X is Ala;

PAGLLDLRQG MFAQLVAQNV LLIGGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RAXHAWQLTQ GATVLGLFRV TPEIPAGLPS PRSE (SEQ ID NO: 236), where X is any amino acid other than Arg. In some cases, X is Ala;

PAGLLDLRQG MFAQLVAQNV LLIGGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARXAWQLTQ GATVLGLFRV TPEIPAGLPS PRSE (SEQ ID NO: 237), where X is any amino acid other than His. In some cases, X is Ala;

PAGLLDLRQG MFAQLVAQNV LLIGGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAXQLTQ GATVLGLFRV TPEIPAGLPS PRSE (SEQ ID NO: 238), where X is any amino acid other than Trp. In some cases, X is Ala;

PAGLLDLRQG MFAQLVAQNV LLIGGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQXTQ GATVLGLFRV TPEIPAGLPS PRSE (SEQ ID NO: 239), where X is any amino acid other than Leu. In some cases, X is Ala;

PAGLLDLRQG MFAQLVAQNV LLIGGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLXQ GATVLGLFRV TPEIPAGLPS PRSE (SEQ ID NO: 240), where X is any amino acid other than Thr. In some cases, X is Ala;

PAGLLDLRQG MFAQLVAQNV LLIGGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTX GATVLGLFRV TPEIPAGLPS PRSE (SEQ ID NO: 241), where X is any amino acid other than Gln. In some cases, X is Ala;

PAGLLDLRQG MFAQLVAQNV LLIGGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ XATVLGLFRV TPEIPAGLPS PRSE (SEQ ID NO: 242), where X is any amino acid other than Gly. In some cases, X is Ala;

PAGLLDLRQG MFAQLVAQNV LLIGGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GAXVLGLFRV TPEIPAGLPS PRSE (SEQ ID NO: 243), where X is any amino acid other than Thr. In some cases, X is Ala; and

PAGLLDLRQG MFAQLVAQNV LLIGGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATXLGLFRV TPEIPAGLPS PRSE (SEQ ID NO: 244), where X is any amino acid other than Val. In some cases, X is Ala.

### IL-2 Variants

In some cases, a variant immunomodulatory polypeptide present in a TMAPP of the present disclosure is a variant IL-2 polypeptide. Wild-type IL-2 binds to IL-2 receptor (IL-2R).

A wild-type IL-2 amino acid sequence can be as follows: APTSSSTKKT QLQLEHLLLD LQMILNGINN YKNPKLTRML TFKFYMPKKA TELKHLQCLEEELKPLEEVL NLAQSKNFHL RPRDLISNIN VIVLELKGSE TTFMCEYADE TATIVEFLNRWITFCQSIIS TLT (SEQ ID NO: 245).

Wild-type IL2 binds to an IL2 receptor (IL2R) on the surface of a cell. An IL2 receptor is in some cases a heterotrimeric polypeptide comprising an alpha chain (IL-2Rα; also referred to as CD25), a beta chain (IL-2Rβ; also referred to as CD122: and a gamma chain (IL-2Rγ; also referred to as CD132) Amino acid sequences of human IL-2Rα, IL2Rβ, and IL-2Rγ can be as follows.

Human IL-2Rα:
(SEQ ID NO: 246)
ELCDDDPPE IPHATFKAMA YKEGTMLNCE CKRGFRRIKS
GSLYMLCTGN SSHSSWDNQC QCTSSATRNT TKQVTPQPEE
QKERKTTEMQ SPMQPVDQAS LPGHCREPPP WENEATERIY
HFVVGQMVYY QCVQGYRALH RGPAESVCKM THGKTRWTQP
QLICTGEMET SQFPGEEKPQ ASPEGRPESE TSCLVTTTDF
QIQTEMAATM ETSIFTTEYQ VAVAGCVFLL ISVLLLSGLT
WQRRQRKSRR TI.
Human IL-2Rβ:
(SEQ ID NO: 247)
VNG TSQFTCFYNS RANISCVWSQ DGALQDTSCQ VHAWPDRRRW
NQTCELLPVS QASWACNLIL GAPDSQKLTT VDIVTLRVLC
REGVRWRVMA IQDFKPFENL RLMAPISLQV VHVETHRCNI
SWEISQASHY FERHLEFEAR TLSPGHTWEE APLLTLKQKQ
EWICLETLTP DTQYEFQVRV KPLQGEFTTW SPWSQPLAFR
TKPAALGKDT IPWLGHLLVG LSGAFGFIIL VYLLINCRNT
GPWLKKVLKC NTPDPSKFFS QLSSEHGGDV QKWLSSPFPS
SSFSPGGLAP EISPLEVLER DKVTQLLLQQ DKVPEPASLS
SNHSLTSCFT NQGYFFFHLP DALEIEACQV YFTYDPYSEE
DPDEGVAGAP TGSSPQPLQP LSGEDDAYCT FPSRDDLLLF
SPSLLGGPSP PSTAPGGSGA GEERMPPSLQ ERVPRDWDPQ
PLGPPTPGVP DLVDFQPPPE LVLREAGEEV PDAGPREGVS
FPWSRPPGQG EFRALNARLP LNTDAYLSLQ ELQGQDPTHL V.
Human IL-2Rγ:
(SEQ ID NO: 248)
LNTTILTP NGNEDTTADF FLTTMPTDSL SVSTLPLPEV
QCFVFNVEYM NCTWNSSSEP QPTNLTLHYW YKNSDNDKVQ
KCSHYLFSEE ITSGCQLQKK EIHLYQTFVV QLQDPREPRR
QATQMLKLQN LVIPWAPENL TLHKLSESQL ELNWNNRFLN
HCLEHLVQYR TDWDHSWTEQ SVDYRHKFSL PSVDGQKRYT
FRVRSRFNPL CGSAQHWSEW SHPIHWGSNT SKENPFLFAL
EAVVISVGSM GLIISLLCVY FWLERTMPRI PTLKNLEDLV
TEYHGNFSAW SGVSKGLAES LQPDYSERLC LVSEIPPKGG
ALGEGPGASP CNQHSPYWAP PCYTLKPET.

In some cases, where a TMAPP of the present disclosure comprises a variant IL-2 polypeptide, a “cognate co-immunomodulatory polypeptide” is an IL-2R comprising polypeptides comprising the amino acid sequences of SEQ ID NO: 246, 247, and 248.

In some cases, a variant IL-2 polypeptide exhibits reduced binding affinity to IL-2R, compared to the binding affinity of a IL-2 polypeptide comprising the amino acid sequence set forth in SEQ ID NO: 245. For example, in some cases, a variant IL-2 polypeptide binds IL-2R with a binding affinity that is at least 10% less, at least 15% less, at least 20% less, at least 25%, at least 30% less, at least 35% less, at least 40% less, at least 45% less, at least 50% less, at least 55% less, at least 60% less, at least 65% less, at least 70% less, at least 75% less, at least 80% less, at least 85% less, at least 90% less, at least 95% less, or more than 95% less, than the binding affinity of an IL-2 polypeptide comprising the amino acid sequence set forth in SEQ ID NO: 245 for an IL-2R (e.g., an IL-2R comprising polypeptides comprising the amino acid sequence set forth in SEQ ID NOs: 246-248), when assayed under the same conditions.

In some cases, a variant IL-2 polypeptide has a binding affinity to IL-2R that is from 100 nM to 100 μM. As another example, in some cases, a variant IL-2 polypeptide has a binding affinity for IL-2R (e.g., an IL-2R comprising polypeptides comprising the amino acid sequence set forth in SEQ ID NOs: 246-248) that is from about 100 nM to 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

In some cases, a variant IL-2 polypeptide has a single amino acid substitution compared to the IL-2 amino acid sequence set forth in SEQ ID NO: 245. In some cases, a variant IL-2 polypeptide has from 2 to 10 amino acid substitutions compared to the IL-2 amino acid sequence set forth in SEQ ID NO: 245. In some cases, a variant IL-2 polypeptide has 2 amino acid substitutions compared to the IL-2 amino acid sequence set forth in SEQ ID NO: 245. In some cases, a variant IL-2 polypeptide has 3 amino acid substitutions compared to the IL-2 amino acid sequence set forth in SEQ ID NO: 245. In some cases, a variant IL-2 polypeptide has 4 amino acid substitutions compared to the IL-2 amino acid sequence set forth in SEQ ID NO: 245. In some cases, a variant IL-2 polypeptide has 5 amino acid substitutions compared to the IL-2 amino acid sequence set forth in SEQ ID NO: 245. In some cases, a variant IL-2 polypeptide has 6 amino acid substitutions compared to the IL-2 amino acid sequence set forth in SEQ ID NO: 245. In some cases, a variant IL-2 polypeptide has 7 amino acid substitutions compared to the IL-2 amino acid sequence set forth in SEQ ID NO: 245. In some cases, a variant IL-2 polypeptide has 8 amino acid substitutions compared to the IL-2 amino acid sequence set forth in SEQ ID NO: 245. In some cases, a variant IL-2 polypeptide has 9 amino acid substitutions compared to the IL-2 amino acid sequence set forth in SEQ ID NO: 245. In some cases, a variant IL-2 polypeptide has 10 amino acid substitutions compared to the IL-2 amino acid sequence set forth in SEQ ID NO: 245.

Suitable IL-2 variants include a polypeptide that comprises an amino acid sequence having at least 90%, at least 95%, at least 98%, at least 99%, or 100%, amino acid sequence identity to any one of the following amino acid sequences:

APTSSSTKKT QLQLEHLLLD LQMILNGINN YKNPKLTRML TXKFYMPKKA TELKHLQCLE EELKPLEEVL NLAQSKNFHL RPRDLISNIN VIVLELKGSE TTFMCEYADE TATIVEFLNR WITFCQSIIS TLT (SEQ ID NO: 249), where X is any amino acid other than Phe. In some cases, X is Ala;

APTSSSTKKT QLQLEHLLLX LQMILNGINN YKNPKLTRML TFKFYMPKKA TELKHLQCLE EELKPLEEVL NLAQSKNFHL RPRDLISNIN VIVLELKGSE TTFMCEYADE TATIVEFLNR WITFCQSIIS TLT (SEQ ID NO: 250), where X is any amino acid other than Asp. In some cases, X is Ala;

APTSSSTKKT QLQLXHLLLD LQMILNGINN YKNPKLTRML TFKFYMPKKA TELKHLQCLE EELKPLEEVL NLAQSKNFHL RPRDLISNIN VIVLELKGSE TTFMCEYADE TATIVEFLNR WITFCQSIIS TLT (SEQ ID NO: 251), where X is any amino acid other than Glu. In some cases, X is Ala;

APTSSSTKKT QLQLEXLLLD LQMILNGINN YKNPKLTRML TFKFYMPKKA TELKHLQCLE EELKPLEEVL NLAQSKNFHL RPRDLISNIN VIVLELKGSE TTFMCEYADE TATIVEFLNR WITFCQSIIS TLT (SEQ ID NO: 252), where X is any amino acid other than His. In some cases, X is Ala. In some cases, X is Arg. In some cases, X is Asn. In some cases, X is Asp. In some cases, X is Cys. In some cases, X is Glu. In some cases, X is Gln. In some cases, X is Gly. In some cases, X is Ile. In some cases, X is Lys. In some cases, X is Leu. In some cases, X is Met. In some cases, X is Phe. In some cases, X is Pro. In some cases, X is Ser. In some cases, X is Thr. In some cases, X is Tyr. In some cases, X is Trp. In some cases, X is Val;

APTSSSTKKT QLQLEHLLLD LQMILNGINN YKNPKLTRML TFKFXMPKKA TELKHLQCLE EELKPLEEVL NLAQSKNFHL RPRDLISNIN VIVLELKGSE TTFMCEYADE TATIVEFLNR WITFCQSIIS TLT (SEQ ID NO: 253), where X is any amino acid other than Tyr. In some cases, X is Ala;

APTSSSTKKT QLQLEHLLLD LQMILNGINN YKNPKLTRML TFKFYMPKKA TELKHLQCLE EELKPLEEVL NLAQSKNFHL RPRDLISNIN VIVLELKGSE TTFMCEYADE TATIVEFLNR WITFCXSIIS TLT (SEQ ID NO: 254), where X is any amino acid other than Gln. In some cases, X is Ala;

APTSSSTKKT QLQLEX₁LLLD LQMILNGINN YKNPKLTRML TX₂KFYMPKKA TELKHLQCLE EELKPLEEVL NLAQSKNFHL RPRDLISNIN VIVLELKGSE TTFMCEYADE TATIVEFLNR WITFCQSIIS TLT (SEQ ID NO: 255), where X₁ is any amino acid other than His, and where X₂ is any amino acid other than Phe. In some cases, X₁ is Ala. In some cases, X₂ is Ala. In some cases, X₁ is Ala; and X₂ is Ala. In some cases, X₁ is Thr; and X₂ is Ala;

APTSSSTKKT QLQLEHLLLX₁ LQMILNGINN YKNPKLTRML TX₂KFYMPKKA TELKHLQCLE EELKPLEEVL NLAQSKNFHL RPRDLISNIN VIVLELKGSE TTFMCEYADE TATIVEFLNR WITFCQSIIS TLT (SEQ ID NO: 256), where X₁ is any amino acid other than Asp; and where X₂ is any amino acid other than Phe. In some cases, X₁ is Ala. In some cases, X₂ is Ala. In some cases, X₁ is Ala; and X₂ is Ala;

APTSSSTKKT QLQLX₁HLLLX₂ LQMILNGINN YKNPKLTRML TX₃KFYMPKKA TELKHLQCLE EELKPLEEVL NLAQSKNFHL RPRDLISNIN VIVLELKGSE TTFMCEYADE TATIVEFLNR WITFCQSIIS TLT (SEQ ID NO: 257), where X₁ is any amino acid other than Glu; where X₂ is any amino acid other than Asp; and where X₃ is any amino acid other than Phe. In some cases, X₁ is Ala. In some cases, X₂ is Ala. In some cases, X₃ is Ala. In some cases, X₁ is Ala; X₂ is Ala; and X₃ is Ala;

APTSSSTKKT QLQLEX₁LLLX₂ LQMILNGINN YKNPKLTRML TX₃KFYMPKKA TELKHLQCLE EELKPLEEVL NLAQSKNFHL RPRDLISNIN VIVLELKGSE TTFMCEYADE TATIVEFLNR WITFCQSIIS TLT (SEQ ID NO: 258), where X₁ is any amino acid other than His; where X₂ is any amino acid other than Asp; and where X₃ is any amino acid other than Phe. In some cases, X₁ is Ala. In some cases, X₂ is Ala. In some cases, X₃ is Ala. In some cases, X₁ is Ala; X₂ is Ala; and X₃ is Ala;

APTSSSTKKT QLQLEHLLLX₁ LQMILNGINN YKNPKLTRML TX₂KFYMPKKA TELKHLQCLE EELKPLEEVL NLAQSKNFHL RPRDLISNIN VIVLELKGSE TTFMCEYADE TATIVEFLNR WITFCX₃SIIS TLT (SEQ ID NO: 259), where X₁ is any amino acid other than Asp; where X₂ is any amino acid other than Phe; and where X₃ is any amino acid other than Gln. In some cases, X₁ is Ala. In some cases, X₂ is Ala. In some cases, X₃ is Ala. In some cases, X₁ is Ala; X₂ is Ala; and X₃ is Ala;

APTSSSTKKT QLQLEHLLLX₁ LQMILNGINN YKNPKLTRML TX₂KFX₃MPKKA TELKHLQCLE EELKPLEEVL NLAQSKNFHL RPRDLISNIN VIVLELKGSE TTFMCEYADE TATIVEFLNR WITFCQSIIS TLT (SEQ ID NO: 260), where X₁ is any amino acid other than Asp; where X₂ is any amino acid other than Phe; and where X₃ is any amino acid other than Tyr. In some cases, X₁ is Ala. In some cases, X₂ is Ala. In some cases, X₃ is Ala. In some cases, X₁ is Ala; X₂ is Ala; and X₃ is Ala;

APTSSSTKKT QLQLEX₁LLLX₂ LQMILNGINN YKNPKLTRML TX₃KFX₄MPKKA TELKHLQCLE EELKPLEEVL NLAQSKNFHL RPRDLISNIN VIVLELKGSE TTFMCEYADE TATIVEFLNR WITFCQSIIS TLT (SEQ ID NO: 261), where X₁ is any amino acid other than His; where X₂ is any amino acid other than Asp; where X₃ is any amino acid other than Phe; and where X₄ is any amino acid other than Tyr. In some cases, X₁ is Ala. In some cases, X₂ is Ala. In some cases, X₃ is Ala. In some cases, X₄ is Ala. In some cases, X₁ is Ala; X₂ is Ala; X₃ is Ala; and X₄ is Ala;

APTSSSTKKT QLQLEHLLLX₁ LQMILNGINN YKNPKLTRML TX₂KFX₃MPKKA TELKHLQCLE EELKPLEEVL NLAQSKNFHL RPRDLISNIN VIVLELKGSE TTFMCEYADE TATIVEFLNR WITFCX₄SIIS TLT (SEQ ID NO: 262), where X₁ is any amino acid other than Asp; where X₂ is any amino acid other than Phe; where X₃ is any amino acid other than Tyr; and where X₄ is any amino acid other than Gln. In some cases, X₁ is Ala. In some cases, X₂ is Ala. In some cases, X₃ is Ala. In some cases, X₄ is Ala. In some cases, X₁ is Ala; X₂ is Ala; X₃ is Ala; and X₄ is Ala;

APTSSSTKKT QLQLEX₁LLLX₂ LQMILNGINN YKNPKLTRML TX₃KFX₄MPKKA TELKHLQCLE EELKPLEEVL NLAQSKNFHL RPRDLISNIN VIVLELKGSE TTFMCEYADE TATIVEFLNR WITFCX₅SIIS TLT (SEQ ID NO: 263), where X₁ is any amino acid other than His; where X₂ is any amino acid other than Asp; where X₃ is any amino acid other than Phe; where X₄ is any amino acid other than Tyr; and where X₅ is any amino acid other than Gln. In some cases, X₁ is Ala. In some cases, X₂ is Ala. In some cases, X₃ is Ala. In some cases, X₄ is Ala. In some cases, X₅ is Ala. In some cases, X₁ is Ala; X₂ is Ala; X₃ is Ala; X₄ is Ala; X₅ is Ala; and

APTSSSTKKT QLQLEX₁LLLD LQMILNGINN YKNPKLTRML TX₂KFYMPKKA TELKHLQCLE EELKPLEEVL NLAQSKNFHL RPRDLISNIN VIVLELKGSE TTFMCEYADE TATIVEFLNR WITFCX₃SIIS TLT (SEQ ID NO: 264), where X₁ is any amino acid other than His; where X₂ is any amino acid other than Phe; and where X₃ is any amino acid other than Gln. In some cases, X₁ is Ala. In some cases, X₂ is Ala. In some cases, X₃ is Ala. In some cases, X₁ is Ala; X₂ is Ala; and X₃ is Ala.

### TGF-β

As noted above, in some cases, the immunomodulatory polypeptide present in a TMAPP of the present disclosure is a TGF-β polypeptide Amino acid sequences of TGF-β polypeptides are known in the art. In some cases, the immunomodulatory polypeptide present in a TMAPP of the present disclosure is a TGF-β1 polypeptide immunomodulatory polypeptide present in a TMAPP of the present disclosure is a TGF-β2 polypeptide immunomodulatory polypeptide present in a TMAPP of the present disclosure is a TGF-β3 polypeptide. A suitable TGF-β polypeptide can comprise an amino acid sequence having at least 60%, at least 70%, at least 80%, at least 90%, at least 95%, at least 98%, at least 99%, or 100%, amino acid sequence identity to the mature form of a human TGF-β1 polypeptide, a human TGF-β2 polypeptide, or a human TGF-β3 polypeptide. A suitable TGF-β polypeptide can have a length of from about 100 amino acids to about 125 amino acids; for example, a suitable TGF-β polypeptide can have a length of from about 100 amino acids to about 105 amino acids, from about 105 amino acids to about 110 amino acids, from about 110 amino acids to about 115 amino acids, from about 115 amino acids to about 120 amino acids, or from about 120 amino acids to about 125 amino acids.

A suitable TGF-β1 polypeptide can comprise an amino acid sequence having at least 60%, at least 70%, at least 80%, at least 90%, at least 95%, at least 98%, at least 99%, or 100%, amino acid sequence identity to the following TGF-β1 amino acid sequence: AL DTNYCFSSTE KNCCVRQLYI DFRKDLGWKW IHEPKGYHAN FCLGPCPYIW SLDTQYSKVL ALYNQHNPGA SAAPCCVPQA LEPLPIVYYV GRKPKVEQLS NMIVRSCKCS (SEQ ID NO: 265); where the TGF-β1 polypeptide has a length of about 112 amino acids.

In some cases, a suitable TGF-β1 polypeptide comprises a C77S substitution. Thus, in some cases, a suitable TGF-β1 polypeptide comprises an amino acid sequence having at least 60%, at least 70%, at least 80%, at least 90%, at least 95%, at least 98%, at least 99%, or 100%, amino acid sequence identity to the following TGF-β1 amino acid sequence: AL DTNYCFSSTE KNCCVRQLYI DFRKDLGWKW IHEPKGYHAN FCLGPCPYIW SLDTQYSKVL ALYNQHNPGA SAAPSCVPQA LEPLPIVYYV GRKPKVEQLS NMIVRSCKCS (SEQ ID NO: 266), where amino acid 77 is Ser.

A suitable TGF-β2 polypeptide can comprise an amino acid sequence having at least 60%, at least 70%, at least 80%, at least 90%, at least 95%, at least 98%, at least 99%, or 100%, amino acid sequence identity to the following TGF-β2 amino acid sequence: ALDAAYCF RNVQDNCCLR PLYIDFKRDL GWKWIHEPKG YNANFCAGAC PYLWSSDTQH SRVLSLYNTI NPEASASPCC VSQDLEPLTI LYYIGKTPKI EQLSNMIVKS CKCS (SEQ ID NO: 267), where the TGF-β2 polypeptide has a length of about 112 amino acids.

In some cases, a suitable TGF-β2 polypeptide comprises a C77S substitution. Thus, in some cases, a suitable TGF-β2 polypeptide comprises an amino acid sequence having at least 60%, at least 70%, at least 80%, at least 90%, at least 95%, at least 98%, at least 99%, or 100%, amino acid sequence identity to the following TGF-β2 amino acid sequence: ALDAAYCF RNVQDNCCLR PLYIDFKRDL GWKWIHEPKG YNANFCAGAC PYLWSSDTQH SRVLSLYNTI NPEASASPSC VSQDLEPLTI LYYIGKTPKI EQLSNMIVKS CKCS (SEQ ID NO: 268), where amino acid 77 is Ser.

A suitable TGF-β3 polypeptide can comprise an amino acid sequence having at least 60%, at least 70%, at least 80%, at least 90%, at least 95%, at least 98%, at least 99%, or 100%, amino acid sequence identity to the following TGF-β3 amino acid sequence: ALDTNYCFRN LEENCCVRPL YIDFRQDLGW KWVHEPKGYY ANFCSGPCPY LRSADTTHST VLGLYNTLNP EASASPCCVP QDLEPLTILY YVGRTPKVEQ LSNMVVKSCK CS (SEQ ID NO: 269), where the TGF-β3 polypeptide has a length of about 112 amino acids.

In some cases, a suitable TGF-β3 polypeptide comprises a C77S substitution. Thus, in some cases, a suitable TGF-β3 polypeptide comprises an amino acid sequence having at least 60%, at least 70%, at least 80%, at least 90%, at least 95%, at least 98%, at least 99%, or 100%, amino acid sequence identity to the following TGF-β3 amino acid sequence: ALDTNYCFRN LEENCCVRPL YIDFRQDLGW KWVHEPKGYY ANFCSGPCPY LRSADTTHST VLGLYNTLNP EASASPSCVP QDLEPLTILY YVGRTPKVEQ LSNMVVKSCK CS (SEQ ID NO: 270), where amino acid 77 is Ser.

### Dimerizer Pairs

As noted above, in some cases, an antigen-presenting polypeptide of the present disclosure (including a TMAPP of the present disclosure) comprises a dimerizer pair of polypeptides. For example, where an antigen-presenting polypeptide of the present disclosure (including a TMAPP of the present disclosure) is a multimeric polypeptide comprising at least a first and a second polypeptide, in some cases, the first polypeptide comprises a first member of a dimerization pair, and the second polypeptide comprising a second member of the dimerization pair.

Dimerization peptides are known in the art; and any known dimerization peptide is suitable for use. Dimerization peptides include polypeptides of the collectin family (e.g., ACRP30 or ACRP30-like proteins) which contain collagen domains consisting of collagen repeats Gly-Xaa-Xaa. Other dimerization peptides include coiled-coil domains and leucine-zipper domains. A collagen domain can comprise (Gly-Xaa-Xaa)ₙ (SEQ ID NO: 431), where Xaa is any amino acid, and where n is an integer from 10 to 40. In some cases, a collagen domain comprises (Gly-Xaa-Pro)ₙ (SEQ ID NO: 432), where Xaa is any amino acid and n is an integer from 10 to 40. Dimerization peptides are well known in the art; see, e.g., U.S. Patent Publication No. 2003/0138440.

In some cases, a dimerization pair includes two leucine zipper polypeptides that bind to one another. Non-limiting examples of leucine-zipper polypeptides include, e.g., a peptide of any one of the following amino acid sequences: RMKQIEDKIEEILSKIYHIENEIARIKKLIGER (SEQ ID NO: 271); LSSIEKKQEEQTSWLIWISNELTLIRNELAQS (SEQ ID NO: 272); LSSIEKKLEEITSQLIQISNELTLIRNELAQ (SEQ ID NO: 273); LSSIEKKLEEITSQLIQIRNELTLIRNELAQ (SEQ ID NO: 274); LSSIEKKLEEITSQLQQIRNELTLIRNELAQ (SEQ ID NO: 275); LSSLEKKLEELTSQLIQLRNELTLLRNELAQ (SEQ ID NO: 276); ISSLEKKIEELTSQIQQLRNEITLLRNEIAQ (SEQ ID NO: 277).

In some cases, a leucine zipper polypeptide comprises the following amino acid sequence: LEIEAAFLERENTALETRVAELRQRVQRLRNRVSQYRTRYGPLGGGK (SEQ ID NO: 278).

Additional leucine-zipper polypeptides are known in the art, any of which is suitable for use in an antigen-presenting polypeptide of the present disclosure.

A collagen oligomerization peptide can comprise the following amino acid sequence:

(SEQ ID NO: 279)
VTAFSNMDDMLQKAHLVIEGTFIYLRDSTEFFIRVRDGWKKLQLGELIPIP
ADSPPPPALSSNP.

Coiled-coil dimerization peptides are known in the art. For example, a coiled-coil dimerization peptide can be a peptide of any one of the following amino acid sequences:

(SEQ ID NO: 280)
LKSVENRLAVVENQLKTVIEELKTVKDLLSN;
(SEQ ID NO: 281)
LARIEEKLKTIKAQLSEIASTLNMIREQLAQ;
(SEQ ID NO: 282)
VSRLEEKVKTLKSQVTELASTVSLLREQVAQ;
(SEQ ID NO: 283)
IQSEKKIEDISSLIGQIQSEITLIRNEIAQ;
(SEQ ID NO: 284)
LMSLEKKLEELTQTLMQLQNELSMLKNELAQ.

In some cases, a dimerization peptide comprises at least one cysteine residue. Examples include, e.g.: VDLEGSTSNGRQCAGIRL (SEQ ID NO: 285); EDDVTTTEELAPALVPPPKGTCAGWMA (SEQ ID NO: 286); and GHDQETTTQGPGVLLPLPKGACTGQMA (SEQ ID NO: 287).

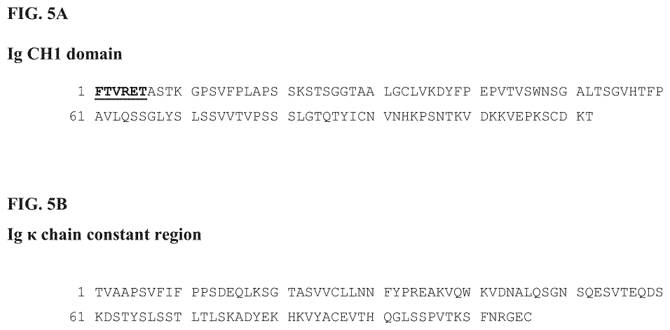

Ig CH1 domains and Ig κ chain constant regions, such as those shown in FIG. 5A and FIG. 5B can also serve as dimerization peptides.

### Additional Polypeptides

A polypeptide chain of a TMAPP of the present disclosure (including a TMAPP of the present disclosure) can include one or more polypeptides in addition to those described above. Suitable additional polypeptides include epitope tags and affinity domains. The one or more additional polypeptide can be included at the N-terminus of a polypeptide chain of a TMAPP of the present disclosure, at the C-terminus of a polypeptide chain of a TMAPP of the present disclosure, or internally within a polypeptide chain of a TMAPP of the present disclosure.

### Epitope Tag

Suitable epitope tags include, but are not limited to, hemagglutinin (HA; e.g., YPYDVPDYA (SEQ ID NO: 288); FLAG (e.g., DYKDDDDK (SEQ ID NO: 289); c-myc (e.g., EQKLISEEDL; SEQ ID NO: 290), and the like.

### Affinity Domain

Affinity domains include peptide sequences that can interact with a binding partner, e.g., such as one immobilized on a solid support, useful for identification or purification. DNA sequences encoding multiple consecutive single amino acids, such as histidine, when fused to the expressed protein, may be used for one-step purification of the recombinant protein by high affinity binding to a resin column, such as nickel sepharose. Exemplary affinity domains include His5 (HHHHH) (SEQ ID NO: 291), HisX6 (HHHHHH) (SEQ ID NO: 292), C-myc (EQKLISEEDL) (SEQ ID NO: 290), Flag (DYKDDDDK) (SEQ ID NO: 289), StrepTag (WSHPQFEK) (SEQ ID NO: 293), hemagglutinin, e.g., HA Tag (YPYDVPDYA) (SEQ ID NO: 288), glutathione-S-transferase (GST), thioredoxin, cellulose binding domain, RYIRS (SEQ ID NO: 294), Phe-His-His-Thr (SEQ ID NO: 295), chitin binding domain, S-peptide, T7 peptide, SH2 domain, C-end RNA tag, WEAAAREACCRECCARA (SEQ ID NO: 296), metal binding domains, e.g., zinc binding domains or calcium binding domains such as those from calcium-binding proteins, e.g., calmodulin, troponin C, calcineurin B, myosin light chain, recoverin, S-modulin, visinin, VILIP, neurocalcin, hippocalcin, frequenin, caltractin, calpain large-subunit, S100 proteins, parvalbumin, calbindin D9K, calbindin D28K, and calretinin, inteins, biotin, streptavidin, MyoD, Id, leucine zipper sequences, and maltose binding protein.

### Drug Conjugates

A polypeptide chain of a TMAPP of the present disclosure can comprise a small molecule drug linked (e.g., covalently attached) to the polypeptide chain. For example, where a TMAPP of the present disclosure comprises an Fc polypeptide, the Fc polypeptide can comprise a covalently linked small molecule drug. In some cases, the small molecule drug is an agent that treats an autoimmune disease, or an agent that relieves a symptom of an autoimmune disease. A polypeptide chain of a TMAPP of the present disclosure can comprise an agent linked (e.g., covalently attached) to the polypeptide chain. For example, where a TMAPP of the present disclosure comprises an Fc polypeptide, the Fc polypeptide can comprise a covalently linked agent.

A drug can be linked directly or indirectly to a polypeptide chain of a TMAPP of the present disclosure. For example, where a TMAPP of the present disclosure comprises an Fc polypeptide, a drug can be linked directly or indirectly to the Fc polypeptide. Direct linkage can involve linkage directly to an amino acid side chain. Indirect linkage can be linkage via a linker. A drug can be linked to a polypeptide chain (e.g., an Fc polypeptide) of a TMAPP of the present disclosure via a thioether bond, an amide bond, a carbamate bond, a disulfide bond, or an ether bond.

Linkers include cleavable linkers and non-cleavable linkers. In some cases, the linker is a protease-cleavable linker. Suitable linkers include, e.g., peptides (e.g., from 2 to 10 amino acids in length; e.g., 2, 3, 4, 5, 6, 7, 8, 9, or 10 amino acids in length), alkyl chains, poly(ethylene glycol), disulfide groups, thioether groups, acid labile groups, photolabile groups, peptidase labile groups, and esterase labile groups. Non-limiting example of suitable linkers are: i) N-succinimidyl-[(N-maleimidopropionamido)-tetraethyleneglycol]ester (NHS-PEG4-maleimide); ii) N-succinimidyl 4-(2-pyridyldithio)butanoate (SPDB); N-succinimidyl 4-(2-pyridyldithio)2-sulfobutanoate (sulfo-SPDB); N-succinimidyl 4-(2-pyridyldithio) pentanoate (SPP); N-succinimidyl-4-(N-maleimidomethyl)-cyclohexane-1-carboxy-(6-amidocaproate) (LC-SMCC); κ-maleimidoundecanoic acid N-succinimidyl ester (KMUA); γ-maleimide butyric acid N-succinimidyl ester (GMBS); ε-maleimidocaproic acid N-hydroxysuccinimide ester (EMCS); m-maleimide benzoyl-N-hydroxysuccinimide ester (MB S); N-(α-maleimidoacetoxy)-succinimide ester (AMAS); succinimidyl-6-(β-maleimidopropionamide)hexanoate (SMPH); N-succinimidyl 4-(p-maleimidophenyl)butyrate (SMPB); N-(p-maleimidophenyl)isocyanate (PMPI); N-succinimidyl 4(2-pyridylthio)pentanoate (SPP); N-succinimidyl(4-iodo-acetyl)aminobenzoate (SIAB); 6-maleimidocaproyl (MC); maleimidopropanoyl (MP); p-aminobenzyloxycarbonyl (PAB); N-succinimidyl 4-(maleimidomethyl)cyclohexanecarboxylate (SMCC); N-succinimidyl-4-(N-maleimidomethyl)-cyclohexane-1-carboxy-(6-amidocaproate), a “long chain” analog of SMCC (LC-SMCC); 3-maleimidopropanoic acid N-succinimidyl ester (BMPS); N-succinimidyl iodoacetate (SIA); N-succinimidyl bromoacetate (SBA); and N-succinimidyl 3-(bromoacetamido)propionate (SBAP).

A polypeptide (e.g., an Fc polypeptide) can be modified with crosslinking reagents such as succinimidyl 4-(N-maleimidomethyl)-cyclohexane-1-carboxylate (SMCC), sulfo-SMCC, maleimidobenzoyl-N-hydroxysuccinimide ester (MBS), sulfo-MBS or succinimidyl-iodoacetate, as described in the literature, to introduce 1-10 reactive groups. The modified Fc polypeptide is then reacted with a thiol-containing agent to produce a conjugate.

For example, where a TMAPP of the present disclosure comprises an Fc polypeptide, the polypeptide chain comprising the Fc polypeptide can be of the formula (A)-(L)-(C), where (A) is the polypeptide chain comprising the Fc polypeptide; where (L), if present, is a linker; and where (C) is an agent. (L), if present, links (A) to (C). In some cases, the polypeptide chain comprising the Fc polypeptide can comprise more than one agent (e.g., 2, 3, 4, or 5, or more than 5, agents).

Suitable drugs include, e.g., sulfasalazine, azathioprine, cyclophosphamide, antimalarials, D-penicillamine, cyclosporine. Suitable drugs include non-steroidal anti-inflammatory drugs; glucocorticoids; leflunomide; methotrexate; and the like.

### Exemplary T-Cell Modulatory Antigen-Presenting Polypeptides

The following are non-limiting examples of multimeric TMAPPs of the present disclosure.

1) In some cases, a multimeric TMAPP of the present disclosure comprises: a) a first polypeptide comprising, in order from N-terminus to C-terminus: i) an immunomodulatory polypeptide; ii) a linker; iii) an HLA α1 polypeptide; iv) an HLA α2 polypeptide; and v) an Ig Fc polypeptide; and b) a second polypeptide comprising, in order from N-terminus to C-terminus: i) a peptide that presents an epitope capable of binding to a TCR (e.g., a “self” epitope”); ii) a linker; iii) an HLA β1 polypeptide; and iv) an HLA β2 polypeptide. In some cases, the immunomodulatory polypeptide is a PD-L1 polypeptide. In some cases, the PD-L1 polypeptide comprises the following amino acid sequence: FTVTVPKDLYVVEYGSNMTIECKFPVEKQLDLAALIVYWEMEDKNIIQFVHGEEDLKVQ HSSYRQRARLLKDQLSLGNAALQITDVKLQDAGVYRCMISYGGADYKRITVKVNAPYN KINQRILVVDPVTSEHELTCQAEGYPKAEVIWTSSDHQVLSGKTTTTNSKREEKLFNVTS TLRINTTTNEIFYCTFRRLDPEENHTAELVIPELPLAHPPNER (SEQ ID NO: 297) and has a length of 220 amino acids. In some cases, the HLA α1 and HLA α2 polypeptides together comprise the following amino acid sequence: IKEEHVIIQAEFYLNPDQSGEFMFDFDGDEIFHVDMAKKETVWRLEEFGRFASFEAQGAL ANIAVDKANLEIMTKRSNYTPITNVPPEVTVLTNSPVELREPNVLICFIDKFTPPVVNVTW LRNGKPVTTGVSETVFLPREDHLFRKFHYLPFLPSTEDVYDCRVEHWGLDEPLLKHWEF DAPSPLPET (SEQ ID NO: 298) and have a length of 189 amino acids. In some cases, the Ig Fc polypeptide is an IgG1 Fc polypeptide with L14A and L15A substitutions, e.g., where the Ig Fc polypeptide comprises the following amino acid sequence: DKTHTCPPCPAPEAAGGPSVFLFPPKPKDTLMISRTPEVTCVVVDVSHEDPEVKFNWYV DGVEVHNAKTKPREEQYNSTYRVVSVLTVLHQDWLNGKEYKCKVSNKALPAPIEKTIS KAKGQPREPQVYTLPPSREEMTKNQVSLTCLVKGFYPSDIAVEWESNGQPENNYKTTPP VLDSDGSFFLYSKLTVDKSRWQQGNVFSCSVMHEALHNHYTQKSLSLSPG (SEQ ID NO: 299) and has a length of 226 amino acids. In some cases, the peptide epitope is an autoantigen associated with multiple sclerosis. In some cases, the HLA β1 and β2 polypeptides together comprise the following amino acid sequence: GDTRPRFLEQVKHECHFFNGTERVRFLDRYFYHQEEYVRFDSDVGEYRAVTELGRPDA EYWNSQKDLLEQKRAAVDTYCRHNYGVGESFTVQRRVYPEVTVYPAKTQPLQHHNLL VCSVNGFYPASIEVRWFRNGQEEKTGVVSTGLIQNGDWTFQTLVMLETVPRSGEVYTC QVEHPSLTSPLTVEWRARSESAQSKM (SEQ ID NO: 300), and have a length of 199 amino acids. A suitable linker can be GGGGSGGGGSGGGGS (SEQ ID NO: 301) or GGGGSGGGGSGGGGSGGGGS (SEQ ID NO: 302).

2) In some cases, a multimeric TMAPP of the present disclosure comprises: a) a first polypeptide comprising, in order from N-terminus to C-terminus: i) an HLA α1 polypeptide; ii) an HLA α2 polypeptide; iii) a linker; iv) an immunomodulatory polypeptide; v) a linker; and vi) an Ig Fc polypeptide; and b) a second polypeptide comprising, in order from N-terminus to C-terminus: i) a peptide that presents an epitope capable of binding to a TCR (e.g., a “self” epitope); ii) a linker; iii) an HLA β1 polypeptide; and iv) an HLA β2 polypeptide. In some cases, the immunomodulatory polypeptide is a PD-L1 polypeptide. In some cases, the PD-L1 polypeptide comprises the following amino acid sequence: FTVTVPKDLYVVEYGSNMTIECKFPVEKQLDLAALIVYWEMEDKNIIQFVHGEEDLKVQ HSSYRQRARLLKDQLSLGNAALQITDVKLQDAGVYRCMISYGGADYKRITVKVNAPYN KINQRILVVDPVTSEHELTCQAEGYPKAEVIWTSSDHQVLSGKTTTTNSKREEKLFNVTS TLRINTTTNEIFYCTFRRLDPEENHTAELVIPELPLAHPPNER (SEQ ID NO: 297) and has a length of 220 amino acids. In some cases, the HLA α1 and HLA α2 polypeptides together comprise the following amino acid sequence: IKEEHVIIQAEFYLNPDQSGEFMFDFDGDEIFHVDMAKKETVWRLEEFGRFASFEAQGAL ANIAVDKANLEIMTKRSNYTPITNVPPEVTVLTNSPVELREPNVLICFIDKFTPPVVNVTW LRNGKPVTTGVSETVFLPREDHLFRKFHYLPFLPSTEDVYDCRVEHWGLDEPLLKHWEF DAPSPLPET (SEQ ID NO: 298) and have a length of 189 amino acids. In some cases, the Ig Fc polypeptide is an IgG1 Fc polypeptide with L14A and L15A substitutions, e.g., where the Ig Fc polypeptide comprises the following amino acid sequence: DKTHTCPPCPAPEAAGGPSVFLFPPKPKDTLMISRTPEVTCVVVDVSHEDPEVKFNWYV DGVEVHNAKTKPREEQYNSTYRVVSVLTVLHQDWLNGKEYKCKVSNKALPAPIEKTIS KAKGQPREPQVYTLPPSREEMTKNQVSLTCLVKGFYPSDIAVEWESNGQPENNYKTTPP VLDSDGSFFLYSKLTVDKSRWQQGNVFSCSVMHEALHNHYTQKSLSLSPG (SEQ ID NO: 299) and has a length of 226 amino acids. In some cases, the peptide epitope is a self peptide. For example, in some cases, the peptide epitope is an autoantigen associated with rheumatoid arthritis. In some cases, the HLA β1 and β2 polypeptides together comprise the following amino acid sequence: GDTRPRFLEQVKHECHFFNGTERVRFLDRYFYHQEEYVRFDSDVGEYRAVTELGRPDA EYWNSQKDLLEQKRAAVDTYCRHNYGVGESFTVQRRVYPEVTVYPAKTQPLQHHNLL VCSVNGFYPASIEVRWFRNGQEEKTGVVSTGLIQNGDWTFQTLVMLETVPRSGEVYTC QVEHPSLTSPLTVEWRARSESAQSKM (SEQ ID NO: 300), and have a length of 199 amino acids. A suitable linker can be GGGGSGGGGSGGGGS (SEQ ID NO: 301) or GGGGSGGGGSGGGGSGGGGS (SEQ ID NO: 302).

3) In some cases, a multimeric TMAPP of the present disclosure comprises: a) a first polypeptide comprising, in order from N-terminus to C-terminus: i) an HLA α1 polypeptide; ii) an HLA α2 polypeptide; iii) a linker; iv) an Ig Fc polypeptide; and v) an immunomodulatory polypeptide; and b) a second polypeptide comprising, in order from N-terminus to C-terminus: i) a peptide that presents an epitope capable of binding to a TCR (e.g., a “self” epitope); ii) a linker; iii) an HLA β1 polypeptide; and iv) an HLA β2 polypeptide. In some cases, the immunomodulatory polypeptide is a PD-L1 polypeptide. In some cases, the PD-L1 polypeptide comprises the following amino acid sequence: FTVTVPKDLYVVEYGSNMTIECKFPVEKQLDLAALIVYWEMEDKNIIQFVHGEEDLKVQ HSSYRQRARLLKDQLSLGNAALQITDVKLQDAGVYRCMISYGGADYKRITVKVNAPYN KINQRILVVDPVTSEHELTCQAEGYPKAEVIWTSSDHQVLSGKTTTTNSKREEKLFNVTS TLRINTTTNEIFYCTFRRLDPEENHTAELVIPELPLAHPPNER (SEQ ID NO: 297) and has a length of 220 amino acids. In some cases, the HLA α1 and HLA α2 polypeptides together comprise the following amino acid sequence: IKEEHVIIQAEFYLNPDQSGEFMFDFDGDEIFHVDMAKKETVWRLEEFGRFASFEAQGAL ANIAVDKANLEIMTKRSNYTPITNVPPEVTVLTNSPVELREPNVLICFIDKFTPPVVNVTW LRNGKPVTTGVSETVFLPREDHLFRKFHYLPFLPSTEDVYDCRVEHWGLDEPLLKHWEF DAPSPLPET (SEQ ID NO: 298) and have a length of 189 amino acids. In some cases, the Ig Fc polypeptide is an IgG1 Fc polypeptide with L14A and L15A substitutions, e.g., where the Ig Fc polypeptide comprises the following amino acid sequence: DKTHTCPPCPAPEAAGGPSVFLFPPKPKDTLMISRTPEVTCVVVDVSHEDPEVKFNWYV DGVEVHNAKTKPREEQYNSTYRVVSVLTVLHQDWLNGKEYKCKVSNKALPAPIEKTIS KAKGQPREPQVYTLPPSREEMTKNQVSLTCLVKGFYPSDIAVEWESNGQPENNYKTTPP VLDSDGSFFLYSKLTVDKSRWQQGNVFSCSVMHEALHNHYTQKSLSLSPG (SEQ ID NO: 299) and has a length of 226 amino acids. In some cases, the peptide epitope is a self peptide. For example, in some cases, the peptide epitope associated with SLE. In some cases, the HLA β1 and β2 polypeptides together comprise the following amino acid sequence: GDTRPRFLEQVKHECHFFNGTERVRFLDRYFYHQEEYVRFDSDVGEYRAVTELGRPDA EYWNSQKDLLEQKRAAVDTYCRHNYGVGESFTVQRRVYPEVTVYPAKTQPLQHHNLL VCSVNGFYPASIEVRWFRNGQEEKTGVVSTGLIQNGDWTFQTLVMLETVPRSGEVYTC QVEHPSLTSPLTVEWRARSESAQSKM (SEQ ID NO: 300), and have a length of 199 amino acids. A suitable linker can be GGGGSGGGGSGGGGS (SEQ ID NO: 301) or GGGGSGGGGSGGGGSGGGGS (SEQ ID NO: 302).

4) In some cases, a multimeric TMAPP of the present disclosure comprises: a) a first polypeptide comprising, in order from N-terminus to C-terminus: i) an HLA α1 polypeptide; ii) an HLA α2 polypeptide; iii) a linker; and iv) an Ig Fc polypeptide; and b) a second polypeptide comprising, in order from N-terminus to C-terminus: i) an immunomodulatory polypeptide; ii) a linker; iii) a peptide that presents an epitope capable of binding to a TCR (e.g., a “self” epitope); iv) a linker; v) an HLA β1 polypeptide; and vi) an HLA β2 polypeptide. In some cases, the immunomodulatory polypeptide is a PD-L1 polypeptide. In some cases, the PD-L1 polypeptide comprises the following amino acid sequence: FTVTVPKDLYVVEYGSNMTIECKFPVEKQLDLAALIVYWEMEDKNIIQFVHGEEDLKVQ HSSYRQRARLLKDQLSLGNAALQITDVKLQDAGVYRCMISYGGADYKRITVKVNAPYN KINQRILVVDPVTSEHELTCQAEGYPKAEVIWTSSDHQVLSGKTTTTNSKREEKLFNVTS TLRINTTTNEIFYCTFRRLDPEENHTAELVIPELPLAHPPNER (SEQ ID NO: 297) and has a length of 220 amino acids. In some cases, the HLA α1 and HLA α2 polypeptides together comprise the following amino acid sequence: IKEEHVIIQAEFYLNPDQSGEFMFDFDGDEIFHVDMAKKETVWRLEEFGRFASFEAQGAL ANIAVDKANLEIMTKRSNYTPITNVPPEVTVLTNSPVELREPNVLICFIDKFTPPVVNVTW LRNGKPVTTGVSETVFLPREDHLFRKFHYLPFLPSTEDVYDCRVEHWGLDEPLLKHWEF DAPSPLPET (SEQ ID NO: 298) and have a length of 189 amino acids. In some cases, the Ig Fc polypeptide is an IgG1 Fc polypeptide with L14A and L15A substitutions, e.g., where the Ig Fc polypeptide comprises the following amino acid sequence: DKTHTCPPCPAPEAAGGPSVFLFPPKPKDTLMISRTPEVTCVVVDVSHEDPEVKFNWYV DGVEVHNAKTKPREEQYNSTYRVVSVLTVLHQDWLNGKEYKCKVSNKALPAPIEKTIS KAKGQPREPQVYTLPPSREEMTKNQVSLTCLVKGFYPSDIAVEWESNGQPENNYKTTPP VLDSDGSFFLYSKLTVDKSRWQQGNVFSCSVMHEALHNHYTQKSLSLSPG (SEQ ID NO: 299) and has a length of 226 amino acids. In some cases, the peptide epitope is a self peptide. For example, in some cases, the peptide epitope is associated with Sjögren's syndrome. In some cases, the HLA β1 and β2 polypeptides together comprise the following amino acid sequence: GDTRPRFLEQVKHECHFFNGTERVRFLDRYFYHQEEYVRFDSDVGEYRAVTELGRPDA EYWNSQKDLLEQKRAAVDTYCRHNYGVGESFTVQRRVYPEVTVYPAKTQPLQHHNLL VCSVNGFYPASIEVRWFRNGQEEKTGVVSTGLIQNGDWTFQTLVMLETVPRSGEVYTC QVEHPSLTSPLTVEWRARSESAQSKM (SEQ ID NO: 300), and have a length of 199 amino acids. A suitable linker can be GGGGSGGGGSGGGGS (SEQ ID NO: 301) or GGGGSGGGGSGGGGSGGGGS (SEQ ID NO: 302).

5) In some cases, a multimeric TMAPP of the present disclosure comprises: a) a first polypeptide comprising, in order from N-terminus to C-terminus: i) an HLA α1 polypeptide; ii) an HLA α2 polypeptide; iii) a linker; and iv) an Ig Fc polypeptide; and b) a second polypeptide comprising, in order from N-terminus to C-terminus: i) a peptide that presents an epitope capable of binding to a TCR (e.g., a “self” epitope); ii) a linker; iii) an HLA β1 polypeptide; iv) an HLA β2 polypeptide; and v) an immunomodulatory polypeptide. In some cases, the immunomodulatory polypeptide is a PD-L1 polypeptide. In some cases, the PD-L1 polypeptide comprises the following amino acid sequence: FTVTVPKDLYVVEYGSNMTIECKFPVEKQLDLAALIVYWEMEDKNIIQFVHGEEDLKVQ HSSYRQRARLLKDQLSLGNAALQITDVKLQDAGVYRCMISYGGADYKRITVKVNAPYN KINQRILVVDPVTSEHELTCQAEGYPKAEVIWTSSDHQVLSGKTTTTNSKREEKLFNVTS TLRINTTTNEIFYCTFRRLDPEENHTAELVIPELPLAHPPNER (SEQ ID NO: 297) and has a length of 220 amino acids. In some cases, the HLA α1 and HLA α2 polypeptides together comprise the following amino acid sequence: IKEEHVIIQAEFYLNPDQSGEFMFDFDGDEIFHVDMAKKETVWRLEEFGRFASFEAQGAL ANIAVDKANLEIMTKRSNYTPITNVPPEVTVLTNSPVELREPNVLICFIDKFTPPVVNVTW LRNGKPVTTGVSETVFLPREDHLFRKFHYLPFLPSTEDVYDCRVEHWGLDEPLLKHWEF DAPSPLPET (SEQ ID NO: 298) and have a length of 189 amino acids. In some cases, the Ig Fc polypeptide is an IgG1 Fc polypeptide with L14A and L15A substitutions, e.g., where the Ig Fc polypeptide comprises the following amino acid sequence: DKTHTCPPCPAPEAAGGPSVFLFPPKPKDTLMISRTPEVTCVVVDVSHEDPEVKFNWYV DGVEVHNAKTKPREEQYNSTYRVVSVLTVLHQDWLNGKEYKCKVSNKALPAPIEKTIS KAKGQPREPQVYTLPPSREEMTKNQVSLTCLVKGFYPSDIAVEWESNGQPENNYKTTPP VLDSDGSFFLYSKLTVDKSRWQQGNVFSCSVMHEALHNHYTQKSLSLSPG (SEQ ID NO: 299) and has a length of 226 amino acids. In some cases, the peptide epitope is a self peptide. For example, in some cases, the peptide epitope is associated with Grave's disease. In some cases, the HLA β1 and β2 polypeptides together comprise the following amino acid sequence: GDTRPRFLEQVKHECHFFNGTERVRFLDRYFYHQEEYVRFDSDVGEYRAVTELGRPDA EYWNSQKDLLEQKRAAVDTYCRHNYGVGESFTVQRRVYPEVTVYPAKTQPLQHHNLL VCSVNGFYPASIEVRWFRNGQEEKTGVVSTGLIQNGDWTFQTLVMLETVPRSGEVYTC QVEHPSLTSPLTVEWRARSESAQSKM (SEQ ID NO: 300), and have a length of 199 amino acids. A suitable linker can be GGGGSGGGGSGGGGS (SEQ ID NO: 301) or GGGGSGGGGSGGGGSGGGGS (SEQ ID NO: 302).

### Additional Exemplary Multimeric TMAPPs

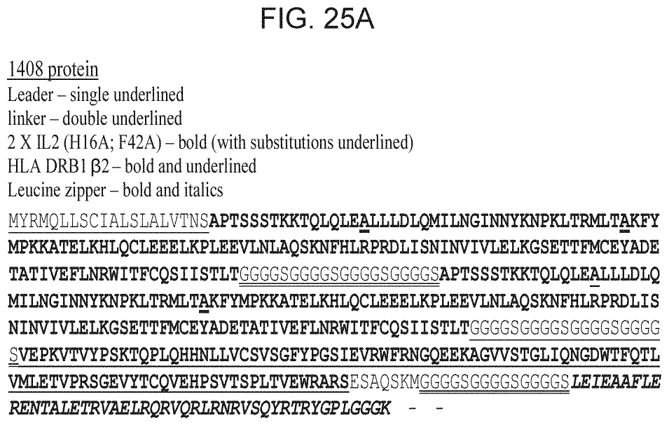

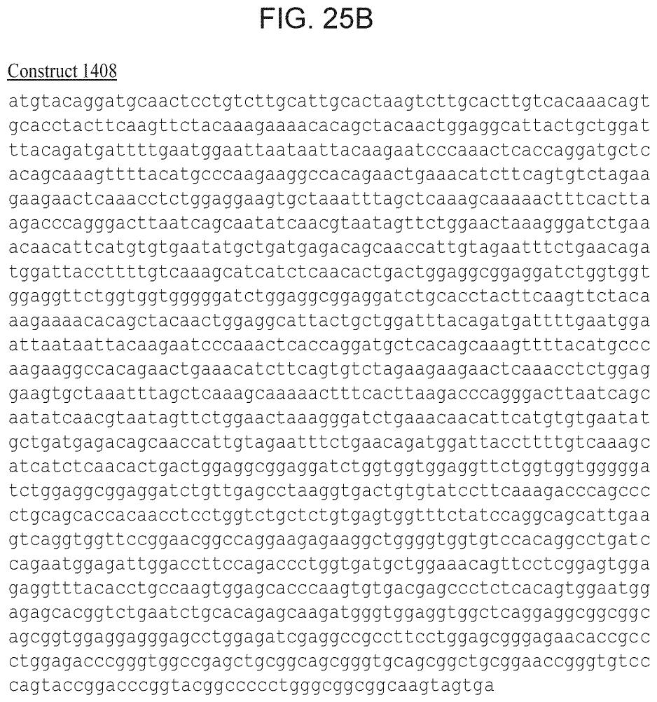

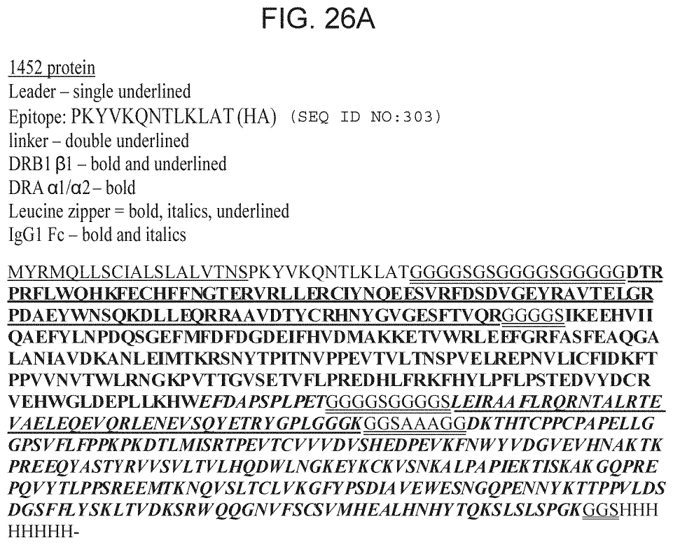

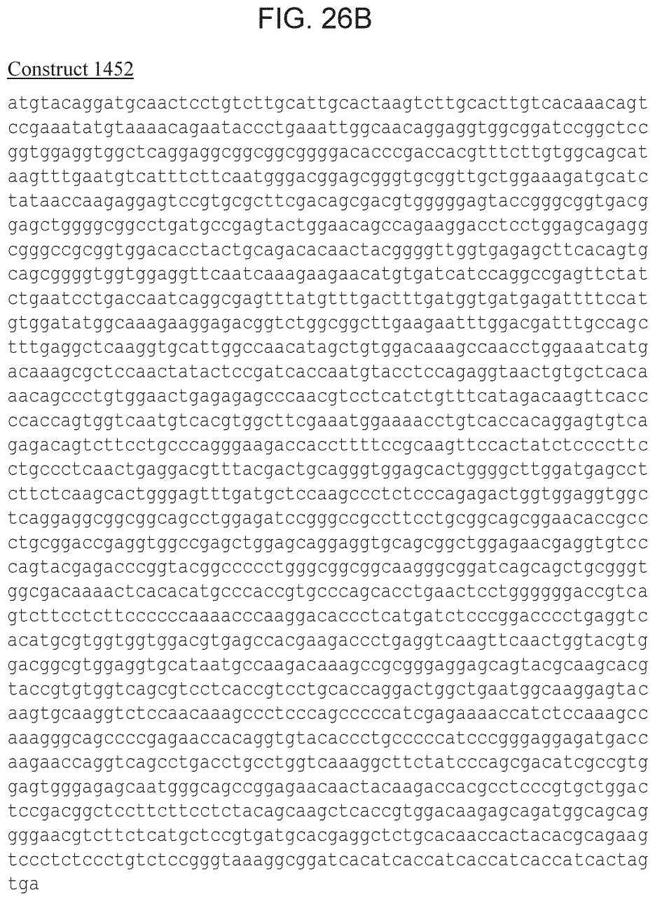

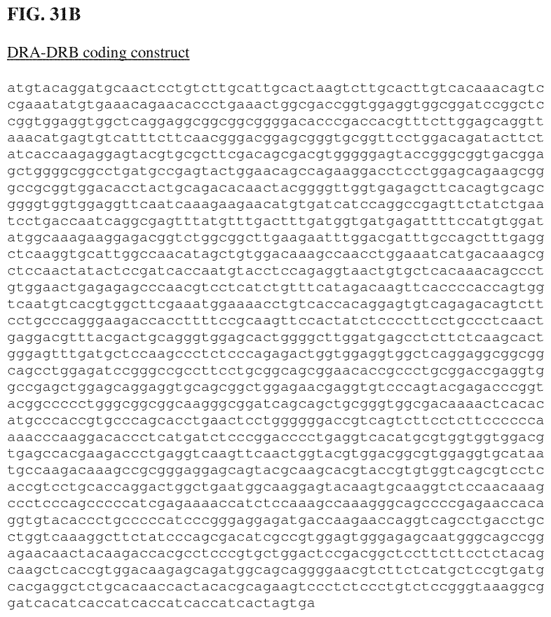

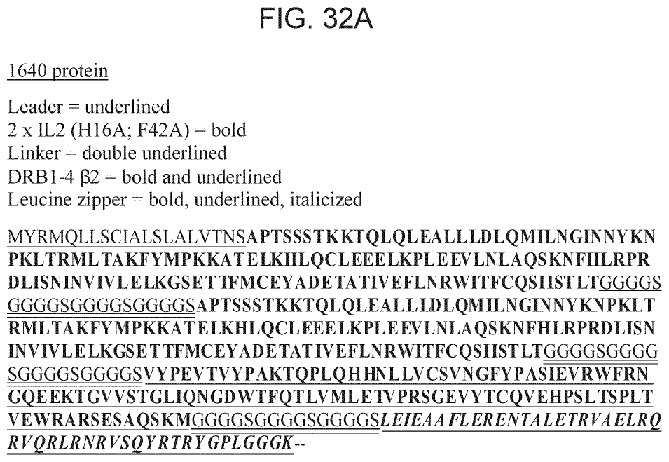

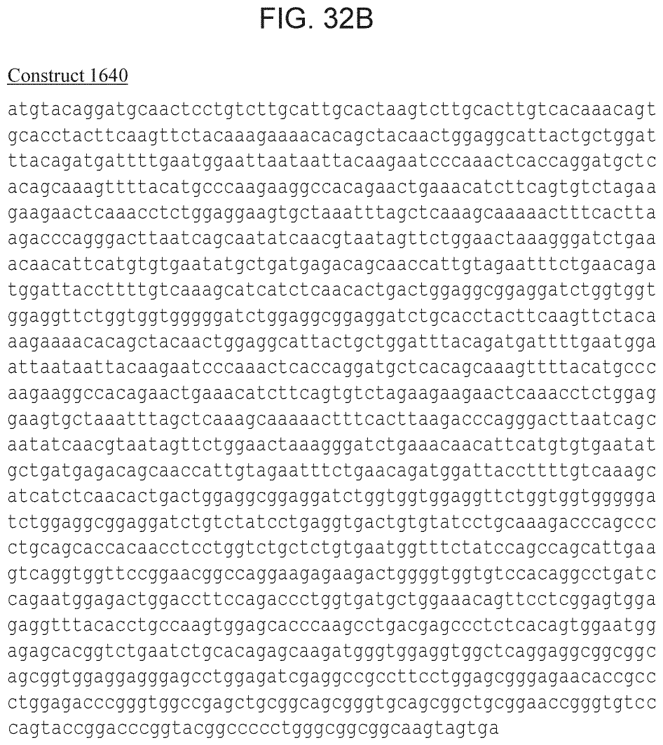

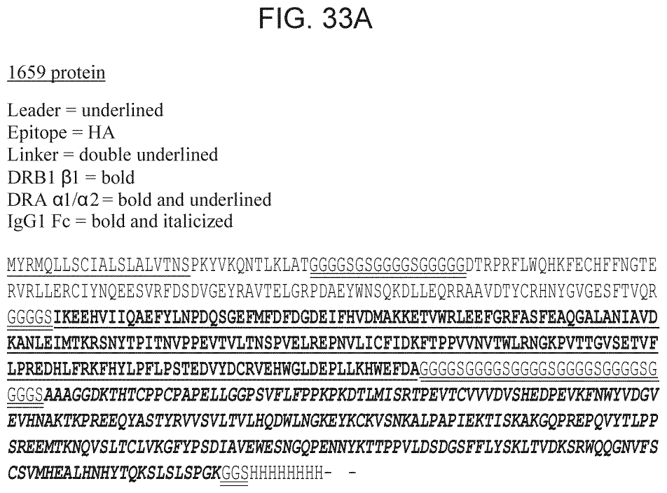

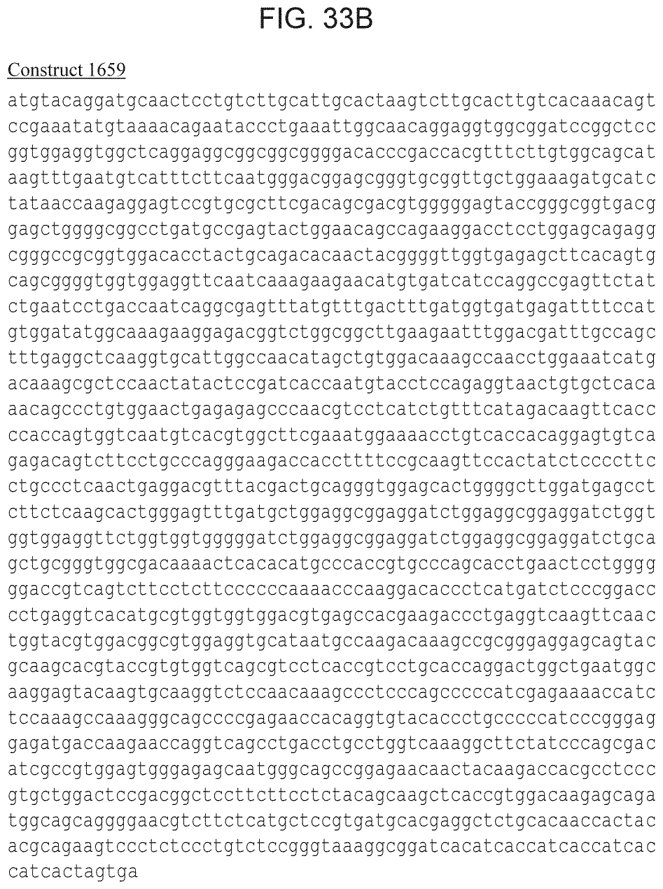

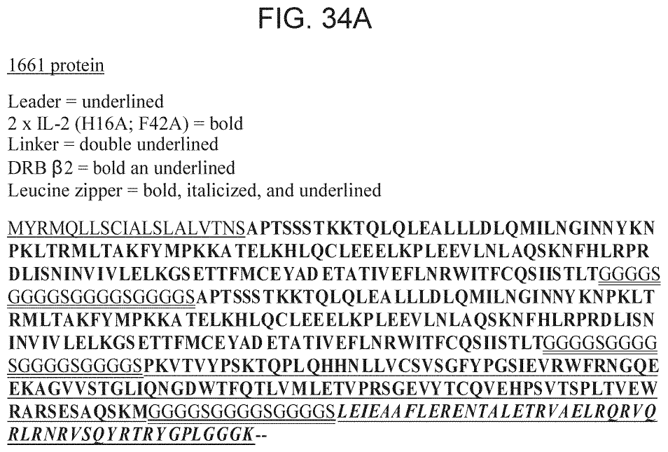

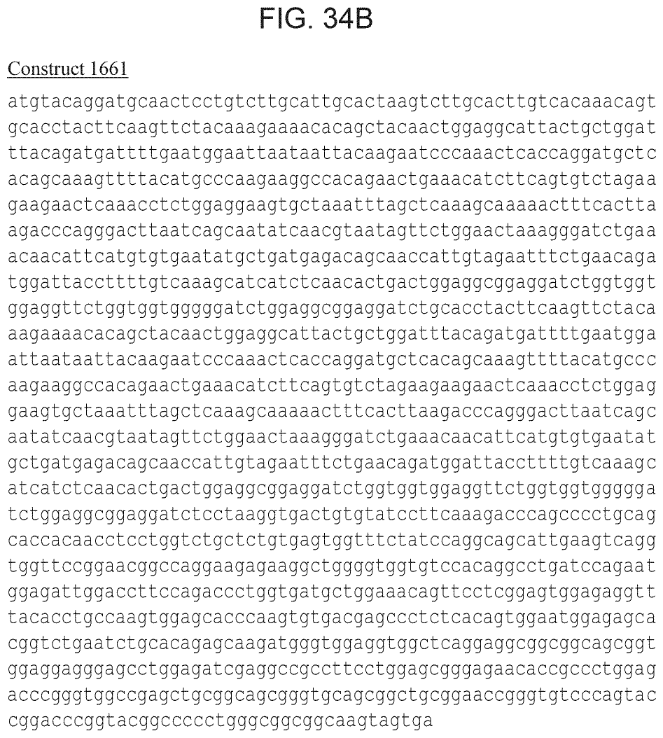

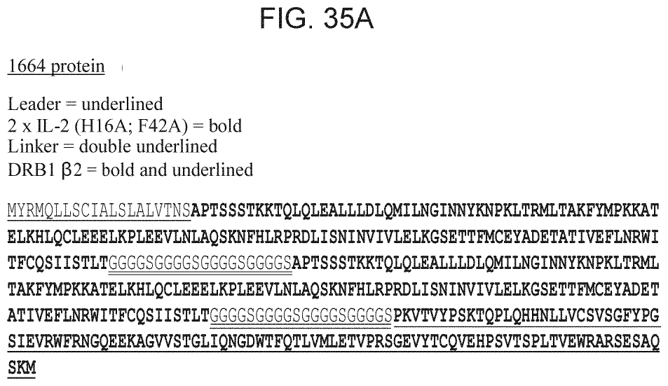

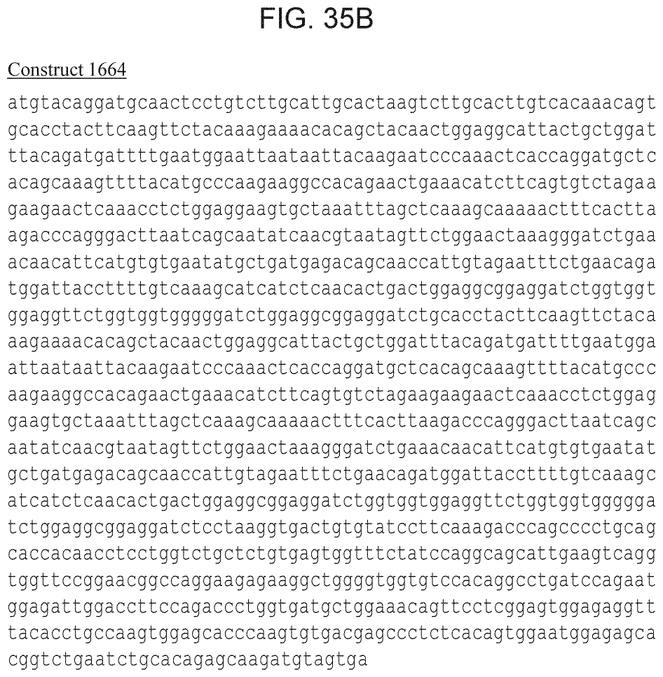

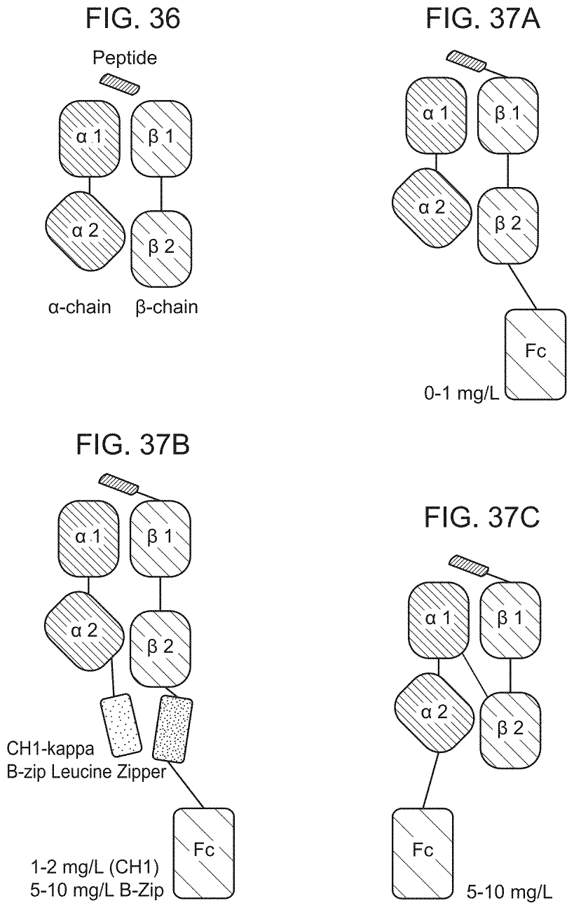

The following are non-limiting examples of multimeric TMAPPs of the present disclosure employing the following pairs of polypeptides: 1) the 1452 polypeptide depicted in FIG. 26A and the 1661 polypeptide depicted in FIG. 34A; 2) the 1659 polypeptide depicted in FIG. 33A and the 1664 polypeptide depicted in FIG. 35A; 3) the 1637 polypeptide depicted in FIG. and the 1408 polypeptide depicted in FIG. 25A; and the “DRA-DRB-epitope construct” polypeptide depicted in FIG. 31A and the 1640 polypeptide depicted in FIG. 32A. A TMAPP to be administered to an individual in need thereof will generally not include a leader sequence or a histidine tag as depicted in the aforementioned figures.

1) 1452+1661. In some cases, a multimeric TMAPP of the present disclosure comprises: a) a first polypeptide comprising, in order from N-terminus to C-terminus: i) an epitope; ii) a linker; iii) an HLA β1 polypeptide; iv) an HLA α1 polypeptide; v) an HLA α2 polypeptide; vi) a dimerizer polypeptide; and vii) an Ig Fc polypeptide; and b) a second polypeptide comprising, in order from N-terminus to C-terminus: i) a first immunomodulatory polypeptide (e.g., a variant immunomodulatory polypeptide with reduced affinity for its cognate co-immunomodulatory polypeptide); ii) a second immunomodulatory polypeptide (e.g., a variant immunomodulatory polypeptide with reduced affinity for its cognate co-immunomodulatory polypeptide); iii) an HLA β2 polypeptide; and iv) a dimerizer polypeptide. As one non-limiting example, a multimeric TMAPP of the present disclosure can comprise: a) a first polypeptide comprising, in order from N-terminus to C-terminus: i) an epitope; ii) a linker; iii) an HLA DRB1 β1 polypeptide; iv) an HLA DRA α1 polypeptide; v) an HLA DRA α2 polypeptide; vi) a leucine zipper dimerizer polypeptide; and vii) an IgG1 Fc polypeptide; and b) a second polypeptide comprising, in order from N-terminus to C-terminus: i) a first immunomodulatory polypeptide (e.g., a variant IL-2 polypeptide comprising H16A and F42A substitutions); ii) a second immunomodulatory polypeptide (e.g., a variant IL-2 polypeptide comprising H16A and F42A substitutions); iii) an HLA DRB β2 polypeptide; and iv) a leucine zipper dimerizer polypeptide. In some cases, the epitope is a hemagglutinin epitope, e.g., PKYVKQNTLKLAT (SEQ ID NO: 303). In some cases, the variant IL-2 polypeptide comprises the following amino acid sequence: APTSSSTKKTQLQLEALLLDLQMILNGINNYKNPKLTRMLTAKFYMPKKATELKHLQCL EEELKPLEEVLNLAQSKNFHLRPRDLISNINVIVLELKGSETTFMCEYADETATIVEFLNR WITFCQSIISTLT (SEQ ID NO: 304), where the H16A and F42A substitutions are underlined. In some cases, the HLA-DRB1 β1 polypeptide comprises the following amino acid sequence: DTRPRFLWQHKFECHFFNGTERVRLLERCIYNQEESVRFDSDVGEYRAVTELGRPDAEY WNSQKDLLEQRRAAVDTYCRHNYGVGESFTVQR (SEQ ID NO: 305). In some cases, the HLA DRA α1 polypeptide comprises the following amino acid sequence IKEEHVIIQAEFYLNPDQSGEFMFDFDGDEIFHVDMAKKETVWRLEEFGRFASFEAQGAL ANIAVDKANLEIMTKRSNYTPITN (SEQ ID NO: 306). In some cases, the HLA DRA α2 polypeptide comprises the following amino acid sequence VPPEVTVLTNSPVELREPNVLICFIDKFTPPVVNVTWLRNGKPVTTGVSETVFLPREDHL FRKFHYLPFLPSTEDVYDCRVEHWGLDEPLLKHWEFDAPSPLPET (SEQ ID NO: 307). In some cases, the leucine zipper dimerizer polypeptide comprises the following amino acid sequence: LEIRAAFLRQRNTALRTEVAELEQEVQRLENEVSQYETRYGPLGGGK (SEQ ID NO: 308). In some cases, the IgG1 Fc polypeptide comprises the following amino acid sequence: DKTHTCPPCPAPELLGGPSVFLFPPKPKDTLMISRTPEVTCVVVDVSHEDPEVKFNWYV DGVEVHNAKTKPREEQYASTYRVVSVLTVLHQDWLNGKEYKCKVSNKALPAPIEKTIS KAKGQPREPQVYTLPPSREEMTKNQVSLTCLVKGFYPSDIAVEWESNGQPENNYKTTPP VLDSDGSFFLYSKLTVDKSRWQQGNVFSCSVMHEALHNHYTQKSLSLSPGK (SEQ ID NO: 309). In some cases, the first polypeptide comprises the 1452 amino acid sequence depicted in FIG. 26A, without the leader sequence and without the C-terminal linker and histidine tag. For example, in some cases, the first polypeptide comprises amino acids 21 to 628 of the 1452 amino acid sequence depicted in FIG. 26A. In some cases, the second polypeptide comprises the 1661 amino acid sequence depicted in FIG. 34A, without the leader sequence. For example, in some cases, the second polypeptide comprises amino acids 21 to 491 of the amino acid sequence depicted in FIG. 34A. In some cases, the epitope of the first polypeptide is not PKYVKQNTLKLAT (SEQ ID NO: 303), but instead is substituted with a different epitope.

2) 1659+1664. In some cases, a multimeric TMAPP of the present disclosure comprises: a) a first polypeptide comprising, in order from N-terminus to C-terminus: i) an epitope; ii) an HLA β1 polypeptide; iii) an HLA α1 polypeptide; iv) an HLA α2 polypeptide; and v) an Ig Fc polypeptide; and b) a second polypeptide comprising, in order from N-terminus to C-terminus: i) a first immunomodulatory polypeptide (e.g., a variant immunomodulatory polypeptide with reduced affinity for its cognate co-immunomodulatory polypeptide); ii) a second immunomodulatory polypeptide (e.g., a variant immunomodulatory polypeptide with reduced affinity for its cognate co-immunomodulatory polypeptide); and iii) an HLA β2 polypeptide. As one non-limiting example, a multimeric TMAPP of the present disclosure can comprise: a) a first polypeptide comprising, in order from N-terminus to C-terminus: i) an epitope; ii) an HLA DRB1 β1 polypeptide; iii) an HLA DRA α1 polypeptide; iv) an HLA DRA α2 polypeptide; and v) an IgG1 Fc polypeptide; and b) a second polypeptide comprising, in order from N-terminus to C-terminus: i) a first immunomodulatory polypeptide (e.g., a variant IL-2 polypeptide comprising H16A and F42A substitutions); ii) a second immunomodulatory polypeptide (e.g., a variant IL-2 polypeptide comprising H16A and F42A substitutions); and iii) an HLA DRB1 β2 polypeptide. In some cases, the epitope is a hemagglutinin epitope, e.g., PKYVKQNTLKLAT (SEQ ID NO: 303). In some cases, the HLA DRB1 β1 polypeptide comprises the following amino acid sequence: DTRPRFLWQHKFECHFFNGTERVRLLERCIYNQEESVRFDSDVGEYRAVTELGRPDAEY WNSQKDLLEQRRAAVDTYCRHNYGVGESFTVQR (SEQ ID NO: 305). In some cases, the DRA α1 polypeptide comprises the following amino acid sequence: IKEEHVIIQAEFYLNPDQSGEFMFDFDGDEIFHVDMAKKETVWRLEEFGRFASFEAQGAL ANIAVDKANLEIMTKRSNYTPITN (SEQ ID NO: 306). In some cases, the DRA α2 polypeptide comprises the following amino acid sequence: VPPEVTVLTNSPVELREPNVLICFIDKFTPPVVNVTWLRNGKPVTTGVSETVFLPREDHL FRKFHYLPFLPSTEDVYDCRVEHWGLDEPLLKHWEFDA (SEQ ID NO: 320). In some cases, the IgG1 Fc polypeptide comprises the following amino acid sequence: DKTHTCPPCPAPELLGGPSVFLFPPKPKDTLMISRTPEVTCVVVDVSHEDPEVKFNWYV DGVEVHNAKTKPREEQYASTYRVVSVLTVLHQDWLNGKEYKCKVSNKALPAPIEKTIS KAKGQPREPQVYTLPPSREEMTKNQVSLTCLVKGFYPSDIAVEWESNGQPENNYKTTPP VLDSDGSFFLYSKLTVDKSRWQQGNVFSCSVMHEALHNHYTQKSLSLSPGK (SEQ ID NO: 309). In some cases, the variant IL-2 polypeptide comprises the following amino acid sequence: APTSSSTKKTQLQLEALLLDLQMILNGINNYKNPKLTRMLTAKFYMPKKATELKHLQCL EEELKPLEEVLNLAQSKNFHLRPRDLISNINVIVLELKGSETTFMCEYADETATIVEFLNR WITFCQSIISTLT (SEQ ID NO: 304), where the H16A and F42A substitutions are underlined. In some cases, the HLA DRB1 β2 polypeptide comprises the following amino acid sequence: PKVTVYPSKTQPLQHHNLLVCSVSGFYPGSIEVRWFRNGQEEKAGVVSTGLIQNGDWTF QTLVMLETVPRSGEVYTCQVEHPSVTSPLTVEWRARSESAQSKM (SEQ ID NO: 310). In some cases, the first polypeptide comprises the 1659 amino acid sequence depicted in FIG. 33A, without the leader peptide and without the C-terminal linker and histidine tag. For example, in some cases, the first polypeptide comprises amino acids 21 to 591 of the 1659 amino acid sequence depicted in FIG. 33A. In some cases, the epitope is not PKYVKQNTLKLAT (SEQ ID NO: 303), but instead is substituted with a different epitope. In some cases, the second polypeptide comprises the 1664 amino acid sequence depicted in FIG. 35A, without the leader sequence. For example, in some cases, the second polypeptide comprises amino acids 21 to 429 of the 1664 amino acid sequence depicted in FIG. 35A.

3) 1637-1408. In some cases, a multimeric TMAPP of the present disclosure comprises: a) a first polypeptide comprising, in order from N-terminus to C-terminus: i) an epitope; ii) an HLA β1 polypeptide; iii) an HLA α1 polypeptide; iv) an HLA α2 polypeptide; v) a dimerizer polypeptide; and vi) an Ig Fc polypeptide; and b) a second polypeptide comprising, in order from N-terminus to C-terminus: i) a first immunomodulatory polypeptide (e.g., a variant immunomodulatory polypeptide with reduced affinity for its cognate co-immunomodulatory polypeptide); ii) a second immunomodulatory polypeptide (e.g., a variant immunomodulatory polypeptide with reduced affinity for its cognate co-immunomodulatory polypeptide); iii) an HLA β2 polypeptide; and iv) a dimerizer polypeptide. As one non-limiting example, a multimeric TMAPP of the present disclosure can comprise: a) a first polypeptide comprising, in order from N-terminus to C-terminus: i) an epitope; ii) an HLA DRB1 β1 polypeptide; iii) an HLA DRA α1 polypeptide; iv) an HLA DRA α2 polypeptide; v) a leucine zipper dimerizer polypeptide; and vi) an IgG1 Fc polypeptide; and b) a second polypeptide comprising, in order from N-terminus to C-terminus: i) a first immunomodulatory polypeptide (e.g., a variant IL-2 polypeptide comprising H16A and F42A substitutions); ii) a second immunomodulatory polypeptide (e.g., a variant IL-2 polypeptide comprising H16A and F42A substitutions); iii) an HLA DRB1β2 polypeptide; and iv) a leucine zipper dimerizer polypeptide. In some cases, the epitope is a cytomegalovirus (CMV) pp65 epitope (LPLKMLNIPSINVH; SEQ ID NO: 312). In some cases, the HLA DRB β1 polypeptide comprises the following amino acid sequence: DTRPRFLWQHKFECHFFNGTERVRLLERCIYNQEESVRFDSDVGEYRAVTELGRPAAEY WNSQKDLLEQRRAAVDTYCRHNYGVGESFTVQR (SEQ ID NO: 433). In some cases, the HLA DRA α1 polypeptide comprises the following amino acid sequence: IKEEHVIIQAEFYLNPDQSGEFMFDFDGDEIFHVDMAKKETVWRLEEFGRFASFEAQGAL ANIAVDKANLEIMTKRSNYTPITN (SEQ ID NO: 306). In some cases, the HLA DRA α2 polypeptide comprises the following amino acid sequence: VPPEVTVLTNSPVELREPNVLICFIDKFTPPVVNVTWLRNGKPVTTGVSETVFLPREDHL FRKFHYLPFLPSTEDVYDCRVEHWGLDEPLLKHWEFDAPSPLPET (SEQ ID NO: 307). In some cases, the leucine zipper polypeptide comprises the following amino acid sequence: LEIRAAFLRQRNTALRTEVAELEQEVQRLENEVSQYETRYGPLGGGK (SEQ ID NO: 308).

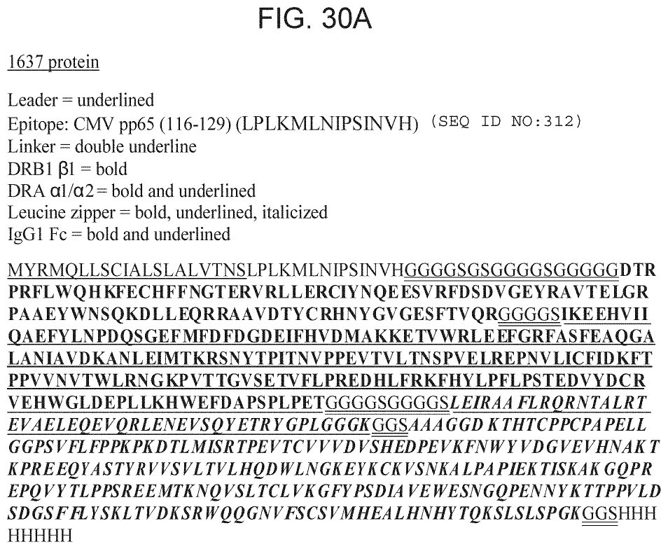

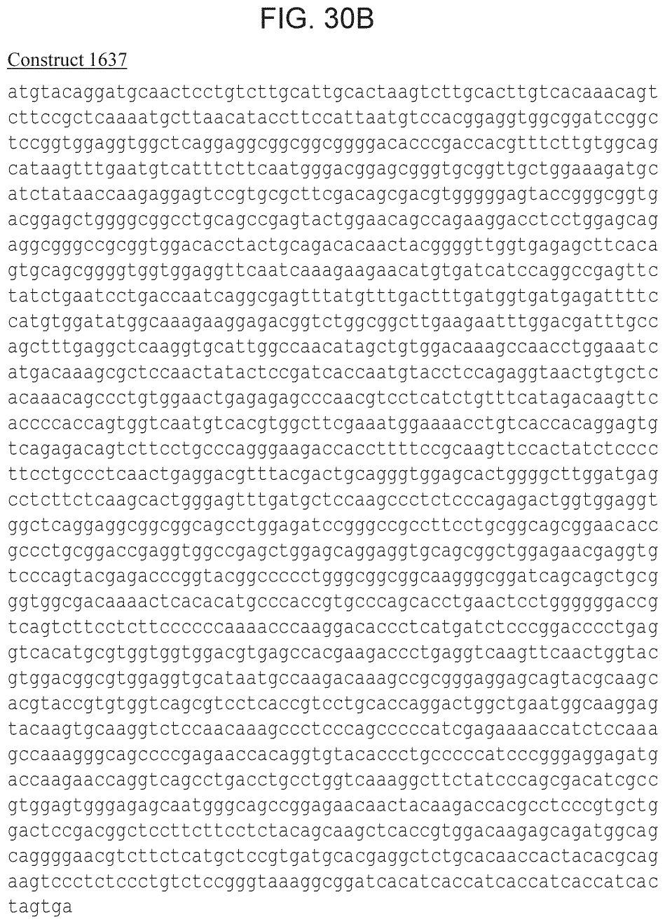

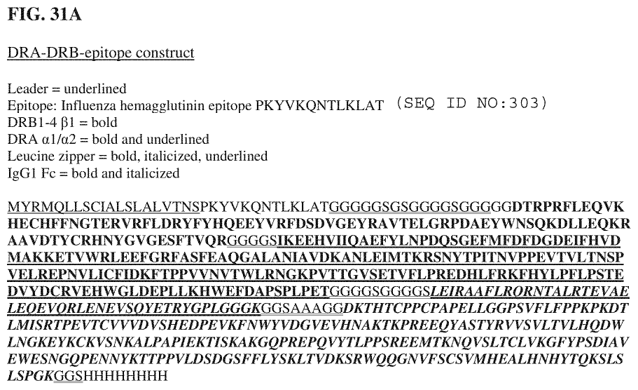

In some cases, the IgG1 Fc polypeptide comprises the following amino acid sequence: DKTHTCPPCPAPELLGGPSVFLFPPKPKDTLMISRTPEVTCVVVDVSHEDPEVKFNWYV DGVEVHNAKTKPREEQYASTYRVVSVLTVLHQDWLNGKEYKCKVSNKALPAPIEKTIS KAKGQPREPQVYTLPPSREEMTKNQVSLTCLVKGFYPSDIAVEWESNGQPENNYKTTPP VLDSDGSFFLYSKLTVDKSRWQQGNVFSCSVMHEALHNHYTQKSLSLSPGK (SEQ ID NO: 309). In some cases, the variant IL-2 polypeptide comprises the following amino acid sequence: APTSSSTKKTQLQLEALLLDLQMILNGINNYKNPKLTRMLTAKFYMPKKATELKHLQCL EEELKPLEEVLNLAQSKNFHLRPRDLISNINVIVLELKGSETTFMCEYADETATIVEFLNR WITFCQSIISTLT (SEQ ID NO: 304), where the H16A and F42A substitutions are underlined. In some cases, the HLA DRB1132 polypeptide comprises the following amino acid sequence: VEPKVTVYPSKTQPLQHHNLLVCSVSGFYPGSIEVRWFRNGQEEKAGVVSTGLIQNGD WTFQTLVMLETVPRSGEVYTCQVEHPSVTSPLTVEWRARSESAQSKM (SEQ ID NO: 311). In some cases, the leucine zipper polypeptide comprises the following amino acid sequence: LEIEAAFLERENTALETRVAELRQRVQRLRNRVSQYRTRYGPLGGGK (SEQ ID NO: 278). In some cases, the first polypeptide comprises the 1637 amino acid sequence depicted in FIG. 30A, without the leader sequence and without the C-terminal linker and histidine tag. For example, in some cases, the first polypeptide comprises amino acids 21-629 of the 1637 amino acid sequence depicted in FIG. 30A. In some cases, the first polypeptide does not include the epitope LPLKMLNIPSINVH (SEQ ID NO: 312); instead, the epitope is substituted with a different epitope. In some cases, the second polypeptide comprises the amino acid sequence depicted in FIG. 25A, but without the leader peptide. Thus, for example, in some cases, the second polypeptide comprises amino acids 21-493 of the amino acid sequence depicted in FIG. 25A.

4) “DRA-DRB-epitope”+1640. In some cases, a TMAPP of the present disclosure comprises: a) a first polypeptide comprising, in order from N-terminus to C-terminus: i) a peptide epitope; ii) a HLA β1 polypeptide; iii) an HLA α1 polypeptide; iv) an HLA α2 polypeptide; v) a dimerizer polypeptide; and vi) an Ig Fc polypeptide; and b) a second polypeptide comprising, in order from N-terminus to C-terminus: i) a first immunomodulatory polypeptide (e.g., a variant immunomodulatory polypeptide with reduced affinity for its cognate co-immunomodulatory polypeptide); ii) a second, independently selected, immunomodulatory polypeptide (e.g., a variant immunomodulatory polypeptide with reduced affinity for its cognate co-immunomodulatory polypeptide); iii) an HLA β2 polypeptide; and iv) a dimerizer polypeptide. As one non-limiting example, a TMAPP of the present disclosure can comprise: a) a first polypeptide comprising, in order from N-terminus to C-terminus: i) a peptide epitope; ii) an HLA DRB1-4 β1 polypeptide; iii) an HLA DRA α1 polypeptide; iv) an HLA DRA α2 polypeptide; v) a leucine zipper dimerizer polypeptide; and vi) an IgG1 Fc polypeptide; and b) a second polypeptide comprising, in order from N-terminus to C-terminus: i) a first immunomodulatory polypeptide (e.g., a variant IL-2 polypeptide comprising H16A and F42A substitutions); ii) a second, independently selected, immunomodulatory polypeptide (e.g., a variant IL-2 polypeptide comprising H16A and F42A substitutions); iii) an HLA DRB1-4 β2 polypeptide; and iv) a leucine zipper dimerizer polypeptide. In some cases, the epitope is a hemagglutinin epitope, e.g., PKYVKQNTLKLAT (SEQ ID NO: 303). In some cases, the epitope is not a hemagglutinin peptide; instead, the epitope is substituted with a different epitope. In some cases, the HLA DRB1-4 β1 polypeptide comprises the following amino acid sequence: DTRPRFLEQVKHECHFFNGTERVRFLDRYFYHQEEYVRFDSDVGEYRAVTELGRPDAE YWNSQKDLLEQKRAAVDTYCRHNYGVGESFTVQR (SEQ ID NO: 434). In some cases, the HLA DRA α1 polypeptide comprises the following amino acid sequence: IKEEHVIIQAEFYLNPDQSGEFMFDFDGDEIFHVDMAKKETVWRLEEFGRFASFEAQGAL ANIAVDKANLEIMTKRSNYTPITN (SEQ ID NO: 306). In some cases, the HLA DRA α2 polypeptide comprises the following amino acid sequence: VPPEVTVLTNSPVELREPNVLICFIDKFTPPVVNVTWLRNGKPVTTGVSETVFLPREDHL FRKFHYLPFLPSTEDVYDCRVEHWGLDEPLLKHWEFDAPSPLPET (SEQ ID NO: 307). In some cases, the leucine zipper polypeptide comprises the following amino acid sequence: LEIRAAFLRQRNTALRTEVAELEQEVQRLENEVSQYETRYGPLGGGK (SEQ ID NO: 308). In some cases, the IgG1 Fc polypeptide comprises the following amino acid sequence: DKTHTCPPCPAPELLGGPSVFLFPPKPKDTLMISRTPEVTCVVVDVSHEDPEVKFNWYV DGVEVHNAKTKPREEQYASTYRVVSVLTVLHQDWLNGKEYKCKVSNKALPAPIEKTIS KAKGQPREPQVYTLPPSREEMTKNQVSLTCLVKGFYPSDIAVEWESNGQPENNYKTTPP VLDSDGSFFLYSKLTVDKSRWQQGNVFSCSVMHEALHNHYTQKSLSLSPGK (SEQ ID NO: 309). In some cases, the variant IL-2 polypeptide comprises the following amino acid sequence: APTSSSTKKTQLQLEALLLDLQMILNGINNYKNPKLTRMLTAKFYMPKKATELKHLQCL EEELKPLEEVLNLAQSKNFHLRPRDLISNINVIVLELKGSETTFMCEYADETATIVEFLNR WITFCQSIISTLT (SEQ ID NO: 304), where the H16A and F42A substitutions are underlined. In some cases, the HLA DRB1-4 β2 polypeptide comprises the following amino acid sequence: VYPEVTVYPAKTQPLQHHNLLVCSVNGFYPASIEVRWFRNGQEEKTGVVSTGLIQNGD WTFQTLVMLETVPRSGEVYTCQVEHPSLTSPLTVEWRARSESAQSKM (SEQ ID NO: 313). In some cases, the leucine zipper polypeptide comprises the following amino acid sequence: LEIEAAFLERENTALETRVAELRQRVQRLRNRVSQYRTRYGPLGGGK (SEQ ID NO: 278). The amino acid sequences of the first polypeptide may be organized in a fashion similar to amino acids 21-629 of the DRA-DRB-epitope construct depicted in FIG. 31A e.g., without the leader sequence and without the C-terminal linker and histidine tag). The amino acid sequence of the second polypeptide may be organized in a fashion similar to amino acids 21-493 of protein/polypeptide construct 1640 depicted in FIG. 32A (without the leader sequence).

### Single-Chain TMAPPs

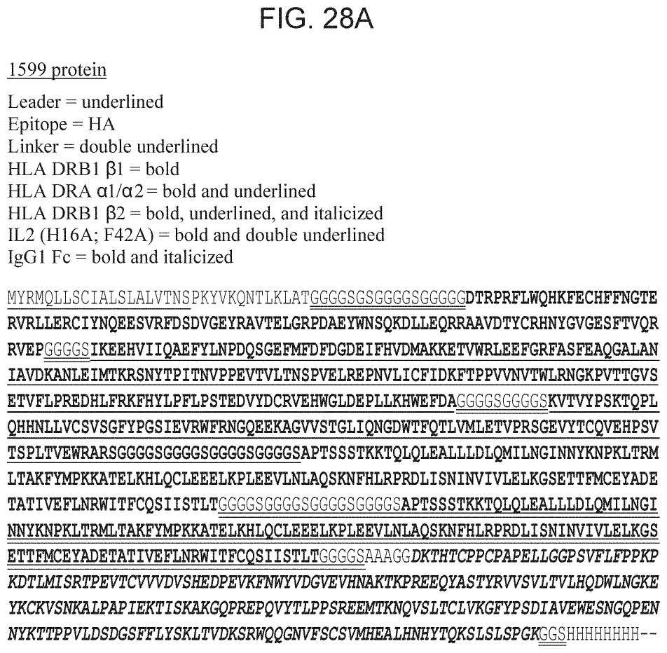

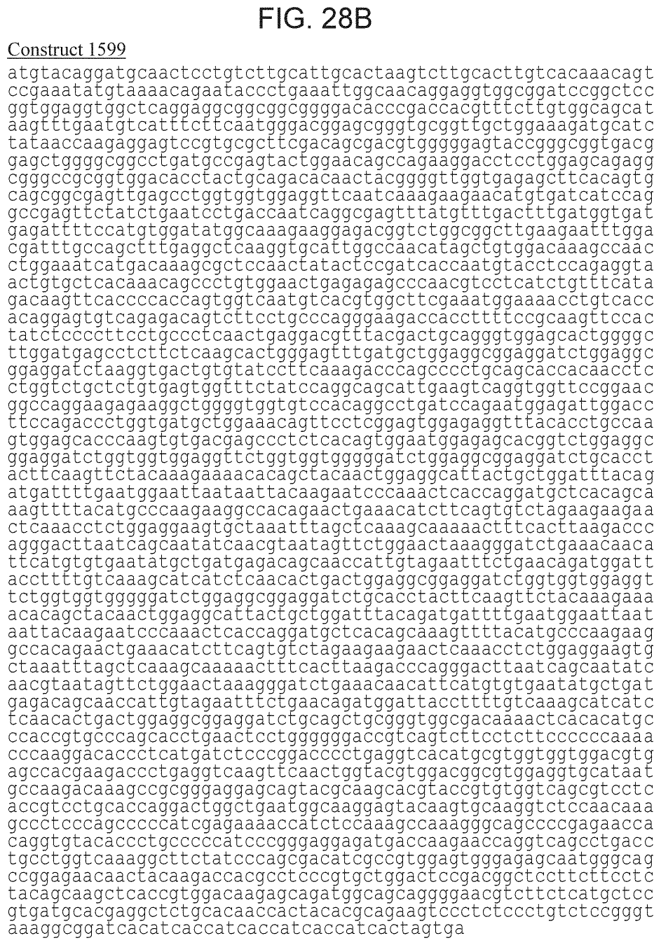

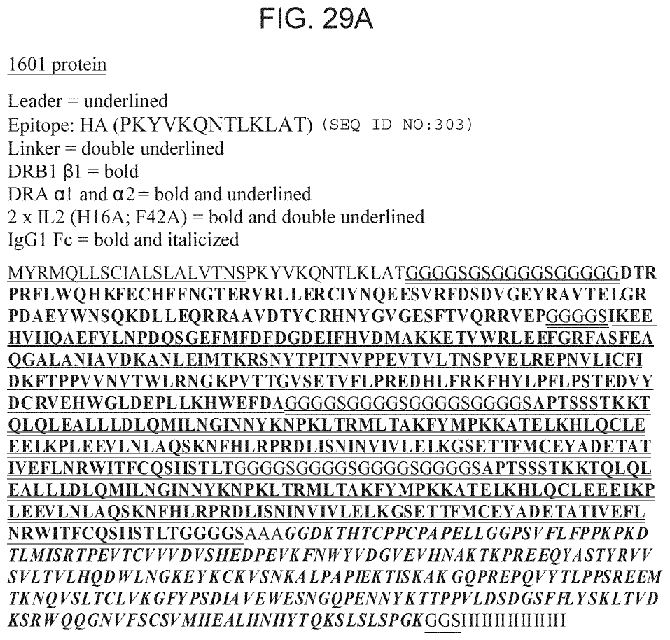

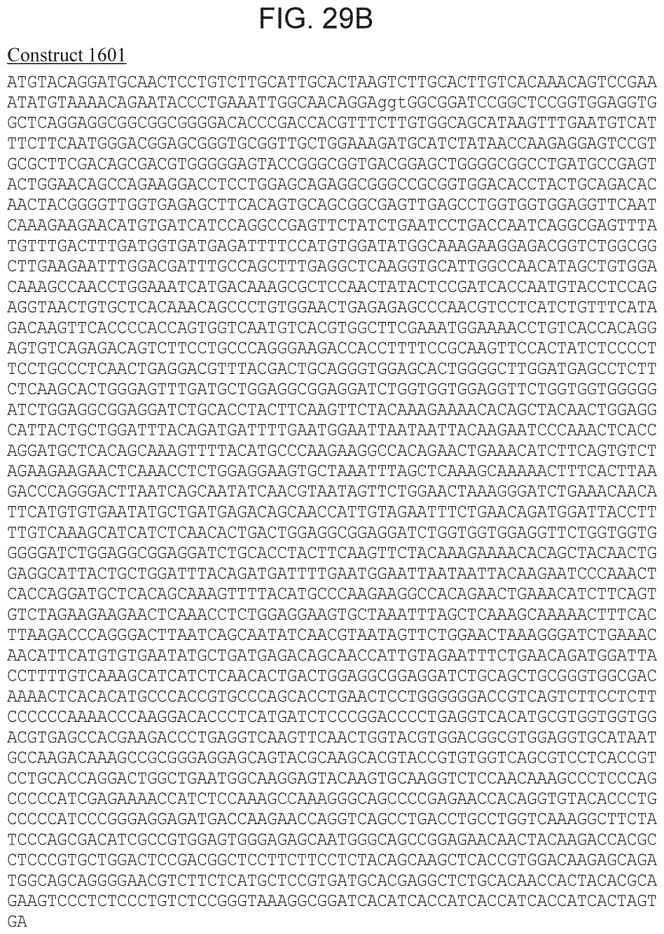

The following are non-limiting examples of single-chain TMAPPs of the present disclosure. See, e.g., FIG. 28A (1599 polypeptide); and FIG. 29A (1601 polypeptide). A TMAPP to be administered to an individual in need thereof will generally not include a leader sequence or a histidine tag as depicted in the aforementioned figures.

1) 1599. In some cases, a single-chain TMAPP of the present disclosure comprises, in order from N-terminus to C-terminus: i) an epitope; ii) an HLA β1 polypeptide; iii) an HLA α1 polypeptide; iv) an HLA α2 polypeptide; v) an HLA β2 polypeptide; vi) an immunomodulatory polypeptide (e.g., a variant immunomodulatory polypeptide with reduced affinity for its cognate co-immunomodulatory polypeptide); and vii) an Ig Fc polypeptide. As one non-limiting example, a single-chain TMAPP of the present disclosure can comprise, in order from N-terminus to C-terminus: i) an epitope; ii) an HLA DRB1 β1 polypeptide; iii) an HLA DRA α1 polypeptide; iv) an HLA DRA α2 polypeptide; v) an HLA DRB β2 polypeptide; vi) an immunomodulatory polypeptide (e.g., a variant IL-2 polypeptide comprising H16A and F42A substitutions); and vii) an IgG1 Fc polypeptide. In some cases, the epitope is a hemagglutinin epitope (e.g., PKYVKQNTLKLAT; SEQ ID NO: 303). In some cases, the HLA DRB1 β1 polypeptide comprises the following amino acid sequence: DTRPRFLWQHKFECHFFNGTERVRLLERCIYNQEESVRFDSDVGEYRAVTELGRPDAEY WNSQKDLLEQRRAAVDTYCRHNYGVGESFTVQRRVEP (SEQ ID NO: 315). In some cases, the HLA DRA α1 polypeptide comprises the following amino acid sequence: IKEEHVIIQAEFYLNPDQSGEFMFDFDGDEIFHVDMAKKETVWRLEEFGRFASFEAQGAL ANIAVDKANLEIMTKRSNYTPITN (SEQ ID NO: 306). In some cases, the HLA DRB β2 polypeptide comprises the following amino acid sequence: KVTVYPSKTQPLQHHNLLVCSVSGFYPGSIEVRWFRNGQEEKAGVVSTGLIQNGDWTF QTLVMLETVPRSGEVYTCQVEHPSVTSPLTVEWRARS (SEQ ID NO: 319). In some cases, the variant IL-2 polypeptide comprises the following amino acid sequence: APTSSSTKKTQLQLEALLLDLQMILNGINNYKNPKLTRMLTAKFYMPKKATELKHLQCL EEELKPLEEVLNLAQSKNFHLRPRDLISNINVIVLELKGSETTFMCEYADETATIVEFLNR WITFCQSIISTLT (SEQ ID NO: 304), where the H16A and F42A substitutions are underlined. In some cases, the IgG1 Fc polypeptide comprises the following amino acid sequence: DKTHTCPPCPAPELLGGPSVFLFPPKPKDTLMISRTPEVTCVVVDVSHEDPEVKFNWYV DGVEVHNAKTKPREEQYASTYRVVSVLTVLHQDWLNGKEYKCKVSNKALPAPIEKTIS KAKGQPREPQVYTLPPSREEMTKNQVSLTCLVKGFYPSDIAVEWESNGQPENNYKTTPP VLDSDGSFFLYSKLTVDKSRWQQGNVFSCSVMHEALHNHYTQKSLSLSPGK (SEQ ID NO: 309). In some cases, the single-chain polypeptide comprises the amino acid sequence depicted in FIG. 28A, without the leader peptide and without the C-terminal linker and histidine tag. For example, in some cases, the single-chain polypeptide comprises amino acids 21-981 of the amino acid sequence depicted in FIG. 28A. In some cases, the single-chain polypeptide does not include a hemagglutinin epitope (e.g., PKYVKQNTLKLAT; SEQ ID NO: 303); instead, the epitope is substituted with a different epitope.

2) 1601. In some cases, a single-chain TMAPP of the present disclosure comprises, in order from N-terminus to C-terminus: i) an epitope; ii) an HLA β1 polypeptide; iii) an HLA α1 polypeptide; iv) an HLA α2 polypeptide; v) a first immunomodulatory polypeptide (e.g., a variant immunomodulatory polypeptide with reduced affinity for its cognate co-immunomodulatory polypeptide); vi) a second immunomodulatory polypeptide (e.g., a variant immunomodulatory polypeptide with reduced affinity for its cognate co-immunomodulatory polypeptide); and vii) an Ig Fc polypeptide. As one non-limiting example, a single-chain TMAPP of the present disclosure can comprise, in order from N-terminus to C-terminus: i) an epitope; ii) an HLA DRB1β1 polypeptide; iii) an HLA DRA α1 polypeptide; iv) an HLA DRA α2 polypeptide; v) a first immunomodulatory polypeptide (e.g., a variant IL-2 polypeptide comprising H16A and F42A substitutions); vi) a second immunomodulatory polypeptide (e.g., a variant IL-2 polypeptide comprising H16A and F42A substitutions); and vii) an IgG1 Fc polypeptide. In some cases, the epitope is a hemagglutinin epitope (e.g., PKYVKQNTLKLAT; SEQ ID NO: 303). In some cases, the HLA DRB1 β polypeptide comprises the following amino acid sequence: DTRPRFLWQHKFECHFFNGTERVRLLERCIYNQEESVRFDSDVGEYRAVTELGRPDAEY WNSQKDLLEQRRAAVDTYCRHNYGVGESFTVQRRVEP (SEQ ID NO: 315). In some cases, the HLA DRA α1 polypeptide comprises the following amino acid sequence: IKEEHVIIQAEFYLNPDQSGEFMFDFDGDEIFHVDMAKKETVWRLEEFGRFASFEAQGAL ANIAVDKANLEIMTKRSNYTPITN (SEQ ID NO: 306). In some cases, the HLA DRA α2 polypeptide comprises the following amino acid sequence: VPPEVTVLTNSPVELREPNVLICFIDKFTPPVVNVTWLRNGKPVTTGVSETVFLPREDHL FRKFHYLPFLPSTEDVYDCRVEHWGLDEPLLKHWEFDA (SEQ ID NO: 320). In some cases, the variant IL-2 polypeptide comprises the following amino acid sequence: APTSSSTKKTQLQLEALLLDLQMILNGINNYKNPKLTRMLTAKFYMPKKATELKHLQCL EEELKPLEEVLNLAQSKNFHLRPRDLISNINVIVLELKGSETTFMCEYADETATIVEFLNR WITFCQSIISTLT (SEQ ID NO: 304), where the H16A and F42A substitutions are underlined. In some cases, the IgG1 Fc polypeptide comprises the following amino acid sequence: DKTHTCPPCPAPELLGGPSVFLFPPKPKDTLMISRTPEVTCVVVDVSHEDPEVKFNWYV DGVEVHNAKTKPREEQYASTYRVVSVLTVLHQDWLNGKEYKCKVSNKALPAPIEKTIS KAKGQPREPQVYTLPPSREEMTKNQVSLTCLVKGFYPSDIAVEWESNGQPENNYKTTPP VLDSDGSFFLYSKLTVDKSRWQQGNVFSCSVMHEALHNHYTQKSLSLSPGK (SEQ ID NO: 309). In some cases, the single-chain polypeptide comprises the amino acid sequence depicted in FIG. 29A, without the leader peptide and without the C-terminal linker and histidine tag. For example, in some cases, the single-chain polypeptide comprises amino acids 21-876 of the amino acid sequence depicted in FIG. 29A.

### Antigen-Presenting Polypeptides

The present disclosure provides an antigen-presenting polypeptide (APP) that does not include an immunomodulatory polypeptide. An APP of the present disclosure can be a single chain polypeptide or a multi-chain (multimeric) polypeptide. An APP of the present disclosure is useful for diagnostic applications and therapeutic applications.

### Multimeric Antigen-Presenting Polypeptides

In some cases, an APP of the present disclosure comprises two polypeptide chains. In some cases, the two polypeptide chains are covalently linked to one another, e.g., via a disulfide bond. In other instances, the two polypeptide chains are not covalently linked to one another. In some cases, the two polypeptide chains are not covalently linked to one another; and in some of these cases, each of the two polypeptide chains comprises a member of a dimerization pair. Examples of multimeric APPs of the present disclosure are depicted schematically in FIG. 37A and FIG. 37B.

In some cases, an antigen-presenting multimeric polypeptide (multimeric APP) of the present disclosure comprises: a) a first polypeptide comprising, in order from N-terminus to C-terminus: i) an MHC Class II α1 polypeptide; and ii) an MHC Class II α2 polypeptide; and b) a second polypeptide comprising, in order from N-terminus to C-terminus: i) a peptide antigen (an “epitope”) that is recognized (e.g., is capable of being recognized and bound) by a T-cell receptor (TCR); ii) an MHC Class II β1 polypeptide; and iii) an MHC Class II β2 polypeptide. In some cases, an APP of the present disclosure comprises: a) a first polypeptide comprising, in order from N-terminus to C-terminus: i) an MHC Class II α1 polypeptide; and ii) an MHC Class II α2 polypeptide; and b) a second polypeptide comprising, in order from N-terminus to C-terminus: i) a peptide antigen (an “epitope”) that is recognized (e.g., is capable of being recognized and bound) by a T-cell receptor (TCR); ii) an MHC Class II β1 polypeptide; iii) an MHC Class II β2 polypeptide; and iv) an immunoglobulin or non-immunoglobulin scaffold polypeptide. In some cases, an APP of the present disclosure comprises: a) a first polypeptide comprising, in order from N-terminus to C-terminus: i) an MHC Class II α1 polypeptide; and ii) an MHC Class II α2 polypeptide; and b) a second polypeptide comprising, in order from N-terminus to C-terminus: i) a peptide antigen (an “epitope”) that is recognized (e.g., is capable of being recognized and bound) by a T-cell receptor (TCR); ii) an MHC Class II β1 polypeptide; iii) an MHC Class II β2 polypeptide; and iv) an immunoglobulin (Ig) Fc polypeptide. In some cases, the second polypeptide comprises a linker between the peptide antigen and the MHC Class II β1 polypeptide. In some cases, the second polypeptide comprises a linker between the MHC Class II β1 polypeptide and the immunoglobulin or non-immunoglobulin scaffold polypeptide.

In some cases, an antigen-presenting multimeric polypeptide (a multimeric APP) of the present disclosure comprises: a) a first polypeptide comprising, in order from N-terminus to C-terminus: i) an MHC Class II α1 polypeptide; ii) an MHC Class II α2 polypeptide; and iii) a first member of a dimerizer pair; and b) a second polypeptide comprising, in order from N-terminus to C-terminus: i) a peptide antigen (an “epitope”) that is recognized (e.g., is capable of being recognized and bound) by a TCR; ii) an MHC Class II β1 polypeptide; iii) an MHC Class II β2 polypeptide; and iv) a second member of the dimerizer pair. The first and the second members of the dimerizer pair bind to one another non-covalently. In some cases, the first and the second members of the dimerizer pair bind to one another non-covalently without the need for a dimerization agent. In some cases, the first and the second members of the dimerizer pair bind to one another non-covalently in the presence of a dimerizer agent. In some cases, an APP of the present disclosure comprises: a) a first polypeptide comprising, in order from N-terminus to C-terminus: i) an MHC Class II α1 polypeptide; ii) an MHC Class II α2 polypeptide; and iii) a first member of a dimerizer pair; and b) a second polypeptide comprising, in order from N-terminus to C-terminus: i) a peptide antigen (an “epitope”) that is recognized (e.g., is capable of being recognized and bound) by a TCR; ii) an MHC Class II β1 polypeptide; iii) an MHC Class II β2 polypeptide; iv) a second member of the dimerizer pair; and v) an immunoglobulin or non-immunoglobulin scaffold polypeptide. In some cases, an APP of the present disclosure comprises: a) a first polypeptide comprising, in order from N-terminus to C-terminus: i) an MHC Class II α1 polypeptide; ii) an MHC Class II α2 polypeptide; and iii) a first member of a dimerizer pair; and b) a second polypeptide comprising, in order from N-terminus to C-terminus: i) a peptide antigen (an “epitope”) that is recognized (e.g., is capable of being recognized and bound) by a TCR; ii) an MHC Class II β1 polypeptide; iii) an MHC Class II β2 polypeptide; iv) a second member of the dimerizer pair; and v) an Ig Fc polypeptide. In some cases, an APP of the present disclosure comprises: a) a first polypeptide comprising, in order from N-terminus to C-terminus: i) an MHC Class II α1 polypeptide; ii) an MHC Class II α2 polypeptide; and iii) a first leucine zipper polypeptide; and b) a second polypeptide comprising, in order from N-terminus to C-terminus: i) a peptide antigen (an “epitope”) that is recognized (e.g., is capable of being recognized and bound) by a TCR; ii) an MHC Class II β1 polypeptide; iii) an MHC Class II β2 polypeptide; iv) a second leucine zipper polypeptide; and v) an Ig Fc polypeptide. In some cases, the second polypeptide comprises a linker between the peptide antigen and the MHC Class II β1 polypeptide. In some cases, the second polypeptide comprises a linker between the MHC Class II β1 polypeptide and the second member of the dimerizing pair. In some cases, the first polypeptide comprises a linker between the MHC Class II α2 polypeptide and the first member of the dimerizing pair.

In some cases, an antigen-presenting multimeric polypeptide (a multimeric APP) of the present disclosure comprises: a) a first polypeptide comprising, in order from N-terminus to C-terminus: i) a peptide antigen (an “epitope”) that is recognized (e.g., is capable of being recognized and bound) by a TCR; ii) an MHC Class II β1 polypeptide; iii) an MHC Class II α1 polypeptide; iv) an MHC Class II α2 polypeptide; and v) a first member of a dimerizing pair; and b) a second polypeptide comprising, in order from N-terminus to C-terminus: i) an MHC Class II β2 polypeptide; and ii) a second member of the dimerizing pair. In some cases, an APP of the present disclosure comprises: a) a first polypeptide comprising, in order from N-terminus to C-terminus: i) a peptide antigen (an “epitope”) that is recognized (e.g., is capable of being recognized and bound) by a TCR; ii) an MHC Class II β1 polypeptide; iii) an MHC Class II α1 polypeptide; iv) an MHC Class II α2 polypeptide; v) a first member of a dimerizing pair; vi) an immunoglobulin or non-immunoglobulin scaffold polypeptide; and b) a second polypeptide comprising, in order from N-terminus to C-terminus: i) an MHC Class II β2 polypeptide; and ii) a second member of the dimerizing pair. In some cases, an APP of the present disclosure comprises: a) a first polypeptide comprising, in order from N-terminus to C-terminus: i) a peptide antigen (an “epitope”) that is recognized (e.g., is capable of being recognized and bound) by a TCR; ii) an MHC Class II β1 polypeptide; iii) an MHC Class II α1 polypeptide; iv) an MHC Class II α2 polypeptide; v) a first member of a dimerizing pair; vi) an Ig Fc polypeptide; and b) a second polypeptide comprising, in order from N-terminus to C-terminus: i) an MHC Class II β2 polypeptide; and ii) a second member of the dimerizing pair. In some cases, an APP of the present disclosure comprises: a) a first polypeptide comprising, in order from N-terminus to C-terminus: i) a peptide antigen (an “epitope”) that is recognized (e.g., is capable of being recognized and bound) by a TCR; ii) an MHC Class II β1 polypeptide; iii) an MHC Class II α1 polypeptide; iv) an MHC Class II α2 polypeptide; v) a first leucine zipper polypeptide; vi) an Ig Fc polypeptide; and b) a second polypeptide comprising, in order from N-terminus to C-terminus: i) an MHC Class II β2 polypeptide; and ii) a second leucine zipper polypeptide. In some cases, the first polypeptide comprises a linker between the peptide antigen and the MHC Class II β1 polypeptide. In some cases, the first polypeptide comprises a linker between the MHC Class II β1 polypeptide and the MHC Class II α1 polypeptide. In some cases, the first polypeptide comprises a linker between the MHC Class II α2 polypeptide and the first member of the dimerizing pair. In some cases, the second polypeptide comprises a linker between the MHC Class II β2 polypeptide and the second member of the dimerizing pair.

### Monomeric Antigen-Presenting Polypeptides

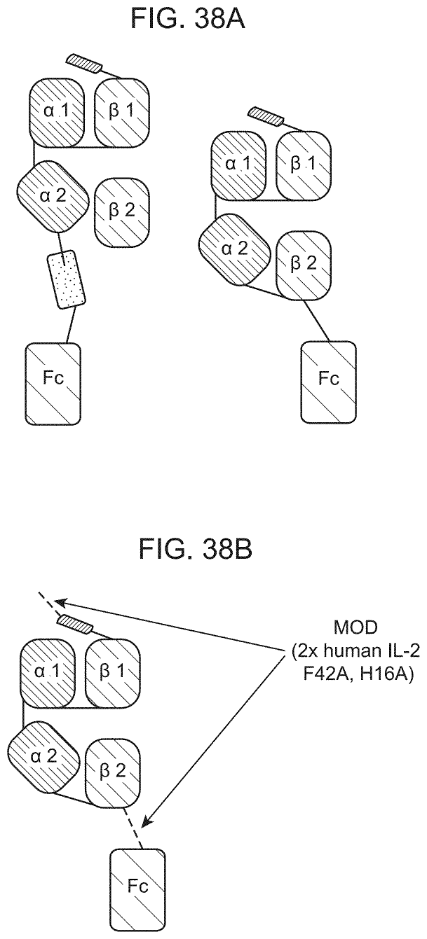

In some cases, an APP of the present disclosure is a single polypeptide chain. Examples are depicted schematically in FIG. 37C and FIG. 38A.

In some cases, an APP (e.g., a single-chain APP) of the present disclosure comprises, in order from N-terminus to C-terminus: i) a peptide antigen (an “epitope”) that is recognized (e.g., is capable of being recognized and bound) by a TCR; ii) an MHC Class II β1 polypeptide; iii) an MHC Class II β2 polypeptide; iv) an MHC Class II α1 polypeptide; and v) an MHC Class II α2 polypeptide. In some cases, an APP of the present disclosure comprises, in order from N-terminus to C-terminus: i) a peptide antigen (an “epitope”) that is recognized (e.g., is capable of being recognized and bound) by a TCR; ii) an MHC Class II β1 polypeptide; iii) an MHC Class II β2 polypeptide; iv) an MHC Class II α1 polypeptide; v) an MHC Class II α2 polypeptide; and vi) an immunoglobulin or non-immunoglobulin scaffold polypeptide. In some cases, an APP of the present disclosure comprises, in order from N-terminus to C-terminus: i) a peptide antigen (an “epitope”) that is recognized (e.g., is capable of being recognized and bound) by a TCR; ii) an MHC Class II β1 polypeptide; iii) an MHC Class II β2 polypeptide; iv) an MHC Class II α1 polypeptide; v) an MHC Class II α2 polypeptide; and vi) an Ig Fc polypeptide. In some cases, the APP comprises a linker between the peptide antigen and the MHC Class II β1 polypeptide. In some cases, the APP comprises a linker between the MHC Class II β2 polypeptide and the MHC Class II α1 polypeptide. In some cases, the APP comprises a linker between the MHC Class II α2 polypeptide and the immunoglobulin or non-immunoglobulin scaffold.

In some cases, an APP of the present disclosure comprises, in order from N-terminus to C-terminus: i) a peptide antigen (an “epitope”) that is recognized (e.g., is capable of being recognized and bound) by a TCR; ii) an MHC Class II β1 polypeptide; iii) an MHC Class II α1 polypeptide; iv) an MHC Class II α2 polypeptide; and v) an MHC Class II β2 polypeptide. In some cases, an APP of the present disclosure comprises, in order from N-terminus to C-terminus: i) a peptide antigen (an “epitope”) that is recognized (e.g., is capable of being recognized and bound) by a TCR; ii) an MHC Class II β1 polypeptide; iii) an MHC Class II α1 polypeptide; iv) an MHC Class II α2 polypeptide; v) an MHC Class II β2 polypeptide; and vi) an immunoglobulin or non-immunoglobulin scaffold polypeptide. In some cases, an APP of the present disclosure comprises, in order from N-terminus to C-terminus: i) a peptide antigen (an “epitope”) that is recognized (e.g., is capable of being recognized and bound) by a TCR; ii) an MHC Class II β1 polypeptide; iii) an MHC Class II α1 polypeptide; iv) an MHC Class II α2 polypeptide; v) an MHC Class II β2 polypeptide; and vi) an Ig Fc polypeptide. In some cases, the APP comprises a linker between the peptide antigen and the MHC Class II β1 polypeptide. In some cases, the APP comprises a linker between the MHC Class II β1 polypeptide and the MHC Class II α1 polypeptide. In some cases, the APP comprises a linker between the MHC Class II α2 polypeptide and the MHC Class II β2 polypeptide. In some cases, the APP comprises a linker between the MHC Class II β2 polypeptide and the Ig or non-Ig scaffold.

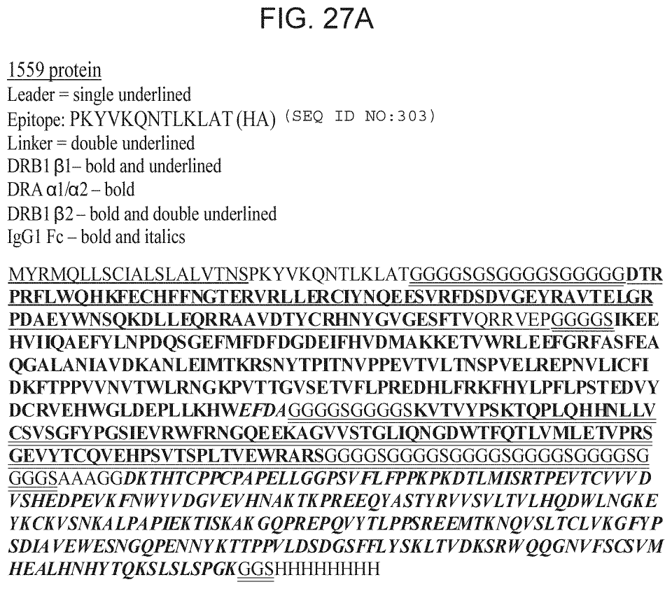

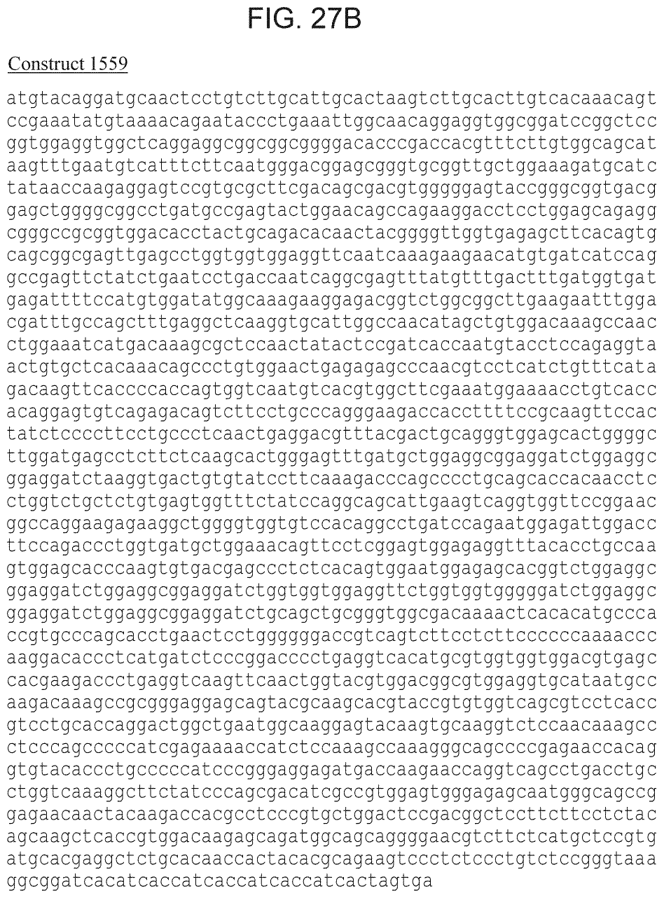

In some cases, a single-chain APP of the present disclosure comprises, in order from N-terminus to C-terminus: i) an epitope; ii) an HLA β1 polypeptide; iii) an HLA α1 polypeptide; iv) an HLA α2 polypeptide; v) an HLA β2 polypeptide; and vi) an Ig Fc polypeptide. As one non-limiting example, a single-chain APP of the present disclosure can comprise, in order from N-terminus to C-terminus: i) an epitope; ii) an HLA DRB1 β1 polypeptide; iii) an HLA DRA α1 polypeptide; iv) an HLA DRA α2 polypeptide; v) an HLA DRB β2 polypeptide; and vi) an IgG1 Fc polypeptide. In some cases, the epitope is a hemagglutinin epitope (PKYVKQNTLKLAT; SEQ ID NO: 303). In other instances, the epitope is not PKYVKQNTLKLAT (SEQ ID NO: 303); instead, the epitope is substituted with a different epitope. In some cases, the single-chain polypeptide comprises the 1559 amino acid sequence depicted in FIG. 27A, without the leader peptide and without the C-terminal linker and histidine tag. For example, in some cases, the single-chain polypeptide comprises amino acids 21-700 of the amino acid sequence depicted in FIG. 27A.

### MHC Class II Polypeptides

As noted above, an APP of the present disclosure comprises MHC Class II polypeptides, i.e., an MHC Class II α chain polypeptide and an MHC Class II β chain polypeptide. Suitable MHC Class II α chain polypeptides and MHC Class II β chain polypeptides are described above.

### Fc Polypeptides

As noted above, in some cases, an APP of the present disclosure can comprise an Ig Fc polypeptide. For example, where the APP is a multimeric polypeptide, in some cases, the first and/or the second polypeptide chain of a multimeric polypeptide comprises an Fc polypeptide. In some cases, an APP of the present disclosure is a monomeric polypeptide and comprises an Ig Fc polypeptide. The Fc polypeptide can be a human IgG1 Fc, a human IgG2 Fc, a human IgG3 Fc, a human IgG4 Fc, etc.

In some cases, the Fc polypeptide comprises an amino acid sequence having at least about 70%, at least about 75%, at least about 80%, at least about 85%, at least about 90%, at least about 95%, at least about 98%, at least about 99%, or 100%, amino acid sequence identity to an amino acid sequence of an Fc region depicted in FIG. 21A-21G. In some cases, the Fc region comprises an amino acid sequence having at least about 70%, at least about 75%, at least about 80%, at least about 85%, at least about 90%, at least about 95%, at least about 98%, at least about 99%, or 100%, amino acid sequence identity to the human IgG1 Fc polypeptide depicted in FIG. 21A. In some cases, the Fc region comprises an amino acid sequence having at least about 70%, at least about 75%, at least about 80%, at least about 85%, at least about 90%, at least about 95%, at least about 98%, at least about 99%, or 100%, amino acid sequence identity to the human IgG1 Fc polypeptide depicted in FIG. 21A; and comprises a substitution of N77; e.g., the Fc polypeptide comprises an N77A substitution. In some cases, the Fc polypeptide comprises an amino acid sequence having at least about 70%, at least about 75%, at least about 80%, at least about 85%, at least about 90%, at least about 95%, at least about 98%, at least about 99%, or 100%, amino acid sequence identity to the human IgG2 Fc polypeptide depicted in FIG. 21A; e.g., the Fc polypeptide comprises an amino acid sequence having at least about 70%, at least about 75%, at least about 80%, at least about 85%, at least about 90%, at least about 95%, at least about 98%, at least about 99%, or 100%, amino acid sequence identity to amino acids 99-325 of the human IgG2 Fc polypeptide depicted in FIG. 21A. In some cases, the Fc polypeptide comprises an amino acid sequence having at least about 70%, at least about 75%, at least about 80%, at least about 85%, at least about 90%, at least about 95%, at least about 98%, at least about 99%, or 100%, amino acid sequence identity to the human IgG3 Fc polypeptide depicted in FIG. 21A; e.g., the Fc polypeptide comprises an amino acid sequence having at least about 70%, at least about 75%, at least about 80%, at least about 85%, at least about 90%, at least about 95%, at least about 98%, at least about 99%, or 100%, amino acid sequence identity to amino acids 19-246 of the human IgG3 Fc polypeptide depicted in FIG. 21A. In some cases, the Fc polypeptide comprises an amino acid sequence having at least about 70%, at least about 75%, at least about 80%, at least about 85%, at least about 90%, at least about 95%, at least about 98%, at least about 99%, or 100%, amino acid sequence identity to the human IgM Fc polypeptide depicted in FIG. 21B; e.g., the Fc polypeptide comprises an amino acid sequence having at least about 70%, at least about 75%, at least about 80%, at least about 85%, at least about 90%, at least about 95%, at least about 98%, at least about 99%, or 100%, amino acid sequence identity to amino acids 1-276 to the human IgM Fc polypeptide depicted in FIG. 21B. In some cases, the Fc polypeptide comprises an amino acid sequence having at least about 70%, at least about 75%, at least about 80%, at least about 85%, at least about 90%, at least about 95%, at least about 98%, at least about 99%, or 100%, amino acid sequence identity to the human IgA Fc polypeptide depicted in FIG. 21C; e.g., the Fc polypeptide comprises an amino acid sequence having at least about 70%, at least about 75%, at least about 80%, at least about 85%, at least about 90%, at least about 95%, at least about 98%, at least about 99%, or 100%, amino acid sequence identity to amino acids 1-234 to the human IgA Fc polypeptide depicted in FIG. 21C.

In some cases, the Fc polypeptide comprises an amino acid sequence having at least about 70%, at least about 75%, at least about 80%, at least about 85%, at least about 90%, at least about 95%, at least about 98%, at least about 99%, or 100%, amino acid sequence identity to the human IgG4 Fc polypeptide depicted in FIG. 21C. In some cases, the Fc polypeptide comprises an amino acid sequence having at least about 70%, at least about 75%, at least about 80%, at least about 85%, at least about 90%, at least about 95%, at least about 98%, at least about 99%, or 100%, amino acid sequence identity to amino acids 100 to 327 of the human IgG4 Fc polypeptide depicted in FIG. 21C.

In some cases, the IgG4 Fc polypeptide comprises the following amino acid sequence:

(SEQ ID NO: 321)
PPCPSCPAPEFLGGPSVFLFPPKPKDTLMISRTPEVTCVVVDVSQEDPEVQ
FNWYVDGVEVHNAKTKPREEQFNSTYRVVSVLTVLHQDWLNGKEYKCKVSN
KGLPSSIEKTISKAKGQPREPQVYTLPPSQEEMTKNQVSLTCLVKGFYPSD
IAVEWESNGQPENNYKTTPPVLDSDGSFFLYSRLTVDKSRWQEGNVFSCSV
MHEALHNHYTQKSLSLSPG.

In some cases, the Fc polypeptide present in an APP of the present disclosure comprises the amino acid sequence depicted in FIG. 21A (human IgG1 Fc). In some cases, the Fc polypeptide present in an APP of the present disclosure comprises the amino acid sequence depicted in FIG. 21A (human IgG1 Fc), except for a substitution of N297 (N77 of the amino acid sequence depicted in FIG. 21A) with an amino acid other than asparagine. In some cases, the Fc polypeptide present in an APP of the present disclosure comprises the amino acid sequence depicted in FIG. 21C (human IgG1 Fc comprising an N297A substitution, which is N77 of the amino acid sequence depicted in FIG. 21A). In some cases, the Fc polypeptide present in an APP of the present disclosure comprises the amino acid sequence depicted in FIG. 21A (human IgG1 Fc), except for a substitution of L234 (L14 of the amino acid sequence depicted in FIG. 21A) with an amino acid other than leucine. In some cases, the Fc polypeptide present in an APP of the present disclosure comprises the amino acid sequence depicted in FIG. 21A (human IgG1 Fc), except for a substitution of L235 (L15 of the amino acid sequence depicted in FIG. 21A) with an amino acid other than leucine.

In some cases, the Fc polypeptide present in an APP of the present disclosure comprises the amino acid sequence depicted in FIG. 21E. In some cases, the Fc polypeptide present in an APP of the present disclosure comprises the amino acid sequence depicted in FIG. 21F. In some cases, the Fc polypeptide present in an APP of the present disclosure comprises the amino acid sequence depicted in FIG. 21G (human IgG1 Fc comprising an L234A substitution and an L235A substitution, corresponding to positions 14 and 15 of the amino acid sequence depicted in FIG. 21G). In some cases, the Fc polypeptide present in an APP of the present disclosure comprises the amino acid sequence depicted in FIG. 21A (human IgG1 Fc), except for a substitution of P331 (P111 of the amino acid sequence depicted in FIG. 21A) with an amino acid other than proline; in some cases, the substitution is a P331S substitution. In some cases, the Fc polypeptide present in an APP of the present disclosure comprises the amino acid sequence depicted in FIG. 21A (human IgG1 Fc), except for substitutions at L234 and L235 (L14 and L15 of the amino acid sequence depicted in FIG. 21A) with amino acids other than leucine. In some cases, the Fc polypeptide present in an APP of the present disclosure comprises the amino acid sequence depicted in FIG. 21A (human IgG1 Fc), except for substitutions at L234 and L235 (L14 and L15 of the amino acid sequence depicted in FIG. 21A) with amino acids other than leucine, and a substitution of P331 (P111 of the amino acid sequence depicted in FIG. 21A) with an amino acid other than proline. In some cases, the Fc polypeptide present in an APP of the present disclosure comprises the amino acid sequence depicted in FIG. 21E (human IgG1 Fc comprising L234F, L235E, and P331S substitutions (corresponding to amino acid positions 14, 15, and 111 of the amino acid sequence depicted in FIG. 21E). In some cases, the Fc polypeptide present in an APP of the present disclosure is an IgG1 Fc polypeptide that comprises L234A and L235A substitutions (substitutions of L14 and L15 of the amino acid sequence depicted in FIG. 21A with Ala), as depicted in FIG. 21G.

### Nucleic Acids

The present disclosure provides a nucleic acid comprising a nucleotide sequence encoding a TMAPP of the present disclosure. In some cases, the nucleic acid is a recombinant expression vector; thus, the present disclosure provides a recombinant expression vector comprising a nucleotide sequence encoding a TMAPP of the present disclosure. The present disclosure provides a nucleic acid comprising a nucleotide sequence encoding an APP of the present disclosure. In some cases, the nucleic acid is a recombinant expression vector; thus, the present disclosure provides a recombinant expression vector comprising a nucleotide sequence encoding an APP of the present disclosure. The discussion, below, of nucleic acids refers to nucleic acids encoding TMAPPs of the present disclosure; however, the discussion applies as well to nucleic acids encoding APPs of the present disclosure.

### Nucleic Acids Encoding Single-Chain Antigen-Presenting Polypeptides of the Present Disclosure

As described above, in some cases, a TMAPP of the present disclosure comprises a single polypeptide chain. Thus, the present disclosure provides a nucleic acid comprising a nucleotide sequence encoding a single-chain TMAPP of the present disclosure. A nucleotide sequence comprising a nucleotide sequence encoding a single-chain TMAPP of the present disclosure can be operably linked to a transcription control element(s), e.g., a promoter.

### Nucleic Acid(s) Encoding Multimeric Polypeptides of the Present Disclosure

As noted above, in some cases, a TMAPP of the present disclosure comprises at least 2 separate polypeptide chains. The present disclosure provides nucleic acids comprising nucleotide sequences encoding a multimeric TMAPP of the present disclosure. In some cases, the individual polypeptide chains of a multimeric TMAPP of the present disclosure are encoded in separate nucleic acids. In some cases, all polypeptide chains of a multimeric polypeptide of the present disclosure are encoded in a single nucleic acid. In some cases, a first nucleic acid comprises a nucleotide sequence encoding a first polypeptide of a multimeric polypeptide of the present disclosure; and a second nucleic acid comprises a nucleotide sequence encoding a second polypeptide of a multimeric polypeptide of the present disclosure. In some cases, single nucleic acid comprises a nucleotide sequence encoding a first polypeptide of a multimeric polypeptide of the present disclosure and a second polypeptide of a multimeric polypeptide of the present disclosure.

### Separate Nucleic Acids Encoding Individual Polypeptide Chains of a Multimeric TMAPP

The present disclosure provides nucleic acids comprising nucleotide sequences encoding a TMAPP of the present disclosure. As noted above, in some cases, the individual polypeptide chains of a multimeric TMAPP of the present disclosure are encoded in separate nucleic acids. In some cases, nucleotide sequences encoding the separate polypeptide chains of a TMAPP of the present disclosure are operably linked to transcriptional control elements, e.g., promoters, such as promoters that are functional in a eukaryotic cell, where the promoter can be a constitutive promoter or an inducible promoter.

For example, the present disclosure provides a first nucleic acid and a second nucleic acid, where the first nucleic acid comprises a nucleotide sequence encoding the first polypeptide of a TMAPP of the present disclosure, and where the second nucleic acid comprises a nucleotide sequence encoding the second polypeptide of the TMAPP. In some cases, the nucleotide sequences encoding the first and the second polypeptides are operably linked to transcriptional control elements. In some cases, the transcriptional control element is a promoter that is functional in a eukaryotic cell. In some cases, the nucleic acids are present in separate expression vectors.

In some cases, the nucleotide sequences encoding the first and the second polypeptides are operably linked to transcriptional control elements. In some cases, the transcriptional control element is a promoter that is functional in a eukaryotic cell. In some cases, the nucleic acids are present in separate expression vectors.

### Nucleic Acid Encoding Two or More Polypeptides Present in a TMAPP

The present disclosure provides a nucleic acid comprising nucleotide sequences encoding at least the first polypeptide and the second polypeptide of a TMAPP of the present disclosure. In some cases, where a TMAPP of the present disclosure includes a first, second, and third polypeptide, the nucleic acid includes a nucleotide sequence encoding the first, second, and third polypeptides. In some cases, the nucleotide sequences encoding the first polypeptide and the second polypeptide of a TMAPP of the present disclosure includes a proteolytically cleavable linker interposed between the nucleotide sequence encoding the first polypeptide and the nucleotide sequence encoding the second polypeptide. In some cases, the nucleotide sequences encoding the first polypeptide and the second polypeptide of a TMAPP of the present disclosure includes an internal ribosome entry site (IRES) interposed between the nucleotide sequence encoding the first polypeptide and the nucleotide sequence encoding the second polypeptide. In some cases, the nucleotide sequences encoding the first polypeptide and the second polypeptide of a TMAPP of the present disclosure includes a ribosome skipping signal (or cis-acting hydrolase element, CHYSEL) interposed between the nucleotide sequence encoding the first polypeptide and the nucleotide sequence encoding the second polypeptide. Examples of nucleic acids are described below, where a proteolytically cleavable linker is provided between nucleotide sequences encoding the first polypeptide and the second polypeptide of a TMAPP of the present disclosure; in any of these embodiments, an IRES or a ribosome skipping signal can be used in place of the nucleotide sequence encoding the proteolytically cleavable linker.

In some cases, a first nucleic acid (e.g., a recombinant expression vector, an mRNA, a viral RNA, etc.) comprises a nucleotide sequence encoding a first polypeptide chain of a TMAPP of the present disclosure; and a second nucleic acid (e.g., a recombinant expression vector, an mRNA, a viral RNA, etc.) comprises a nucleotide sequence encoding a second polypeptide chain of a TMAPP of the present disclosure. In some cases, the nucleotide sequence encoding the first polypeptide, and the second nucleotide sequence encoding the second polypeptide, are each operably linked to transcriptional control elements, e.g., promoters, such as promoters that are functional in a eukaryotic cell, where the promoter can be a constitutive promoter or an inducible promoter.

### Recombinant Expression Vectors

The present disclosure provides recombinant expression vectors comprising nucleic acids of the present disclosure. In some cases, the recombinant expression vector is a non-viral vector. In some cases, the recombinant expression vector is a viral construct, e.g., a recombinant adeno-associated virus construct (see, e.g., U.S. Pat. No. 7,078,387), a recombinant adenoviral construct, a recombinant lentiviral construct, a recombinant retroviral construct, a non-integrating viral vector, etc.

Suitable expression vectors include, but are not limited to, viral vectors (e.g. viral vectors based on vaccinia virus; poliovirus; adenovirus (see, e.g., Li et al., Invest Opthalmol Vis Sci 35:2543 2549, 1994; Borras et al., Gene Ther 6:515 524, 1999; Li and Davidson, PNAS 92:7700 7704, 1995; Sakamoto et al., H Gene Ther 5:1088 1097, 1999; WO 94/12649, WO 93/03769; WO 93/19191; WO 94/28938; WO 95/11984 and WO 95/00655); adeno-associated virus (see, e.g., Ali et al., Hum Gene Ther 9:81 86, 1998, Flannery et al., PNAS 94:6916 6921, 1997; Bennett et al., Invest Opthalmol Vis Sci 38:2857 2863, 1997; Jomary et al., Gene Ther 4:683 690, 1997, Rolling et al., Hum Gene Ther 10:641 648, 1999; Ali et al., Hum Mol Genet 5:591 594, 1996; Srivastava in WO 93/09239, Samulski et al., J. Vir. (1989) 63:3822-3828; Mendelson et al., Virol. (1988) 166:154-165; and Flotte et al., PNAS (1993) 90:10613-10617); SV40; herpes simplex virus; human immunodeficiency virus (see, e.g., Miyoshi et al., PNAS 94:10319 23, 1997; Takahashi et al., J Virol 73:7812 7816, 1999); a retroviral vector (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus); and the like. Numerous suitable expression vectors are known to those of skill in the art, and many are commercially available.

Depending on the host/vector system utilized, any of a number of suitable transcription and translation control elements, including constitutive and inducible promoters, transcription enhancer elements, transcription terminators, etc. may be used in the expression vector (see e.g., Bitter et al. (1987) Methods in Enzymology, 153:516-544).

In some cases, a nucleotide sequence encoding an APP of the present disclosure is operably linked to a control element, e.g., a transcriptional control element, such as a promoter. The transcriptional control element may be functional in either a eukaryotic cell, e.g., a mammalian cell; or a prokaryotic cell (e.g., bacterial or archaeal cell). In some cases, a nucleotide sequence encoding a DNA-targeting RNA and/or a site-directed modifying polypeptide is operably linked to multiple control elements that allow expression of the nucleotide sequence encoding a DNA-targeting RNA and/or a site-directed modifying polypeptide in both prokaryotic and eukaryotic cells.

Non-limiting examples of suitable eukaryotic promoters (promoters functional in a eukaryotic cell) include those from cytomegalovirus (CMV) immediate early, herpes simplex virus (HSV) thymidine kinase, early and late SV40, long terminal repeats (LTRs) from retrovirus, and mouse metallothionein-I. Selection of the appropriate vector and promoter is well within the level of ordinary skill in the art. The expression vector may also contain a ribosome binding site for translation initiation and a transcription terminator. The expression vector may also include appropriate sequences for amplifying expression.

### Genetically Modified Host Cells

The present disclosure provides a genetically modified host cell, where the host cell is genetically modified with a nucleic acid(s) of the present disclosure.

Suitable host cells include eukaryotic cells, such as yeast cells, insect cells, and mammalian cells. In some cases, the host cell is a cell of a mammalian cell line. Suitable mammalian cell lines include human cell lines, non-human primate cell lines, rodent (e.g., mouse, rat) cell lines, and the like. Suitable mammalian cell lines include, but are not limited to, HeLa cells (e.g., American Type Culture Collection (ATCC) No. CCL-2), CHO cells (e.g., ATCC Nos. CRL9618, CCL61, CRL9096), 293 cells (e.g., ATCC No. CRL-1573), Vero cells, NIH 3T3 cells (e.g., ATCC No. CRL-1658), Huh-7 cells, BHK cells (e.g., ATCC No. CCL10), PC12 cells (ATCC No. CRL1721), COS cells, COS-7 cells (ATCC No. CRL1651), RAT1 cells, mouse L cells (ATCC No. CCLI.3), human embryonic kidney (HEK) cells (ATCC No. CRL1573), HLHepG2 cells, and the like.

Genetically modified host cells can be used to produce a TMAPP of the present disclosure. For example, a genetically modified host cell can be used to produce a multimeric TMAPP of the present disclosure, or a single-chain TMAPP of the present disclosure. An expression vector(s) comprising nucleotide sequences encoding the polypeptide(s) is/are introduced into a host cell, generating a genetically modified host cell, which genetically modified host cell produces the polypeptide(s).

### Compositions

The present disclosure provides compositions, including pharmaceutical compositions, comprising a TMAPP or an APP of the present disclosure. The present disclosure provides compositions, including pharmaceutical compositions, comprising a nucleic acid or a recombinant expression vector of the present disclosure. The discussion, below, of compositions refers to compositions comprising a TMAPP of the present disclosure; however, the discussion applies equally to an APP of the present disclosure.

### Compositions Comprising a TMAPP

A composition of the present disclosure can comprise, in addition to a TMAPP of the present disclosure, one or more of: a salt, e.g., NaCl, MgCl₂, KCl, MgSO₄, etc.; a buffering agent, e.g., a Tris buffer, N-(2-Hydroxyethyl)piperazine-N′-(2-ethanesulfonic acid) (HEPES), 2-(N-Morpholino)ethanesulfonic acid (MES), 2-(N-Morpholino)ethanesulfonic acid sodium salt (MES), 3-(N-Morpholino)propanesulfonic acid (MOPS), N-tris[Hydroxymethyl]methyl-3-aminopropanesulfonic acid (TAPS), etc.; a solubilizing agent; a detergent, e.g., a non-ionic detergent such as Tween-20, etc.; a protease inhibitor; glycerol; and the like.

The composition may comprise a pharmaceutically acceptable excipient, a variety of which are known in the art and need not be discussed in detail herein. Pharmaceutically acceptable excipients have been amply described in a variety of publications, including, for example, “Remington: The Science and Practice of Pharmacy”, 19^{th} Ed. (1995), or latest edition, Mack Publishing Co; A. Gennaro (2000) “Remington: The Science and Practice of Pharmacy”, 20th edition, Lippincott, Williams, & Wilkins; Pharmaceutical Dosage Forms and Drug Delivery Systems (1999) H. C. Ansel et al., eds 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

A pharmaceutical composition can comprise: i) a TMAPP of the present disclosure; and ii) a pharmaceutically acceptable excipient. In some cases, a subject pharmaceutical composition will be suitable for administration to a subject, e.g., will be sterile. For example, in some embodiments, a subject pharmaceutical composition will be suitable for administration to a human subject, e.g., where the composition is sterile and is free of detectable pyrogens and/or other toxins.

The protein compositions may comprise other components, such as pharmaceutical grades of mannitol, lactose, starch, magnesium stearate, sodium saccharin, talcum, cellulose, glucose, sucrose, magnesium, carbonate, and the like. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate, hydrochloride, sulfate salts, solvates (e.g., mixed ionic salts, water, organics), hydrates (e.g., water), and the like.

For example, compositions may include aqueous solution, powder form, granules, tablets, pills, suppositories, capsules, suspensions, sprays, and the like. The composition may be formulated according to the various routes of administration described below.

Where a TMAPP of the present disclosure is administered as an injectable (e.g. subcutaneously, intraperitoneally, intramuscularly, intralymphatically, and/or intravenously) directly into a tissue, a formulation can be provided as a ready-to-use dosage form, or as non-aqueous form (e.g. a reconstitutable storage-stable powder) or aqueous form, such as liquid composed of pharmaceutically acceptable carriers and excipients. The protein-containing formulations may also be provided so as to enhance serum half-life of the subject protein following administration. For example, the protein may be provided in a liposome formulation, prepared as a colloid, or other conventional techniques for extending serum half-life. A variety of methods are available for preparing liposomes, as described in, e.g., Szoka et al. 1980 Ann. Rev. Biophys. Bioeng. 9:467, U.S. Pat. Nos. 4,235,871, 4,501,728 and 4,837,028. The preparations may also be provided in controlled release or slow-release forms.

In some cases, a composition of the present disclosure comprises: a) a TMAPP of the present disclosure; and b) saline (e.g., 0.9% NaCl). In some cases, the composition is sterile. In some cases, the composition is suitable for administration to a human subject, e.g., where the composition is sterile and is free of detectable pyrogens and/or other toxins. Thus, the present disclosure provides a composition comprising: a) a TMAPP of the present disclosure; and b) saline (e.g., 0.9% NaCl), where the composition is sterile and is free of detectable pyrogens and/or other toxins.

Other examples of formulations suitable for parenteral administration include isotonic sterile injection solutions, anti-oxidants, bacteriostats, and solutes that render the formulation isotonic with the blood of the intended recipient, suspending agents, solubilizers, thickening agents, stabilizers, and preservatives. For example, a subject pharmaceutical composition can be present in a container, e.g., a sterile container, such as a syringe. The formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets.

The concentration of a TMAPP of the present disclosure in a formulation can vary widely (e.g., from less than about 0.1%, usually at or at least about 2% to as much as 20% to 50% or more by weight) and will usually be selected primarily based on fluid volumes, viscosities, and patient-based factors in accordance with the particular mode of administration selected and the patient's needs.

The present disclosure provides a container comprising a composition of the present disclosure, e.g., a liquid composition. The container can be, e.g., a syringe, an ampoule, and the like. In some cases, the container is sterile. In some cases, both the container and the composition are sterile.

### Compositions Comprising a Nucleic Acid or a Recombinant Expression Vector

The present disclosure provides compositions, e.g., pharmaceutical compositions, comprising a nucleic acid or a recombinant expression vector of the present disclosure. A wide variety of pharmaceutically acceptable excipients is known in the art and need not be discussed in detail herein. Pharmaceutically acceptable excipients have been amply described in a variety of publications, including, for example, A. Gennaro (2000) “Remington: The Science and Practice of Pharmacy”, 20th edition, Lippincott, Williams, & Wilkins; Pharmaceutical Dosage Forms and Drug Delivery Systems (1999) H. C. Ansel et al., eds 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

A composition of the present disclosure can include: a) one or more nucleic acids or one or more recombinant expression vectors comprising nucleotide sequences encoding a TMAPP of the present disclosure; and b) one or more of: a buffer, a surfactant, an antioxidant, a hydrophilic polymer, a dextrin, a chelating agent, a suspending agent, a solubilizer, a thickening agent, a stabilizer, a bacteriostatic agent, a wetting agent, and a preservative. Suitable buffers include, but are not limited to, (such as N,N-bis(2-hydroxyethyl)-2-aminoethanesulfonic acid (BES), bis(2-hydroxyethyl)amino-tris(hydroxymethyl)methane (BIS-Tris), N-(2-hydroxyethyl)piperazine-N′3-propanesulfonic acid (EPPS or HEPPS), glycylglycine, N-2-hydroxyehtylpiperazine-N′-2-ethanesulfonic acid (HEPES), 3-(N-morpholino)propane sulfonic acid (MOPS), piperazine-N,N′-bis(2-ethane-sulfonic acid) (PIPES), sodium bicarbonate, 3-(N-tris(hydroxymethyl)-methyl-amino)-2-hydroxy-propanesulfonic acid) TAPSO, (N-tris(hydroxymethyl)methyl-2-aminoethanesulfonic acid (TES), N-tris(hydroxymethyl)methyl-glycine (Tricine), tris(hydroxymethyl)-aminomethane (Tris), etc.). Suitable salts include, e.g., NaCl, MgCl₂, KCl, MgSO₄, etc.

A pharmaceutical formulation of the present disclosure can include a nucleic acid or recombinant expression vector of the present disclosure in an amount of from about 0.001% to about 90% (w/w). In the description of formulations, below, “subject nucleic acid or recombinant expression vector” will be understood to include a nucleic acid or recombinant expression vector of the present disclosure. For example, in some cases, a subject formulation comprises a nucleic acid or recombinant expression vector of the present disclosure.

A subject nucleic acid or recombinant expression vector can be admixed, encapsulated, conjugated or otherwise associated with other compounds or mixtures of compounds; such compounds can include, e.g., liposomes or receptor-targeted molecules. A subject nucleic acid or recombinant expression vector can be combined in a formulation with one or more components that assist in uptake, distribution and/or absorption.

A subject nucleic acid or recombinant expression vector composition can be formulated into any of many possible dosage forms such as, but not limited to, tablets, capsules, gel capsules, liquid syrups, soft gels, suppositories, and enemas. A subject nucleic acid or recombinant expression vector composition can also be formulated as suspensions in aqueous, non-aqueous or mixed media. Aqueous suspensions may further contain substances which increase the viscosity of the suspension including, for example, sodium carboxymethylcellulose, sorbitol and/or dextran. The suspension may also contain stabilizers.

A formulation comprising a subject nucleic acid or recombinant expression vector can be a liposomal formulation. As used herein, the term “liposome” means a vesicle composed of amphiphilic lipids arranged in a spherical bilayer or bilayers. Liposomes are unilamellar or multilamellar vesicles which have a membrane formed from a lipophilic material and an aqueous interior that contains the composition to be delivered. Cationic liposomes are positively charged liposomes that can interact with negatively charged DNA molecules to form a stable complex. Liposomes that are pH sensitive or negatively charged are believed to entrap DNA rather than complex with it. Both cationic and noncationic liposomes can be used to deliver a subject nucleic acid or recombinant expression vector.

Liposomes also include “sterically stabilized” liposomes, a term which, as used herein, refers to liposomes comprising one or more specialized lipids that, when incorporated into liposomes, result in enhanced circulation lifetimes relative to liposomes lacking such specialized lipids. Examples of sterically stabilized liposomes are those in which part of the vesicle-forming lipid portion of the liposome comprises one or more glycolipids or is derivatized with one or more hydrophilic polymers, such as a polyethylene glycol (PEG) moiety. Liposomes and their uses are further described in U.S. Pat. No. 6,287,860, which is incorporated herein by reference in its entirety.

The formulations and compositions of the present disclosure may also include surfactants. The use of surfactants in drug products, formulations and in emulsions is well known in the art. Surfactants and their uses are further described in U.S. Pat. No. 6,287,860.

In one embodiment, various penetration enhancers are included, to effect the efficient delivery of nucleic acids. In addition to aiding the diffusion of non-lipophilic drugs across cell membranes, penetration enhancers also enhance the permeability of lipophilic drugs. Penetration enhancers may be classified as belonging to one of five broad categories, i.e., surfactants, fatty acids, bile salts, chelating agents, and non-chelating non-surfactants. Penetration enhancers and their uses are further described in U.S. Pat. No. 6,287,860, which is incorporated herein by reference in its entirety.

Compositions and formulations for oral administration include powders or granules, microparticulates, nanoparticulates, suspensions or solutions in water or non-aqueous media, capsules, gel capsules, sachets, tablets, or minitablets. Thickeners, flavoring agents, diluents, emulsifiers, dispersing aids or binders may be desirable. Suitable oral formulations include those in which a subject antisense nucleic acid is administered in conjunction with one or more penetration enhancers surfactants and chelators. Suitable surfactants include, but are not limited to, fatty acids and/or esters or salts thereof, bile acids and/or salts thereof. Suitable bile acids/salts and fatty acids and their uses are further described in U.S. Pat. No. 6,287,860. Also suitable are combinations of penetration enhancers, for example, fatty acids/salts in combination with bile acids/salts. An exemplary suitable combination is the sodium salt of lauric acid, capric acid, and UDCA. Further penetration enhancers include, but are not limited to, polyoxyethylene-9-lauryl ether, and polyoxyethylene-20-cetyl ether. Suitable penetration enhancers also include propylene glycol, dimethylsulfoxide, triethanolamine, N,N-dimethylacetamide, N,N-dimethylformamide, 2-pyrrolidone and derivatives thereof, tetrahydrofurfuryl alcohol, and AZONE™.

### Methods

A TMAPP of the present disclosure is useful for modulating an activity of a T cell. Thus, the present disclosure provides methods of modulating an activity of a T cell, the methods generally involving contacting a target T cell with a TMAPP of the present disclosure.

An APP of the present disclosure is useful for various research, therapeutic, and diagnostic purposes. For example, an APP of the present disclosure can be used to label, directly or indirectly, an antigen-specific T cell.

### Methods of Modulating T Cell Activity

The present disclosure provides a method of selectively modulating the activity of an epitope-specific T cell, the method comprising contacting the T cell with a TMAPP of the present disclosure, where contacting the T cell with a TMAPP of the present disclosure selectively modulates the activity of the epitope-specific T cell. In some cases, the contacting occurs in vitro. In some cases, the contacting occurs in vivo. In some cases, the contacting occurs ex vivo.

In some cases, a TMAPP of the present disclosure reduces activity of an autoreactive T cell and/or an autoreactive B cell. In some cases, a TMAPP of the present disclosure increases the number and/or activity of a regulator T cell (Treg), resulting in reduced activity of an autoreactive T cell and/or an autoreactive B cell.

In some cases, the T cell being contacted with a TMAPP of the present disclosure is a regulatory T cell (Treg). Tregs are CD4⁺, FOXP3⁺, and CD25⁺. Tregs can suppress autoreactive T cells. In some cases, a method of the present disclosure activates Tregs, thereby reducing autoreactive T cell activity.

The present disclosure provides a method of increasing proliferation of Tregs, the method comprising contacting Tregs with a TMAPP of the present disclosure, where the contacting increases proliferation of Tregs. The present disclosure provides a method of increasing the number of Tregs in an individual, the method comprising administering to the individual a TMAPP of the present disclosure, where the administering results in an increase in the number of Tregs in the individual. For example, the number of Tregs can be increased by at least 5%, at least 10%, at least 15%, at least 20%, at least 25%, at least 30%, at least 40%, at least 50%, at least 60%, at least 70%, at least 80%, at least 90%, at least 2-fold, at least 2.5-fold, at least 5-fold, at least 10-fold, or more than 10-fold.

In some cases, the cell being contacted is a helper T cell, where contacting the helper T cell with a TMAPP of the present disclosure results in activation of the helper T cell. In some cases, activation of the helper T cell results in an increase in the activity and/or number of CD8⁺ cytotoxic T cells, e.g., CD8⁺ cytotoxic T cells that target and kill an autoreactive cell.

In some cases (e.g., where a TMAPP of the present disclosure comprises a cancer epitope), a TMAPP of the present disclosure activates a CD8⁺ T cell response, e.g., a CD8⁺ T cell response to a cancer cell.

### Methods of Detecting an Antigen-Specific T Cell

The present disclosure provides a method of detecting an antigen-specific T-cell. The methods comprise contacting a T cell with an APP of the present disclosure; and detecting binding of the APP to the T cell.

The present disclosure provides a method of detecting an antigen-specific T cell, the method comprising contacting a T cell with an APP of the present disclosure, wherein binding of the APP to the T cell indicates that the T cell is specific for the epitope present in the APP.

In some cases, the APP comprises a detectable label. Suitable detectable labels include, but are not limited to, a radioisotope, a fluorescent polypeptide, or an enzyme that generates a fluorescent product, and an enzyme that generates a colored product. Where the APP comprises a detectable label, binding of the APP to the T cell is detected by detecting the detectable label.

Suitable fluorescent proteins include, but are not limited to, green fluorescent protein (GFP) or variants thereof, blue fluorescent variant of GFP (BFP), cyan fluorescent variant of GFP (CFP), yellow fluorescent variant of GFP (YFP), enhanced GFP (EGFP), enhanced CFP (ECFP), enhanced YFP (EYFP), GFPS65T, Emerald, Topaz (TYFP), Venus, Citrine, mCitrine, GFPuv, destabilised EGFP (dEGFP), destabilised ECFP (dECFP), destabilised EYFP (dEYFP), mCFPm, Cerulean, T-Sapphire, CyPet, YPet, mKO, HcRed, t-HcRed, DsRed, DsRed2, DsRed-monomer, J-Red, dimer2, t-dimer2(12), mRFP1, pocilloporin, Renilla GFP, Monster GFP, paGFP, Kaede protein and kindling protein, Phycobiliproteins and Phycobiliprotein conjugates including B-Phycoerythrin, R-Phycoerythrin and Allophycocyanin. Other examples of fluorescent proteins include mHoneydew, mBanana, mOrange, dTomato, tdTomato, mTangerine, mStrawberry, mCherry, mGrape1, mRaspberry, mGrape2, mPlum (Shaner et al. (2005) Nat. Methods 2:905-909), and the like. Any of a variety of fluorescent and colored proteins from Anthozoan species, as described in, e.g., Matz et al. (1999) Nature Biotechnol. 17:969-973, is suitable for use.

Suitable enzymes include, but are not limited to, horse radish peroxidase (HRP), alkaline phosphatase (AP), beta-galactosidase (GAL), glucose-6-phosphate dehydrogenase, beta-N-acetylglucosaminidase, β-glucuronidase, invertase, Xanthine Oxidase, firefly luciferase, glucose oxidase (GO), and the like.

In some cases, binding of the APP to the T cell is detected using a detectably labeled antibody specific for the APP. An antibody specific for the APP can comprise a detectable label such as a radioisotope, a fluorescent polypeptide, or an enzyme that generates a fluorescent product, or an enzyme that generates a colored product.

In some cases, the T cell being detected is present in a sample comprising a plurality of T cells. For example, a T cell being detected can be present in a sample comprising from 10 to 10⁹ T cells, e.g., from 10 to 10², from 10² to 10⁴, from 10⁴ to 10⁶, from 10⁶ to 10⁷, from 10⁷ to 10⁸, or from 10⁸ to 10⁹, or more than 10⁹, T cells.

### Treatment Methods

The present disclosure provides treatment methods, the methods comprising administering to the individual an amount of a TMAPP of the present disclosure, or one or more nucleic acids or expression vectors encoding the TMAPP, effective to selectively modulate the activity of an epitope-specific T cell in an individual and to treat the individual. In some cases, a treatment method of the present disclosure comprises administering to an individual in need thereof one or more recombinant expression vectors comprising nucleotide sequences encoding a TMAPP of the present disclosure. In some cases, a treatment method of the present disclosure comprises administering to an individual in need thereof one or more mRNA molecules comprising nucleotide sequences encoding a TMAPP of the present disclosure. In some cases, a treatment method of the present disclosure comprises administering to an individual in need thereof a TMAPP of the present disclosure. Conditions that can be treated include autoimmune disorders other than T1D and/or celiac disease.

The present disclosure provides a method of selectively modulating the activity of an epitope-specific T cell in an individual, the method comprising administering to the individual an effective amount of a TMAPP of the present disclosure, or one or more nucleic acids (e.g., expression vectors; mRNA; etc.) comprising nucleotide sequences encoding the TMAPP, where the TMAPP selectively modulates the activity of the epitope-specific T cell in the individual. Selectively modulating the activity of an epitope-specific T cell can treat a disease or disorder in the individual. Thus, the present disclosure provides a treatment method comprising administering to an individual in need thereof an effective amount of a TMAPP of the present disclosure (e.g., a multimeric TMAPP of the present disclosure; or a single-chain TMAPP of the present disclosure). The disease or disorder is an autoimmune disease or disorder other than T1D or celiac disease.

In some cases, the immunomodulatory polypeptide is an inhibitory polypeptide, and a T TMAPP of the present disclosure inhibits activity of the epitope-specific T cell. In some cases, the epitope is a self-epitope, and a TMAPP of the present disclosure selectively inhibits the activity of a T cell specific for the self-epitope.

The present disclosure provides a method of treating an autoimmune disorder in an individual, the method comprising administering to the individual an effective amount of a TMAPP of the present disclosure, or one or more nucleic acids comprising nucleotide sequences encoding the TMAPP, where the TMAPP (e.g., a TMAPP of the present disclosure; or a single-chain TMAPP of the present disclosure) comprises a T-cell epitope that is a self epitope, and where the TMAPP comprises an inhibitory immunomodulatory polypeptide. In some cases, an “effective amount” of a TMAPP of the present disclosure is an amount that, when administered in one or more doses to an individual in need thereof, reduces the number self-reactive T cells by at least 10%, at least 15%, at least 20%, at least 25%, at least 30%, at least 40%, at least 50%, at least 60%, at least 70%, at least 80%, at least 90%, or at least 95%, compared to number of self-reactive T cells in the individual before administration of the TMAPP, or in the absence of administration with the TMAPP. In some cases, an “effective amount” of a TMAPP of the present disclosure is an amount that, when administered in one or more doses to an individual in need thereof, reduces production of Th2 cytokines in the individual. In some cases, an “effective amount” of a TMAPP of the present disclosure is an amount that, when administered in one or more doses to an individual in need thereof, ameliorates one or more symptoms associated with an autoimmune disease in the individual. In some instances, the TMAPP reduces the number of CD4⁺ self-reactive T cells, which in turn leads to a reduction in CD8⁺ self-reactive T cells. In some instances, the TMAPP increases the number of CD4⁺ Tregs, which in turn reduces the number of CD4⁺ self-reactive T cells and/or CD8⁺ T self-reactive T cells.

The present disclosure provides a method of treating an allergy in an individual, the method comprising administering to the individual an effective amount of a TMAPP of the present disclosure, or one or more nucleic acids comprising nucleotide sequences encoding the TMAPP, where the TMAPP (e.g., a TMAPP of the present disclosure; or a single-chain TMAPP of the present disclosure) comprises a T-cell epitope that is an allergen-associated peptide epitope, and where the TMAPP comprises an inhibitory immunomodulatory polypeptide. In some cases, an “effective amount” of a TMAPP of the present disclosure is an amount that, when administered in one or more doses to an individual in need thereof, reduces the number and/or activity of allergen-specific CD4⁺ T cells by at least 10%, at least 15%, at least 20%, at least 25%, at least 30%, at least 40%, at least 50%, at least 60%, at least 70%, at least 80%, at least 90%, or at least 95%, compared to number and/or activity of allergen-specific CD4⁺ T cells in the individual before administration of the TMAPP, or in the absence of administration with the TMAPP. In some cases, an “effective amount” of a TMAPP of the present disclosure is an amount that, when administered in one or more doses to an individual in need thereof, reduces the amount of cytokines produced by allergen-specific CD4⁺ T cells. In some cases, an “effective amount” of a TMAPP of the present disclosure is an amount that, when administered in one or more doses to an individual in need thereof, reduces level of IgE in the individual, compared to the level of IgE in the individual before administration of the TMAPP, or in the absence of administration with the TMAPP. In some cases, an “effective amount” of a TMAPP of the present disclosure is an amount that, when administered in one or more doses to an individual in need thereof, ameliorates one or more symptoms associated with an allergic disease in the individual.

An “allergen” is any substance that can cause an undesired (e.g., a Type 1 hypersensitive) immune response (i.e., allergic response or reaction) in a subject. Allergens include, but are not limited to, plant allergens (e.g., pollen, ragweed allergen), insect allergens, insect sting allergens (e.g., bee sting allergens), animal allergens (e.g., pet allergens, such as animal dander or cat Fel d 1 antigen), latex allergens, mold allergens, fungal allergens, cosmetic allergens, drug allergens, food allergens, dust, insect venom, viruses, bacteria, etc. Food allergens include, but are not limited to milk allergens, egg allergens, nut allergens (e.g., peanut or tree nut allergens, etc. (e.g., walnuts, cashews, etc.)), fish allergens, shellfish allergens, soy allergens, legume allergens, seed allergens and wheat allergens. Insect sting allergens include allergens that are or are associated with bee stings, wasp stings, hornet stings, yellow jacket stings, etc. Insect allergens also include house dust mite allergens (e.g., Der P1 antigen) and cockroach allergens. Drug allergens include allergens that are or are associated with antibiotics, non-steroidal ant-inflammatory drugs (NSAIDs), anesthetics, etc. Pollen allergens include grass allergens, tree allergens, weed allergens, flower allergens, etc.

Non-limiting examples of allergens include, but are not limited to, grass pollen allergens such as Phl p 1, Phl p 5, Phl p 6, Poa p 1, Poa p 5, Dac g 1, Fes p 1, Lol p 1 and Lol p 5; dust mite allergens such as Der f 1, Der f 2, Der p 1 and Der p 2; venom allergens such as Api m 1, Api m 2, Ves v 1, Ves v 2, Ves v 5, Dol m 1, Dol m 2, Dol m 5, Dol a 5, Pol a 1, Pol a 2, and Pol a 5; weed allergens such as Amb a 1, Amb a 2, Par j 1, Par o 1 and Par m 1; birch allergens such as Bet v 1; Japanese cedar allergens such as Cry j 1 and Cry j 2; cockroach allergens such as Per a 1; olive pollen allergens such as Ole e 1; cat allergens such as Fel d 1; dog allergens such as Can f 1 and Can f 2; horse allergens such as Equ c 1 and Equ c 2; mugworth allergens such as Art v 1, Art v 2, Art v 3; mold allergens such as Alt a 1, Alt a 3, Alt a 4, Alt a 5, Alt a 6, Cla h 1, Cla h 2 and Cla h 6; and fire ant allergens such as Sol i 2, Sol i 3 and Sol i 4.

As noted above, in some cases, in carrying out a subject treatment method, a TMAPP of the present disclosure is administered to an individual in need thereof, as the polypeptide per se. In other instances, in carrying out a subject treatment method, one or more nucleic acids comprising nucleotide sequences encoding a TMAPP is/are administering to an individual in need thereof. Thus, in other instances, one or more nucleic acids of the present disclosure, e.g., one or more recombinant expression vectors of the present disclosure, is/are administered to an individual in need thereof.

### Formulations

Suitable formulations are described above, where suitable formulations include a pharmaceutically acceptable excipient. In some cases, a suitable formulation comprises: a) a TMAPP of the present disclosure; and b) a pharmaceutically acceptable excipient. In some cases, a suitable formulation comprises: a) a nucleic acid comprising a nucleotide sequence encoding a TMAPP of the present disclosure; and b) a pharmaceutically acceptable excipient; in some instances, the nucleic acid is an mRNA. In some cases, a suitable formulation comprises: a) a first nucleic acid comprising a nucleotide sequence encoding the first polypeptide of a TMAPP of the present disclosure; b) a second nucleic acid comprising a nucleotide sequence encoding the second polypeptide of a TMAPP of the present disclosure; and c) a pharmaceutically acceptable excipient. In some cases, a suitable formulation comprises: a) a recombinant expression vector comprising a nucleotide sequence encoding a TMAPP of the present disclosure; and b) a pharmaceutically acceptable excipient. In some cases, a suitable formulation comprises: a) a first recombinant expression vector comprising a nucleotide sequence encoding the first polypeptide of a TMAPP of the present disclosure; b) a second recombinant expression vector comprising a nucleotide sequence encoding the second polypeptide of a TMAPP of the present disclosure; and c) a pharmaceutically acceptable excipient.

Suitable pharmaceutically acceptable excipients are described above.

### Dosages

A suitable dosage can be determined by an attending physician or other qualified medical personnel, based on various clinical factors. As is well known in the medical arts, dosages for any one patient depend upon many factors, including the patient's size, body surface area, age, the particular polypeptide or nucleic acid to be administered, sex of the patient, time, and route of administration, general health, and other drugs being administered concurrently. A multimeric polypeptide or a single-chain polypeptide of the present disclosure (e.g., a multimeric TMAPP or a single-chain TMAPP) may be administered in amounts between 1 ng/kg body weight and 20 mg/kg body weight per dose, e.g. between 0.1 mg/kg body weight to 10 mg/kg body weight, e.g. between 0.5 mg/kg body weight to 5 mg/kg body weight; however, doses below or above this exemplary range are envisioned, especially considering the aforementioned factors. If the regimen is a continuous infusion, it can also be in the range of 1 μg to 10 mg per kilogram of body weight per minute. A TMAPP of the present disclosure can be administered in an amount of from about 1 mg/kg body weight to 50 mg/kg body weight, e.g., from about 1 mg/kg body weight to about 5 mg/kg body weight, from about 5 mg/kg body weight to about 10 mg/kg body weight, from about 10 mg/kg body weight to about 15 mg/kg body weight, from about 15 mg/kg body weight to about 20 mg/kg body weight, from about 20 mg/kg body weight to about 25 mg/kg body weight, from about 25 mg/kg body weight to about 30 mg/kg body weight, from about 30 mg/kg body weight to about 35 mg/kg body weight, from about 35 mg/kg body weight to about 40 mg/kg body weight, or from about 40 mg/kg body weight to about 50 mg/kg body weight.

In some cases, a suitable dose of a TMAPP of the present disclosure is from 0.01 μg to 100 g per kg of body weight, from 0.1 μg to 10 g per kg of body weight, from 1 μg to 1 g per kg of body weight, from 10 μg to 100 mg per kg of body weight, from 100 μg to 10 mg per kg of body weight, or from 100 μg to 1 mg per kg of body weight. Persons of ordinary skill in the art can easily estimate repetition rates for dosing based on measured residence times and concentrations of the administered agent in bodily fluids or tissues. Following successful treatment, it may be desirable to have the patient undergo maintenance therapy to prevent the recurrence of the disease state, wherein a multimeric polypeptide or a single-chain polypeptide of the present disclosure (e.g., a multimeric TMAPP or a single-chain TMAPP) is administered in maintenance doses, ranging from 0.01 μg to 100 g per kg of body weight, from 0.1 μg to 10 g per kg of body weight, from 1 μg to 1 g per kg of body weight, from 10 μg to 100 mg per kg of body weight, from 100 μg to 10 mg per kg of body weight, or from 100 μg to 1 mg per kg of body weight.

Those of skill will readily appreciate that dose levels can vary as a function of the specific multimeric polypeptide or single-chain polypeptide (multimeric TMAPP or single-chain TMAPP), the severity of the symptoms and the susceptibility of the subject to side effects. Preferred dosages for a given compound are readily determinable by those of skill in the art by a variety of means.

In some cases, multiple doses of a TMAPP of the present disclosure, a nucleic acid of the present disclosure, or a recombinant expression vector of the present disclosure are administered. The frequency of administration of a TMAPP of the present disclosure, a nucleic acid of the present disclosure, or a recombinant expression vector of the present disclosure can vary depending on any of a variety of factors, e.g., severity of the symptoms, etc. For example, in some cases, a TMAPP of the present disclosure, a nucleic acid of the present disclosure, or a recombinant expression vector of the present disclosure is administered once per month, twice per month, three times per month, every other week (qow), once per week (qw), twice per week (biw), three times per week (tiw), four times per week, five times per week, six times per week, every other day (qod), daily (qd), twice a day (qid), or three times a day (tid).

The duration of administration of a TMAPP of the present disclosure, a nucleic acid of the present disclosure, or a recombinant expression vector of the present disclosure, e.g., the period of time over which a TMAPP of the present disclosure, a nucleic acid of the present disclosure, or a recombinant expression vector of the present disclosure is administered, can vary, depending on any of a variety of factors, e.g., patient response, etc. For example, a TMAPP of the present disclosure, a nucleic acid of the present disclosure, or a recombinant expression vector of the present disclosure can be administered over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more.

### Routes of Administration

An active agent (a TMAPP of the present disclosure, a nucleic acid of the present disclosure, or a recombinant expression vector of the present disclosure) is administered to an individual using any available method and route suitable for drug delivery, including in vivo and ex vivo methods, as well as systemic and localized routes of administration.

Conventional and pharmaceutically acceptable routes of administration include intratumoral, peritumoral, intramuscular, intratracheal, intralymphatic, intracranial, subcutaneous, intradermal, topical application, intravenous, intraarterial, rectal, nasal, oral, and other enteral and parenteral routes of administration. Routes of administration may be combined, if desired, or adjusted depending upon the TMAPP and/or the desired effect. A TMAPP of the present disclosure, or a nucleic acid or recombinant expression vector of the present disclosure, can be administered in a single dose or in multiple doses.

In some cases, a TMAPP of the present disclosure, a nucleic acid of the present disclosure, or a recombinant expression vector of the present disclosure is administered intravenously. In some cases, a TMAPP of the present disclosure, a nucleic acid of the present disclosure, or a recombinant expression vector of the present disclosure is administered intramuscularly. In some cases, a TMAPP of the present disclosure, a nucleic acid of the present disclosure, or a recombinant expression vector of the present disclosure is administered intralymphatically. In some cases, a TMAPP of the present disclosure, a nucleic acid of the present disclosure, or a recombinant expression vector of the present disclosure is administered locally. In some cases, a TMAPP of the present disclosure, a nucleic acid of the present disclosure, or a recombinant expression vector of the present disclosure is administered intratumorally. In some cases, a TMAPP of the present disclosure, a nucleic acid of the present disclosure, or a recombinant expression vector of the present disclosure is administered peritumorally. In some cases, a TMAPP of the present disclosure, a nucleic acid of the present disclosure, or a recombinant expression vector of the present disclosure is administered intracranially. In some cases, a TMAPP of the present disclosure, a nucleic acid of the present disclosure, or a recombinant expression vector of the present disclosure is administered subcutaneously.

In some cases, a TMAPP of the present disclosure is administered intravenously. In some cases, a TMAPP of the present disclosure is administered intramuscularly. In some cases, a TMAPP of the present disclosure is administered locally. In some cases, a TMAPP of the present disclosure is administered intratumorally. In some cases, a TMAPP of the present disclosure is administered peritumorally. In some cases, a TMAPP of the present disclosure is administered intracranially. In some cases, a TMAPP of the present disclosure is administered subcutaneously. In some cases, a TMAPP of the present disclosure is administered intralymphatically.

A TMAPP of the present disclosure, a nucleic acid of the present disclosure, or a recombinant expression vector of the present disclosure can be administered to a host using any available conventional methods and routes suitable for delivery of conventional drugs, including systemic or localized routes. In general, routes of administration contemplated for use in a method of the present disclosure include, but are not necessarily limited to, enteral, parenteral, and inhalational routes.

Parenteral routes of administration other than inhalation administration include, but are not necessarily limited to, topical, transdermal, subcutaneous, intramuscular, intraorbital, intracapsular, intraspinal, intrasternal, intratumoral, intralymphatic, peritumoral, and intravenous routes, i.e., any route of administration other than through the alimentary canal. Parenteral administration can be carried to effect systemic or local delivery of a TMAPP of the present disclosure, a nucleic acid of the present disclosure, or a recombinant expression vector of the present disclosure. Where systemic delivery is desired, administration typically involves invasive or systemically absorbed topical or mucosal administration of pharmaceutical preparations.

### Subjects Suitable for Treatment

Subjects suitable for treatment with a method of the present disclosure include individuals who have an autoimmune disease, including individuals who have been diagnosed as having an autoimmune disease, and individuals who have been treated for an autoimmune disease but who failed to respond to the treatment. Autoimmune diseases that can be treated with a method of the present disclosure include, but are not limited to, Addison's disease, alopecia areata, ankylosing spondylitis, autoimmune encephalomyelitis, autoimmune hemolytic anemia, autoimmune hepatitis, autoimmune-associated infertility, autoimmune thrombocytopenic purpura, bullous pemphigoid, Crohn's disease, Goodpasture's syndrome, glomerulonephritis (e.g., crescentic glomerulonephritis, proliferative glomerulonephritis), Grave's disease, Hashimoto's thyroiditis, mixed connective tissue disease, multiple sclerosis, myasthenia gravis (MG), pemphigus (e.g., pemphigus vulgaris), pernicious anemia, polymyositis, psoriasis, psoriatic arthritis, rheumatoid arthritis, scleroderma, Sjögren's syndrome, systemic lupus erythematosus (SLE), vasculitis, and vitiligo.

### Methods of Selectively Delivering a Costimulatory Polypeptide

The present disclosure provides a method of delivering a costimulatory polypeptide such as PD-L1, or a reduced-affinity variant of a naturally occurring costimulatory polypeptide such as a PD-L1 variant disclosed herein, to a selected T cell or a selected T cell population, e.g., in a manner such that a TCR specific for a given epitope is targeted. The present disclosure provides a method of delivering a costimulatory polypeptide such as PD-L1, or a reduced-affinity variant of a naturally occurring costimulatory polypeptide such as a PD-L1 variant disclosed herein, selectively to a target T cell bearing a TCR specific for the epitope present in a TMAPP of the present disclosure. The method comprises contacting a population of T cells with a TMAPP of the present disclosure. The population of T cells can be a mixed population that comprises: i) the target T cell; and ii) non-target T cells that are not specific for the epitope (e.g., T cells that are specific for an epitope(s) other than the epitope to which the epitope-specific T cell binds). The epitope-specific T cell is specific for the epitope-presenting peptide present in the TMAPP, and binds to the peptide HLA complex or peptide MHC complex provided by the TMAPP. Contacting the population of T cells with the TMAPP delivers the costimulatory polypeptide (e.g., PD-L1 or a reduced-affinity variant of PD-L1) present in the TMAPP selectively to the T cell(s) that are specific for the epitope present in the TMAPP.

Thus, the present disclosure provides a method of delivering a costimulatory polypeptide such as PD-L1, or a reduced-affinity variant of a naturally occurring costimulatory polypeptide such as a PD-L1 variant disclosed herein, or a combination of both, selectively to a target T cell, the method comprising contacting a mixed population of T cells with a TMAPP of the present disclosure. The mixed population of T cells comprises the target T cell and non-target T cells. The target T cell is specific for the epitope present within the TMAPP. Contacting the mixed population of T cells with a TMAPP of the present disclosure delivers the costimulatory polypeptide(s) present within the TMAPP to the target T cell.

For example, a TMAPP of the present disclosure is contacted with a population of T cells comprising: i) a target T cell(s) that is specific for the epitope present in the TMAPP; and ii) a non-target T cell(s), e.g., a T cell(s) that is specific for a second epitope(s) that is not the epitope present in the TMAPP. Contacting the population results in selective delivery of the costimulatory polypeptide(s) (e.g., naturally-occurring costimulatory polypeptide (e.g., naturally occurring PD-L1) or reduced-affinity variant of a naturally occurring costimulatory polypeptide (e.g., a PD-L1 variant disclosed herein)), which is present in the TMAPP, to the target T cell. Thus, e.g., less than 50%, less than 40%, less than 30%, less than 25%, less than 20%, less than 15%, less than 10%, less than 5%, or less than 4%, 3%, 2% or 1%, of the non-target T cells bind the TMAPP and, as a result, the costimulatory polypeptide (e.g., PD-L1 or PD-L1 variant) is not delivered to the non-target T cells.

In some cases, the population of T cells is in vitro. In some cases, the population of T cells is in vitro, and a biological response (e.g., T cell activation and/or expansion and/or phenotypic differentiation) of the target T cell population to the TMAPP of the present disclosure is elicited in the context of an in vitro culture. For example, a mixed population of T cells can be obtained from an individual, and can be contacted with the TMAPP in vitro. Such contacting can comprise single or multiple exposures of the population of T cells to a defined dose(s) and/or exposure schedule(s). In some cases, said contacting results in selectively binding/activating and/or expanding target T cells within the population of T cells, and results in generation of a population of activated and/or expanded target T cells. As an example, a mixed population of T cells can be peripheral blood mononuclear cells (PBMC). For example, PBMC from a patient can be obtained by standard blood drawing and PBMC enrichment techniques before being exposed to 0.1-1000 nM of a TMAPP of the present disclosure under standard lymphocyte culture conditions. At time points before, during, and after exposure of the mixed T cell population at a defined dose and schedule, the abundance of target T cells in the in vitro culture can be monitored by specific peptide-MHC multimers and/or phenotypic markers and/or functional activity (e.g. cytokine ELISpot assays). In some cases, upon achieving an optimal abundance and/or phenotype of antigen specific cells in vitro, all or a portion of the population of activated and/or expanded target T cells is administered to the individual (the individual from whom the mixed population of T cells was obtained).

In some cases, the population of T cells is in vitro. For example, a mixed population of T cells is obtained from an individual, and is contacted with a TMAPP of the present disclosure in vitro. Such contacting, which can comprise single or multiple exposures of the T cells to a defined dose(s) and/or exposure schedule(s) in the context of in vitro cell culture, can be used to determine whether the mixed population of T cells includes T cells that are specific for the epitope presented by the TMAPP. The presence of T cells that are specific for the epitope of the TMAPP can be determined by assaying a sample comprising a mixed population of T cells, which population of T cells comprises T cells that are not specific for the epitope (non-target T cells) and may comprise T cells that are specific for the epitope (target T cells). Known assays can be used to detect activation and/or proliferation of the target T cells, thereby providing an ex vivo assay that can determine whether a particular TMAPP possesses an epitope that binds to T cells present in the individual and thus whether the TMAPP has potential use as a therapeutic composition for that individual. Suitable known assays for detection of activation and/or proliferation of target T cells include, e.g., flow cytometric characterization of T cell phenotype and/or antigen specificity and/or proliferation. Such an assay to detect the presence of epitope-specific T cells, e.g., a companion diagnostic, can further include additional assays (e.g. effector cytokine ELISpot assays) and/or appropriate controls (e.g. antigen-specific and antigen-nonspecific multimeric peptide-HLA staining reagents) to determine whether the TMAPP is selectively binding/activating and/or expanding the target T cell. Thus, for example, the present disclosure provides a method of detecting, in a mixed population of T cells obtained from an individual, the presence of a target T cell that binds an epitope of interest, the method comprising: a) contacting in vitro the mixed population of T cells with a TMAPP of the present disclosure, wherein the multimeric polypeptide comprises the epitope of interest; and b) detecting activation and/or proliferation of T cells in response to said contacting, wherein activated and/or proliferated T cells indicates the presence of the target T cell. Alternatively, and/or in addition, if activation and/or expansion (proliferation) of the desired T cell population is obtained using the TMAPP, then all or a portion of the population of T cells comprising the activated/expanded T cells can be administered back to the individual as a therapy.

In some instances, the population of T cells is in vivo in an individual. In such instances, a method of the present disclosure for selectively delivering a costimulatory polypeptide (e.g., PD-L1 or a reduced-affinity PD-L1) to an epitope-specific T cell comprises administering the TMAPP to the individual.

The epitope-specific T cell to which a costimulatory polypeptide (e.g., IL-2 or a reduced-affinity PD-L1) is being selectively delivered is also referred to herein as a “target T cell.” In some cases, the target T cell is a regulatory T cell (Treg). In some cases, the Treg inhibits or suppresses activity of an autoreactive T cell.

### EXAMPLES OF NON-LIMITING ASPECTS OF THE DISCLOSURE

Aspects, including embodiments, of the present subject matter described above may be beneficial alone or in combination, with one or more other aspects or embodiments.

### Aspects Set A

Aspect 1. A multimeric antigen-presenting polypeptide comprising: a) a first polypeptide comprising: i) a first major histocompatibility complex (MHC) Class II polypeptide; and b) a second polypeptide comprising: i) a second MHC Class II polypeptide; and ii) optionally an immunoglobulin (Ig) Fc polypeptide or a non-Ig scaffold, wherein the multimeric polypeptide comprises an epitope capable of being bound by a T-cell receptor (TCR), wherein the epitope is: A) at the N-terminus of the first polypeptide; or B) at the N-terminus of the second polypeptide.

Aspect 2. The multimeric antigen-presenting polypeptide of aspect 1, wherein: a) the first polypeptide comprises, in order from N-terminus to C-terminus: i) the epitope; ii) an MHC Class II α1 polypeptide; and iii) an MHC Class II α2 polypeptide; and b) the second polypeptide comprises, in order from N-terminus to C-terminus: i) an MHC Class II β1 polypeptide; and ii) an MHC Class II β2 polypeptide.

Aspect 3. The multimeric antigen-presenting polypeptide of aspect 1, wherein: a) the first polypeptide comprises, in order from N-terminus to C-terminus: i) the epitope; ii) an MHC Class II β1 polypeptide; and iii) an MHC Class II β2 polypeptide; and b) the second polypeptide comprises, in order from N-terminus to C-terminus: i) an MHC Class II α1 polypeptide; and ii) an MHC Class II α2 polypeptide.

Aspect 4. The multimeric antigen-presenting polypeptide of aspect 1, wherein: a) the first polypeptide comprises, in order from N-terminus to C-terminus: i) the epitope; ii) an MHC Class II β1 polypeptide; iii) an MHC Class II α1 polypeptide; and iv) an MHC Class II α2 polypeptide; and b) the second polypeptide comprises an MHC Class II β2 polypeptide.

Aspect 5. The multimeric antigen-presenting polypeptide of any one of aspects 1-4, wherein the first polypeptide comprises an Ig Fc polypeptide at the C-terminus.

Aspect 6. The multimeric antigen-presenting polypeptide of any one of aspects 1-5, wherein: a) the first polypeptide comprises a first dimerization polypeptide; and b) the second polypeptide comprises a second dimerization polypeptide.

Aspect 7. The multimeric antigen-presenting polypeptide of aspect 6, wherein the first and the second dimerization polypeptides are leucine-zipper polypeptides, collagen dimerization polypeptides, or coiled-coil polypeptides.

Aspect 8. The multimeric antigen-presenting polypeptide of any one of aspects 2-6, wherein the MHC Class II α1 polypeptide comprises an amino acid sequence having at least 95% amino acid sequence identity to an MHC Class II α1 polypeptide depicted in any one of FIGS. 6, 11, 13, 15, 17, and 18.

Aspect 9. The multimeric antigen-presenting polypeptide of any one of aspects 2-6, wherein the MHC Class II α2 polypeptide comprises an amino acid sequence having at least 95% amino acid sequence identity to an MHC Class II α2 polypeptide depicted in any one of FIGS. 6, 11, 13, 15, 17, and 18.

Aspect 10. The multimeric antigen-presenting polypeptide of any one of aspects 2-6, wherein the MHC Class II β1 polypeptide comprises an amino acid sequence having at least 95% amino acid sequence identity to an MHC Class II β1 polypeptide depicted in any one of FIG. 7A-7J, FIG. 8A-8B, FIG. 9, FIG. 10, FIG. 12, FIG. 14, FIG. 16, FIG. 19A-19B, and FIG. 20A-20B.

Aspect 11. The multimeric antigen-presenting polypeptide of any one of aspects 2-6, wherein the MHC Class II β2 polypeptide comprises an amino acid sequence having at least 95% amino acid sequence identity to an MHC Class II β2 polypeptide depicted in any one of FIG. 7A-7J, FIG. 8A-8B, FIG. 9, FIG. 10, FIG. 12, FIG. 14, FIG. 16, FIG. 19A-19B, and FIG. 20A-20B.

Aspect 12. A single-chain antigen-presenting polypeptide comprising: i) a major histocompatibility complex (MHC) Class II α1 polypeptide; ii) a Class II MHC α2 polypeptide; iii) a Class II MHC β1 polypeptide; iv) a Class II MHC β2 polypeptide; v) an epitope capable of being bound by a T-cell receptor (TCR); and vi) optionally an immunoglobulin (Ig) Fc polypeptide or a non-Ig scaffold.

Aspect 13. The single-chain antigen-presenting polypeptide of aspect 12, wherein the polypeptide comprises, in order from N-terminus to C-terminus: i) the epitope; ii) the Class II MHC polypeptide; iii) the Class II MHC α1 polypeptide; iv) the Class II MHC α2 polypeptide; and v) the Class II MHC β2 polypeptide.

Aspect 14. The single-chain antigen-presenting polypeptide of aspect 12, wherein the polypeptide comprises, in order from N-terminus to C-terminus: i) the epitope; ii) the Class II MHC β1 polypeptide; iii) the Class II MHC β2 polypeptide; iv) the Class II MHC α1 polypeptide; and v) the Class II MHC α2 polypeptide.

Aspect 15. The single-chain antigen-presenting polypeptide of aspect 13 or aspect 14, comprising an immunoglobulin Fc polypeptide at the C-terminus.

Aspect 16. The single-chain antigen-presenting polypeptide of aspect 13, comprising a linker.

Aspect 17. The single-chain antigen-presenting polypeptide of aspect 16, wherein the linker is between the epitope and the Class II MHC polypeptide.

Aspect 18. The single-chain antigen-presenting polypeptide of aspect 14, comprising a linker.

Aspect 19. The single-chain antigen-presenting polypeptide of aspect 18, wherein the linker is between the epitope and the Class II MHC polypeptide.

Aspect 20. The single-chain antigen-presenting polypeptide of any one of aspects 12-19, wherein the MHC Class II α1 polypeptide comprises an amino acid sequence having at least 95% amino acid sequence identity to an MHC Class II α1 polypeptide depicted in any one of FIGS. 6, 11, 13, 15, 17, and 18.

Aspect 21. The single-chain antigen-presenting polypeptide of any one of aspects 12-19, wherein the MHC Class II α2 polypeptide comprises an amino acid sequence having at least 95% amino acid sequence identity to an MHC Class II α2 polypeptide depicted in any one of FIGS. 6, 11, 13, 15, 17, and 18.

Aspect 22. The single-chain antigen-presenting polypeptide of any one of aspects 12-19, wherein the MHC Class II β1 polypeptide comprises an amino acid sequence having at least 95% amino acid sequence identity to an MHC Class II β1 polypeptide depicted in any one of FIG. 7A-7J, FIG. 8A-8B, FIG. 9, FIG. 10, FIG. 12, FIG. 14, FIG. 16, FIG. 19A-19B, and FIG. 20A-20B.

Aspect 23. The single-chain antigen-presenting polypeptide of any one of aspects 12-19, wherein the MHC Class II β2 polypeptide comprises an amino acid sequence having at least 95% amino acid sequence identity to an MHC Class II β2 polypeptide depicted in any one of FIG. 7A-7J, FIG. 8A-8B, FIG. 9, FIG. 10, FIG. 12, FIG. 14, FIG. 16, FIG. 19A-19B, and FIG. 20A-20B.

Aspect 24. A multimeric T-cell modulatory antigen-presenting polypeptide comprising: a) a first polypeptide comprising: i) an epitope capable of being bound by a T-cell receptor (TCR); ii) a first major histocompatibility complex (MHC) Class II polypeptide; and b) a second polypeptide comprising: i) a second MHC Class II polypeptide; and wherein one or both polypeptides of the multimeric polypeptide comprises one or more immunomodulatory polypeptides, and wherein one or both polypeptides of the multimeric polypeptide optionally comprise an immunoglobulin (Ig) Fc polypeptide or a non-Ig scaffold.

Aspect 25. The multimeric T-cell modulatory antigen-presenting polypeptide of aspect 24, wherein: a) the first polypeptide comprises, in order from N-terminus to C-terminus: i) the epitope; ii) an MHC Class II β1 polypeptide; iii) an MHC Class II β2 polypeptide; and iv) an immunomodulatory domain; and b) the second polypeptide comprises, in order from N-terminus to C-terminus: i) an MHC Class II α1 polypeptide; ii) an MHC Class II α2 polypeptide.

Aspect 26. The multimeric T-cell modulatory antigen-presenting polypeptide of aspect 24, wherein: a) the first polypeptide comprises, in order from N-terminus to C-terminus: i) the epitope; ii) an MHC Class II β1 polypeptide; iii) an MHC Class II β2 polypeptide; and iv) an immunomodulatory domain; and b) the second polypeptide comprises, in order from N-terminus to C-terminus: i) an MHC Class II α1 polypeptide; ii) an MHC Class II α2 polypeptide; and iii) an Ig Fc polypeptide.

Aspect 27. The multimeric T-cell modulatory antigen-presenting polypeptide of aspect 24, wherein: a) the first polypeptide comprises, in order from N-terminus to C-terminus: i) the epitope; ii) an MHC Class II β1 polypeptide; iii) an MHC Class II β2 polypeptide; iv) an immunomodulatory domain; and v) a first dimerization polypeptide; and b) the second polypeptide comprises, in order from N-terminus to C-terminus: i) an MHC Class II α1 polypeptide; ii) an MHC Class II α2 polypeptide; and iii) a second dimerization polypeptide.

Aspect 28. The multimeric T-cell modulatory antigen-presenting polypeptide of aspect 24, wherein: a) the first polypeptide comprises, in order from N-terminus to C-terminus: i) the epitope; ii) an MHC Class II β1 polypeptide; iii) an MHC Class II β2 polypeptide; and b) the second polypeptide comprises, in order from N-terminus to C-terminus: i) an immunomodulatory domain; ii) an MHC Class II α1 polypeptide; and iii) an MHC Class II α2 polypeptide.

Aspect 29. The multimeric T-cell modulatory antigen-presenting polypeptide of aspect 24, wherein: a) the first polypeptide comprises, in order from N-terminus to C-terminus: i) the epitope; ii) an MHC Class II β1 polypeptide; iii) an MHC Class II β2 polypeptide; and b) the second polypeptide comprises, in order from N-terminus to C-terminus: i) an immunomodulatory domain; ii) an MHC Class II α1 polypeptide; iii) an MHC Class II α2 polypeptide; and iv) an Ig Fc polypeptide.

Aspect 30. The multimeric T-cell modulatory antigen-presenting polypeptide of aspect 24, wherein: a) the first polypeptide comprises, in order from N-terminus to C-terminus: i) the epitope; ii) an MHC Class II β1 polypeptide; iii) an MHC Class II β2 polypeptide; and iv) a first dimerization polypeptide; and b) the second polypeptide comprises, in order from N-terminus to C-terminus: i) an immunomodulatory domain; ii) an MHC Class II α1 polypeptide; iii) an MHC Class II α2 polypeptide; and iv) a second dimerization polypeptide.

Aspect 31. The multimeric T-cell modulatory antigen-presenting polypeptide of aspect 24, wherein: a) the first polypeptide comprises, in order from N-terminus to C-terminus: i) the epitope; ii) an MHC Class II β1 polypeptide; iii) an MHC Class II α1 polypeptide; iv) an MHC Class II α2 polypeptide; and b) the second polypeptide comprises, in order from N-terminus to C-terminus: i) an immunomodulatory domain; and ii) an MHC Class II β2 polypeptide.

Aspect 32. The multimeric T-cell modulatory antigen-presenting polypeptide of aspect 24, wherein: a) the first polypeptide comprises, in order from N-terminus to C-terminus: i) the epitope; ii) an MHC Class II β1 polypeptide; iii) an MHC Class II α1 polypeptide; iv) an MHC Class II α2 polypeptide; and b) the second polypeptide comprises, in order from N-terminus to C-terminus: i) an immunomodulatory domain; ii) an MHC Class II β2 polypeptide; and iii) an Ig Fc polypeptide.

Aspect 33. The multimeric T-cell modulatory antigen-presenting polypeptide of aspect 24, wherein: a) the first polypeptide comprises, in order from N-terminus to C-terminus: i) the epitope; ii) an MHC Class II β1 polypeptide; iii) an MHC Class II α1 polypeptide; iv) an MHC Class II α2 polypeptide; and v) a first dimerization polypeptide; and b) the second polypeptide comprises, in order from N-terminus to C-terminus: i) an immunomodulatory domain; ii) an MHC Class II β2 polypeptide; and iii) a second dimerization polypeptide.

Aspect 34. The multimeric T-cell modulatory antigen-presenting polypeptide of any one of aspects 24-33, comprising a linker.

Aspect 35. The multimeric T-cell modulatory antigen-presenting polypeptide of any one of aspects 24-33, wherein the MHC Class II α1 polypeptide comprises an amino acid sequence having at least 95% amino acid sequence identity to an MHC Class II α1 polypeptide depicted in any one of FIGS. 6, 11, 13, 15, 17, and 18.

Aspect 36. The multimeric T-cell modulatory antigen-presenting polypeptide of any one of aspects 24-33, wherein the MHC Class II α2 polypeptide comprises an amino acid sequence having at least 95% amino acid sequence identity to an MHC Class II α2 polypeptide depicted in any one of FIGS. 6, 11, 13, 15, 17, and 18.

Aspect 37. The multimeric T-cell modulatory antigen-presenting polypeptide of any one of aspects 24-33, wherein the MHC Class II β1 polypeptide comprises an amino acid sequence having at least 95% amino acid sequence identity to an MHC Class II β1 polypeptide depicted in any one of FIG. 7A-7J, FIG. 8A-8B, FIG. 9, FIG. 10, FIG. 12, FIG. 14, FIG. 16, FIG. 19A-19B, and FIG. 20A-20B.

Aspect 38. The multimeric T-cell modulatory antigen-presenting polypeptide of any one of aspects 24-33, wherein the MHC Class II β2 polypeptide comprises an amino acid sequence having at least 95% amino acid sequence identity to an MHC Class II β2 polypeptide depicted in any one of FIG. 7A-7J, FIG. 8A-8B, FIG. 9, FIG. 10, FIG. 12, FIG. 14, FIG. 16, FIG. 19A-19B, and FIG. 20A-20B.

Aspect 39. The multimeric T-cell modulatory antigen-presenting polypeptide of any one of aspects 24-38, wherein the immunomodulatory polypeptide comprises the amino acid sequence of a naturally-occurring immunomodulatory polypeptide.

Aspect 40. The multimeric T-cell modulatory antigen-presenting polypeptide of aspect 39, wherein the immunomodulatory polypeptide is selected from the group consisting of IL-2, 4-1BBL, PD-L1, CD80, CD86, B7-1, ICOS-L, OX-40L, FasL, TGFβ, JAG1, and PD-L2.

Aspect 41. The multimeric T-cell modulatory antigen-presenting polypeptide of any one of aspects 24-38, wherein the immunomodulatory polypeptide is a variant immunomodulatory polypeptide that comprises an amino acid sequence having from 1 to 10 amino acid substitutions compared to the amino acid sequence of a naturally-occurring immunomodulatory polypeptide, wherein the variant immunomodulatory polypeptide has reduced affinity for a co-immunomodulatory polypeptide, compared to the affinity of the naturally-occurring immunomodulatory polypeptide for the co-immunomodulatory polypeptide.

Aspect 42. The multimeric T-cell modulatory antigen-presenting polypeptide of aspect 41, wherein the variant immunomodulatory polypeptide is a variant 4-1BBL polypeptide.

Aspect 43. The multimeric T-cell modulatory antigen-presenting polypeptide of aspect 41, wherein the variant immunomodulatory polypeptide is a variant CD80 polypeptide.

Aspect 44. The multimeric T-cell modulatory antigen-presenting polypeptide of aspect 41, wherein the variant immunomodulatory polypeptide is a variant IL-2 polypeptide.

Aspect 45. The multimeric T-cell modulatory antigen-presenting polypeptide of aspect 41, wherein the variant immunomodulatory polypeptide is a variant CD86 polypeptide.

Aspect 46. The multimeric T-cell modulatory antigen-presenting polypeptide of aspect 41, wherein the variant immunomodulatory polypeptide is a variant PD-L1 polypeptide.

Aspect 47. The multimeric T-cell modulatory antigen-presenting polypeptide of any one of aspects 24-46, wherein the multimeric polypeptide comprises two immunomodulatory polypeptides.

Aspect 48. The multimeric T-cell modulatory antigen-presenting polypeptide of aspect 47, wherein the two immunomodulatory polypeptides are on the same polypeptide chain.

Aspect 49. The multimeric T-cell modulatory antigen-presenting polypeptide of aspect 47, wherein the two immunomodulatory polypeptides are on separate polypeptide chains.

Aspect 50. The multimeric T-cell modulatory antigen-presenting polypeptide of any one of aspects 47-49, wherein the two immunomodulatory polypeptides comprise the same amino acid sequence.

Aspect 51. The multimeric T-cell modulatory antigen-presenting polypeptide of any one of aspects 24-50, wherein the multimeric polypeptide comprises a peptide linker between one or more of: a) the epitope and the MHC polypeptide; b) any two adjacent MHC polypeptides; c) the MHC polypeptide and the Fc polypeptide; and d) two adjacent immunomodulatory polypeptides.

Aspect 52. The multimeric T-cell modulatory antigen-presenting polypeptide of aspect 51, wherein the linker has a length of from 20 amino acids to 40 amino acids.

Aspect 53. The multimeric T-cell modulatory antigen-presenting polypeptide of 51 or 52, wherein the linker is a peptide of the formula (GGGGS)n (SEQ ID NO: 1), where n is 1, 2, 3, 4, 5, 6, 7, or 8.

Aspect 54. A single-chain T-cell modulatory antigen-presenting polypeptide comprising: i) an epitope capable of being bound by a T-cell receptor (TCR); ii) an major histocompatibility complex (MHC) Class II α1 polypeptide; iii) an MHC Class II α2 polypeptide; iv) an MHC Class II β1 polypeptide; v) an MHC Class II β2 polypeptide; vi) an immunomodulatory polypeptide; and vii) optionally an immunoglobulin (Ig) Fc polypeptide or a non-Ig scaffold.

Aspect 55. The single-chain T-cell modulatory antigen-presenting polypeptide of aspect 54 comprising, in order from N-terminus to C-terminus: i) the epitope; ii) the MHC Class II β1 polypeptide; iii) the MHC Class II α1 polypeptide; iv) the MHC Class II α2 polypeptide; v) the MHC Class II β2 polypeptide; and vi) the immunomodulatory polypeptide.

Aspect 56. The single-chain T-cell modulatory antigen-presenting polypeptide of aspect 54 comprising, in order from N-terminus to C-terminus: i) the epitope; ii) a first immunomodulatory polypeptide; iii) the MHC Class II β1 polypeptide; iv) the MHC Class II α1 polypeptide; v) the MHC Class II α2 polypeptide; vi) the MHC Class II β2 polypeptide; and vii) a second immunomodulatory polypeptide, wherein the first and the second immunomodulatory polypeptides comprise the same amino acid sequence.

Aspect 57. The single-chain T-cell modulatory antigen-presenting polypeptide of aspect 54 comprising, in order from N-terminus to C-terminus: i) the immunomodulatory polypeptide; ii) the epitope; iii) the MHC Class II β1 polypeptide; iv) the MHC Class II α1 polypeptide; v) the MHC Class II α2 polypeptide; and vi) the MHC Class II β2 polypeptide.

Aspect 58. The single-chain T-cell modulatory antigen-presenting polypeptide of aspect 54 comprising, in order from N-terminus to C-terminus: i) the epitope; ii) the MHC Class II β1 polypeptide; iii) the MHC Class II β2 polypeptide; iv) the MHC Class II α1 polypeptide; v) the MHC Class II α2 polypeptide; and vi) the immunomodulatory polypeptide.

Aspect 59. The single-chain T-cell modulatory antigen-presenting polypeptide of aspect 54 comprising, in order from N-terminus to C-terminus: i) the epitope; ii) the immunomodulatory polypeptide; iii) the MHC Class II β1 polypeptide; iv) the MHC Class II β2 polypeptide; v) the MHC Class II α1 polypeptide; and vi) the MHC Class II α2 polypeptide.

Aspect 60. The single-chain T-cell modulatory antigen-presenting polypeptide of aspect 54 comprising, in order from N-terminus to C-terminus: i) the immunomodulatory polypeptide; ii) the epitope; iii) the MHC Class II β1 polypeptide; iv) the MHC Class II β2 polypeptide; v) the MHC Class II α1 polypeptide; and vi) the MHC Class II α2 polypeptide.

Aspect 61. The single-chain T-cell modulatory antigen-presenting polypeptide of any one of aspects 54-60, wherein the MHC Class II α1 polypeptide comprises an amino acid sequence having at least 95% amino acid sequence identity to an MHC Class II α1 polypeptide depicted in any one of FIGS. 6, 11, 13, 15, 17, and 18.

Aspect 62. The single-chain T-cell modulatory antigen-presenting polypeptide of any one of aspects 54-60, wherein the MHC Class II α2 polypeptide comprises an amino acid sequence having at least 95% amino acid sequence identity to an MHC Class II α2 polypeptide depicted in any one of FIGS. 6, 11, 13, 15, 17, and 18.

Aspect 63. The single-chain T-cell modulatory antigen-presenting polypeptide of any one of aspects 54-60, wherein the MHC Class II β1 polypeptide comprises an amino acid sequence having at least 95% amino acid sequence identity to an MHC Class II β1 polypeptide depicted in any one of FIG. 7A-7J, FIG. 8A-8B, FIG. 9, FIG. 10, FIG. 12, FIG. 14, FIG. 16, FIG. 19A-19B, and FIG. 20A-20B.

Aspect 64. The single-chain T-cell modulatory antigen-presenting polypeptide of any one of aspects 54-60, wherein the MHC Class II β2 polypeptide comprises an amino acid sequence having at least 95% amino acid sequence identity to an MHC Class II β2 polypeptide depicted in any one of FIG. 7A-7J, FIG. 8A-8B, FIG. 9, FIG. 10, FIG. 12, FIG. 14, FIG. 16, FIG. 19A-19B, and FIG. 20A-20B.

Aspect 65. The single-chain T-cell modulatory antigen-presenting polypeptide of any one of aspects 54-64, wherein the immunomodulatory polypeptide comprises the amino acid sequence of a naturally-occurring immunomodulatory polypeptide.

Aspect 66. The single-chain T-cell modulatory antigen-presenting polypeptide of aspect 65, wherein the immunomodulatory polypeptide is selected from the group consisting of IL-2, 4-1BBL, PD-L1, CD80, CD86, B7-1, ICOS-L, OX-40L, FasL, JAG1, TGFβ, and PD-L2.

Aspect 67. The single-chain T-cell modulatory antigen-presenting polypeptide of any one of aspects 54-64, wherein the immunomodulatory polypeptide is a variant immunomodulatory polypeptide that comprises an amino acid sequence having from 1 to 10 amino acid substitutions compared to the amino acid sequence of a naturally-occurring immunomodulatory polypeptide, wherein the variant immunomodulatory polypeptide has reduced affinity for a co-immunomodulatory polypeptide, compared to the affinity of the naturally-occurring immunomodulatory polypeptide for the co-immunomodulatory polypeptide.

Aspect 68. The single-chain T-cell modulatory antigen-presenting polypeptide of aspect 67, wherein the variant immunomodulatory polypeptide is a variant 4-1BBL polypeptide.

Aspect 69. The single-chain T-cell modulatory antigen-presenting polypeptide of aspect 67, wherein the variant immunomodulatory polypeptide is a variant CD80 polypeptide.

Aspect 70. The single-chain T-cell modulatory antigen-presenting polypeptide of aspect 67, wherein the variant immunomodulatory polypeptide is a variant IL-2 polypeptide.

Aspect 71. The single-chain T-cell modulatory antigen-presenting polypeptide of aspect 67, wherein the variant immunomodulatory polypeptide is a variant CD86 polypeptide.

Aspect 72. The single-chain T-cell modulatory antigen-presenting polypeptide of aspect 67, wherein the variant immunomodulatory polypeptide is a variant PD-L1 polypeptide.

Aspect 73. The single-chain T-cell modulatory antigen-presenting polypeptide of any one of aspects 54-72, wherein the polypeptide comprises two immunomodulatory polypeptides.

Aspect 74. The single-chain T-cell modulatory antigen-presenting polypeptide of aspect 73, wherein the two immunomodulatory polypeptides comprise the same amino acid sequence.

Aspect 75. The single-chain T-cell modulatory antigen-presenting polypeptide of any one of aspects 54-74, wherein the multimeric polypeptide comprises a peptide linker between one or more of: a) the epitope and the MHC polypeptide; b) any two adjacent MHC polypeptides; c) the MHC polypeptide and the Fc polypeptide; and d) two adjacent immunomodulatory polypeptides.

Aspect 76. The single-chain T-cell modulatory antigen-presenting polypeptide of aspect 75, wherein the linker has a length of from 20 amino acids to 40 amino acids.

Aspect 77. The single-chain T-cell modulatory antigen-presenting polypeptide of aspect 75 or 76, wherein the linker is a peptide of the formula (GGGGS)n (SEQ ID NO:1), where n is 1, 2, 3, 4, 5, 6, 7, or 8.

Aspect 78. The multimeric T-cell modulatory antigen-presenting polypeptide of any one of aspects 24-53, or the single-chain T-cell modulatory antigen-presenting polypeptide of any one of aspects 54-77, comprising an Ig Fc polypeptide, and wherein the Ig Fc polypeptide is an IgG1 Fc polypeptide, an IgG2 Fc polypeptide, an IgG3 Fc polypeptide, an IgG4 Fc polypeptide, an IgA Fc polypeptide, or an IgM Fc polypeptide.

Aspect 79. The multimeric T-cell modulatory antigen-presenting polypeptide or single-chain T-cell modulatory antigen-presenting polypeptide of aspect 78, wherein a drug is conjugated to the Ig Fc polypeptide.

Aspect 80. The multimeric T-cell modulatory antigen-presenting polypeptide of any one of aspects 24-53, or the single-chain T-cell modulatory antigen-presenting polypeptide of any one of aspects 54-77, wherein the epitope is a cancer epitope.

Aspect 81. The multimeric T-cell modulatory antigen-presenting polypeptide of any one of aspects 24-53, or the single-chain T-cell modulatory antigen-presenting polypeptide of any one of aspects 54-77, wherein the epitope is an auto-epitope.

Aspect 82. The multimeric T-cell modulatory antigen-presenting polypeptide of any one of aspects 24-53, or the single-chain T-cell modulatory antigen-presenting polypeptide of any one of aspects 54-77, wherein the first and the second MHC class II polypeptides comprise:

i) an MHC class II α chain polypeptide having at least 90% amino acid sequence identity to a DRA1*01:01 polypeptide; and an MHC class II β chain polypeptide having at least 90% amino acid sequence identity to a DRB1 polypeptide; or

ii) an MHC class II α chain polypeptide having at least 90% amino acid sequence identity to a DRA1 polypeptide; and an MHC class II DRB1 chain polypeptide having at least 90% amino acid sequence identity to a DRB1 polypeptide of one of the following alleles: DRB1*01:01, DRB1*01:02, DRB1*01:03, DRB1*0301, DRB1*03:02, DRB1*0304, DRB1*04:01, DRB1*04:02, DRB1*0403, DRB1*04:04, DRB1*04:05, DRB1*04:06, DRB1*04:08, DRB1*08:03, DRB1*09:01, DRB1*10:01, DRB1*11:04, DRB1*13:01, DRB1*14:02, DRB1*1501, DRB1*1502, and DRB1*1503; or

iii) an MHC class II α chain polypeptide having at least 90% amino acid sequence identity to a DQA1 polypeptide of one of the following alleles: DQA1*01:01, DQA*01:02, DQA1*01:04, DQA1*03:01, DQA1*03:02, DQA1*04:01, DQA1*05:01; and an MHC class II β chain polypeptide having at least 90% amino acid sequence identity to a DQB1 polypeptide; or

iv) an MHC class II α chain polypeptide having at least 90% amino acid sequence identity to a DQA1 polypeptide; and an MHC class II β chain polypeptide having at least 90% amino acid sequence identity to a DQB1 polypeptide of one of the following alleles: DQB1*02:01, DQB1*03:01, DQB1*03:03, DQB1*04:01, DQB1*04:02, DQB1*05:01, DQB1*05:03, DQB1*06:01, and DQB1*0602; or

v) an MHC class II α chain polypeptide having at least 90% amino acid sequence identity to a DRA3 polypeptide; and an MHC class II β chain polypeptide having at least 90% amino acid sequence identity to a DRB3 polypeptide of one of the following alleles: DRB3*01:01 and DRB3*03:01; or

vi) an MHC class II α chain polypeptide having at least 90% amino acid sequence identity to a DRA4 polypeptide; and an MHC class II β chain polypeptide having at least 90% amino acid sequence identity to a DRB4*01:01 polypeptide; or

vii) an MHC class II α chain polypeptide having at least 90% amino acid sequence identity to a DRA1 polypeptide; and an MHC class II β chain polypeptide having at least 90% amino acid sequence identity to a DRB5*01:01 polypeptide; or

viii) an MHC class II α chain polypeptide having at least 90% amino acid sequence identity to a DPA1 polypeptide; and an MHC class II β chain polypeptide having at least 90% amino acid sequence identity to a DPB1 polypeptide of one of the following alleles: DPB1*03:01, DPB1*09:01, and DPB1*13:01.

Aspect 83. A composition comprising: a) an antigen-presenting polypeptide of any one of aspects 1-82; and b) a buffer.

Aspect 84. A composition comprising: a) the T-cell modulatory antigen-presenting polypeptide of any one of aspects 24-77 and 82; and b) a pharmaceutically acceptable excipient.

Aspect 85. A composition comprising: a) the T-cell modulatory antigen-presenting polypeptide of any one of aspects 24-77 and 82; and b) saline.

Aspect 86. The composition of aspect 85, wherein the saline is 0.9% NaCl.

Aspect 87. The composition of aspect 85 or aspect 86, wherein the composition is sterile.

Aspect 88. One or more nucleic acids comprising nucleotide sequences encoding the antigen-presenting polypeptide of any one of aspects 1-23.

Aspect 89. One or more recombinant expression vectors comprising the one or more nucleic acids of aspect 88.

Aspect 90. A host cell genetically modified with the one or more nucleic acids of aspect 88 or the one or more recombinant expression vectors of aspect 89.

Aspect 91. The host cell of aspect 90, wherein the host cell is a eukaryotic cell.

Aspect 92. One or more nucleic acids comprising nucleotide sequences encoding the T-cell modulatory antigen-presenting polypeptide of any one of aspects 24-77.

Aspect 93. One or more recombinant expression vectors comprising the one or more nucleic acids of aspect 92.

Aspect 94. A host cell genetically modified with the one or more nucleic acids of aspect 91 or the one or more recombinant expression vectors of aspect 93.

Aspect 95. The host cell of aspect 93, wherein the host cell is a eukaryotic cell.

Aspect 96. A method of detecting an antigen-specific T cell, the method comprising contacting a T cell with the antigen-presenting polypeptide of any one of aspects 1-23, wherein binding of the antigen-presenting polypeptide to the T cell indicates that the T cell is specific for the epitope present in the antigen-presenting polypeptide.

Aspect 97. The method of aspect 96, wherein the antigen-presenting polypeptide comprises a detectable label.

Aspect 98. The method of aspect 97, wherein the detectable label is a radioisotope, a fluorescent polypeptide, or an enzyme that generates a fluorescent product, an enzyme that generates a colored product.

Aspect 99. The method of aspect 96, wherein binding of the antigen-presenting polypeptide to the T cell is detected using a detectably labeled antibody specific for the antigen-presenting polypeptide.

Aspect 100. The method of any one of aspects 96-99, wherein the T cell is present in a sample comprising a plurality of T cells.

Aspect 101. A method of selectively modulating the activity of an epitope-specific T cell, the method comprising contacting the T cell with the T-cell modulatory antigen-presenting polypeptide of any one of aspects 24-77 and 82, wherein said contacting selectively modulates the activity of the epitope-specific T cell.

Aspect 102. The method of aspect 101, wherein said contacting is in vitro.

Aspect 103. The method of aspect 101, wherein said contacting is in vivo.

Aspect 104. The method of any one of aspects 101-103, wherein the T-cell is a regulatory T cell (Treg).

Aspect 105. The method of aspect 104, wherein said contacting activates the Treg and reduces activity of an autoreactive T cell.

Aspect 106. The method of any one of aspects 101-105, wherein the T-cell is a CD4⁺ T helper cell, and wherein said contacting activates the CD4⁺ T cell.

Aspect 107. The method of aspect 106, wherein said activated CD4⁺ T cell activates a CD8⁺ T cell.

Aspect 108. The method of aspect 107, wherein the CD8⁺ T cell is specific for a cancer epitope presented by the T-cell modulatory antigen-presenting polypeptide.

Aspect 109. The method of any one of aspects 101 and 103-108, comprising administering the T-cell modulatory antigen-presenting polypeptide to an individual in need thereof.

Aspect 110. The method of aspect 109, wherein said administering is systemic.

Aspect 111. The method of aspect 109, wherein said administering is local.

Aspect 112. The method of aspect 109, wherein said administering is peritumoral.

Aspect 113. The method of aspect 109, wherein said administering is via intravenous administration.

Aspect 114. The method of any one of aspects 101-113, wherein the individual is a human

Aspect 115. The method of aspect 114, wherein the individual has an autoimmune disease.

Aspect 116. The method of aspect 114, wherein the individual has a cancer.

Aspect 117. A treatment method, the method comprising administering to an individual in need thereof an effective amount of the T-cell modulatory antigen-presenting polypeptide of any one of aspects 24-77 and 82, wherein said administering treats the individual.

Aspect 118. The method of aspect 117, wherein the individual has cancer, and wherein said administering treats the cancer.

Aspect 119. The method of aspect 117, wherein the individual has an autoimmune disorder, and wherein said administering treats the autoimmune disorder.

Aspect 120. The method of any one of aspects 117-119, wherein said administering is via intravenous administration.

Aspect 121. The method of any one of aspects 117-119, wherein said administering is via local administration.

Aspect 122. The method of any one of aspects 117-119, wherein said administering is via systemic administration.

### Aspects Set B

Without limiting the foregoing description, certain non-limiting aspects of the disclosure numbered 1-23 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

Aspect 1. A multimeric T-cell modulatory antigen-presenting polypeptide comprising:

a) a first polypeptide comprising: i) a peptide that displays an autoimmune disease-associated epitope useful for treating an autoimmune disease other than, or in addition to, Type 1 Diabetes or celiac disease, wherein the epitope is capable of being bound by a T-cell receptor (TCR); ii) a first major histocompatibility complex (MHC) class II polypeptide; and

b) a second polypeptide comprising: i) a second MHC Class II polypeptide; and

wherein one or both polypeptides of the multimeric polypeptide comprises one or more immunomodulatory polypeptides,

wherein the first and the second MHC class II polypeptides comprise:

i) an MHC class II α chain polypeptide having at least 90% amino acid sequence identity to a DRA1*01:01 polypeptide; and an MHC class II β chain polypeptide having at least 90% amino acid sequence identity to a DRB1 polypeptide; or

ii) an MHC class II α chain polypeptide having at least 90% amino acid sequence identity to a DRA1 polypeptide; and an MHC class II DRB1 chain polypeptide having at least 90% amino acid sequence identity to a DRB1 polypeptide of one of the following alleles: DRB1*01:01, DRB1*01:02, DRB1*01:03, DRB1*0301, DRB1*03:02, DRB1*0304, DRB1*04:01, DRB1*04:02, DRB1*0403, DRB1*04:04, DRB1*04:05, DRB1*04:06, DRB1*04:08, DRB1*08:03, DRB1*09:01, DRB1*10:01, DRB1*11:04, DRB1*13:01, DRB1*14:02, DRB1*1501, DRB1*1502, and DRB1*1503; or

iii) an MHC class II α chain polypeptide having at least 90% amino acid sequence identity to a DQA1 polypeptide of one of the following alleles: DQA1*01:01, DQA*01:02, DQA1*01:04, DQA1*03:01, DQA1*03:02, DQA1*04:01, DQA1*05:01; and an MHC class II β chain polypeptide having at least 90% amino acid sequence identity to a DQB1 polypeptide; or

iv) an MHC class II α chain polypeptide having at least 90% amino acid sequence identity to a DQA1 polypeptide; and an MHC class II β chain polypeptide having at least 90% amino acid sequence identity to a DQB1 polypeptide of one of the following alleles: DQB1*02:01, DQB1*03:01, DQB1*03:03, DQB1*04:01, DQB1*04:02, DQB1*05:01, DQB1*05:03, DQB1*06:01, and DQB1*0602; or

v) an MHC class II α chain polypeptide having at least 90% amino acid sequence identity to a DRA3 polypeptide; and an MHC class II β chain polypeptide having at least 90% amino acid sequence identity to a DRB3 polypeptide of one of the following alleles: DRB3*01:01 and DRB3*03:01; or

vi) an MHC class II α chain polypeptide having at least 90% amino acid sequence identity to a DRA4 polypeptide; and an MHC class II β chain polypeptide having at least 90% amino acid sequence identity to a DRB4*01:01 polypeptide; or

vii) an MHC class II α chain polypeptide having at least 90% amino acid sequence identity to a DRA1 polypeptide; and an MHC class II β chain polypeptide having at least 90% amino acid sequence identity to a DRB5*01:01 polypeptide; or

viii) an MHC class II α chain polypeptide having at least 90% amino acid sequence identity to a DPA1 polypeptide; and an MHC class II β chain polypeptide having at least 90% amino acid sequence identity to a DPB1 polypeptide of one of the following alleles: DPB1*03:01, DPB1*09:01, and DPB1*13:01,

wherein one or both polypeptides of the multimeric polypeptide optionally comprises an immunoglobulin (Ig) Fc polypeptide or a non-Ig scaffold.

Aspect 2. The multimeric T-cell modulatory antigen-presenting polypeptide of aspect 1, wherein:

a1) the first polypeptide comprises, in order from N-terminus to C-terminus:
i) the peptide epitope;
ii) an MHC Class II β1 polypeptide; and
iii) an MHC Class II β2 polypeptide; and

b1) the second polypeptide comprises, in order from N-terminus to C-terminus:
i) the one or more immunomodulatory polypeptides;
ii) an MHC Class II α1 polypeptide;
iii) an MHC Class II α2 polypeptide; and
iv) an Ig Fc polypeptide; or

a2) the first polypeptide comprises, in order from N-terminus to C-terminus:
i) the peptide epitope;
ii) an MHC Class II β1 polypeptide; and
iii) an MHC Class II β2 polypeptide; and

b2) the second polypeptide comprises, in order from N-terminus to C-terminus:
i) an MHC Class II α1 polypeptide;
ii) an MHC Class II α2 polypeptide;
iii) an Ig Fc polypeptide; and
iv) the one or more immunomodulatory polypeptides; or

a3) the first polypeptide comprises, in order from N-terminus to C-terminus:
i) the peptide epitope;
ii) an MHC Class II β1 polypeptide; and
iii) an MHC Class II β2 polypeptide; and

b3) the second polypeptide comprises, in order from N-terminus to C-terminus:
i) an MHC Class II α1 polypeptide;
ii) an MHC Class II α2 polypeptide;
iii) the one or more immunomodulatory polypeptides; and
iv) an Ig Fc polypeptide; or

a4) the first polypeptide comprises, in order from N-terminus to C-terminus:
i) an MHC Class II α1 polypeptide;
ii) an MHC Class II α2 polypeptide; and
iii) an Ig Fc polypeptide; and

b4) the second polypeptide comprises, in order from N-terminus to C-terminus:
i) the one or more immunomodulatory polypeptides;
ii) the peptide epitope;
iii) an MHC Class II β1 polypeptide; and
iv) an MHC Class II β2 polypeptide; or

a5) the first polypeptide comprises, in order from N-terminus to C-terminus:
i) an MHC Class II α1 polypeptide;
ii) an MHC Class II α2 polypeptide; and
iii) an Ig Fc polypeptide; and

b5) the second polypeptide comprises, in order from N-terminus to C-terminus:
i) the peptide epitope;
ii) an MHC Class II β1 polypeptide;
iii) an MHC Class II β2 polypeptide; and
iv) the one or more immunomodulatory polypeptides.

Aspect 3. The multimeric T-cell modulatory antigen-presenting polypeptide of aspect 1 or aspect 2, wherein:

a) the MHC class II α polypeptide comprises an amino acid sequence having at least 95% amino acid sequence identity to a DQA1*01:01 polypeptide; and the MHC class II β polypeptide comprises an amino acid sequence having at least 95% amino acid sequence identity to a DQB1*05:01 polypeptide; or

b) the MHC class II α polypeptide comprises an amino acid sequence having at least 95% amino acid sequence identity to a DRA1*01:01 polypeptide; and the MHC class II β polypeptide comprises an amino acid sequence having at least 95% amino acid sequence identity to a DRB1*04:01 polypeptide; or

c) the MHC class II α polypeptide comprises an amino acid sequence having at least 95% amino acid sequence identity to a DRA1*01:01 polypeptide; and the MHC class II β polypeptide comprises an amino acid sequence having at least 95% amino acid sequence identity to a DRB5*01:01 polypeptide; or

d) the MHC class II α polypeptide comprises an amino acid sequence having at least 95% amino acid sequence identity to a DQA1*01:02 polypeptide; and the MHC class II β polypeptide comprises an amino acid sequence having at least 95% amino acid sequence identity to a DQB1*06:02 polypeptide; or

e) the MHC class II α polypeptide comprises an amino acid sequence having at least 95% amino acid sequence identity to a DQA1*03:01 polypeptide; and the MHC class II β polypeptide comprises an amino acid sequence having at least 95% amino acid sequence identity to a DQB1*03:03 polypeptide; or

f) the MHC class II α polypeptide comprises an amino acid sequence having at least 95% amino acid sequence identity to a DQA1*01:04 polypeptide; and the MHC class II β polypeptide comprises an amino acid sequence having at least 95% amino acid sequence identity to a DQB1*05:01 polypeptide; or

g) the MHC class II α polypeptide comprises an amino acid sequence having at least 95% amino acid sequence identity to a DRA1*01:01 polypeptide; and the MHC class II β polypeptide comprises an amino acid sequence having at least 95% amino acid sequence identity to a DRB1*01:01 polypeptide; or

h) the MHC class II α polypeptide comprises an amino acid sequence having at least 95% amino acid sequence identity to a DQA1*04:01 polypeptide; and the MHC class II β polypeptide comprises an amino acid sequence having at least 95% amino acid sequence identity to a DQB1*04:02 polypeptide; or

i) the MHC class II α polypeptide comprises an amino acid sequence having at least 95% amino acid sequence identity to a DQA1*03:02 polypeptide; and the MHC class II β polypeptide comprises an amino acid sequence having at least 95% amino acid sequence identity to a DQB1*03:01 polypeptide.

Aspect 4. The T-cell modulatory antigen-presenting polypeptide of any one of aspects 1-3, wherein the immunomodulatory polypeptide:

a) comprises the amino acid sequence of a naturally-occurring immunomodulatory polypeptide; or

b) is a variant immunomodulatory polypeptide that comprises an amino acid sequence having from 1 to 10 amino acid substitutions compared to the amino acid sequence of a naturally-occurring immunomodulatory polypeptide, wherein the variant immunomodulatory polypeptide has reduced affinity for a co-immunomodulatory polypeptide, compared to the affinity of the naturally-occurring immunomodulatory polypeptide for the co-immunomodulatory polypeptide.

Aspect 5. The T-cell modulatory antigen-presenting polypeptide of any one of aspects 1-4, wherein the immunomodulatory polypeptide is a PD-L1 polypeptide, a FasL polypeptide, or a TGF-β polypeptide.

Aspect 6. The T-cell modulatory antigen-presenting polypeptide of any one of aspects 1-4, wherein the immunomodulatory polypeptide is a PD-L1 polypeptide.

Aspect 7. The T-cell modulatory antigen-presenting polypeptide of aspect 1, wherein the autoimmune disease-associated peptide has a length of from about 4 amino acids to about 25 amino acids.

Aspect 8. The T-cell modulatory antigen-presenting polypeptide any one of aspects 1-7, wherein the peptide is a multiple sclerosis-associated peptide.

Aspect 9. The T-cell modulatory antigen-presenting polypeptide of any one of aspects 1-7, wherein the peptide is a rheumatoid arthritis-associated peptide.

Aspect 10. The T-cell modulatory antigen-presenting polypeptide of aspect 1, wherein the peptide is a systemic lupus erythematosus-associated peptide.

Aspect 11. The T-cell modulatory antigen-presenting polypeptide of aspect 1, wherein the peptide is an Addison's disease-associated peptide.

Aspect 12. The T-cell modulatory antigen-presenting polypeptide of aspect 1, wherein the peptide is a myasthenia gravis-associated peptide.

Aspect 13. The T-cell modulatory antigen-presenting polypeptide of aspect 1, wherein the peptide is a Sjögren's syndrome-associated peptide.

Aspect 14. The T-cell modulatory antigen-presenting polypeptide of aspect 1, wherein the peptide is a psoriasis-associated peptide.

Aspect 15. A composition comprising: a) the T-cell modulatory antigen-presenting polypeptide of any one of aspects 1-14; and b) a pharmaceutically acceptable excipient.

Aspect 16. One or more nucleic acids comprising nucleotide sequences encoding the T-cell modulatory antigen-presenting polypeptide of any one of aspects 1-14.

Aspect 17. One or more expression vectors comprising the one or more nucleic acids of aspect 16.

Aspect 18. A host cell genetically modified with the one or more nucleic acids of aspect 16 or the one or more expression vectors of aspect 17.

Aspect 19. A method of reducing the number and/or activity of CD4⁺ T cells specific for an autoimmune disease in an individual, the method comprising contacting the CD4⁺ T cells with the T-cell modulatory antigen-presenting polypeptide of any one of aspects 1-14, wherein said contacting reduces the number and/or activity of the CD4⁺ T cells, and wherein the autoimmune disease is other than T1D or celiac disease.

Aspect 20. A method of treating an autoimmune disease in an individual, the method comprising administering to an individual in need thereof an effective amount of the T-cell modulatory antigen-presenting polypeptide of any one of aspects 1-14, wherein said administering treats the autoimmune disease in the individual, and wherein the autoimmune disease is other than T1D or celiac disease.

Aspect 21. The method of aspect 20, wherein the autoimmune disease is Addison's disease, alopecia areata, ankylosing spondylitis, autoimmune encephalomyelitis, autoimmune hemolytic anemia, autoimmune hepatitis, autoimmune-associated infertility, autoimmune thrombocytopenic purpura, bullous pemphigoid, Crohn's disease, Goodpasture's syndrome, glomerulonephritis (e.g., crescentic glomerulonephritis, proliferative glomerulonephritis), Grave's disease, Hashimoto's thyroiditis, mixed connective tissue disease, multiple sclerosis, myasthenia gravis (MG), pemphigus (e.g., pemphigus vulgaris), pernicious anemia, polymyositis, psoriasis, psoriatic arthritis, rheumatoid arthritis, scleroderma, Sjögren's syndrome, systemic lupus erythematosus (SLE), vasculitis, or vitiligo.

Aspect 22. A method of reducing the number and/or activity of pathogenic CD4⁺ and/or CD8⁺ self-reactive T cells specific for an autoimmune disease in an individual, the method comprising contacting the CD4⁺ T cells with the T-cell modulatory antigen-presenting polypeptide of any one of aspects 1-14, wherein said contacting reduces the number and/or activity of the pathogenic CD4⁺ and/or CD8+ T cells, and wherein the autoimmune disease is other than T1D or celiac disease.

Aspect 23. A method of reducing the number and/or activity of CD4⁺ T cells and/or CD8⁺ self-reactive T cells specific for autoimmune disease in an individual, the method comprising contacting the CD4⁺ T cells with the T-cell modulatory antigen-presenting polypeptide of any one of aspects 1-14, wherein said contacting increases the number of CD4⁺ Treg cells, which in turn reduces the number and/or activity of the pathogenic CD4⁺ T cells and/or CD8+ T cells, and wherein the autoimmune disease is other than T1D or celiac disease.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1: Production of Antigen-Presenting Polypeptides

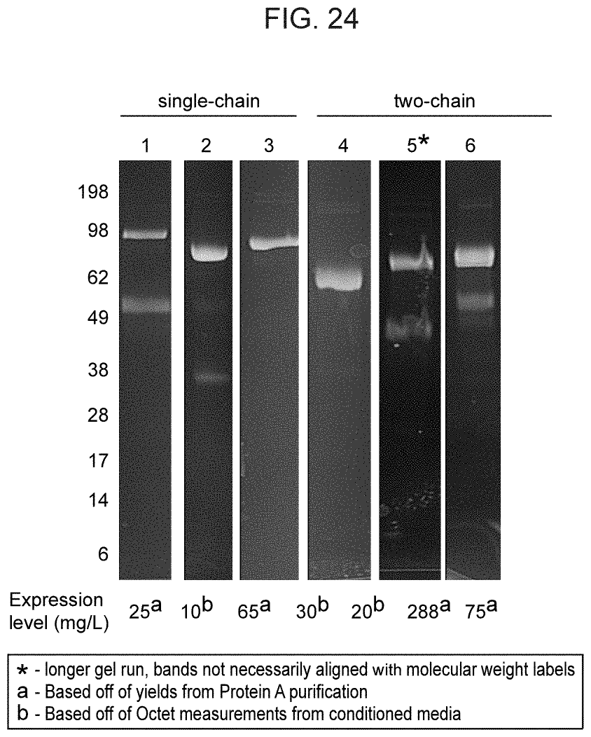

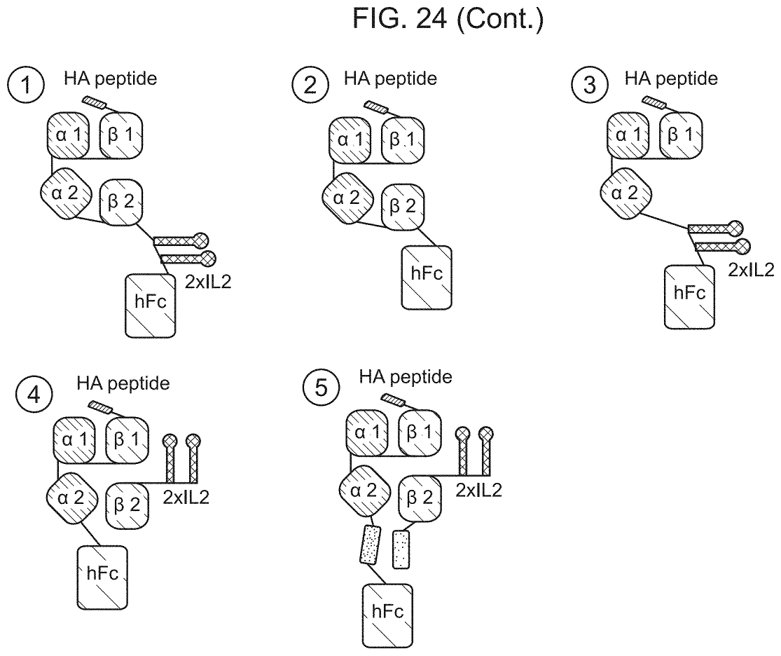

To optimize production of intact and stable MHC Class II antigen-presenting polypeptides, various structural arrangements of antigen-presenting polypeptides comprising MHC Class II polypeptides were synthesized, expressed, and purified using Protein A affinity chromatography. For the design of the MHC Class II synTacs, parameters varied included orientation of the MHC Class II alpha and beta chains, Fc placement, IL2 (MOD) placement, and length and content of the various linkers. The variants presented include single-chain as well as two-chain versions, each with the MHC Class II β-1 domain linked N-terminal to the α-1 domain, and with β-2 either C-terminal to α-2 or on a separate chain, as shown schematically in FIG. 24. Single-chain variants with and without the β-2 domain or the IL2 fusion are shown, as well as two-chain versions with and without the bZIP dimerization domain

Antigen-presenting polypeptides, with or without immunomodulatory polypeptides, were generated Amino acid sequences of the antigen-presenting polypeptides, and nucleotide sequences encoding the polypeptides, are provided in FIG. 25-35. The polypeptides included single-chain polypeptides and multimeric polypeptides. The antigen-presenting polypeptides are as follows:

1) 1599—This is a single-chain polypeptide comprising a variant IL-2 immunomodulatory polypeptide. The 1599 polypeptide also includes an HLA β2 polypeptide. The 1599 polypeptide includes: i) an epitope (a hemagglutinin epitope); ii) HLA DRB1 β1; iii) HLA DRA α1 and α2; iv) HLA DRB1β2; v) a variant IL-2 immunomodulatory polypeptide; and v) an IgG1 Fc.

2) 1559—This is a single-chain polypeptide comprising an HLA β2 polypeptide. The 1559 polypeptide lacks an immunomodulatory polypeptide. The 1559 polypeptide includes: i) an epitope (a hemagglutinin (HA) epitope); ii) HLA DRB1 β1; iii) HLA DRA α1 and α2; iv) HLA DRB1 β2; and v) an IgG1 Fc.

3) 1601—This is a single-chain polypeptide a variant IL-2 immunomodulatory polypeptide. The 1601 polypeptide lacks an HLA β2 polypeptide. The 1601 polypeptide includes: i) an epitope (a hemagglutinin epitope); ii) HLA DRB1 β1; iii) HLA DRA α1 and α2; iv) 2 copies of a variant IL-2 immunomodulatory polypeptide; and v) an IgG1 Fc polypeptide.

4) 1452+1661—This is a multimeric antigen-presenting polypeptide. The epitope is a hemagglutinin epitope. It includes HLA DRB1 and DRA MHC Class II polypeptides. Both polypeptide chains include leucine zipper dimerizer peptides. The 1452 polypeptide includes an IgG1 Fc polypeptide.

5) 1659+1664—This is a multimeric antigen-presenting polypeptide. It includes HLA DRB1 and DRA MHC Class II polypeptides. The epitope is a hemagglutinin epitope. The 1664 polypeptide includes 2 copies of a variant IL-2 immunomodulatory polypeptide. Both polypeptide chains lack leucine zipper dimerizer peptides. The 1659 polypeptide includes an IgG1 Fc polypeptide.

6) 1637+1408—This is a multimeric antigen-presenting polypeptide. It includes HLA DRB1 and DRA MHC Class II polypeptides. The epitope is a CMV epitope. The 1408 polypeptide includes 2 copies of a variant IL-2 immunomodulatory polypeptide. Both chains include a leucine zipper (bZIP). The 1637 polypeptide includes an IgG1 Fc polypeptide.

7) “DRA-DRB-epitope”+1640—This is a multimeric antigen-presenting polypeptide. It includes HLA DRB1-4 and DRA MHC Class II polypeptides. The epitope is a hemagglutinin epitope. Both chains include a leucine zipper (bZIP). The DRA-DRB-epitope polypeptide includes an IgG1 Fc polypeptide.

Expression constructs comprising nucleotide sequences encoding the above-described polypeptides were introduced into a mammalian cell line. The produced polypeptides were loaded onto a reducing polyacrylamide gel. As shown in FIG. 24, left panel, all polypeptides were produced in detectable amounts. The various constructs are depicted schematically in FIG. 24, right panel.

The single-chain version without the β-2 domain (and with IL2) demonstrated a robust expression level and an intact, homogeneous product upon Protein A purification (lane 3). The addition of the β-2 domain resulted in lower expression as well as de-stabilization of the molecule as indicated by a prominent breakdown product observed on the analytical gel (lane 1). Removal of the IL2 (while retaining the β-2) resulted in even lower expression, but with minimal breakdown product (lane 2).

With the β-2 domain on a separate chain, robust assembly was observed with incorporation of the bZIP leucine zipper dimerization domain (lane 5). Without the bZIP domain, modest expression and production of intact Fc-containing chain was observed; β-2 chain was incorporated (lane 4). Switching from the HA peptide to the CMV peptide in the two-chain bZIP model resulted in very robust expression of intact product (lane 6).

Therefore, intact, stable MHC Class II antigen-presenting polypeptides were synthesized, expressed and purified. These represented design models that incorporated either a single-chain or a two-chain system. Incorporation of both the β-2 domain and a dimerization domain resulted in robust expression using the two-chain system. The peptide chosen for binding to the MHC can provide stabilization. Further, intact, stable MHC Class II antigen-presenting polypeptides were generated with MHC Class II polypeptides of two different MHC alleles.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1.-21. (canceled)

22. A multimeric T-cell modulatory antigen-presenting polypeptide comprising:
a) a first polypeptide comprising:
i) a peptide that displays an autoimmune disease-associated epitope (peptide epitope) useful for treating an autoimmune disease other than Type 1 Diabetes (T1D) or celiac disease, wherein the epitope is capable of being bound by a T-cell receptor (TCR);
ii) a first major histocompatibility complex (MHC) class II polypeptide; and
b) a second polypeptide comprising:
i) a second MHC Class II polypeptide; and
wherein one or both polypeptides of the multimeric polypeptide comprises one or more immunomodulatory polypeptides,
wherein the first and the second MHC class II polypeptides comprise:
i) an MHC class II α chain polypeptide having at least 90% amino acid sequence identity to a DRA1*01:01 polypeptide; and an MHC class II β chain polypeptide having at least 90% amino acid sequence identity to a DRB1 polypeptide; or
ii) an MHC class II α chain polypeptide having at least 90% amino acid sequence identity to a DRA1 polypeptide; and an MHC class II DRB1 chain polypeptide having at least 90% amino acid sequence identity to a DRB1 polypeptide of one of the following alleles: DRB1*01:01, DRB1*01:02, DRB1*01:03, DRB1*0301, DRB1*03:02, DRB1*0304, DRB1*04:01, DRB1*04:02, DRB1*0403, DRB1*04:04, DRB1*04:05, DRB1*04:06, DRB1*04:08, DRB1*08:03, DRB1*09:01, DRB1*10:01, DRB1*11:04, DRB1*13:01, DRB1*14:02, DRB1*1501, DRB1*1502, and DRB1*1503; or
iii) an MHC class II α chain polypeptide having at least 90% amino acid sequence identity to a DQA1 polypeptide of one of the following alleles: DQA1*01:01, DQA*01:02, DQA1*01:04, DQA1*03:01, DQA1*03:02, DQA1*04:01, DQA1*05:01; and an MHC class II β chain polypeptide having at least 90% amino acid sequence identity to a DQB1 polypeptide; or
iv) an MHC class II α chain polypeptide having at least 90% amino acid sequence identity to a DQA1 polypeptide; and an MHC class II R chain polypeptide having at least 90% amino acid sequence identity to a DQB1 polypeptide of one of the following alleles: DQB1*02:01, DQB1*03:01, DQB1*03:03, DQB1*04:01, DQB1*04:02, DQB1*05:01, DQB1*05:03, DQB1*06:01, and DQB1*0602; or
v) an MHC class II α chain polypeptide having at least 90% amino acid sequence identity to a DRA3 polypeptide; and an MHC class II β chain polypeptide having at least 90% amino acid sequence identity to a DRB3 polypeptide of one of the following alleles: DRB3*01:01 and DRB3*03:01; or
vi) an MHC class II α chain polypeptide having at least 90% amino acid sequence identity to a DRA4 polypeptide; and an MHC class II β chain polypeptide having at least 90% amino acid sequence identity to a DRB4*01:01 polypeptide; or
vii) an MHC class II α chain polypeptide having at least 90% amino acid sequence identity to a DRA1 polypeptide; and an MHC class II β chain polypeptide having at least 90% amino acid sequence identity to a DRB5*01:01 polypeptide; or
viii) an MHC class II α chain polypeptide having at least 90% amino acid sequence identity to a DPA1 polypeptide; and an MHC class II β chain polypeptide having at least 90% amino acid sequence identity to a DPB1 polypeptide of one of the following alleles: DPB1*03:01, DPB1*09:01, and DPB1*13:01,
wherein one or both polypeptides of the multimeric polypeptide optionally comprises an immunoglobulin (Ig) Fc polypeptide or a non-Ig scaffold.

23. The multimeric T-cell modulatory antigen-presenting polypeptide of claim 22, wherein:
a1) the first polypeptide comprises, in order from N-terminus to C-terminus:
i) the peptide epitope;
ii) an MHC Class II β1 polypeptide; and
iii) an MHC Class II β2 polypeptide; and
b1) the second polypeptide comprises, in order from N-terminus to C-terminus:
i) the one or more immunomodulatory polypeptides;
ii) an MHC Class II α1 polypeptide;
iii) an MHC Class II α2 polypeptide; and
iv) an Ig Fc polypeptide; or
a2) the first polypeptide comprises, in order from N-terminus to C-terminus:
i) the peptide epitope;
ii) an MHC Class II β1 polypeptide; and
iii) an MHC Class II β2 polypeptide; and
b2) the second polypeptide comprises, in order from N-terminus to C-terminus:
i) an MHC Class II α1 polypeptide;
ii) an MHC Class II α2 polypeptide;
iii) an Ig Fc polypeptide; and
iv) the one or more immunomodulatory polypeptides; or
a3) the first polypeptide comprises, in order from N-terminus to C-terminus:
i) the peptide epitope;
ii) an MHC Class II β1 polypeptide; and
iii) an MHC Class II β2 polypeptide; and
b3) the second polypeptide comprises, in order from N-terminus to C-terminus:
i) an MHC Class II α1 polypeptide;
ii) an MHC Class II α2 polypeptide;
iii) the one or more immunomodulatory polypeptides; and
iv) an Ig Fc polypeptide; or
a4) the first polypeptide comprises, in order from N-terminus to C-terminus:
i) an MHC Class II α1 polypeptide;
ii) an MHC Class II α2 polypeptide; and
iii) an Ig Fc polypeptide; and
b4) the second polypeptide comprises, in order from N-terminus to C-terminus:
i) the one or more immunomodulatory polypeptides;
ii) the peptide epitope;
iii) an MHC Class II β1 polypeptide; and
iv) an MHC Class II β2 polypeptide; or
a5) the first polypeptide comprises, in order from N-terminus to C-terminus:
i) an MHC Class II α1 polypeptide;
ii) an MHC Class II α2 polypeptide; and
iii) an Ig Fc polypeptide; and
b5) the second polypeptide comprises, in order from N-terminus to C-terminus:
i) the peptide epitope;
ii) an MHC Class II β1 polypeptide;
iii) an MHC Class II β2 polypeptide; and
iv) the one or more immunomodulatory polypeptides.

24. The multimeric T-cell modulatory antigen-presenting polypeptide of claim 22, wherein:
a) the MHC class II α polypeptide comprises an amino acid sequence having at least 95% amino acid sequence identity to a DQA1*01:01 polypeptide; and the MHC class II β polypeptide comprises an amino acid sequence having at least 95% amino acid sequence identity to a DQB1*05:01 polypeptide; or
b) the MHC class II α polypeptide comprises an amino acid sequence having at least 95% amino acid sequence identity to a DRA1*01:01 polypeptide; and the MHC class II β polypeptide comprises an amino acid sequence having at least 95% amino acid sequence identity to a DRB1*04:01 polypeptide; or
c) the MHC class II α polypeptide comprises an amino acid sequence having at least 95% amino acid sequence identity to a DRA1*01:01 polypeptide; and the MHC class II β polypeptide comprises an amino acid sequence having at least 95% amino acid sequence identity to a DRB5*01:01 polypeptide; or
d) the MHC class II α polypeptide comprises an amino acid sequence having at least 95% amino acid sequence identity to a DQA1*01:02 polypeptide; and the MHC class II β polypeptide comprises an amino acid sequence having at least 95% amino acid sequence identity to a DQB1*06:02 polypeptide; or
e) the MHC class II α polypeptide comprises an amino acid sequence having at least 95% amino acid sequence identity to a DQA1*03:01 polypeptide; and the MHC class II β polypeptide comprises an amino acid sequence having at least 95% amino acid sequence identity to a DQB1*03:03 polypeptide; or
f) the MHC class II α polypeptide comprises an amino acid sequence having at least 95% amino acid sequence identity to a DQA1*01:04 polypeptide; and the MHC class II β polypeptide comprises an amino acid sequence having at least 95% amino acid sequence identity to a DQB1*05:01 polypeptide; or
g) the MHC class II α polypeptide comprises an amino acid sequence having at least 95% amino acid sequence identity to a DRA1*01:01 polypeptide; and the MHC class II β polypeptide comprises an amino acid sequence having at least 95% amino acid sequence identity to a DRB1*01:01 polypeptide; or
h) the MHC class II α polypeptide comprises an amino acid sequence having at least 95% amino acid sequence identity to a DQA1*04:01 polypeptide; and the MHC class II β polypeptide comprises an amino acid sequence having at least 95% amino acid sequence identity to a DQB1*04:02 polypeptide; or
i) the MHC class II α polypeptide comprises an amino acid sequence having at least 95% amino acid sequence identity to a DQA1*03:02 polypeptide; and the MHC class II β polypeptide comprises an amino acid sequence having at least 95% amino acid sequence identity to a DQB1*03:01 polypeptide.

25. The T-cell modulatory antigen-presenting polypeptide of claim 22, wherein the immunomodulatory polypeptide:
a) comprises the amino acid sequence of a naturally-occurring immunomodulatory polypeptide; or
b) is a variant immunomodulatory polypeptide that comprises an amino acid sequence having from 1 to 10 amino acid substitutions compared to the amino acid sequence of a naturally-occurring immunomodulatory polypeptide, wherein the variant immunomodulatory polypeptide has reduced affinity for a co-immunomodulatory polypeptide, compared to the affinity of the naturally-occurring immunomodulatory polypeptide for the co-immunomodulatory polypeptide.

26. The T-cell modulatory antigen-presenting polypeptide of claim 22, wherein the immunomodulatory polypeptide is a PD-L1 polypeptide, a FasL polypeptide, or a TGF-β polypeptide.

27. The T-cell modulatory antigen-presenting polypeptide of claim 22, wherein the immunomodulatory polypeptide is a PD-L1 polypeptide.

28. The T-cell modulatory antigen-presenting polypeptide of claim 22, wherein the peptide epitope has a length of from about 4 amino acids to about 25 amino acids.

29. The T-cell modulatory antigen-presenting polypeptide of claim 22, wherein the peptide epitope is a multiple sclerosis-associated peptide epitope, a rheumatoid arthritis-associated peptide epitope, or a systemic lupus erythematosus-associated peptide epitope.

30. The T-cell modulatory antigen-presenting polypeptide of claim 22, wherein the peptide epitope is an Addison's disease-associated peptide epitope, a myasthenia gravis-associated peptide epitope, a Sjögren's syndrome-associated peptide epitope, or a psoriasis-associated peptide epitope.

31. The T-cell modulatory antigen-presenting polypeptide of claim 22, wherein
a) the first polypeptide comprises from N-terminus to C-terminus:
i) a peptide epitope;
ii) a linker; and
iii) an MHC class II beta-chain polypeptide, and
b) the second polypeptide comprises from N-terminus to C-terminus:
i) a PD-L1 immunomodulatory polypeptide;
ii) a linker;
iii) an MHC class II alpha-chain polypeptide;
iv) a linker; and
v) an Ig Fc polypeptide comprising an amino acid sequence having at least 90% amino acid sequence identity to the IgG1 Fc amino acid sequence depicted in FIG. 21A.

32. The T-cell modulatory antigen-presenting polypeptide of claim 31, wherein the Ig Fc polypeptide comprises an Ala at position 14 and an Ala at position 15, based on the amino acid number of FIG. 21A.

33. A pharmaceutical composition comprising the T-cell modulatory antigen-presenting polypeptide of claim 22.

34. A pharmaceutical composition comprising the T-cell modulatory antigen-presenting polypeptide of claim 31.

35. A pharmaceutical composition comprising the T-cell modulatory antigen-presenting polypeptide of claim 32.

36. One or more nucleic acids comprising nucleotide sequences encoding the T-cell modulatory antigen-presenting polypeptide of claim 22.

37. An expression vector or host cell comprising the one or more nucleic acids of claim 36.

38. A method of reducing the number and/or activity of CD⁴⁺ and/or CD8⁺ self-reactive T cells specific for an autoimmune disease other than Type 1 Diabetes (T1D) or celiac disease in an individual, the method comprising contacting the CD⁴⁺ T cells with the T-cell modulatory antigen-presenting polypeptide of claim 22, wherein said contacting reduces the number and/or activity of the CD⁴⁺ and/or CD8+ T cells.

39. A method of treating an autoimmune disease other than Type 1 Diabetes (T1D) or celiac disease in an individual, the method comprising administering to an individual in need thereof an effective amount of the T-cell modulatory antigen-presenting polypeptide of claim 22, wherein said administering treats the type 1 diabetes or celiac disease in the individual.

40. A method of treating an autoimmune disease other than Type 1 Diabetes (T1D) or celiac disease in an individual, the method comprising administering to an individual in need thereof an effective amount of the T-cell modulatory antigen-presenting polypeptide of claim 31.

41. A method of treating an autoimmune disease other than Type 1 Diabetes (T1D) or celiac disease in an individual, the method comprising administering to an individual in need thereof an effective amount of the T-cell modulatory antigen-presenting polypeptide of claim 32.
